SEQUENCE LISTING (102 sequences)

SEQ ID NO: 1 - DNA, 3 bp, Artificial Sequence
  Synthetic
  misc_feature (1)..(1): n is a, c, g, or t
  misc_feature (2)..(2): r is a, or g
nrg

SEQ ID NO: 2 - DNA, 3 bp, Artificial Sequence
  Synthetic
  misc_feature (1)..(1): n is a, c, g, or t
ngg

SEQ ID NO: 3 - DNA, 3 bp, Artificial Sequence
  Synthetic
  misc_feature (1)..(1): n is a, c, g, or t
nag

SEQ ID NO: 4 - DNA, 5 bp, Artificial Sequence
  Synthetic
  misc_feature (1)..(1): n is a, c, g, or t
  misc_feature (4)..(4): n is a, c, g, or t
nggng

SEQ ID NO: 5 - DNA, 7 bp, Artificial Sequence
  Synthetic
  misc_feature (1)..(2): n is a, c, g, or t
  misc_feature (7)..(7): w is a, or t
nnagaaw

SEQ ID NO: 6 - DNA, 4 bp, Artificial Sequence
  Synthetic
  misc_feature (1)..(1): n is a, c, g, or t
  misc_feature (4)..(4): r is a, or g
naar

SEQ ID NO: 7 - DNA, 5 bp, Artificial Sequence
  Synthetic
  misc_feature (1)..(2): n is a, c, g, or t
  misc_feature (4)..(5): r is a, or g
nngrr

SEQ ID NO: 8 - DNA, 6 bp, Artificial Sequence
  Synthetic
  misc_feature (1)..(2): n is a, c, g, or t
  misc_feature (4)..(5): r is a, or g
  misc_feature (6)..(6): n is a, c, g, or t
nngrrn

SEQ ID NO: 9 - DNA, 6 bp, Artificial Sequence
  Synthetic
  misc_feature (1)..(2): n is a, c, g, or t
  misc_feature (4)..(5): r is a, or g
nngrrt

SEQ ID NO: 10 - DNA, 6 bp, Artificial Sequence
  Synthetic
  misc_feature (1)..(2): n is a, c, g, or t
  misc_feature (4)..(5): r is a, or g
  misc_feature (6)..(6): v is a, c, or g
nngrrv

SEQ ID NO: 11 - DNA, 8 bp, Artificial Sequence
  Synthetic
  misc_feature (1)..(4): n is a, c, g, or t
nnnngatt

SEQ ID NO: 12 - DNA, 8 bp, Artificial Sequence
  Synthetic
  misc_feature (1)..(4): n is a, c, g, or t
  misc_feature (6)..(8): n is a, c, g, or t
nnnngnnn

SEQ ID NO: 13 - DNA, 3 bp, Artificial Sequence
  Synthetic
  misc_feature (1)..(1): n is a, c, g, or t
nga

SEQ ID NO: 14 - DNA, 6 bp, Artificial Sequence
  Synthetic
  misc_feature (1)..(3): n is a, c, g, or t
  misc_feature (4)..(5): r is a, or g
nnnrrt

SEQ ID NO: 15 - DNA, 6 bp, Artificial Sequence
  Synthetic
attcct

SEQ ID NO: 16 - DNA, 4 bp, Artificial Sequence
  Synthetic
  misc_feature (1)..(1): n is a, c, g, or t
  misc_feature (4)..(4): n is a, c, g, or t
ngan

SEQ ID NO: 17 - DNA, 4 bp, Artificial Sequence
  Synthetic
  misc_feature (1)..(1): n is a, c, g, or t
  misc_feature (3)..(3): n is a, c, g, or t
ngng

SEQ ID NO: 18 - DNA, 86 bp, Artificial Sequence
  Synthetic
gtttaagagctatgctggaaacagcatagcaagtttaaataaggctagtccgttatcaacttgaaaaagtggcaccgagtcggtgc

SEQ ID NO: 19 - RNA, 86 nt, Artificial Sequence
  Synthetic
guuuaagagcuaugcuggaaacagcauagcaaguuuaaauaaggcuaguccguuaucaacuugaaaaaguggcaccgagucggugc

SEQ ID NO: 20 - protein, 1368 aa, Streptococcus pyogenes [sequence omitted]

SEQ ID NO: 21 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 22 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 23 - protein, 1368 aa, Artificial Sequence
  Synthetic
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 24 - DNA, 3159 bp, Artificial Sequence
  Synthetic
atgaaaaggaactacattctggggctggccatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 25 - DNA, 3159 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagaggcctctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 26 - DNA, 4107 bp, Artificial Sequence
  Synthetic
atggataaaaagtacagcatcgggctggacatcggtacaaactcagtggggtgggccgtgattacggacgagtacaaggtaccctccaaaaaatttaaagtgctgggtaacacggacagacactctataaagaaaaatcttattggagccttgctgttcgactcaggcgagacagccgaagccacaaggttgaagcggaccgccaggaggcggtataccaggagaaagaaccgcatatgctacctgcaagaaatcttcagtaacgagatggcaaaggttgacgatagctttttccatcgcctggaagaatcctttcttgttgaggaagacaagaagcacgaacggcaccccatctttggcaatattgtcgacgaagtggcatatcacgaaaagtacccgactatctaccacctcaggaagaagctggtggactctaccgataaggcggacctcagacttatttatttggcactcgcccacatgattaaatttagaggacatttcttgatcgagggcgacctgaacccggacaacagtgacgtcgataagctgttcatccaacttgtgcagacctacaatcaactgttcgaagaaaaccctataaatgcttcaggagtcgacgctaaagcaatcctgtccgcgcgcctctcaaaatctagaagacttgagaatctgattgctcagttgcccggggaaaagaaaaatggattgtttggcaacctgatcgccctcagtctcggactgaccccaaatttcaaaagtaacttcgacctggccgaagacgctaagctccagctgtccaaggacacatacgatgacgacctcgacaatctgctggcccagattggggatcagtacgccgatctctttttggcagcaaagaacctgtccgacgccatcctgttgagcgatatcttgagagtgaacaccgaaattactaaagcaccccttagcgcatctatgatcaagcggtacgacgagcatcatcaggatctgaccctgctgaaggctcttgtgaggcaacagctccccgaaaaatacaaggaaatcttctttgaccagagcaaaaacggctacgctggctatatagatggtggggccagtcaggaggaattctataaattcatcaagcccattctcgagaaaatggacggcacagaggagttgctggtcaaacttaacagggaggacctgctgcggaagcagcggacctttgacaacgggtctatcccccaccagattcatctgggcgaactgcacgcaatcctgaggaggcaggaggatttttatccttttcttaaagataaccgcgagaaaatagaaaagattcttacattcaggatcccgtactacgtgggacctctcgcccggggcaattcacggtttgcctggatgacaaggaagtcagaggagactattacaccttggaacttcgaagaagtggtggacaagggtgcatctgcccagtctttcatcgagcggatgacaaattttgacaagaacctccctaatgagaaggtgctgcccaaacattctctgctctacgagtactttaccgtctacaatgaactgactaaagtcaagtacgtcaccgagggaatgaggaagccggcattccttagtggagaacagaagaaggcgattgtagacctgttgttcaagaccaacaggaaggtgactgtgaagcaacttaaagaagactactttaagaagatcgaatgttttgacagtgtggaaatttcaggggttgaagaccgcttcaatgcgtcattggggacttaccatgatcttctcaagatcataaaggacaaagacttcctggacaacgaagaaaatgaggatattctcgaagacatcgtcctcaccctgaccctgttcgaagacagggaaatgatagaagagcgcttgaaaacctatgcccacctcttcgacgataaagttatgaagcagctgaagcgcaggagatacacaggatggggaagattgtcaaggaagctgatcaatggaattagggataaacagagtggcaagaccatactggatttcctcaaatctgatggcttcgccaataggaacttcatgcaactgattcacgatgactctcttaccttcaaggaggacattcaaaaggctcaggtgagcgggcagggagactcccttcatgaacacatcgcgaatttggcaggttcccccgctattaaaaagggcatccttcaaactgtcaaggtggtggatgaattggtcaaggtaatgggcagacataagccagaaaatattgtgatcgagatggcccgcgaaaaccagaccacacagaagggccagaaaaatagtagagagcggatgaagaggatcgaggagggcatcaaagagctgggatctcagattctcaaagaacaccccgtagaaaacacacagctgcagaacgaaaaattgtacttgtactatctgcagaacggcagagacatgtacgtcgaccaagaacttgatattaatagactgtccgactatgacgtagaccatatcgtgccccagtccttcctgaaggacgactccattgataacaaagtcttgacaagaagcgacaagaacaggggtaaaagtgataatgtgcctagcgaggaggtggtgaaaaaaatgaagaactactggcgacagctgcttaatgcaaagctcattacacaacggaagttcgataatctgacgaaagcagagagaggtggcttgtctgagttggacaaggcagggtttattaagcggcagctggtggaaactaggcagatcacaaagcacgtggcgcagattttggacagccggatgaacacaaaatacgacgaaaatgataaactgatacgagaggtcaaagttatcacgctgaaaagcaagctggtgtccgattttcggaaagacttccagttctacaaagttcgcgagattaataactaccatcatgctcacgatgcgtacctgaacgctgttgtcgggaccgccttgataaagaagtacccaaagctggaatccgagttcgtatacggggattacaaagtgtacgatgtgaggaaaatgatagccaagtccgagcaggagattggaaaggccacagctaagtacttcttttattctaacatcatgaatttttttaagacggaaattaccctggccaacggagagatcagaaagcggccccttatagagacaaatggtgaaacaggtgaaatcgtctgggataagggcagggatttcgctactgtgaggaaggtgctgagtatgccacaggtaaatatcgtgaaaaaaaccgaagtacagaccggaggattttccaaggaaagcattttgcctaaaagaaactcagacaagctcatcgcccgcaagaaagattgggaccctaagaaatacgggggatttgactcacccaccgtagcctattctgtgctggtggtagctaaggtggaaaaaggaaagtctaagaagctgaagtccgtgaaggaactcttgggaatcactatcatggaaagatcatcctttgaaaagaaccctatcgatttcctggaggctaagggttacaaggaggtcaagaaagacctcatcattaaactgccaaaatactctctcttcgagctggaaaatggcaggaagagaatgttggccagcgccggagagctgcaaaagggaaacgagcttgctctgccctccaaatatgttaattttctctatctcgcttcccactatgaaaagctgaaagggtctcccgaagataacgagcagaagcagctgttcgtcgaacagcacaagcactatctggatgaaataatcgaacaaataagcgagttcagcaaaagggttatcctggcggatgctaatttggacaaagtactgtctgcttataacaagcaccgggataagcctattagggaacaagccgagaatataattcacctctttacactcacgaatctcggagcccccgccgccttcaaatactttgatacgactatcgaccggaaacggtataccagtaccaaagaggtcctcgatgccaccctcatccaccagtcaattactggcctgtacgaaacacggatcgacctctctcaactgggcggcgactag

SEQ ID NO: 27 - DNA, 3158 bp, Artificial Sequence
  Synthetic
atgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggcctccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggc

SEQ ID NO: 28 - DNA, 3159 bp, Artificial Sequence
  Synthetic
atgaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 29 - DNA, 3159 bp, Artificial Sequence
  Synthetic
atgaagcgcaactacatcctcggactggacatcggcattacctccgtgggatacggcatcatcgattacgaaactagggatgtgatcgacgctggagtcaggctgttcaaagaggcgaacgtggagaacaacgaggggcggcgctcaaagaggggggcccgccggctgaagcgccgccgcagacatagaatccagcgcgtgaagaagctgctgttcgactacaaccttctgaccgaccactccgaactttccggcatcaacccatatgaggctagagtgaagggattgtcccaaaagctgtccgaggaagagttctccgccgcgttgctccacctcgccaagcgcaggggagtgcacaatgtgaacgaagtggaagaagataccggaaacgagctgtccaccaaggagcagatcagccggaactccaaggccctggaagagaaatacgtggcggaactgcaactggagcggctgaagaaagacggagaagtgcgcggctcgatcaaccgcttcaagacctcggactacgtgaaggaggccaagcagctcctgaaagtgcaaaaggcctatcaccaacttgaccagtcctttatcgatacctacatcgatctgctcgagactcggcggacttactacgagggtccaggggagggctccccatttggttggaaggatattaaggagtggtacgaaatgctgatgggacactgcacatacttccctgaggagctgcggagcgtgaaatacgcatacaacgcagacctgtacaacgcgctgaacgacctgaacaatctcgtgatcacccgggacgagaacgaaaagctcgagtattacgaaaagttccagattattgagaacgtgttcaaacagaagaagaagccgacactgaagcagattgccaaggaaatcctcgtgaacgaagaggacatcaagggctatcgagtgacctcaacgggaaagccggagttcaccaatctgaaggtctaccacgacatcaaagacattaccgcccggaaggagatcattgagaacgcggagctgttggaccagattgcgaagattctgaccatctaccaatcctccgaggatattcaggaagaactcaccaacctcaacagcgaactgacccaggaggagatagagcaaatctccaacctgaagggctacaccggaactcataacctgagcctgaaggccatcaacttgatcctggacgagctgtggcacaccaacgataaccagatcgctattttcaatcggctgaagctggtccccaagaaagtggacctctcacaacaaaaggagatccctactacccttgtggacgatttcattctgtcccccgtggtcaagagaagcttcatacagtcaatcaaagtgatcaatgccattatcaagaaatacggtctgcccaacgacattatcattgagctcgcccgcgagaagaactcgaaggacgcccagaagatgattaacgaaatgcagaagaggaaccgacagactaacgaacggatcgaagaaatcatccggaccaccgggaaggaaaacgcgaagtacctgatcgaaaagatcaagctccatgacatgcaggaaggaaagtgtctgtactcgctggaggccattccgctggaggacttgctgaacaacccttttaactacgaagtggatcatatcattccgaggagcgtgtcattcgacaattccttcaacaacaaggtcctcgtgaagcaggaggaaaactcgaagaagggaaaccgcacgccgttccagtacctgagcagcagcgactccaagatttcctacgaaaccttcaagaagcacatcctcaacctggcaaaggggaagggtcgcatctccaagaccaagaaggaatatctgctggaagaaagagacatcaacagattctccgtgcaaaaggacttcatcaaccgcaacctcgtggatactagatacgctactcggggtctgatgaacctcctgagaagctactttagagtgaacaatctggacgtgaaggtcaagtcgattaacggaggtttcacctccttcctgcggcgcaagtggaagttcaagaaggaacggaacaagggctacaagcaccacgccgaggacgccctgatcattgccaacgccgacttcatcttcaaagaatggaagaaacttgacaaggctaagaaggtcatggaaaaccagatgttcgaagaaaagcaggccgagtctatgcctgaaatcgagactgaacaggagtacaaggaaatctttattacgccacaccagatcaaacacatcaaggatttcaaggattacaagtactcacatcgcgtggacaaaaagccgaacagggaactgatcaacgacaccctctactccacccggaaggatgacaaagggaataccctcatcgtcaacaaccttaacggcctgtacgacaaggacaacgataagctgaagaagctcattaacaagtcgcccgaaaagttgctgatgtaccaccacgaccctcagacttaccagaagctcaagctgatcatggagcagtatggggacgagaaaaacccgttgtacaagtactacgaagaaactgggaattatctgactaagtactccaagaaagataacggccccgtgattaagaagattaagtactacggcaacaagctgaacgcccatctggacatcaccgatgactaccctaattcccgcaacaaggtcgtcaagctgagcctcaagccctaccggtttgatgtgtaccttgacaatggagtgtacaagttcgtgactgtgaagaaccttgacgtgatcaagaaggagaactactacgaagtcaactccaagtgctacgaggaagcaaagaagttgaagaagatctcgaaccaggccgagttcattgcctccttctataacaacgacctgattaagatcaacggcgaactgtaccgcgtcattggcgtgaacaacgatctcctgaaccgcatcgaagtgaacatgatcgacatcacttaccgggaatacctggagaatatgaacgacaagcgcccgccccggatcattaagactatcgcctcaaagacccagtcgatcaagaagtacagcaccgacatcctgggcaacctgtacgaggtcaaatcgaagaagcacccccagatcatcaagaaggga

SEQ ID NO: 30 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccagagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaggcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 31 - DNA, 3242 bp, Artificial Sequence
  Synthetic
accggtgccaccatgtacccatacgatgttccagattacgcttcgccgaagaaaaagcgcaaggtcgaagcgtccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatcttacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggctaagaattc

SEQ ID NO: 32 - DNA, 3255 bp, Artificial Sequence
  Synthetic
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaag

SEQ ID NO: 33 - DNA, 3156 bp, Artificial Sequence
  Synthetic
aagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggc

SEQ ID NO: 34 - DNA, 7009 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacctttaattctagtactatgcatgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatgaaaaggaactacattctggggctggacatcgggattacaagcgtggggtatgggattattgactatgaaacaagggacgtgatcgacgcaggcgtcagactgttcaaggaggccaacgtggaaaacaatgagggacggagaagcaagaggggagccaggcgcctgaaacgacggagaaggcacagaatccagagggtgaagaaactgctgttcgattacaacctgctgaccgaccattctgagctgagtggaattaatccttatgaagccagggtgaaaggcctgagtcagaagctgtcagaggaagagttttccgcagctctgctgcacctggctaagcgccgaggagtgcataacgtcaatgaggtggaagaggacaccggcaacgagctgtctacaaaggaacagatctcacgcaatagcaaagctctggaagagaagtatgtcgcagagctgcagctggaacggctgaagaaagatggcgaggtgagagggtcaattaataggttcaagacaagcgactacgtcaaagaagccaagcagctgctgaaagtgcagaaggcttaccaccagctggatcagagcttcatcgatacttatatcgacctgctggagactcggagaacctactatgagggaccaggagaagggagccccttcggatggaaagacatcaaggaatggtacgagatgctgatgggacattgcacctattttccagaagagctgagaagcgtcaagtacgcttataacgcagatctgtacaacgccctgaatgacctgaacaacctggtcatcaccagggatgaaaacgagaaactggaatactatgagaagttccagatcatcgaaaacgtgtttaagcagaagaaaaagcctacactgaaacagattgctaaggagatcctggtcaacgaagaggacatcaagggctaccgggtgacaagcactggaaaaccagagttcaccaatctgaaagtgtatcacgatattaaggacatcacagcacggaaagaaatcattgagaacgccgaactgctggatcagattgctaagatcctgactatctaccagagctccgaggacatccaggaagagctgactaacctgaacagcgagctgacccaggaagagatcgaacagattagtaatctgaaggggtacaccggaacacacaacctgtccctgaaagctatcaatctgattctggatgagctgtggcatacaaacgacaatcagattgcaatctttaaccggctgaagctggtcccaaaaaaggtggacctgagtcagcagaaagagatcccaaccacactggtggacgatttcattctgtcacccgtggtcaagcggagcttcatccagagcatcaaagtgatcaacgccatcatcaagaagtacggcctgcccaatgatatcattatcgagctggctagggagaagaacagcaaggacgcacagaagatgatcaatgagatgcagaaacgaaaccggcagaccaatgaacgcattgaagagattatccgaactaccgggaaagagaacgcaaagtacctgattgaaaaaatcaagctgcacgatatgcaggagggaaagtgtctgtattctctggaggccatccccctggaggacctgctgaacaatccattcaactacgaggtcgatcatattatccccagaagcgtgtccttcgacaattcctttaacaacaaggtgctggtcaagcaggaagagaactctaaaaagggcaataggactcctttccagtacctgtctagttcagattccaagatctcttacgaaacctttaaaaagcacattctgaatctggccaaaggaaagggccgcatcagcaagaccaaaaaggagtacctgctggaagagcgggacatcaacagattctccgtccagaaggattttattaaccggaatctggtggacacaagatacgctactcgcggcctgatgaatctgctgcgatcctatttccgggtgaacaatctggatgtgaaagtcaagtccatcaacggcgggttcacatcttttctgaggcgcaaatggaagtttaaaaaggagcgcaacaaagggtacaagcaccatgccgaagatgctctgattatcgcaaatgccgacttcatctttaaggagtggaaaaagctggacaaagccaagaaagtgatggagaaccagatgttcgaagagaagcaggccgaatctatgcccgaaatcgagacagaacaggagtacaaggagattttcatcactcctcaccagatcaagcatatcaaggatttcaaggactacaagtactctcaccgggtggataaaaagcccaacagagagctgatcaatgacaccctgtatagtacaagaaaagacgataaggggaataccctgattgtgaacaatctgaacggactgtacgacaaagataatgacaagctgaaaaagctgatcaacaaaagtcccgagaagctgctgatgtaccaccatgatcctcagacatatcagaaactgaagctgattatggagcagtacggcgacgagaagaacccactgtataagtactatgaagagactgggaactacctgaccaagtatagcaaaaaggataatggccccgtgatcaagaagatcaagtactatgggaacaagctgaatgcccatctggacatcacagacgattaccctaacagtcgcaacaaggtggtcaagctgtcactgaagccatacagattcgatgtctatctggacaacggcgtgtataaatttgtgactgtcaagaatctggatgtcatcaaaaaggagaactactatgaagtgaatagcaagtgctacgaagaggctaaaaagctgaaaaagattagcaaccaggcagagttcatcgcctccttttacaacaacgacctgattaagatcaatggcgaactgtatagggtcatcggggtgaacaatgatctgctgaaccgcattgaagtgaatatgattgacatcacttaccgagagtatctggaaaacatgaatgataagcgcccccctcgaattatcaaaacaattgcctctaagactcagagtatcaaaaagtactcaaccgacattctgggaaacctgtatgaggtgaagagcaaaaagcaccctcagattatcaaaaagggcagcggaggcaagcgtcctgctgctactaagaaagctggtcaagctaagaaaaagaaaggatcctacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtagcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 35 - protein, 7 aa, Artificial Sequence
  Synthetic
PKKKRKV

SEQ ID NO: 36 - DNA, 6 bp, Artificial Sequence
  Synthetic
ttgggt

SEQ ID NO: 37 - DNA, 6 bp, Artificial Sequence
  Synthetic
ctgaat

SEQ ID NO: 38 - DNA, 6 bp, Artificial Sequence
  Synthetic
cagggt

SEQ ID NO: 39 - DNA, 6 bp, Artificial Sequence
  Synthetic
gagaat

SEQ ID NO: 40 - DNA, 6 bp, Artificial Sequence
  Synthetic
tttgat

SEQ ID NO: 41 - DNA, 6 bp, Artificial Sequence
  Synthetic
tagaat

SEQ ID NO: 42 - DNA, 6 bp, Artificial Sequence
  Synthetic
ttcaat

SEQ ID NO: 43 - DNA, 6 bp, Artificial Sequence
  Synthetic
aatgat

SEQ ID NO: 44 - DNA, 6 bp, Artificial Sequence
  Synthetic
agagat

SEQ ID NO: 45 - DNA, 6 bp, Artificial Sequence
  Synthetic
atgaat

SEQ ID NO: 46 - DNA, 6 bp, Artificial Sequence
  Synthetic
tgaaat

SEQ ID NO: 47 - DNA, 6 bp, Artificial Sequence
  Synthetic
ataagt

SEQ ID NO: 48 - DNA, 6 bp, Artificial Sequence
  Synthetic
cgaaat

SEQ ID NO: 49 - DNA, 17 bp, Artificial Sequence
  Synthetic
tgtctactatttctggc

SEQ ID NO: 50 - DNA, 20 bp, Artificial Sequence
  Synthetic
tagggagtggcaagttgcct

SEQ ID NO: 51 - DNA, 20 bp, Artificial Sequence
  Synthetic
gaattaagttacattgaaac

SEQ ID NO: 52 - DNA, 20 bp, Artificial Sequence
  Synthetic
tgtgttcctttggtttgatt

SEQ ID NO: 53 - DNA, 21 bp, Artificial Sequence
  Synthetic
ccaaaggaacacagagaatgt

SEQ ID NO: 54 - DNA, 21 bp, Artificial Sequence
  Synthetic
ctaattctggcacaaaatagc

SEQ ID NO: 55 - DNA, 21 bp, Artificial Sequence
  Synthetic
acattctctgtgttcctttgg

SEQ ID NO: 56 - DNA, 21 bp, Artificial Sequence
  Synthetic
ctactaattctggcacaaaat

SEQ ID NO: 57 - DNA, 21 bp, Artificial Sequence
  Synthetic
gttctgtaggcaacttgccac

SEQ ID NO: 58 - DNA, 21 bp, Artificial Sequence
  Synthetic
agccagaaatagtagacatga

SEQ ID NO: 59 - DNA, 21 bp, Artificial Sequence
  Synthetic
attaaccctggaattaagtta

SEQ ID NO: 60 - DNA, 17 bp, Artificial Sequence
  Synthetic
gccagaaatagtagaca

SEQ ID NO: 61 - DNA, 20 bp, Artificial Sequence
  Synthetic
aggcaacttgccactcccta

SEQ ID NO: 62 - DNA, 20 bp, Artificial Sequence
  Synthetic
gtttcaatgtaacttaattc

SEQ ID NO: 63 - DNA, 20 bp, Artificial Sequence
  Synthetic
aatcaaaccaaaggaacaca

SEQ ID NO: 64 - DNA, 21 bp, Artificial Sequence
  Synthetic
acattctctgtgttcctttgg

SEQ ID NO: 65 - DNA, 21 bp, Artificial Sequence
  Synthetic
gctattttgtgccagaattag

SEQ ID NO: 66 - DNA, 21 bp, Artificial Sequence
  Synthetic
ccaaaggaacacagagaatgt

SEQ ID NO: 67 - DNA, 21 bp, Artificial Sequence
  Synthetic
attttgtgccagaattagtag

SEQ ID NO: 68 - DNA, 21 bp, Artificial Sequence
  Synthetic
gtggcaagttgcctacagaac

SEQ ID NO: 69 - DNA, 21 bp, Artificial Sequence
  Synthetic
tcatgtctactatttctggct

SEQ ID NO: 70 - DNA, 21 bp, Artificial Sequence
  Synthetic
taacttaattccagggttaat

SEQ ID NO: 71 - RNA, 17 nt, Artificial Sequence
  Synthetic
gccagaaauaguagaca

SEQ ID NO: 72 - RNA, 20 nt, Artificial Sequence
  Synthetic
aggcaacuugccacucccua

SEQ ID NO: 73 - RNA, 20 nt, Artificial Sequence
  Synthetic
guuucaauguaacuuaauuc

SEQ ID NO: 74 - RNA, 20 nt, Artificial Sequence
  Synthetic
aaucaaaccaaaggaacaca

SEQ ID NO: 75 - RNA, 21 nt, Artificial Sequence
  Synthetic
acauucucuguguuccuuugg

SEQ ID NO: 76 - RNA, 21 nt, Artificial Sequence
  Synthetic
gcuauuuugugccagaauuag

SEQ ID NO: 77 - RNA, 21 nt, Artificial Sequence
  Synthetic
ccaaaggaacacagagaaugu

SEQ ID NO: 78 - RNA, 21 nt, Artificial Sequence
  Synthetic
auuuugugccagaauuaguag

SEQ ID NO: 79 - RNA, 21 nt, Artificial Sequence
  Synthetic
guggcaaguugccuacagaac

SEQ ID NO: 80 - RNA, 21 nt, Artificial Sequence
  Synthetic
ucaugucuacuauuucuggcu

SEQ ID NO: 81 - RNA, 21 nt, Artificial Sequence
  Synthetic
uaacuuaauuccaggguuaau

SEQ ID NO: 82 - DNA, 4484 bp, Artificial Sequence
  Synthetic
tgactgacctgagcgtaatacgactcactatagggcgaattgggtaccgctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggccgcccacaggccggtggaatgggtccaggccgtggtcagccgcttcgacgagcagcttccaataaaaacaggacagcagaacacacataccaaagtcagtactgagcacaacaaggaatgtctaatcaatatttccaaatacaagttttctttggttataagcggcctcactactattttaaagaatgttaacaatatgagaatatttggagaagctgctgaaaaaaatttatatctctctcagttgattatattggatacactggaaaaatgtcttgctgggcaaccaaaggacacaatgagattagatgaaacgatgctggtcaaacagttgctgccagaaatctgccattttcttcacacctgtcgtgaaggaaaccagcatgcagctgaacttcggaattctgcctctggggttttattttctctcagctgcaacaacttcaatgcagtctttagtcgcatttctaccaggttacaggaattaactgtttgttcagaagacaatgttgatgttcatgatatagaattgttacagtatatcaatgtggattgtgcaaaattaaaacgactcctgaaggaaacagcatttaaatttaaagccctaaagaaggttgcgcagttagcagttataaatagcctggaaaaggcattttggaactgggtagaaaattatccagatgaatttacaaaactgtaccagatcccacagactgatatggctgaatgtgcagaaaagctatttgacttggtggatggttttgctgaaagcaccaaacgtaaagcagcagtttggccactacaaatcattctccttatcttgtgtccagaaataatccaggatatatccaaagacgtggttgatgaaaacaacatgaataagaagttatttctggacagtctacgaaaagctcttgctggccatggaggaagtaggcagctgacagaaagtgctgcaattgcctgtgtcaaactgtgtaaagcaagtacttacatcaattgggaagataactctgtcattttcctacttgttcagtccatggtggttgatcttaagaacctgctttttaatccaagtaagccattctcaagaggcagtcagcctgcagatgtggatctaatgattgactgccttgtttcttgctttcgtataagccctcacaacaaccaacactttaagatctgcctggctcagaattcaccttctacatttcactatgtgctggtaaattcactccatcgaatcatcaccaattccgcattggattggtggcctaagattgatgctgtgtattgtcactcggttgaacttcgaaatatgtttggtgaaacacttcataaagcagtgcaaggttgtggagcacacccagcaatacgaatggcaccgagtcttacatttaaagaaaaagtaacaagccttaaatttaaagaaaaacctacagacctggagacaagaagctataagtatcttctcttgtccatggtgaaactaattcatgcagatccaaagctcttgctttgtaatccaagaaaacaggggcccgaaacccaaggcagtacagcagaattaattacagggctcgtccaactggtccctcagtcacacatgccagagattgctcaggaagcaatggaggctctgctggttcttcatcagttagatagcattgatttgtggaatcctgatgctcctgtagaaacattttgggagattagctcacaaatgcttttttacatctgcaagaaattaactagtcatcaaatgcttagtagcacagaaattctcaagtggttgcgggaaatattgatctgcaggaataaatttcttcttaaaaataagcaggcagatagaagttcctgtcactttctccttttttacggggtaggatgtgatattccttctagtggaaataccagtcaaatgtccatggatcatgaagaattactacgtactcctggagcctctctccggaagggaaaagggaactcctctatggatagtgcagcaggatgcagcggaacccccccgatttgccgacaagcccagaccaaactagaagtggccctgtacatgtttctgtggaaccctgacactgaagctgttctggttgccatgtcctgtttccgccacctctgtgaggaagcagatatccggtgtggggtggatgaagtgtcagtgcataacctcttgcccaactataacacattcatggagtttgcctctgtcagcaatatgatgtcaacaggaagagcagcacttcagaaaagagtgatggcactgctgaggcgcattgagcatcccactgcaggaaacactgaggcttgggaagatacacatgcaaaatgggaacaagcaacaaagctaatccttaactatccaaaagccaaaatggaagatggccaggctgctgaaagccttcacaagaccattgttaagaggcgaatgtcccatgtgagtggaggaggatccatagatttgtctgacacagactccctacaggaatggatcaacatgactggcttcctttgtgcccttgggggagtgtgcctccagcagagaagcaattctggcctggcaacctatagcccacccatgggtccagtcagtgaacgtaagggttctatgatttcagtgatgtcttcagagggaaacgcagatacacctgtcagcaaatttatggatcggctgttgtccttaatggtgtgtaaccatgagaaagtgggacttcaaatacggaccaatgttaaggatctggtgggtctagaattgagtcctgctctgtatccaatgctatttaacaaattgaagaataccatcagcaagttttttgactcccaaggacaggttttattgactgataccaatactcaatttgtagaacaaaccatagctataatgaagaacttgctagataatcatactgaaggcagctctgaacatctagggcaagctagcattgaaacaatgatgttaaatctggtcaggtatgttcgtgtgcttgggaatatggtccatgcaattcaaataaaaacgaaactgtgtcaattagttgaagtaatgatggcaaggagagatgacctctcattttgccaagagatgaaatttaggaataagatggtagaatacctgacagactgggttatgggaacatcaaaccaagcagcagatgatgatgtaaaatgtcttacaagagatttggaccaggcaagcatggaagcagtagtttcacttctagctggtctccctctgcagcctgaagaaggagatggtgtggaattgatggaagccaaatcacagttatttcttaaatacttcacattatttatgaaccttttgaatgactgcagtgaagttgaagatgaaagtgcgcaaacaggtggcaggaaacgtggcatgtctcggaggctggcatcactgaggcactgtacggtccttgcaatgtcaaacttactcaatgccaacgtagacagtggtctcatgcactccataggcttaggttaccacaaggatctccagacaagagctacatttatggaagttctgacaaaaatccttcaacaaggcacagaatttgacacacttgcagaaacagtattggctgatcggtttgagagattggtggaactggtcacaatgatgggtgatcaaggagaactccctatagcgatggctctggccaatgtggttccttgttctcagtgggatgaactagctcgagttctggttactctgtttgattctcggcatttactctaccaactgctctggaacatgttttctaaagaagtagaattggcagactccatgcagactctcttccgaggcaacagcttggccagtaaaataatgacattctgtttcaaggtatatggtgctacctatctacaaaaactcctggatcctttattacgaattgtgatcacatcctctgattggcaacatgttagctttgaagtggatcctaccaggttagaaccatcagagagccttgaggaaaaccagcggaacctccttcagatgactgaaaagttcttccatgccatcatcagttcctcctcagaattcccccctcaacttcgaagtgtgtgccactgcttataccaggtaagtatggaggaaggaattgcgtgttatgttgacccgtgcctaggtattacgca

SEQ ID NO: 83 - DNA, 7472 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagacggatgtgctgacccaatcatgtctactatttctggctgactgacctgagcgtaatacgactcactatagggcgaattgggtaccgctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggccgcccacaggccggtggaatgggtccaggccgtggtcagccgcttcgacgagcagcttccaataaaaacaggacagcagaacacacataccaaagtcagtactgagcacaacaaggaatgtctaatcaatatttccaaatacaagttttctttggttataagcggcctcactactattttaaagaatgttaacaatatgagaatatttggagaagctgctgaaaaaaatttatatctctctcagttgattatattggatacactggaaaaatgtcttgctgggcaaccaaaggacacaatgagattagatgaaacgatgctggtcaaacagttgctgccagaaatctgccattttcttcacacctgtcgtgaaggaaaccagcatgcagctgaacttcggaattctgcctctggggttttattttctctcagctgcaacaacttcaatgcagtctttagtcgcatttctaccaggttacaggaattaactgtttgttcagaagacaatgttgatgttcatgatatagaattgttacagtatatcaatgtggattgtgcaaaattaaaacgactcctgaaggaaacagcatttaaatttaaagccctaaagaaggttgcgcagttagcagttataaatagcctggaaaaggcattttggaactgggtagaaaattatccagatgaatttacaaaactgtaccagatcccacagactgatatggctgaatgtgcagaaaagctatttgacttggtggatggttttgctgaaagcaccaaacgtaaagcagcagtttggccactacaaatcattctccttatcttgtgtccagaaataatccaggatatatccaaagacgtggttgatgaaaacaacatgaataagaagttatttctggacagtctacgaaaagctcttgctggccatggaggaagtaggcagctgacagaaagtgctgcaattgcctgtgtcaaactgtgtaaagcaagtacttacatcaattgggaagataactctgtcattttcctacttgttcagtccatggtggttgatcttaagaacctgctttttaatccaagtaagccattctcaagaggcagtcagcctgcagatgtggatctaatgattgactgccttgtttcttgctttcgtataagccctcacaacaaccaacactttaagatctgcctggctcagaattcaccttctacatttcactatgtgctggtaaattcactccatcgaatcatcaccaattccgcattggattggtggcctaagattgatgctgtgtattgtcactcggttgaacttcgaaatatgtttggtgaaacacttcataaagcagtgcaaggttgtggagcacacccagcaatacgaatggcaccgagtcttacatttaaagaaaaagtaacaagccttaaatttaaagaaaaacctacagacctggagacaagaagctataagtatcttctcttgtccatggtgaaactaattcatgcagatccaaagctcttgctttgtaatccaagaaaacaggggcccgaaacccaaggcagtacagcagaattaattacagggctcgtccaactggtccctcagtcacacatgccagagattgctcaggaagcaatggaggctctgctggttcttcatcagttagatagcattgatttgtggaatcctgatgctcctgtagaaacattttgggagattagctcacaaatgcttttttacatctgcaagaaattaactagtcatcaaatgcttagtagcacagaaattctcaagtggttgcgggaaatattgatctgcaggaataaatttcttcttaaaaataagcaggcagatagaagttcctgtcactttctccttttttacggggtaggatgtgatattccttctagtggaaataccagtcaaatgtccatggatcatgaagaattactacgtactcctggagcctctctccggaagggaaaagggaactcctctatggatagtgcagcaggatgcagcggaacccccccgatttgccgacaagcccagaccaaactagaagtggccctgtacatgtttctgtggaaccctgacactgaagctgttctggttgccatgtcctgtttccgccacctctgtgaggaagcagatatccggtgtggggtggatgaagtgtcagtgcataacctcttgcccaactataacacattcatggagtttgcctctgtcagcaatatgatgtcaacaggaagagcagcacttcagaaaagagtgatggcactgctgaggcgcattgagcatcccactgcaggaaacactgaggcttgggaagatacacatgcaaaatgggaacaagcaacaaagctaatccttaactatccaaaagccaaaatggaagatggccaggctgctgaaagccttcacaagaccattgttaagaggcgaatgtcccatgtgagtggaggaggatccatagatttgtctgacacagactccctacaggaatggatcaacatgactggcttcctttgtgcccttgggggagtgtgcctccagcagagaagcaattctggcctggcaacctatagcccacccatgggtccagtcagtgaacgtaagggttctatgatttcagtgatgtcttcagagggaaacgcagatacacctgtcagcaaatttatggatcggctgttgtccttaatggtgtgtaaccatgagaaagtgggacttcaaatacggaccaatgttaaggatctggtgggtctagaattgagtcctgctctgtatccaatgctatttaacaaattgaagaataccatcagcaagttttttgactcccaaggacaggttttattgactgataccaatactcaatttgtagaacaaaccatagctataatgaagaacttgctagataatcatactgaaggcagctctgaacatctagggcaagctagcattgaaacaatgatgttaaatctggtcaggtatgttcgtgtgcttgggaatatggtccatgcaattcaaataaaaacgaaactgtgtcaattagttgaagtaatgatggcaaggagagatgacctctcattttgccaagagatgaaatttaggaataagatggtagaatacctgacagactgggttatgggaacatcaaaccaagcagcagatgatgatgtaaaatgtcttacaagagatttggaccaggcaagcatggaagcagtagtttcacttctagctggtctccctctgcagcctgaagaaggagatggtgtggaattgatggaagccaaatcacagttatttcttaaatacttcacattatttatgaaccttttgaatgactgcagtgaagttgaagatgaaagtgcgcaaacaggtggcaggaaacgtggcatgtctcggaggctggcatcactgaggcactgtacggtccttgcaatgtcaaacttactcaatgccaacgtagacagtggtctcatgcactccataggcttaggttaccacaaggatctccagacaagagctacatttatggaagttctgacaaaaatccttcaacaaggcacagaatttgacacacttgcagaaacagtattggctgatcggtttgagagattggtggaactggtcacaatgatgggtgatcaaggagaactccctatagcgatggctctggccaatgtggttccttgttctcagtgggatgaactagctcgagttctggttactctgtttgattctcggcatttactctaccaactgctctggaacatgttttctaaagaagtagaattggcagactccatgcagactctcttccgaggcaacagcttggccagtaaaataatgacattctgtttcaaggtatatggtgctacctatctacaaaaactcctggatcctttattacgaattgtgatcacatcctctgattggcaacatgttagctttgaagtggatcctaccaggttagaaccatcagagagccttgaggaaaaccagcggaacctccttcagatgactgaaaagttcttccatgccatcatcagttcctcctcagaattcccccctcaacttcgaagtgtgtgccactgcttataccaggtaagtatggaggaaggaattgcgtgttatgttgacccgtgcctaggtattacgcaacccaatcatgtctactatttctggcgcggccgcccgcggtggagctccagcttttgttccctttggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 84 - DNA, 7465 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagacggatgtgctggtttcaatgtaacttaattccagggttgactgacctgagcgtaatacgactcactatagggcgaattgggtaccgctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggccgcccacaggccggtggaatgggtccaggccgtggtcagccgcttcgacgagcagcttccaataaaaacaggacagcagaacacacataccaaagtcagtactgagcacaacaaggaatgtctaatcaatatttccaaatacaagttttctttggttataagcggcctcactactattttaaagaatgttaacaatatgagaatatttggagaagctgctgaaaaaaatttatatctctctcagttgattatattggatacactggaaaaatgtcttgctgggcaaccaaaggacacaatgagattagatgaaacgatgctggtcaaacagttgctgccagaaatctgccattttcttcacacctgtcgtgaaggaaaccagcatgcagctgaacttcggaattctgcctctggggttttattttctctcagctgcaacaacttcaatgcagtctttagtcgcatttctaccaggttacaggaattaactgtttgttcagaagacaatgttgatgttcatgatatagaattgttacagtatatcaatgtggattgtgcaaaattaaaacgactcctgaaggaaacagcatttaaatttaaagccctaaagaaggttgcgcagttagcagttataaatagcctggaaaaggcattttggaactgggtagaaaattatccagatgaatttacaaaactgtaccagatcccacagactgatatggctgaatgtgcagaaaagctatttgacttggtggatggttttgctgaaagcaccaaacgtaaagcagcagtttggccactacaaatcattctccttatcttgtgtccagaaataatccaggatatatccaaagacgtggttgatgaaaacaacatgaataagaagttatttctggacagtctacgaaaagctcttgctggccatggaggaagtaggcagctgacagaaagtgctgcaattgcctgtgtcaaactgtgtaaagcaagtacttacatcaattgggaagataactctgtcattttcctacttgttcagtccatggtggttgatcttaagaacctgctttttaatccaagtaagccattctcaagaggcagtcagcctgcagatgtggatctaatgattgactgccttgtttcttgctttcgtataagccctcacaacaaccaacactttaagatctgcctggctcagaattcaccttctacatttcactatgtgctggtaaattcactccatcgaatcatcaccaattccgcattggattggtggcctaagattgatgctgtgtattgtcactcggttgaacttcgaaatatgtttggtgaaacacttcataaagcagtgcaaggttgtggagcacacccagcaatacgaatggcaccgagtcttacatttaaagaaaaagtaacaagccttaaatttaaagaaaaacctacagacctggagacaagaagctataagtatcttctcttgtccatggtgaaactaattcatgcagatccaaagctcttgctttgtaatccaagaaaacaggggcccgaaacccaaggcagtacagcagaattaattacagggctcgtccaactggtccctcagtcacacatgccagagattgctcaggaagcaatggaggctctgctggttcttcatcagttagatagcattgatttgtggaatcctgatgctcctgtagaaacattttgggagattagctcacaaatgcttttttacatctgcaagaaattaactagtcatcaaatgcttagtagcacagaaattctcaagtggttgcgggaaatattgatctgcaggaataaatttcttcttaaaaataagcaggcagatagaagttcctgtcactttctccttttttacggggtaggatgtgatattccttctagtggaaataccagtcaaatgtccatggatcatgaagaattactacgtactcctggagcctctctccggaagggaaaagggaactcctctatggatagtgcagcaggatgcagcggaacccccccgatttgccgacaagcccagaccaaactagaagtggccctgtacatgtttctgtggaaccctgacactgaagctgttctggttgccatgtcctgtttccgccacctctgtgaggaagcagatatccggtgtggggtggatgaagtgtcagtgcataacctcttgcccaactataacacattcatggagtttgcctctgtcagcaatatgatgtcaacaggaagagcagcacttcagaaaagagtgatggcactgctgaggcgcattgagcatcccactgcaggaaacactgaggcttgggaagatacacatgcaaaatgggaacaagcaacaaagctaatccttaactatccaaaagccaaaatggaagatggccaggctgctgaaagccttcacaagaccattgttaagaggcgaatgtcccatgtgagtggaggaggatccatagatttgtctgacacagactccctacaggaatggatcaacatgactggcttcctttgtgcccttgggggagtgtgcctccagcagagaagcaattctggcctggcaacctatagcccacccatgggtccagtcagtgaacgtaagggttctatgatttcagtgatgtcttcagagggaaacgcagatacacctgtcagcaaatttatggatcggctgttgtccttaatggtgtgtaaccatgagaaagtgggacttcaaatacggaccaatgttaaggatctggtgggtctagaattgagtcctgctctgtatccaatgctatttaacaaattgaagaataccatcagcaagttttttgactcccaaggacaggttttattgactgataccaatactcaatttgtagaacaaaccatagctataatgaagaacttgctagataatcatactgaaggcagctctgaacatctagggcaagctagcattgaaacaatgatgttaaatctggtcaggtatgttcgtgtgcttgggaatatggtccatgcaattcaaataaaaacgaaactgtgtcaattagttgaagtaatgatggcaaggagagatgacctctcattttgccaagagatgaaatttaggaataagatggtagaatacctgacagactgggttatgggaacatcaaaccaagcagcagatgatgatgtaaaatgtcttacaagagatttggaccaggcaagcatggaagcagtagtttcacttctagctggtctccctctgcagcctgaagaaggagatggtgtggaattgatggaagccaaatcacagttatttcttaaatacttcacattatttatgaaccttttgaatgactgcagtgaagttgaagatgaaagtgcgcaaacaggtggcaggaaacgtggcatgtctcggaggctggcatcactgaggcactgtacggtccttgcaatgtcaaacttactcaatgccaacgtagacagtggtctcatgcactccataggcttaggttaccacaaggatctccagacaagagctacatttatggaagttctgacaaaaatccttcaacaaggcacagaatttgacacacttgcagaaacagtattggctgatcggtttgagagattggtggaactggtcacaatgatgggtgatcaaggagaactccctatagcgatggctctggccaatgtggttccttgttctcagtgggatgaactagctcgagttctggttactctgtttgattctcggcatttactctaccaactgctctggaacatgttttctaaagaagtagaattggcagactccatgcagactctcttccgaggcaacagcttggccagtaaaataatgacattctgtttcaaggtatatggtgctacctatctacaaaaactcctggatcctttattacgaattgtgatcacatcctctgattggcaacatgttagctttgaagtggatcctaccaggttagaaccatcagagagccttgaggaaaaccagcggaacctccttcagatgactgaaaagttcttccatgccatcatcagttcctcctcagaattcccccctcaacttcgaagtgtgtgccactgcttataccaggtaagttatggaggaaggaattgcgtgttatgttgacccgtgcctaggtattacgcgtttcaatgtaacttaattccagggtcaagcttgtcagctccagcttttgttccctttggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 85 - DNA, 7472 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagacggatgtgctgaatcaaaccaaaggaacacagagaattgactgacctgagcgtaatacgactcactatagggcgaattgggtaccgctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggccgcccacaggccggtggaatgggtccaggccgtggtcagccgcttcgacgagcagcttccaataaaaacaggacagcagaacacacataccaaagtcagtactgagcacaacaaggaatgtctaatcaatatttccaaatacaagttttctttggttataagcggcctcactactattttaaagaatgttaacaatatgagaatatttggagaagctgctgaaaaaaatttatatctctctcagttgattatattggatacactggaaaaatgtcttgctgggcaaccaaaggacacaatgagattagatgaaacgatgctggtcaaacagttgctgccagaaatctgccattttcttcacacctgtcgtgaaggaaaccagcatgcagctgaacttcggaattctgcctctggggttttattttctctcagctgcaacaacttcaatgcagtctttagtcgcatttctaccaggttacaggaattaactgtttgttcagaagacaatgttgatgttcatgatatagaattgttacagtatatcaatgtggattgtgcaaaattaaaacgactcctgaaggaaacagcatttaaatttaaagccctaaagaaggttgcgcagttagcagttataaatagcctggaaaaggcattttggaactgggtagaaaattatccagatgaatttacaaaactgtaccagatcccacagactgatatggctgaatgtgcagaaaagctatttgacttggtggatggttttgctgaaagcaccaaacgtaaagcagcagtttggccactacaaatcattctccttatcttgtgtccagaaataatccaggatatatccaaagacgtggttgatgaaaacaacatgaataagaagttatttctggacagtctacgaaaagctcttgctggccatggaggaagtaggcagctgacagaaagtgctgcaattgcctgtgtcaaactgtgtaaagcaagtacttacatcaattgggaagataactctgtcattttcctacttgttcagtccatggtggttgatcttaagaacctgctttttaatccaagtaagccattctcaagaggcagtcagcctgcagatgtggatctaatgattgactgccttgtttcttgctttcgtataagccctcacaacaaccaacactttaagatctgcctggctcagaattcaccttctacatttcactatgtgctggtaaattcactccatcgaatcatcaccaattccgcattggattggtggcctaagattgatgctgtgtattgtcactcggttgaacttcgaaatatgtttggtgaaacacttcataaagcagtgcaaggttgtggagcacacccagcaatacgaatggcaccgagtcttacatttaaagaaaaagtaacaagccttaaatttaaagaaaaacctacagacctggagacaagaagctataagtatcttctcttgtccatggtgaaactaattcatgcagatccaaagctcttgctttgtaatccaagaaaacaggggcccgaaacccaaggcagtacagcagaattaattacagggctcgtccaactggtccctcagtcacacatgccagagattgctcaggaagcaatggaggctctgctggttcttcatcagttagatagcattgatttgtggaatcctgatgctcctgtagaaacattttgggagattagctcacaaatgcttttttacatctgcaagaaattaactagtcatcaaatgcttagtagcacagaaattctcaagtggttgcgggaaatattgatctgcaggaataaatttcttcttaaaaataagcaggcagatagaagttcctgtcactttctccttttttacggggtaggatgtgatattccttctagtggaaataccagtcaaatgtccatggatcatgaagaattactacgtactcctggagcctctctccggaagggaaaagggaactcctctatggatagtgcagcaggatgcagcggaacccccccgatttgccgacaagcccagaccaaactagaagtggccctgtacatgtttctgtggaaccctgacactgaagctgttctggttgccatgtcctgtttccgccacctctgtgaggaagcagatatccggtgtggggtggatgaagtgtcagtgcataacctcttgcccaactataacacattcatggagtttgcctctgtcagcaatatgatgtcaacaggaagagcagcacttcagaaaagagtgatggcactgctgaggcgcattgagcatcccactgcaggaaacactgaggcttgggaagatacacatgcaaaatgggaacaagcaacaaagctaatccttaactatccaaaagccaaaatggaagatggccaggctgctgaaagccttcacaagaccattgttaagaggcgaatgtcccatgtgagtggaggaggatccatagatttgtctgacacagactccctacaggaatggatcaacatgactggcttcctttgtgcccttgggggagtgtgcctccagcagagaagcaattctggcctggcaacctatagcccacccatgggtccagtcagtgaacgtaagggttctatgatttcagtgatgtcttcagagggaaacgcagatacacctgtcagcaaatttatggatcggctgttgtccttaatggtgtgtaaccatgagaaagtgggacttcaaatacggaccaatgttaaggatctggtgggtctagaattgagtcctgctctgtatccaatgctatttaacaaattgaagaataccatcagcaagttttttgactcccaaggacaggttttattgactgataccaatactcaatttgtagaacaaaccatagctataatgaagaacttgctagataatcatactgaaggcagctctgaacatctagggcaagctagcattgaaacaatgatgttaaatctggtcaggtatgttcgtgtgcttgggaatatggtccatgcaattcaaataaaaacgaaactgtgtcaattagttgaagtaatgatggcaaggagagatgacctctcattttgccaagagatgaaatttaggaataagatggtagaatacctgacagactgggttatgggaacatcaaaccaagcagcagatgatgatgtaaaatgtcttacaagagatttggaccaggcaagcatggaagcagtagtttcacttctagctggtctccctctgcagcctgaagaaggagatggtgtggaattgatggaagccaaatcacagttatttcttaaatacttcacattatttatgaaccttttgaatgactgcagtgaagttgaagatgaaagtgcgcaaacaggtggcaggaaacgtggcatgtctcggaggctggcatcactgaggcactgtacggtccttgcaatgtcaaacttactcaatgccaacgtagacagtggtctcatgcactccataggcttaggttaccacaaggatctccagacaagagctacatttatggaagttctgacaaaaatccttcaacaaggcacagaatttgacacacttgcagaaacagtattggctgatcggtttgagagattggtggaactggtcacaatgatgggtgatcaaggagaactccctatagcgatggctctggccaatgtggttccttgttctcagtgggatgaactagctcgagttctggttactctgtttgattctcggcatttactctaccaactgctctggaacatgttttctaaagaagtagaattggcagactccatgcagactctcttccgaggcaacagcttggccagtaaaataatgacattctgtttcaaggtatatggtgctacctatctacaaaaactcctggatcctttattacgaattgtgatcacatcctctgattggcaacatgttagctttgaagtggatcctaccaggttagaaccatcagagagccttgaggaaaaccagcggaacctccttcagatgactgaaaagttcttccatgccatcatcagttcctcctcagaattcccccctcaacttcgaagtgtgtgccactgcttataccaggtaagtatggaggaaggaattgcgtgttatgttgacccgtgcctaggtattacgcaatcaaaccaaaggaacacagagaatcgcggccgcccgcggtggagctccagcttttgttccctttggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 86 - DNA, 7474 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagacgggggatgtggctattttgtgccagaattagtagaattgactgacctgagcgtaatacgactcactatagggcgaattgggtaccgctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggccgcccacaggccggtggaatgggtccaggccgtggtcagccgcttcgacgagcagcttccaataaaaacaggacagcagaacacacataccaaagtcagtactgagcacaacaaggaatgtctaatcaatatttccaaatacaagttttctttggttataagcggcctcactactattttaaagaatgttaacaatatgagaatatttggagaagctgctgaaaaaaatttatatctctctcagttgattatattggatacactggaaaaatgtcttgctgggcaaccaaaggacacaatgagattagatgaaacgatgctggtcaaacagttgctgccagaaatctgccattttcttcacacctgtcgtgaaggaaaccagcatgcagctgaacttcggaattctgcctctggggttttattttctctcagctgcaacaacttcaatgcagtctttagtcgcatttctaccaggttacaggaattaactgtttgttcagaagacaatgttgatgttcatgatatagaattgttacagtatatcaatgtggattgtgcaaaattaaaacgactcctgaaggaaacagcatttaaatttaaagccctaaagaaggttgcgcagttagcagttataaatagcctggaaaaggcattttggaactgggtagaaaattatccagatgaatttacaaaactgtaccagatcccacagactgatatggctgaatgtgcagaaaagctatttgacttggtggatggttttgctgaaagcaccaaacgtaaagcagcagtttggccactacaaatcattctccttatcttgtgtccagaaataatccaggatatatccaaagacgtggttgatgaaaacaacatgaataagaagttatttctggacagtctacgaaaagctcttgctggccatggaggaagtaggcagctgacagaaagtgctgcaattgcctgtgtcaaactgtgtaaagcaagtacttacatcaattgggaagataactctgtcattttcctacttgttcagtccatggtggttgatcttaagaacctgctttttaatccaagtaagccattctcaagaggcagtcagcctgcagatgtggatctaatgattgactgccttgtttcttgctttcgtataagccctcacaacaaccaacactttaagatctgcctggctcagaattcaccttctacatttcactatgtgctggtaaattcactccatcgaatcatcaccaattccgcattggattggtggcctaagattgatgctgtgtattgtcactcggttgaacttcgaaatatgtttggtgaaacacttcataaagcagtgcaaggttgtggagcacacccagcaatacgaatggcaccgagtcttacatttaaagaaaaagtaacaagccttaaatttaaagaaaaacctacagacctggagacaagaagctataagtatcttctcttgtccatggtgaaactaattcatgcagatccaaagctcttgctttgtaatccaagaaaacaggggcccgaaacccaaggcagtacagcagaattaattacagggctcgtccaactggtccctcagtcacacatgccagagattgctcaggaagcaatggaggctctgctggttcttcatcagttagatagcattgatttgtggaatcctgatgctcctgtagaaacattttgggagattagctcacaaatgcttttttacatctgcaagaaattaactagtcatcaaatgcttagtagcacagaaattctcaagtggttgcgggaaatattgatctgcaggaataaatttcttcttaaaaataagcaggcagatagaagttcctgtcactttctccttttttacggggtaggatgtgatattccttctagtggaaataccagtcaaatgtccatggatcatgaagaattactacgtactcctggagcctctctccggaagggaaaagggaactcctctatggatagtgcagcaggatgcagcggaacccccccgatttgccgacaagcccagaccaaactagaagtggccctgtacatgtttctgtggaaccctgacactgaagctgttctggttgccatgtcctgtttccgccacctctgtgaggaagcagatatccggtgtggggtggatgaagtgtcagtgcataacctcttgcccaactataacacattcatggagtttgcctctgtcagcaatatgatgtcaacaggaagagcagcacttcagaaaagagtgatggcactgctgaggcgcattgagcatcccactgcaggaaacactgaggcttgggaagatacacatgcaaaatgggaacaagcaacaaagctaatccttaactatccaaaagccaaaatggaagatggccaggctgctgaaagccttcacaagaccattgttaagaggcgaatgtcccatgtgagtggaggaggatccatagatttgtctgacacagactccctacaggaatggatcaacatgactggcttcctttgtgcccttgggggagtgtgcctccagcagagaagcaattctggcctggcaacctatagcccacccatgggtccagtcagtgaacgtaagggttctatgatttcagtgatgtcttcagagggaaacgcagatacacctgtcagcaaatttatggatcggctgttgtccttaatggtgtgtaaccatgagaaagtgggacttcaaatacggaccaatgttaaggatctggtgggtctagaattgagtcctgctctgtatccaatgctatttaacaaattgaagaataccatcagcaagttttttgactcccaaggacaggttttattgactgataccaatactcaatttgtagaacaaaccatagctataatgaagaacttgctagataatcatactgaaggcagctctgaacatctagggcaagctagcattgaaacaatgatgttaaatctggtcaggtatgttcgtgtgcttgggaatatggtccatgcaattcaaataaaaacgaaactgtgtcaattagttgaagtaatgatggcaaggagagatgacctctcattttgccaagagatgaaatttaggaataagatggtagaatacctgacagactgggttatgggaacatcaaaccaagcagcagatgatgatgtaaaatgtcttacaagagatttggaccaggcaagcatggaagcagtagtttcacttctagctggtctccctctgcagcctgaagaaggagatggtgtggaattgatggaagccaaatcacagttatttcttaaatacttcacattatttatgaaccttttgaatgactgcagtgaagttgaagatgaaagtgcgcaaacaggtggcaggaaacgtggcatgtctcggaggctggcatcactgaggcactgtacggtccttgcaatgtcaaacttactcaatgccaacgtagacagtggtctcatgcactccataggcttaggttaccacaaggatctccagacaagagctacatttatggaagttctgacaaaaatccttcaacaaggcacagaatttgacacacttgcagaaacagtattggctgatcggtttgagagattggtggaactggtcacaatgatgggtgatcaaggagaactccctatagcgatggctctggccaatgtggttccttgttctcagtgggatgaactagctcgagttctggttactctgtttgattctcggcatttactctaccaactgctctggaacatgttttctaaagaagtagaattggcagactccatgcagactctcttccgaggcaacagcttggccagtaaaataatgacattctgtttcaaggtatatggtgctacctatctacaaaaactcctggatcctttattacgaattgtgatcacatcctctgattggcaacatgttagctttgaagtggatcctaccaggttagaaccatcagagagccttgaggaaaaccagcggaacctccttcagatgactgaaaagttcttccatgccatcatcagttcctcctcagaattcccccctcaacttcgaagtgtgtgccactgcttataccaggtaagtatggaggaaggaattgcgtgttatgttgacccgtgcctaggtattacgcgctattttgtgccagaattagtagaatcgcggccgcccgcggtggagctccagcttttgttccctttggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 87 - DNA, 7450 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccagaaatagtagacatgagttttagtactctggaaacagaatctactaaaacaaggcaaaatgccgtgtttatctcgtcaacttgttggcgagatttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 88 - DNA, 7450 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtttcaatgtaacttaattcgttttagtactctggaaacagaatctactaaaacaaggcaaaatgccgtgtttatctcgtcaacttgttggcgagatttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 89 - DNA, 7451 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaatcaaaccaaaggaacacagttttagtactctggaaacagaatctactaaaacaaggcaaaatgccgtgtttatctcgtcaacttgttggcgagatttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 90 - DNA, 7451 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagactcgaggcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactaccggtgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatcctacccatacgatgttccagattacgcttacccatacgatgttccagattacgcttacccatacgatgttccagattacgcttaagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggaggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctattttgtgccagaattaggttttagtactctggaaacagaatctactaaaacaaggcaaaatgccgtgtttatctcgtcaacttgttggcgagatttttttgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 91 - DNA, 3235 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccagaaatagtagacatgagttttagtactctggaaacagaatctactaaaacaaggcaaaatgccgtgtttatctcgtcaacttgttggcgagatttttttgcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 92 - DNA, 3236 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaggcaacttgccactccctagttttagtactctggaaacagaatctactaaaacaaggcaaaatgccgtgtttatctcgtcaacttgttggcgagatttttttgcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 93 - DNA, 3235 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtttcaatgtaacttaattcgttttagtactctggaaacagaatctactaaaacaaggcaaaatgccgtgtttatctcgtcaacttgttggcgagatttttttgcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 94 - DNA, 3236 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaatcaaaccaaaggaacacagttttagtactctggaaacagaatctactaaaacaaggcaaaatgccgtgtttatctcgtcaacttgttggcgagatttttttgcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 95 - DNA, 3237 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgacattctctgtgttcctttgggttttagtactctggaaacagaatctactaaaacaaggcaaaatgccgtgtttatctcgtcaacttgttggcgagatttttttgcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 96 - DNA, 3236 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctattttgtgccagaattaggttttagtactctggaaacagaatctactaaaacaaggcaaaatgccgtgtttatctcgtcaacttgttggcgagatttttttgcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 97 - DNA, 3237 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgccaaaggaacacagagaatgtgttttagtactctggaaacagaatctactaaaacaaggcaaaatgccgtgtttatctcgtcaacttgttggcgagatttttttgcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 98 - DNA, 3237 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgattttgtgccagaattagtaggttttagtactctggaaacagaatctactaaaacaaggcaaaatgccgtgtttatctcgtcaacttgttggcgagatttttttgcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 99 - DNA, 3236 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtggcaagttgcctacagaacgttttagtactctggaaacagaatctactaaaacaaggcaaaatgccgtgtttatctcgtcaacttgttggcgagatttttttgcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 100 - DNA, 3237 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtcatgtctactatttctggctgttttagtactctggaaacagaatctactaaaacaaggcaaaatgccgtgtttatctcgtcaacttgttggcgagatttttttgcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 101 - DNA, 3237 bp, Artificial Sequence
  Synthetic
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccaagcttgcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtaacttaattccagggttaatgttttagtactctggaaacagaatctactaaaacaaggcaaaatgccgtgtttatctcgtcaacttgttggcgagatttttttgcggccgcccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 102 - DNA, 7472 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggcctctagacggatgtgctgattccttgagttagctctggtagtgctgactgacctgagcgtaatacgactcactatagggcgaattgggtaccgctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggtgccaccatggccgcccacaggccggtggaatgggtccaggccgtggtcagccgcttcgacgagcagcttccaataaaaacaggacagcagaacacacataccaaagtcagtactgagcacaacaaggaatgtctaatcaatatttccaaatacaagttttctttggttataagcggcctcactactattttaaagaatgttaacaatatgagaatatttggagaagctgctgaaaaaaatttatatctctctcagttgattatattggatacactggaaaaatgtcttgctgggcaaccaaaggacacaatgagattagatgaaacgatgctggtcaaacagttgctgccagaaatctgccattttcttcacacctgtcgtgaaggaaaccagcatgcagctgaacttcggaattctgcctctggggttttattttctctcagctgcaacaacttcaatgcagtctttagtcgcatttctaccaggttacaggaattaactgtttgttcagaagacaatgttgatgttcatgatatagaattgttacagtatatcaatgtggattgtgcaaaattaaaacgactcctgaaggaaacagcatttaaatttaaagccctaaagaaggttgcgcagttagcagttataaatagcctggaaaaggcattttggaactgggtagaaaattatccagatgaatttacaaaactgtaccagatcccacagactgatatggctgaatgtgcagaaaagctatttgacttggtggatggttttgctgaaagcaccaaacgtaaagcagcagtttggccactacaaatcattctccttatcttgtgtccagaaataatccaggatatatccaaagacgtggttgatgaaaacaacatgaataagaagttatttctggacagtctacgaaaagctcttgctggccatggaggaagtaggcagctgacagaaagtgctgcaattgcctgtgtcaaactgtgtaaagcaagtacttacatcaattgggaagataactctgtcattttcctacttgttcagtccatggtggttgatcttaagaacctgctttttaatccaagtaagccattctcaagaggcagtcagcctgcagatgtggatctaatgattgactgccttgtttcttgctttcgtataagccctcacaacaaccaacactttaagatctgcctggctcagaattcaccttctacatttcactatgtgctggtaaattcactccatcgaatcatcaccaattccgcattggattggtggcctaagattgatgctgtgtattgtcactcggttgaacttcgaaatatgtttggtgaaacacttcataaagcagtgcaaggttgtggagcacacccagcaatacgaatggcaccgagtcttacatttaaagaaaaagtaacaagccttaaatttaaagaaaaacctacagacctggagacaagaagctataagtatcttctcttgtccatggtgaaactaattcatgcagatccaaagctcttgctttgtaatccaagaaaacaggggcccgaaacccaaggcagtacagcagaattaattacagggctcgtccaactggtccctcagtcacacatgccagagattgctcaggaagcaatggaggctctgctggttcttcatcagttagatagcattgatttgtggaatcctgatgctcctgtagaaacattttgggagattagctcacaaatgcttttttacatctgcaagaaattaactagtcatcaaatgcttagtagcacagaaattctcaagtggttgcgggaaatattgatctgcaggaataaatttcttcttaaaaataagcaggcagatagaagttcctgtcactttctccttttttacggggtaggatgtgatattccttctagtggaaataccagtcaaatgtccatggatcatgaagaattactacgtactcctggagcctctctccggaagggaaaagggaactcctctatggatagtgcagcaggatgcagcggaacccccccgatttgccgacaagcccagaccaaactagaagtggccctgtacatgtttctgtggaaccctgacactgaagctgttctggttgccatgtcctgtttccgccacctctgtgaggaagcagatatccggtgtggggtggatgaagtgtcagtgcataacctcttgcccaactataacacattcatggagtttgcctctgtcagcaatatgatgtcaacaggaagagcagcacttcagaaaagagtgatggcactgctgaggcgcattgagcatcccactgcaggaaacactgaggcttgggaagatacacatgcaaaatgggaacaagcaacaaagctaatccttaactatccaaaagccaaaatggaagatggccaggctgctgaaagccttcacaagaccattgttaagaggcgaatgtcccatgtgagtggaggaggatccatagatttgtctgacacagactccctacaggaatggatcaacatgactggcttcctttgtgcccttgggggagtgtgcctccagcagagaagcaattctggcctggcaacctatagcccacccatgggtccagtcagtgaacgtaagggttctatgatttcagtgatgtcttcagagggaaacgcagatacacctgtcagcaaatttatggatcggctgttgtccttaatggtgtgtaaccatgagaaagtgggacttcaaatacggaccaatgttaaggatctggtgggtctagaattgagtcctgctctgtatccaatgctatttaacaaattgaagaataccatcagcaagttttttgactcccaaggacaggttttattgactgataccaatactcaatttgtagaacaaaccatagctataatgaagaacttgctagataatcatactgaaggcagctctgaacatctagggcaagctagcattgaaacaatgatgttaaatctggtcaggtatgttcgtgtgcttgggaatatggtccatgcaattcaaataaaaacgaaactgtgtcaattagttgaagtaatgatggcaaggagagatgacctctcattttgccaagagatgaaatttaggaataagatggtagaatacctgacagactgggttatgggaacatcaaaccaagcagcagatgatgatgtaaaatgtcttacaagagatttggaccaggcaagcatggaagcagtagtttcacttctagctggtctccctctgcagcctgaagaaggagatggtgtggaattgatggaagccaaatcacagttatttcttaaatacttcacattatttatgaaccttttgaatgactgcagtgaagttgaagatgaaagtgcgcaaacaggtggcaggaaacgtggcatgtctcggaggctggcatcactgaggcactgtacggtccttgcaatgtcaaacttactcaatgccaacgtagacagtggtctcatgcactccataggcttaggttaccacaaggatctccagacaagagctacatttatggaagttctgacaaaaatccttcaacaaggcacagaatttgacacacttgcagaaacagtattggctgatcggtttgagagattggtggaactggtcacaatgatgggtgatcaaggagaactccctatagcgatggctctggccaatgtggttccttgttctcagtgggatgaactagctcgagttctggttactctgtttgattctcggcatttactctaccaactgctctggaacatgttttctaaagaagtagaattggcagactccatgcagactctcttccgaggcaacagcttggccagtaaaataatgacattctgtttcaaggtatatggtgctacctatctacaaaaactcctggatcctttattacgaattgtgatcacatcctctgattggcaacatgttagctttgaagtggatcctaccaggttagaaccatcagagagccttgaggaaaaccagcggaacctccttcagatgactgaaaagttcttccatgccatcatcagttcctcctcagaattcccccctcaacttcgaagtgtgtgccactgcttataccaggtaagtatggaggaaggaattgcgtgttatgttgacccgtgcctaggtattacgcaattccttgagttagctctggtagtgcgcggccgcccgcggtggagctccagcttttgttccctttggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2023/0201375 A1
(43) Pub. Date: Jun. 29, 2023

(54) TARGETED GENOMIC INTEGRATION TO RESTORE NEUROFIBROMIN CODING SEQUENCE IN NEUROFIBROMATOSIS TYPE 1 (NF1)

(71) Applicant: Duke University, Durham, NC (US)

(72) Inventors: Charles A. Gersbach, Chapel Hill, NC (US); Madeleine Sitton, Chapel Hill, NC (US)

(21) Appl. No.: 17/921,338

(22) PCT Filed: Apr. 27, 2021
(86) PCT No.: PCT/US2021/029500

§ 371 Date: Oct. 25, 2022

Related U.S. Application Data
(60) Provisional application No. 63/015,866, filed on Apr. 27, 2020.
(60) Provisional application No. 63/015,740, filed on Apr. 27, 2020.

(51) Int. Cl. A61K 48/00; C12N 15/11; C12N 9/22; C12N 15/90; A61K 31/7088; A61K 38/46; A61P 35/00; A61P 25/00; A61K 38/17

(52) U.S. Cl. CPC A61K 48/0066; A61K 31/7088; A61K 38/465; A61K 38/1709; A61P 25/00; A61P 35/00; C12N 9/22; C12N 15/11; C12N 15/907; C12N 2310/20; C12N 2800/80

(57) ABSTRACT

Disclosed herein are systems and methods for the treatment of NF1 in a subject. The CRISPR/Cas-based genome editing systems may include a polynucleotide sequence encoding a guide RNA (gRNA) targeting a fragment of a mutant NF1 gene, a polynucleotide sequence encoding a Cas protein or a fusion protein comprising the Cas protein, and a polynucleotide sequence encoding a donor sequence comprising a fragment of a wild-type NF1 gene.

### CROSS-REFERENCE TO RELATED APPLICATIONS

This application claims priority to U.S. Provisional Pat. Application No. 63/015,740, filed Apr. 27, 2020, and U.S. Provisional Pat. Application No. 63/015,866, filed Apr. 27, 2020, each of which is incorporated herein by reference in its entirety.

### FIELD

This disclosure relates to Neurofibromatosis Type I (NF1) as well as compositions and methods for using CRISPR/Cas-based systems to treat the same.

### INTRODUCTION

Neurofibromatosis Type I (NF1) is an autosomal dominant disease caused by the loss of function of the protein neurofibromin, a GTPase-activating protein that negatively regulates the Ras signaling pathway. Loss of neurofibromin leads to the formation of malignant and benign neurofibromas originating in non-dividing cells that form the myelin sheath of peripheral nerves, called Schwann cells. This disease affects 1 in 3,000 people worldwide, however, there is no effective treatment to reduce the size or number of neurofibromas. Gene editing technology could address the root cause of NF1 by correcting mutations in the NF1 gene, however, current approaches are not broadly applicable due to a series of limitations. First, the 8.6 kb NF1 coding sequence is too large to be delivered in its entirety via traditional gene therapy vectors. Second, no single patient mutation occurs in the population at a frequency greater than 2% and mutations are distributed along the full coding sequence of the gene, such that no single conventional gene editing approach can address a significant portion of the population. Finally, post-mitotic Schwann cells do not efficiently utilize homologous recombination pathways. Hence, there is still a great need for novel therapies and treatments for NF1.

### SUMMARY

In an aspect, the disclosure relates to a CRISPR/Cas-based genome editing system. The system may include (a) a polynucleotide sequence encoding a guide RNA (gRNA) targeting a fragment of a mutant NF1 gene; (b) a polynucleotide sequence encoding a Cas protein or a fusion protein comprising the Cas protein; and (c) a polynucleotide sequence encoding a donor sequence comprising a fragment of a wild-type NF1 gene. In some embodiments, the system comprises one or more vectors. In some embodiments, the system comprises a first vector and a second vector, wherein the first vector comprises the polynucleotide sequence encoding the gRNA, and the polynucleotide sequence encoding the Cas protein or the fusion protein, and the second vector comprises the polynucleotide sequence encoding the donor sequence. In some embodiments, the polynucleotide sequence encoding the gRNA and the polynucleotide sequence encoding the Cas protein or the fusion protein are operably linked. In some embodiments, the mutant NF1 gene comprises a mutation in the 5′ portion of the mutant NF1 gene, and the polynucleotide sequence encoding the donor sequence further comprises a stop codon that is 5′ to the donor sequence. In some embodiments, the gRNA targets a sequence upstream of the stop codon that is 5′ to the donor sequence and targets a sequence downstream of the donor sequence. In some embodiments, the polynucleotide sequence encoding the donor sequence further comprises a promoter in between the stop codon and the donor sequence. In some embodiments, the stop codon, the promoter, and the donor sequence are flanked on both ends (the 5′ and 3′ ends) with a sequence the gRNA targets. In some embodiments, the stop codon, the donor sequence, and the 3′ portion of the mutant NF1 gene are in the same reading frame. In some embodiments, the mutant NF1 gene comprises a mutation in the 3′ portion of the mutant NF1 gene, and the polynucleotide sequence encoding the donor sequence further comprises a stop codon that is 3′ to the donor sequence. In some embodiments, the gRNA targets a sequence upstream of the donor sequence and targets a sequence downstream of the stop codon that is 3′ to the donor sequence. In some embodiments, the 5′ portion of the mutant NF1 gene, the donor sequence, and the stop codon are in the same reading frame. In some embodiments, the gRNA targets a sequence flanking both sides of the polynucleotide sequence encoding the donor sequence and the stop codon. In some embodiments, the donor sequence comprises multiple exons of the wild-type NF1 gene or a functional equivalent thereof. In some embodiments, the donor sequence comprises one or more exons selected from exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, and exon 57 of the wild-type NF1 gene or a functional equivalent thereof. In some embodiments, the donor sequence comprises one or more contiguous exons of the wild-type NF1 gene or a functional equivalent thereof. In some embodiments, the donor sequence comprises exons 1-30 of the wild-type NF1 gene, and the gRNA targets a fragment of a mutant NF1 gene between exon 30 and exon 31. In some embodiments, the gRNA comprises a polynucleotide sequence selected from SEQ ID NOs: 71-81 or a complement thereof or a truncation thereof. In some embodiments, the gRNA is encoded by a polynucleotide sequence selected from SEQ ID NOs: 60-70 or a complement thereof or a truncation thereof, and/or hybridizes to a polynucleotide sequence selected from SEQ ID NOs: 49-59 or a complement thereof or a truncation thereof. In some embodiments, the donor sequence comprises a polynucleotide sequence of SEQ ID NO: 82. In some embodiments, the Cas protein is a Streptococcus pyogenes Cas9 protein or a Staphylococcus aureus Cas9 protein. In some embodiments, the Cas protein comprises an amino acid sequence of SEQ ID NO: 20 or SEQ ID NO: 21. In some embodiments, the vector is a viral vector. In some embodiments, the vector is an Adeno-associated virus (AAV) vector. In some embodiments, the AAV vector is AAV1, AAV2, AAV3, AAV4, AAV5, AAV6, AAV7, AAV8, AAV9, AAV-10, AAV-11, AAV-12, AAV-13, or AAVrh.74 vector. In some embodiments, one of the one or more vectors comprises a polynucleotide sequence selected from SEQ ID NOs: 83-102. In some embodiments, the molar ratio between the gRNA and the donor sequence is 1:1, or 1:5, or from 5:1 to 1:10, or from 1:1 to 1:5.

In a further aspect, the disclosure relates to a cell comprising a system as detailed herein.

Another aspect of the disclosure provides a composition for restoring NF1 function in a cell having a mutant NF1 gene, the composition comprising a system as detailed herein or a cell as detailed herein.

Another aspect of the disclosure provides a kit comprising a system as detailed herein, a cell as detailed herein, or a composition as detailed herein.

Another aspect of the disclosure provides a method for restoring NF1 function in a cell or a subject having a mutant NF1 gene. The method may include contacting the cell or the subject with a system as detailed herein, a cell as detailed herein, or a composition as detailed herein. In some embodiments, NF1 function is restored by inserting one or more wild-type exons of NF1 gene corresponding to the mutant NF1 gene.

The disclosure provides for other aspects and embodiments that will be apparent in light of the following detailed description and accompanying figures.

### BRIEF DESCRIPTION OF THE DRAWINGS

FIG. 1 is schematic diagram of the mechanism to insert the correct sequence of the NF1 allele. Two vectors are delivered: the first containing SaCas9 and a gRNA targeting an intronic region between exon 30 and exon 31, and the second containing the coding sequence of exons 1-30, since the patient mutation is in the 5′ half of the gene (denoted by the star). The second vector includes a donor sequence comprising exons 1-30 of the wild-type NF1 gene with a stop codon upstream of a promoter at the 5′-end, flanked on both sides by a target site for the gRNA. The SaCas9-gRNA complex would then cut in three locations: in the middle of the NF1 gene and on either side of the donor sequence, liberating it from the vector, and allowing it to be used to repair the genomic double-strand break via non-homologous end-joining to restore the correct NF1 gene sequence. The donor sequence and the remaining 3′ half of the chromosomal NF1 gene will then be transcribed, spliced together, and translated, creating functional neurofibromin.

FIG. 2A is a graph of percent indels for each gRNA, showing the results from the Surveyor assay for gene editing efficiency for six wild-type SaCas9 gRNAs (gRNAs 1-4, 6, and 10) and five gRNAs for use with a relaxed PAM requiring KKH SaCas9 (gRNAs 5, 7, 8, 9, and 11) identified by in silico analysis. gRNAs were chosen based on their location in the intronic region between exon 30 and exon 31, no predicted off targets, and cross-reactivity between mice and humans. Following plasmid transfection in HEK293T cells, editing efficiency was measured using the Surveyor assay. *P<0.001 compared to 0% indel, One-way ANOVA, Turkey-Kramer HSD; error bars, s.e.m.; n=2; ND=not detectable. FIG. 2B is a graph comparing the editing efficiency results for the gRNAs 1-4, 6, and 10, as determined by Sanger sequencing analyzed by Tracking of Indels by Decomposition (TIDE) and next-generation sequencing analyzed by the CRISPResso software pipeline. Error bars, s.e.m.; n=2.

FIG. 3A is schematic diagram of the location of the PCR primers used to confirm the integration of the donor sequence with In-Out PCR. FIG. 3B is a gel from In-Out PCR, showing insertion for both the 5′ and 3′ primer pairs for all three replicates of SaCas9, gRNA 1, and Donor, but no band for the negative controls as expected. Shown in FIG. 3C are pie graphs of the classification of next-generation sequencing reads of the three biological replicates of SaCas9, gRNA 1, and Donor 1.

FIG. 4A is schematic diagram of the location of the PCR primers used to quantify and classify RNA transcripts from the integrated donor sequence. FIG. 4B is a graph showing results from deep sequencing, confirming that the donor sequence is transcribed and spliced into exon 31. *P<0.001, Student’s t-test; error bars, s.e.m. FIG. 4C is a graph showing the percent of Isoform 1 or Isoform 2 reads containing the donor sequence.

FIG. 5A is a schematic of the experiment to evaluate donor insertion in vivo. FIG. 5B is a gel showing the amplified band from In-Out PCR using the 5′ primer pair for mouse #1-4, and a schematic (bottom) of the sequencing results from mouse #3. FIG. 5C is a gel showing the amplified band from Nested In-Out PCR from mouse #1, and a schematic (bottom) of the sequencing results from mouse #1. FIG. 5D is a gel showing the amplified band from In-Out PCR using the 3′ primer pair for mouse #1-4, and a schematic (bottom) of the sequencing results from mouse #4, to confirm the donor insertion at the 3′-end of the NF1 gene.

### DETAILED DESCRIPTION

Provided herein are systems and methods that utilize a CRISPR/Cas-based strategy to restore the correct NF1 gene sequence, for example, through the nonhomologous end joining repair process in a subject suffering from NF1. The CRISPR/Cas-based genome editing systems detailed herein may include a polynucleotide sequence encoding a guide RNA (gRNA) targeting a fragment of a mutant NF1 gene, a polynucleotide sequence encoding a Cas protein or a fusion protein comprising the Cas protein, and a polynucleotide sequence encoding a donor sequence comprising a fragment of a wild-type NF1 gene. The systems may be used in compositions, kits, and methods for restoring NF1 function and/or treating Neurofibromatosis Type I (NF1). In some embodiments, the systems and methods comprise the delivery of one or two ~5 kb donor cassettes or vectors, encoding either the 5′ or the 3′ portion of the NF1 cDNA sequence, depending on the patient mutation, flanked by a S. aureus Cas9 (SaCas9) guide RNA (gRNA) target site that corresponds to a sequence in a middle portion of the NF1 gene. For mutations occurring in either the 5′ or the 3′ portion of the gene, the corresponding donor would be used. Co-delivery of SaCas9 and the gRNA with the appropriate donor leads to a double-strand break in the middle portion of the NF1 gene and on both sides of the donor sequence, creating free DNA ends for ligation of the donor sequence into the genomic double-strand break, thereby restoring a wild-type NF1 coding sequence. The systems and methods provided herein result in the production of functional neurofibromin and lead to a reduction in Ras signaling and a decrease in the size and number of tumors in a NF1 mouse model. Further provided herein are methods of treating a subject suffering from, or at risk of developing, NF1 using the systems and methods provided herein. The systems and methods provided herein are estimated to be applicable to over 90% of NF1 patients.

The compositions and methods detailed herein have several advantages over conventional gene replacement and gene editing approaches. For example, the compositions and methods detailed herein include the ability for the gene to be regulated by endogenous machinery, and the ability to correct a large portion of the mutations in the patient population with a single donor sequence. Additionally, for diseases like NF1 that lack a prevalent mutation or a hot spot, the compositions and methods detailed here provide a viable strategy to treat a significant portion of the patient population.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Binding region” as used herein refers to the region within a target region that is recognized and bound by the CRISPR/Cas-based gene editing system.

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea.

“Coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The regulatory elements may include, for example, a promoter, an enhancer, an initiation codon, a stop codon, or a polyadenylation signal. The coding sequence may be codon optimized.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P.J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be a subject or cell without a composition as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Correcting”, “gene editing,” and “restoring” as used herein refers to changing a mutant gene that encodes a dysfunctional protein or truncated protein or no protein at all, such that a full-length functional or partially full-length functional protein expression is obtained. Correcting or restoring a mutant gene may include replacing the region of the gene that has the mutation or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include disrupting an aberrant splice acceptor site or splice donor sequence. Correcting or restoring a mutant gene may also include deleting a non-essential gene segment by the simultaneous action of two nucleases on the same DNA strand in order to restore the proper reading frame by removing the DNA between the two nuclease target sites and repairing the DNA break by NHEJ.

“Donor DNA”, “donor template,” and “repair template” as used interchangeably herein refers to a double-stranded DNA fragment or molecule that includes at least a portion of the gene of interest. The donor DNA may encode a full-functional protein or a partially functional protein.

“Enhancer” as used herein refers to non-coding DNA sequences containing multiple activator and repressor binding sites. Enhancers range from 200 bp to 1 kb in length and may be either proximal, 5′ upstream to the promoter or within the first intron of the regulated gene, or distal, in introns of neighboring genes or intergenic regions far away from the locus. Through DNA looping, active enhancers contact the promoter dependently of the core DNA binding motif promoter specificity. 4 to 5 enhancers may interact with a promoter. Similarly, enhancers may regulate more than one gene without linkage restriction and may “skip” neighboring genes to regulate more distant ones. Transcriptional regulation may involve elements located in a chromosome different to one where the promoter resides. Proximal enhancers or promoters of neighboring genes may serve as platforms to recruit more distal elements.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Functional” and “full-functional” as used herein describes protein that has biological activity. A “functional gene” refers to a gene transcribed to mRNA, which is translated to a functional protein.

“Fusion protein” as used herein refers to a chimeric protein created through the joining of two or more genes that originally coded for separate proteins. The translation of the fusion gene results in a single polypeptide with functional properties derived from each of the original proteins.

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed. The regulatory elements may include, for example, a promoter, an enhancer, an initiation codon, a stop codon, or a polyadenylation signal.

“Genome editing” or “gene editing” as used herein refers to changing the DNA sequence of a gene. Genome editing may include correcting or restoring a mutant gene or adding additional mutations. Genome editing may include knocking out a gene, such as a mutant gene or a normal gene. Genome editing may be used to treat disease or, for example, enhance muscle repair, by changing the gene of interest. In some embodiments, the compositions and methods detailed herein are for use in somatic cells and not germ line cells.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more subsequences that are not found in the same relationship to each other in nature (for example, a “fusion protein,” where the two subsequences are encoded by a single nucleic acid sequence).

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas-based gene editing system, then the cellular machinery will repair the break by homologous recombination, which is enhanced several orders of magnitude in the presence of DNA cleavage. When the homologous DNA piece is absent, non-homologous end joining may take place instead.

“Identical” or “identity” as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” as used herein refers to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible. “Nuclease mediated NHEJ” as used herein refers to NHEJ that is initiated after a nuclease cuts double stranded DNA.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo-and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function. Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter. Promoters that target muscle-specific stem cells may include the CK8 promoter, the Spc5-12 promoter, and the MHCK7 promoter.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Sample” or “test sample” as used herein can mean any sample in which the presence and/or level of a target is to be detected or determined or any sample comprising a DNA targeting or gene editing system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” as used herein refers to any nucleotide sequence encoding a known or putative gene product. The target gene may be a mutated gene involved in a genetic disease. The target gene may encode a known or putative gene product that is intended to be corrected or for which its expression is intended to be modulated. In certain embodiments, the target gene is the NF1 gene.

“Target region” as used herein refers to the region of the target gene to which the CRISPR/Cas-based gene editing or targeting system is designed to bind. In some embodiments, the target region is within an intronic region between exon 30 and exon 31 of the NF1 gene.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism’s genetic code. The introduction of a transgene has the potential to change the phenotype of an organism.

“Transcriptional regulatory elements” or “regulatory elements” refers to a genetic element which can control the expression of nucleic acid sequences, such as activate, enhancer, or decrease expression, or alter the spatial and/or temporal expression of a nucleic acid sequence. Examples of regulatory elements include, for example, promoters, enhancers, splicing signals, polyadenylation signals, and termination signals. A regulatory element can be “endogenous,” “exogenous,” or “heterologous” with respect to the gene to which it is operably linked. An “endogenous” regulatory element is one which is naturally linked with a given gene in the genome. An “exogenous” or “heterologous” regulatory element is one which is not normally linked with a given gene but is placed in operable linkage with a gene by genetic manipulation.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid sequence containing an origin of replication. A vector may be capable of directing the delivery or transfer of a polynucleotide sequence to target cells, where it can be replicated or expressed. A vector may contain an origin of replication, one or more regulatory elements, and/or one or more coding sequences. A vector may be a viral vector, bacteriophage, bacterial artificial chromosome, plasmid, cosmid, or yeast artificial chromosome. A vector may be a DNA or RNA vector. A vector may be a self-replicating extrachromosomal vector. Viral vectors include, but are not limited to, adenovirus vector, adeno-associated virus (AAV) vector, retrovirus vector, or lentivirus vector. A vector may be an adeno-associated virus (AAV) vector. The vector may encode a Cas9 protein and at least one gRNA molecule.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Neurofibromatosis Type I (NF1)

Neurofibromatosis Type I (NF1; also known as von Recklinghausen syndrome) is an autosomal dominant disease caused by the loss of function of the protein neurofibromin (which may also be referred to a NF1). Loss of neurofibromin leads to the formation of malignant and benign neurofibromas originating in non-dividing cells that form the myelin sheath of peripheral nerves, called Schwann cells. Additional symptoms of NF1 may include disfiguring cutaneous neurofibromas (CNF), café au lait pigment spots, plexiform neurofibromas (PN), skeletal defects, optic nerve gliomas, life-threatening malignant peripheral nerve sheath tumors (MPNST), pheochromocytoma, attention deficits, learning deficits, and other cognitive disabilities. NF1 affects 1 in 3,000 people worldwide, however, there is currently no effective treatment to reduce the size or number of neurofibromas.

Neurofibromin is a GTPase-activating protein (GAP) that negatively regulates RAS/MAPK cellular growth and proliferation pathway activity by accelerating the hydrolysis of Ras-bound GTP. Neurofibromin primarily regulates the protein Ras. Human neurofibromin is a 320-kDa protein that includes 2,818 amino acids. Neurofibromin localizes in the cytoplasm, however, some studies have found neurofibromin or fragments of it in the nucleus. Neurofibromin is ubiquitously expressed, but expression levels may vary depending on the tissue type and developmental stage of the organism. Expression may be at its highest level in adult neurons, Schwann cells, astrocytes, leukocytes, and oligodendrocytes. Schwann cells (also referred to as neurolemmocytes) are the principal glia of the peripheral nervous system (PNS). Glial cells function to support neurons. The two types of Schwann cells are myelinating and nonmyelinating. Myelinating Schwann cells wrap around axons of motor and sensory neurons to form the myelin sheath. Schwann cells are involved in many aspects of peripheral nerve biology, such as, for example, the conduction of nervous impulses along axons, nerve development and regeneration, trophic support for neurons, production of the nerve extracellular matrix, modulation of neuromuscular synaptic activity, and presentation of antigens to T-lymphocytes.

NF1 is located on the long arm of chromosome 17, position q11.2, in humans. NF1 spans over 350-kb of genomic DNA and contains 57 exons in humans. NF1 has one of the highest mutation rates amongst known human genes, however, mutation detection is often difficult because of its large size and the variety of possible mutations. The NF1 locus has a high incidence of de novo mutations, meaning that the mutations are not inherited maternally or paternally. Although the mutation rate is high, there are no mutation “hot spot” regions. Mutations tend to be distributed within the gene, although exons 3, 5, and 27 are common sites for mutations. In some embodiments, mutations in the NF1 gene affect splicing.

An NF1 gene may be a mutant NF1 gene. An NF1 gene may be a wild-type NF1 gene. An NF1 gene may have a sequence that is functionally identical to a wild-type NF1 gene, for example, the sequence may be codon-optimized but still encode for the same protein as the wild-type NF1. A mutant NF1 gene may include one or more mutations relative to the wild-type NF1 gene. Mutations may include, for example, nucleotide deletions, substitutions, additions, transversions, or combinations thereof. A mutation in the NF1 gene may affect splicing of the NF1 gene. A mutation in the NF1 gene may be a functional deletion of the NF1 gene. In some embodiments, the mutation in the NF1 gene comprises an insertion or deletion in the NF1 gene that prevents protein expression from the NF1 gene. Mutations may be in one or more exons and/or introns. Mutations may include deletions of all or parts of at least one intron and/or exon. An exon of a mutant NF1 gene may be mutated or at least partially deleted from the NF1 gene. An exon of a mutant NF1 gene may be fully deleted. A mutant NF1 gene may have a portion or fragment thereof that corresponds to the corresponding sequence in the wild-type NF1 gene. In some embodiments, a disrupted NF1 gene caused by a deleted or mutated exon can be restored in NF1 patients by adding back the corresponding wild-type exon. In some embodiments, the mutation(s) in the mutant NF1 gene is present in the 5′ portion of the gene. In some embodiments, the mutation(s) in the mutant NF1 gene is present in the 3′ portion of the gene. In some embodiments, one or more exons may be added and inserted so as to restore the corresponding mutated or deleted exon(s) in NF1.

### 3. CRISPR/Cas-Based Gene Editing System

Provided herein are CRISPR/Cas-based gene editing systems (also referred to as CRISPR/Cas-based genome editing systems). The CRISPR/Cas-based gene editing system may be used to insert a donor sequence to correct a mutant gene sequence on a chromosome and result in expression of a functional protein. The CRISPR/Cas-based gene editing system may include a Cas protein or a fusion protein, and at least one gRNA, and may also be referred to as a “CRISPR-Cas system.”

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a “memory” of past exposures. Cas proteins include, for example, Cas12a, Cas9, and Cascade proteins. Cas12a may also be referred to as “Cpf1.” Cas12a causes a staggered cut in double stranded DNA, while Cas9 produces a blunt cut. In some embodiments, the Cas protein comprises Cas12a. In some embodiments, the Cas protein comprises Cas9. Cas9 forms a complex with the 3′ end of the sgRNA (which may be referred interchangeably herein as “gRNA”), and the protein-RNA pair recognizes its genomic target by complementary base pairing between the 5′ end of the gRNA sequence and a predefined 20 bp DNA sequence, known as the protospacer. This complex is directed to homologous loci of pathogen DNA via regions encoded within the crRNA, i.e., the protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed gRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and III effector systems) are known. The Type II effector system carries out targeted DNA double-strand break in four sequential steps, using a single effector enzyme, Cas9, to cleave dsDNA. Compared to the Type I and Type III effector systems, which require multiple distinct effectors acting as a complex, the Type II effector system may function in alternative contexts such as eukaryotic cells. The Type II effector system consists of a long pre-crRNA, which is transcribed from the spacer-containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9:crRNA-tracrRNA complex. Cas12a systems include crRNA for successful targeting, whereas Cas9 systems include both crRNA and tracrRNA.

The Cas9:crRNA-tracrRNA complex unwinds the DNA duplex and searches for sequences matching the crRNA to cleave. Target recognition occurs upon detection of complementarity between a “protospacer” sequence in the target DNA and the remaining spacer sequence in the crRNA. Cas9 mediates cleavage of target DNA if a correct protospacer-adjacent motif (PAM) is also present at the 3′ end of the protospacer. For protospacer targeting, the sequence must be immediately followed by the protospacer-adjacent motif (PAM), a short sequence recognized by the Cas9 nuclease that is required for DNA cleavage. Different Cas and Cas Type II systems have differing PAM requirements. For example, Cas12a may function with PAM sequences rich in thymine “T.”

An engineered form of the Type II effector system of S. pyogenes was shown to function in human cells for genome engineering. In this system, the Cas9 protein was directed to genomic target sites by a synthetically reconstituted “guide RNA” (“gRNA”, also used interchangeably herein as a chimeric single guide RNA (“sgRNA”)), which is a crRNA-tracrRNA fusion that obviates the need for RNase III and crRNA processing in general. Provided herein are CRISPR/Cas-based engineered systems for use in gene editing and treating genetic diseases. The CRISPR/Cas-based engineered systems can be designed to target any gene, including genes involved in, for example, a genetic disease, aging, tissue regeneration, or wound healing. The CRISPR/Cas-based gene editing system can include a Cas9 protein or a Cas9 fusion protein.

### A. Cas9 Protein

Cas9 protein is an endonuclease that cleaves nucleic acid and is encoded by the CRISPR loci and is involved in the Type II CRISPR system. The Cas9 protein can be from any bacterial or archaea species, including, but not limited to, Streptococcus pyogenes, Staphylococcus aureus (S. aureus), Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni, Campylobacter lari, Candidatus Puniceispirillum, Clostridium cellulolyticum, Clostridium perfringens. Corynebacterium accolens, Corynebacterium diphtheria, Corynebacterium matruchotii, Dinoroseobacter shibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae. In certain embodiments, the Cas9 molecule is a Streptococcus pyogenes Cas9 molecule (also referred herein as “SpCas9”). SpCas9 may comprise an amino acid sequence of SEQ ID NO: 20. In certain embodiments, the Cas9 molecule is a Staphylococcus aureus Cas9 molecule (also referred herein as “SaCas9”). SaCas9 may comprise an amino acid sequence of SEQ ID NO: 21.

A Cas9 molecule or a Cas9 fusion protein can interact with one or more gRNA molecule(s) and, in concert with the gRNA molecule(s), can localize to a site which comprises a target domain, and in certain embodiments, a PAM sequence. The Cas9 protein forms a complex with the 3′ end of a gRNA. The ability of a Cas9 molecule or a Cas9 fusion protein to recognize a PAM sequence can be determined, for example, by using a transformation assay as known in the art.

The specificity of the CRISPR-based system may depend on two factors: the target sequence and the protospacer-adjacent motif (PAM). The target sequence is located on the 5′ end of the gRNA and is designed to bond with base pairs on the host DNA at the correct DNA sequence known as the protospacer. By simply exchanging the recognition sequence of the gRNA, the Cas9 protein can be directed to new genomic targets. The PAM sequence is located on the DNA to be altered and is recognized by a Cas9 protein. PAM recognition sequences of the Cas9 protein can be species specific.

In certain embodiments, the ability of a Cas9 molecule or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 molecules from different bacterial species can recognize different sequence motifs (for example, PAM sequences). A Cas9 molecule of S. pyogenes may recognize the PAM sequence of NRG (5′-NRG-3′, where R is any nucleotide residue, and in some embodiments, R is either A or G, SEQ ID NO: 1). In certain embodiments, a Cas9 molecule of S. pyogenes may naturally prefer and recognize the sequence motif NGG (SEQ ID NO: 2) and directs cleavage of a target nucleic acid sequence 1 to 10, for example, 3 to 5, bp upstream from that sequence. In some embodiments, a Cas9 molecule of S. pyogenes accepts other PAM sequences, such as NAG (SEQ ID NO: 3) in engineered systems (Hsu et al., Nature Biotechnology 2013 doi:10.1038/nbt.2647). In certain embodiments, a Cas9 molecule of S. thermophilus recognizes the sequence motif NGGNG (SEQ ID NO: 4) and/or NNAGAAW (W = A or T) (SEQ ID NO: 5) and directs cleavage of a target nucleic acid sequence 1 to 10, for example, 3 to 5, bp upstream from these sequences. In certain embodiments, a Cas9 molecule of S. mutans recognizes the sequence motif NGG (SEQ ID NO: 2) and/or NAAR (R = A or G) (SEQ ID NO: 6) and directs cleavage of a target nucleic acid sequence 1 to 10, for example, 3 to 5 bp, upstream from this sequence. In certain embodiments, a Cas9 molecule of S. aureus recognizes the sequence motif NNGRR (R = A or G) (SEQ ID NO: 7) and directs cleavage of a target nucleic acid sequence 1 to 10, for example, 3 to 5, bp upstream from that sequence. In certain embodiments, a Cas9 molecule of S. aureus recognizes the sequence motif NNGRRN (R = A or G) (SEQ ID NO: 8) and directs cleavage of a target nucleic acid sequence 1 to 10, for example, 3 to 5, bp upstream from that sequence. In certain embodiments, a Cas9 molecule of S. aureus recognizes the sequence motif NNGRRT (R = A or G) (SEQ ID NO: 9) and directs cleavage of a target nucleic acid sequence 1 to 10, for example, 3 to 5, bp upstream from that sequence. In certain embodiments, a Cas9 molecule of S. aureus recognizes the sequence motif NNGRRV (R = A or G; V = A or C or G) (SEQ ID NO: 10) and directs cleavage of a target nucleic acid sequence 1 to 10, for example, 3 to 5, bp upstream from that sequence. A Cas9 molecule derived from Neisseria meningitidis (NmCas9) normally has a native PAM of NNNNGATT (SEQ ID NO: 11), but may have activity across a variety of PAMs, including a highly degenerate NNNNGNNN PAM (SEQ ID NO: 12) (Esvelt et al. Nature Methods 2013 doi:10.1038/nmeth.2681). In the aforementioned embodiments, N can be any nucleotide residue, for example, any of A, G, C, or T. Cas9 molecules can be engineered to alter the PAM specificity of the Cas9 molecule.

In some embodiments, the Cas9 protein recognizes a PAM sequence NGG (SEQ ID NO: 2) or NGA (SEQ ID NO: 13) or NNNRRT (R = A or G) (SEQ ID NO: 14) or ATTCCT (SEQ ID NO: 15) or NGAN (SEQ ID NO: 16) or NGNG (SEQ ID NO: 17). In some embodiments, the Cas9 protein is a Cas9 protein of S. aureus and recognizes the sequence motif NNGRR (R = A or G) (SEQ ID NO: 7), NNGRRN (R = A or G) (SEQ ID NO: 8), NNGRRT (R = A or G) (SEQ ID NO: 9), or NNGRRV (R = A or G; V = A or C or G) (SEQ ID NO: 10). In the aforementioned embodiments, N can be any nucleotide residue, for example, any of A, G, C, or T.

Additionally or alternatively, a nucleic acid encoding a Cas9 molecule or Cas9 polypeptide may comprise a nuclear localization sequence (NLS). Nuclear localization sequences are known in the art, for example, SV40 NLS (Pro-Lys-Lys-Lys-Arg-Lys-Val; SEQ ID NO: 35).

In some embodiments, the Cas9 protein is a VQR variant. The VQR variant of Cas9 is a mutant with a different PAM recognition, as detailed in Kleinstiver, et al. (Nature 2015, 523, 481-485, incorporated herein by reference).

A polynucleotide encoding a Cas9 molecule can be a synthetic polynucleotide. For example, the synthetic polynucleotide can be chemically modified. The synthetic polynucleotide can be codon optimized, for example, at least one non-common codon or less-common codon has been replaced by a common codon. For example, the synthetic polynucleotide can direct the synthesis of an optimized messenger mRNA, for example, optimized for expression in a mammalian expression system, as described herein. An exemplary codon optimized nucleic acid sequence encoding a Cas9 molecule of S. pyogenes is set forth in SEQ ID NO: 26. Exemplary codon optimized nucleic acid sequences encoding a Cas9 molecule of S. aureus, and optionally containing nuclear localization sequences (NLSs), are set forth in SEQ ID NOs: 27-33. Another exemplary codon optimized nucleic acid sequence encoding a Cas9 molecule of S. aureus comprises the nucleotides 1293-4451 of SEQ ID NO: 34.

### B. Guide RNA (gRNA)

The CRISPR/Cas-based gene editing system includes at least one gRNA molecule. For example, the CRISPR/Cas-based gene editing system may include two gRNA molecules. The at least one gRNA molecule can recognize and bind a target region. The gRNA is the part of the CRISPR-Cas system that provides DNA targeting specificity to the CRISPR/Cas-based gene editing system. The gRNA is a fusion of two noncoding RNAs: a crRNA and a tracrRNA. gRNA mimics the naturally occurring crRNA:tracrRNA duplex involved in the Type II Effector system. This duplex, which may include, for example, a 42-nucleotide crRNA and a 75-nucleotide tracrRNA, acts as a guide for the Cas9 to bind, and in some cases, cleave the target nucleic acid. The gRNA may target any desired DNA sequence by exchanging the sequence encoding a 20 bp protospacer which confers targeting specificity through complementary base pairing with the desired DNA target. The “target region” or “target sequence” or “protospacer” refers to the region of the target gene to which the CRISPR/Cas-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas-based gene editing system targets and binds; “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The constant region of the gRNA may include the sequence of SEQ ID NO: 19 (RNA), which is encoded by a sequence comprising SEQ ID NO: 18 (DNA). The CRISPR/Cas-based gene editing system may include at least one gRNA, wherein the gRNAs target different DNA sequences. The target DNA sequences may be overlapping. The gRNA may comprise at its 5′ end the targeting domain that is sufficiently complementary to the target region to be able to hybridize to, for example, about 10 to about 20 nucleotides of the target region of the target gene, when it is followed by an appropriate Protospacer Adjacent Motif (PAM). The target region or protospacer is followed by a PAM sequence at the 3′ end of the protospacer in the genome. Different Type II systems have differing PAM requirements, as detailed above.

The targeting domain of the gRNA does not need to be perfectly complementary to the target region of the target DNA. In some embodiments, the targeting domain of the gRNA is at least 80%, 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19, or 20 nucleotides. For example, the DNA-targeting domain of the gRNA may be at least 80% complementary over at least 18 nucleotides of the target region. The target region may be on either strand of the target DNA.

The gRNA may target a region within the NF1 gene. The gRNA may target a fragment or portion of a wild-type NF1 gene. The gRNA may target a fragment or portion of a mutant NF1 gene. The gRNA may target a sequence in a middle region of the NF1 gene. The middle region of the NF1 gene may include the middle 5%, 10%, 15%, 20%, 25%, 30%, 33%, or 35% of the nucleotide sequence of the gene. For example, the gRNA may target a sequence within an intronic region between exon 30 and exon 31 of the NF1 gene. As further detailed below, the gRNA may target a sequence in a vector. The gRNA may bind and target and/or hybridize to a polynucleotide sequence comprising at least one of SEQ ID NOs: 49-59, or a complement thereof, or a variant thereof, or a truncation thereof, as shown in TABLE 1. The gRNA may be encoded by a polynucleotide sequence comprising at least one of SEQ ID NOs: 60-70, or a complement thereof, or a variant thereof, or a truncation thereof (TABLE 2). The gRNA may comprise a polynucleotide sequence selected from SEQ ID NOs: 71-81, or a complement thereof, or a variant thereof, or a truncation thereof (TABLE 2). The gRNA may bind and target and/or hybridize to, and/or be encoded by, and/or comprise, a polynucleotide sequence comprising at least one of SEQ ID NOs: 49-81, or a complement thereof, or a variant thereof, or a truncation thereof. A truncation may be 1, 2, 3, 4, 5, 6, 7, 8, or 9 nucleotides shorter than the reference sequence.

TABLE 1

| | | | |
|---|---|---|---|
| | PAM (Human) | PAM (Mouse) | Sequence the gRNA binds to (5′ to 3′) |
| g1 | TTGGGT (SEQ ID NO: 36) | TTGGGT (SEQ ID NO: 36) | TGTCTACTATTTCTGGC (SEQ ID NO: 49) |
| g2 | CTGAAT (SEQ ID NO: 37) | CTGAAT (SEQ ID NO: 37) | TAGGGAGTGGCAAGTTGCCT (SEQ ID NO: 50) |
| g3 | CAGGGT (SEQ ID NO: 38) | CAGGGT (SEQ ID NO: 38) | GAATTAAGTTACATTGAAAC (SEQ ID NO: 51) |
| g4 | GAGAAT (SEQ ID NO: 39) | GAGAAT (SEQ ID NO: 39) | TGTGTTCCTTTGGTTTGATT (SEQ ID NO: 52) |
| g5 | TTTGAT (SEQ ID NO: 40) | TTTGAT (SEQ ID NO: 40) | CCAAAGGAACACAGAGAATGT (SEQ ID NO: 53) |
| g6 | TAGAAT (SEQ ID NO: 41) | TAGAAT (SEQ ID NO: 41) | CTAATTCTGGCACAAAATAGC (SEQ ID NO: 54) |
| g7 | TTCAAT (SEQ ID NO: 42) | TTCAAT (SEQ ID NO: 42) | ACATTCTCTGTGTTCCTTTGG (SEQ ID NO: 55) |
| g8 | AATGAT (SEQ ID NO: 43) | AATGAT (SEQ ID NO: 43) | CTACTAATTCTGGCACAAAAT (SEQ ID NO: 56) |
| g9 | AGAGAT (SEQ ID NO: 44) | AGAGAT (SEQ ID NO: 44) | GTTCTGTAGGCAACTTGCCAC (SEQ ID NO: 57) |
| g10 | ATGAAT (SEQ ID NO: 45) | ATAAGT (SEQ ID NO: 47) | AGCCAGAAATAGTAGACATGA (SEQ ID NO: 58) |
| g11 | TGAAAT (SEQ ID NO: 46) | CGAAAT (SEQ ID NO: 48) | ATTAACCCTGGAATTAAGTTA (SEQ ID NO: 59) |

TABLE 2

| | | |
|---|---|---|
| | DNA encoding gRNA (5′ to 3′) (protospacer) | gRNA (5′ to 3′) |
| g1 | GCCAGAAATAGTAGACA (SEQ ID NO: 60) | GCCAGAAAUAGUAGACA (SEQ ID NO: 71) |
| g2 | AGGCAACTTGCCACTCCCTA (SEQ ID NO: 61) | AGGCAACUUGCCACUCCCUA (SEQ ID NO: 72) |
| g3 | GTTTCAATGTAACTTAATTC (SEQ ID NO: 62) | GUUUCAAUGUAACUUAAUUC (SEQ ID NO: 73) |
| g4 | AATCAAACCAAAGGAACACA (SEQ ID NO: 63) | AAUCAAACCAAAGGAACACA (SEQ ID NO: 74) |
| g5 | ACATTCTCTGTGTTCCTTTGG (SEQ ID NO: 64) | ACAUUCUCUGUGUUCCUUUGG (SEQ ID NO: 75) |
| g6 | GCTATTTTGTGCCAGAATTAG (SEQ ID NO: 65) | GCUAUUUUGUGCCAGAAUUAG (SEQ ID NO: 76) |
| g7 | CCAAAGGAACACAGAGAATGT (SEQ ID NO: 66) | CCAAAGGAACACAGAGAAUGU (SEQ ID NO: 77) |
| g8 | ATTTTGTGCCAGAATTAGTAG (SEQ ID NO: 67) | AUUUUGUGCCAGAAUUAGUAG (SEQ ID NO: 78) |
| g9 | GTGGCAAGTTGCCTACAGAAC (SEQ ID NO: 68) | GUGGCAAGUUGCCUACAGAAC (SEQ ID NO: 79) |
| g10 | TCATGTCTACTATTTCTGGCT (SEQ ID NO: 69) | UCAUGUCUACUAUUUCUGGCU (SEQ ID NO: 80) |
| g11 | TAACTTAATTCCAGGGTTAAT (SEQ ID NO: 70) | UAACUUAAUUCCAGGGUUAAU (SEQ ID NO: 81) |

As described above, the gRNA molecule comprises a targeting domain (also referred to as targeted or targeting sequence), which is a polynucleotide sequence complementary to the target DNA sequence. The gRNA may comprise a “G” at the 5′ end of the targeting domain or complementary polynucleotide sequence. The CRISPR/Cas-based gene editing system may use gRNAs of varying sequences and lengths. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The number of gRNA molecules that may be included in the CRISPR/Cas-based gene editing system can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA molecules that may be included in the CRISPR/Cas-based gene editing system can be less than 50 different gRNAs, less than 45 different gRNAs, less than 40 different gRNAs, less than 35 different gRNAs, less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs, less than 12 different gRNAs, less than 11 different gRNAs, less than 10 different gRNAs, less than 9 different gRNAs, less than 8 different gRNAs, less than 7 different gRNAs, less than 6 different gRNAs, less than 5 different gRNAs, less than 4 different gRNAs, less than 3 different gRNAs, or less than 2 different gRNAs. The number of gRNAs that may be included in the CRISPR/Cas-based gene editing system can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs.

### C. Donor Sequence

The CRISPR/Cas-based gene editing system may include at least one donor sequence. A donor sequence comprises a polynucleotide sequence to be inserted into a genome. A donor sequence may comprise a wild-type sequence of a gene. A donor sequence may comprise the wild-type NF1 gene. A donor sequence may comprise a fragment or portion of the wild-type NF1 gene. The fragment or portion of the NF1 gene may be, for example, about 50% of the full nucleotide sequence of the gene. The fragment or portion of the NF1 gene may be at least about 30%, at least about 35%, at least about 40%, at least about 45%, or at least about 50% of the full nucleotide sequence of the gene. The fragment or portion of the NF1 gene may be less than about 70%, less than about 65%, less than about 60%, less than about 55%, or less than about 50% of the full nucleotide sequence of the gene. The fragment or portion of the NF1 gene may include about 20-80%, about 30-70%, about 40-60%, or about 45-55% of the full nucleotide sequence of the gene. The donor sequence may include a 5′ portion or fragment of the wild-type NF1 gene. The 5′ portion or fragment of the NF1 gene may include the 5′ end of the NF1 gene including about 20-80%, about 30-70%, about 40-60%, or about 45-55% of the full nucleotide sequence of the gene. The donor sequence may include the 5′ end of the wild-type NF1 gene including about 20-80%, about 30-70%, about 40-60%, or about 45-55% of the full nucleotide sequence of the gene. The donor sequence may include a 3′ portion or fragment of the wild-type NF1 gene. The 3′ portion or fragment of the NF1 gene may include the 3′ end of the NF1 gene including about 20-80%, about 30-70%, about 40-60%, or about 45-55% of the full nucleotide sequence of the gene. The donor sequence may include the 3′ end of the wild-type NF1 gene including about 20-80%, about 30-70%, about 40-60%, or about 45-55% of the full nucleotide sequence of the gene.

A donor sequence may comprise multiple exons of the wild-type NF1 gene. In some embodiments, the donor sequence comprises one or more exons selected from exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, and exon 57 of the wild-type NF1 gene or a functional equivalent thereof. In some embodiments, the donor sequence comprises one or more contiguous exons of the wild-type NF1 gene or a functional equivalent thereof. In some embodiments, the donor sequence comprises exons 1-30 of the wild-type NF1 gene.

The donor sequence may comprise a polynucleotide sequence of SEQ ID NO: 82.

The gRNA and donor sequence may be present in a variety of molar ratios. The molar ratio between the gRNA and donor sequence may be 1:1, or 1:15, or from 5:1 to 1:10, or from 1:1 to 1:5. The molar ratio between the gRNA and donor sequence may be at least 1:1, at least 1:2, at least 1:3, at least 1:4, at least 1:5, at least 1:6, at least 1:7, at least 1:8, at least 1:9, at least 1:10, at least 1:15, or at least 1:20. The molar ratio between the gRNA and donor sequence may be less than 20:1, less than 15:1, less than 10:1, less than 9:1, less than 8:1, less than 7:1, less than 6:1, less than 5:1, less than 4:1, less than 3:1, less than 2:1, or less than 1:1.

### D. Repair Pathways

The CRISPR/Cas-based gene editing system may be used to introduce site-specific double strand breaks at targeted genomic loci, such as a location within the NF1 gene. Site-specific double-strand breaks are created when the CRISPR/Cas-based gene editing system binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

### I) Homology-Directed Repair (HDR)

Restoration of protein expression from a gene may involve homology-directed repair (HDR). A donor template may be administered to a cell. The donor template may include a nucleotide sequence encoding a full-functional protein or a partially functional protein. In such embodiments, the donor template may include fully functional gene construct for restoring a mutant gene, or a fragment of the gene that after homology-directed repair, leads to restoration of the mutant gene. In other embodiments, the donor template may include a nucleotide sequence encoding a mutated version of an inhibitory regulatory element of a gene. Mutations may include, for example, nucleotide substitutions, insertions, deletions, or a combination thereof. In such embodiments, introduced mutation(s) into the inhibitory regulatory element of the gene may reduce the transcription of or binding to the inhibitory regulatory element.

### II) NHEJ

Restoration of protein expression from gene may be through template-free NHEJ-mediated DNA repair. In certain embodiments, NHEJ is a nuclease mediated NHEJ, which in certain embodiments, refers to NHEJ that is initiated a Cas9 molecule that cuts double stranded DNA. The method comprises administering a presently disclosed CRISPR/Cas-based gene editing system or a composition comprising thereof to a subject for gene editing.

Nuclease mediated NHEJ may correct a mutated target gene and offer several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment.

### 4. Genetic Constructs

The CRISPR/Cas-based gene editing system may be encoded by or comprised within one or more genetic constructs. The CRISPR/Cas-based gene editing system may comprise one or more genetic constructs. The genetic construct, such as a plasmid or expression vector, may comprise a nucleic acid that encodes the CRISPR/Cas-based gene editing system and/or at least one gRNA and/or a donor sequence.

In some embodiments, the CRISPR/Cas-based gene editing system includes two vectors: a first vector and a second vector. The first vector may encode a Cas protein or a fusion protein, and at least one gRNA. The polynucleotide sequence encoding the gRNA and the polynucleotide sequence encoding the Cas protein or the fusion protein may be operably linked. The second vector may encode a donor sequence.

In some embodiments, the polynucleotide sequence encoding the donor sequence further comprises a stop codon. The stop codon may be 5′ or 3′ to the donor sequence. The stop codon may be upstream or downstream of the donor sequence. The stop codon and the donor sequence may be in the same reading frame.

The vector may further encode a promoter. The first vector may encode a promoter 5′ to or upstream of the polynucleotide encoding the Cas protein or the fusion protein. The first vector may encode a promoter 5′ to or upstream of the polynucleotide encoding the at least one gRNA. The second vector may encode a promoter 5′ to or upstream of the donor sequence. The promoters may be the same or different.

In some embodiments, the mutant NF1 gene comprises a mutation in the 5′ portion of the mutant NF1 gene, and the polynucleotide sequence encoding the donor sequence further comprises a stop codon that is 5′ to or upstream of the donor sequence. As detailed above, the 5′ portion or fragment of the NF1 gene may include the 3′ end of the NF1 gene including about 20-80%, about 30-70%, about 40-60%, or about 45-55% of the full nucleotide sequence of the gene. The polynucleotide sequence may further include a promoter in between the stop codon and the donor sequence. The stop codon, the promoter, and the donor sequence may be flanked on both ends (the 5′ and 3′ ends) with a sequence the gRNA targets. In such embodiments, the gRNA targets a sequence upstream of the stop codon that is 5′ to or upstream of the donor sequence in the second vector, and also targets a sequence 3′ to or downstream of the donor sequence in the second vector. In some embodiments, upon integration of the donor sequence into the chromosome, the stop codon, the donor sequence, and the 3′ portion of the mutant NF1 gene are in the same reading frame.

In some embodiments, the mutant NF1 gene comprises a mutation in the 3′ portion of the mutant NF1 gene, and the polynucleotide sequence encoding the donor sequence further comprises a stop codon that is 3′ to or downstream of the donor sequence. As detailed above, the 3′ portion or fragment of the NF1 gene may include the 3′ end of the NF1 gene including about 20-80%, about 30-70%, about 40-60%, or about 45-55% of the full nucleotide sequence of the gene. The stop codon and the donor sequence may be flanked on both ends (the 5′ and 3′ ends) with a sequence the gRNA targets. In such embodiments, the gRNA targets a sequence 5′ to or upstream of the donor sequence in the second vector, and also targets a sequence 3′ to or downstream of the stop codon that is 5′ to or upstream of the donor sequence in the second vector. In some embodiments, upon integration of the donor sequence into the chromosome, the 5′ portion of the mutant NF1 gene, the donor sequence, and the stop codon are in the same reading frame. In some embodiments, upon integration of the donor sequence into the chromosome, the 5′ portion of the mutant NF1 gene, the donor sequence, and the stop codon are in the same reading frame, such that the donor sequence and 5′ portion of the chromosomal NF1 gene would be transcribed together and rely on the endogenous promoter.

The promoters may be a constitutive promoter, an inducible promoter, a repressible promoter, or a regulatable promoter. The promoter may be a ubiquitous promoter. The promoter may be a tissue-specific promoter. The tissue specific promoter may be a muscle specific promoter. The tissue specific promoter may be a skin specific promoter. The CRISPR/Cas-based gene editing system may be under the light-inducible or chemically inducible control to enable the dynamic control of gene/genome editing in space and time. The promoter operably linked to the CRISPR/Cas-based gene editing system coding sequence may be a promoter from simian virus 40 (SV40), a mouse mammary tumor virus (MMTV) promoter, a human immunodeficiency virus (HIV) promoter such as the bovine immunodeficiency virus (BlV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. Examples of a tissue specific promoter, such as a muscle or skin specific promoter, natural or synthetic, are described in U.S. Pat. Application Publication No. US20040175727, the contents of which are incorporated herein in its entirety. The promoter may be a CK8 promoter, a Spc512 promoter, a MHCK7 promoter, for example. In some embodiments, the vector may also comprise an additional promoter that is operably linked to the CRISPR/Cas-based gene editing system coding sequence.

In certain embodiments, a genetic construct encodes one gRNA molecule, i.e., a first gRNA molecule, and optionally a Cas9 molecule or fusion protein. In some embodiments, a genetic construct encodes two gRNA molecules, i.e., a first gRNA molecule and a second gRNA molecule, and optionally a Cas9 molecule or fusion protein. In some embodiments, a first genetic construct encodes one gRNA molecule, i.e., a first gRNA molecule, and optionally a Cas9 molecule or fusion protein, and a second genetic construct encodes one gRNA molecule, i.e., a second gRNA molecule, and optionally a Cas9 molecule or fusion protein. In some embodiments, a first genetic construct encodes one gRNA molecule and one donor sequence, and a second genetic construct encodes a Cas9 molecule or fusion protein. In some embodiments, a first genetic construct encodes one gRNA molecule and a Cas9 molecule or fusion protein, and a second genetic construct encodes one donor sequence.

Genetic constructs may include polynucleotides such as vectors and plasmids. The genetic construct may be a linear minichromosome including centromere, telomeres, or plasmids or cosmids. The vector may be an expression vectors or system to produce protein by routine techniques and readily available starting materials including Sambrook et al., Molecular Cloning and Laboratory Manual, Second Ed., Cold Spring Harbor (1989), which is incorporated fully by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may comprise heterologous nucleic acid encoding the CRISPR/Cas-based gene editing system and may further comprise an initiation codon, which may be upstream of the CRISPR/Cas-based gene editing system coding sequence, and another stop codon, which may be downstream of the CRISPR/Cas-based gene editing system coding sequence. The genetic construct may include more than one stop codon, which may be downstream of the CRISPR/Cas-based gene editing system coding sequence. In some embodiments, the genetic construct includes 1, 2, 3, 4, or 5 stop codons. In some embodiments, the genetic construct includes 1, 2, 3, 4, or 5 stop codons downstream of the sequence encoding the donor sequence. A stop codon may be in-frame with a coding sequence in the CRISPR/Cas-based gene editing system. For example, one or more stop codons may be in-frame with the donor sequence. The genetic construct may include one or more stop codons that are out of frame of a coding sequence in the CRISPR/Cas-based gene editing system. For example, one stop codon may be in-frame with the donor sequence, and two other stop codons may be included that are in the other two possible reading frames. A genetic construct may include a stop codon for all three potential reading frames. The initiation and termination codon may be in frame with the CRISPR/Cas-based gene editing system coding sequence.

The genetic construct may also comprise a polyadenylation signal, which may be downstream of the CRISPR/Cas-based gene editing system. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 vector (Invitrogen, San Diego, CA).

Coding sequences in the genetic construct may be optimized for stability and high levels of expression. In some instances, codons are selected to reduce secondary structure formation of the RNA such as that formed due to intramolecular bonding.

The genetic construct may also comprise an enhancer upstream of the CRISPR/Cas-based gene editing system or gRNAs. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as green fluorescent protein (“GFP”) and/or a selectable marker, such as hygromycin (“Hygro”).

The genetic construct may be useful for transfecting cells with nucleic acid encoding the CRISPR/Cas-based gene editing system, which the transformed host cell is cultured and maintained under conditions wherein expression of the CRISPR/Cas-based gene editing system takes place. The genetic construct may be transformed or transduced into a cell. The genetic construct may be formulated into any suitable type of delivery vehicle including, for example, a viral vector, lentiviral expression, mRNA electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a system or component thereof as detailed herein. Suitable cell types are detailed herein. In some embodiments, the cell is a stem cell. The stem cell may be a human stem cell. In some embodiments, the cell is an embryonic stem cell. The stem cell may be a human pluripotent stem cell (iPSCs). Further provided are stem cell-derived neurons, such as neurons derived from iPSCs transformed or transduced with a DNA targeting system or component thereof as detailed herein.

### A. Viral Vectors

A genetic construct may be a viral vector. Further provided herein is a viral delivery system. Viral delivery systems may include, for example, lentivirus, retrovirus, adenovirus, mRNA electroporation, or nanoparticles. In some embodiments, the vector is a modified lentiviral vector. In some embodiments, the viral vector is an adeno-associated virus (AAV) vector. The AAV vector is a small virus belonging to the genus Dependovirus of the Parvoviridae family that infects humans and some other primate species.

AAV vectors may be used to deliver CRISPR/Cas-based gene editing systems using various construct configurations. For example, AAV vectors may deliver Cas9 or fusion protein and gRNA expression cassettes on separate vectors or on the same vector. Alternatively, if the small Cas9 proteins or fusion proteins, derived from species such as Staphylococcus aureus or Neisseria meningitidis, are used then both the Cas9 and up to two gRNA expression cassettes may be combined in a single AAV vector. In some embodiments, the AAV vector has a 4.7 kb packaging limit.

In some embodiments, the AAV vector is a modified AAV vector. The modified AAV vector may have enhanced cardiac and/or skeletal muscle tissue tropism. The modified AAV vector may be capable of delivering and expressing the CRISPR/Cas-based gene editing system in the cell of a mammal. For example, the modified AAV vector may be an AAV-SASTG vector (Piacentino et al. Human Gene Therapy 2012, 23, 635-646). The modified AAV vector may be based on one or more of several capsid types, including AAV1, AAV2, AAV5, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV2i8G9 (Shen et al. J. Biol. Chem. 2013, 288, 28814-28823).

The genetic construct may comprise a polynucleotide sequence selected from SEQ ID NOs: 83-86, 87-90, 91-102, a complement thereof, or a fragment thereof.

### 5. Pharmaceutical Compositions

Further provided herein are pharmaceutical compositions comprising the above-described genetic constructs or gene editing systems. In some embodiments, the pharmaceutical composition may comprise about 1 ng to about 10 mg of DNA encoding the CRISPR/Cas-based gene editing system. The systems or genetic constructs as detailed herein, or at least one component thereof, may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The term “pharmaceutically acceptable carrier,” may be a non-toxic, inert solid, semi-solid or liquid filler, diluent, encapsulating material or formulation auxiliary of any type. Pharmaceutically acceptable carriers include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate, and more preferably, the poly-L-glutamate may be present in the composition for gene editing in skeletal muscle or cardiac muscle at a concentration less than 6 mg/mL.

### 6. Administration

The systems or genetic constructs as detailed herein, or at least one component thereof, may be administered or delivered to a cell. Methods of introducing a nucleic acid into a host cell are known in the art, and any known method can be used to introduce a nucleic acid (e.g., an expression construct) into a cell. Suitable methods include, for example, viral or bacteriophage infection, transfection, conjugation, protoplast fusion, polycation or lipid:nucleic acid conjugates, lipofection, electroporation, nucleofection, immunoliposomes, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. The system, genetic construct, or composition comprising the same, may be electroporated using BioRad Gene Pulser Xcell or Amaxa Nucleofector IIb devices or other electroporation device. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

The systems or genetic constructs as detailed herein, or at least one component thereof, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the system, genetic construct, or composition comprising the same, is administered to a subject intramuscularly, intravenously, or a combination thereof. The systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the brain or other component of the central nervous system. The composition may be injected into the skeletal muscle or cardiac muscle. For example, the composition may be injected into the tibialis anterior muscle or tail. For veterinary use, the systems, genetic constructs, or compositions comprising the same may be administered as a suitably acceptable formulation in accordance with normal veterinary practice. The veterinarian may readily determine the dosing regimen and route of administration that is most appropriate for a particular animal. The systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound. Alternatively, transient in vivo delivery of CRISPR/Cas-based systems by non-viral or non-integrating viral gene transfer, or by direct delivery of purified proteins and gRNAs containing cell-penetrating motifs may enable highly specific correction and/or restoration in situ with minimal or no risk of exogenous DNA integration.

Upon delivery of the presently disclosed systems or genetic constructs as detailed herein, or at least one component thereof, or the pharmaceutical compositions comprising the same, and thereupon the vector into the cells of the subject, the transfected cells may express the gRNA molecule(s) and the Cas9 molecule or fusion protein.

### A. Cell Types

Any of the delivery methods and/or routes of administration detailed herein can be utilized with a myriad of cell types. Further provided herein is a cell transformed or transduced with a system or component thereof as detailed herein. For example, provided herein is a cell comprising an isolated polynucleotide encoding a CRISPR/Cas system as detailed herein. Suitable cell types are detailed herein. In some embodiments, the cell is an immune cell. Immune cells may include, for example, lymphocytes such as T cells and B cells and natural killer (NK) cells. In some embodiments, the cell is a T cell. T cells may be divided into cytotoxic T cells and helper T cells, which are in turn categorized as TH1 or TH2 helper T cells. Immune cells may further include innate immune cells, adaptive immune cells, tumor-primed T cells, NKT cells, IFN-y producing killer dendritic cells (IKDC), memory T cells (TCMs), and effector T cells (TEs). The cell may be a stem cell such as a human stem cell. In some embodiments, the cell is an embryonic stem cell or a hematopoietic stem cell. The stem cell may be a human induced pluripotent stem cell (iPSCs). Further provided are stem cell-derived neurons, such as neurons derived from iPSCs transformed or transduced with a DNA targeting system or component thereof as detailed herein. The cell may be a muscle cell. Cells may further include, but are not limited to, immortalized myoblast cells, dermal fibroblasts, bone marrow-derived progenitors, skeletal muscle progenitors, human skeletal myoblasts, CD 133+ cells, mesoangioblasts, cardiomyocytes, hepatocytes, chondrocytes, mesenchymal progenitor cells, hematopoietic stem cells, smooth muscle cells, and MyoD- or Pax7-transduced cells, or other myogenic progenitor cells.

### 7. Kits

Provided herein is a kit, which may be used to modify or correct a NF1 gene, or to restore NF1 function, in a cell or a subject in need thereof. The kit comprises genetic constructs or a composition comprising the same, or a component thereof, for modifying or correcting a NF1 gene, or for restoring NF1 function, as described above, and instructions for using said composition or component thereof. In some embodiments, the kit comprises at least one gRNA comprising a polynucleotide sequence of one of SEQ ID NOs: 71-81, a complement thereof, a variant thereof, a truncation thereof, or fragment thereof, and/or at least one gRNA encoded by a polynucleotide comprising a sequence of one of SEQ ID NOs: 60-70, a complement thereof, a truncation thereof, a variant thereof, or fragment thereof, and/or at least one gRNA targeting and binding and/or hybridizing to a polynucleotide comprising a sequence of one of SEQ ID NOs: 49-59, a complement thereof, a variant thereof, a truncation thereof, or fragment thereof. The kit may further include instructions for using the CRISPR/Cas-based gene editing system.

Instructions included in kits may be affixed to packaging material or may be included as a package insert. While the instructions are typically written on printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

The genetic constructs or a composition comprising thereof for modifying or correcting a NF1 gene, or for restoring NF1 function, may include a modified AAV vector that includes a gRNA molecule(s) and a Cas9 protein or fusion protein, as described above, that specifically binds and cleaves a region of the NF1 gene. The CRISPR/Cas-based gene editing system, as described above, may be included in the kit to specifically bind and target a particular region, for example, an intronic region and/or a middle portion of the gene.

### 8. Methods

### A. Methods of Restoring NF1 Function

Provided herein are methods for restoring NF1 function in a cell or a subject having a mutant NF1 gene. The methods may include contacting the cell or the subject with a CRISPR/Cas-based genome editing system as detailed herein, a cell as detailed herein, or a genetic construct as detailed herein. In some embodiments, NF1 function is restored by inserting one or more wild-type exons of the NF1 gene corresponding to the mutant NF1 gene.

Further provided herein are methods for modifying or correcting a NF1 gene. The NF1 gene may be a mutant gene in a cell or in a subject. The methods may include contacting the cell or the subject with a CRISPR/Cas-based genome editing system as detailed herein, a cell as detailed herein, or a genetic construct as detailed herein. In some embodiments, NF1 function is restored by inserting one or more wild-type exons of the NF1 gene corresponding to the mutant NF1 gene.

### B. Methods of Treating Neurofibromatosis Type I

Provided herein are methods of treating Neurofibromatosis Type I (NF1) in a cell or in a subject in need thereof. The methods may include contacting the cell or the subject with a CRISPR/Cas-based genome editing system as detailed herein, a cell as detailed herein, or a genetic construct as detailed herein. In some embodiments, NF1 function is restored by inserting one or more wild-type exons of the NF1 gene corresponding to the mutant NF1 gene.

### 9. EXAMPLES

The foregoing may be better understood by reference to the following examples, which are presented for purposes of illustration and are not intended to limit the scope of the invention. The present disclosure has multiple aspects and embodiments, illustrated by the appended non-limiting examples.

### Example 1

### Developing a Gene-Editing Approach to Correct Neurofibromatosis Type I-Causal Mutations

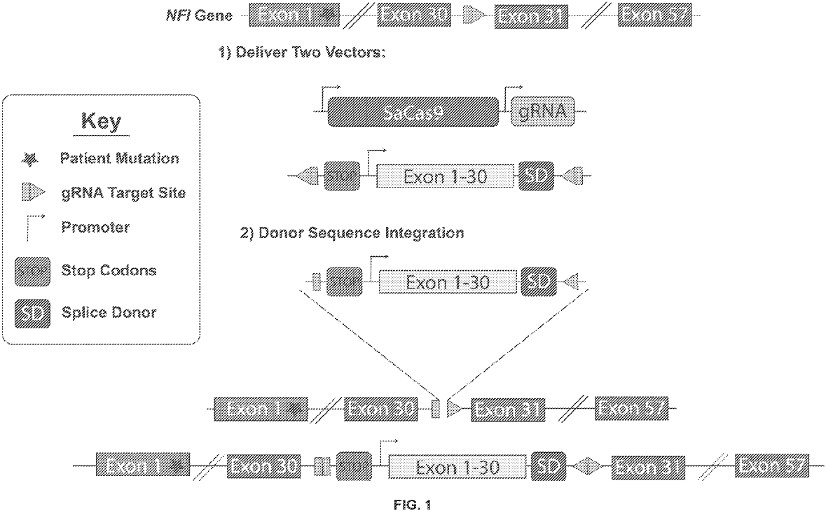

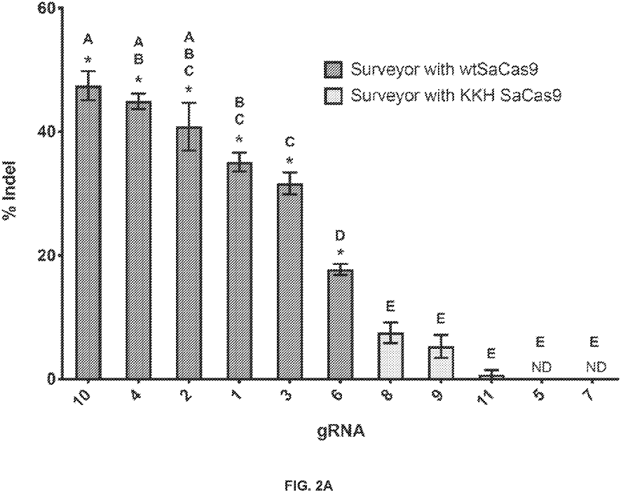

gRNAs were designed to target the middle region of the chromosomal NF1 gene, between exons 30 and 31. Shown in FIG. 1 is schematic diagram of a chromosomal mutant NF1 gene in a subject with exons 1-57 and a gRNA target site in an intronic region between exon 30 and exon 31, with a mutation in exon 1 indicated by a star. Further shown is a schematic of two vectors as detailed herein. One vector encodes SaCas9 and a gRNA. The other vector encodes a donor sequence comprising exons 1-30 of the wild-type NF1 gene with a stop codon upstream of a promoter at the 5′-end, flanked on both sides by a target site for the gRNA. The SaCas9-gRNA complex then cuts in three places: once in a middle portion of the chromosomal mutant NF1 gene, and on either side of the donor sequence. Cutting on either side of the donor sequence thereby liberates the donor sequence from the vector to be used to repair the double-strand break (DSB) in the middle portion of the chromosomal mutant NF1 gene via nonhomologous end joining. Upon integration of the donor sequence in between exon 30 and exon 31 of the chromosomal mutant NF1 gene, introduction of the stop codon results in a truncated protein from exons 1-30 of the mutant NF1 gene, but a full wild-type sequence downstream. The donor sequence and the remaining 3′ portion of the chromosomal NF1 gene is transcribed, spliced together, and translated, creating functional neurofibromin. Functional neurofibromin is expected to decrease Ras signaling and reduce the number and size of neurofibromas.

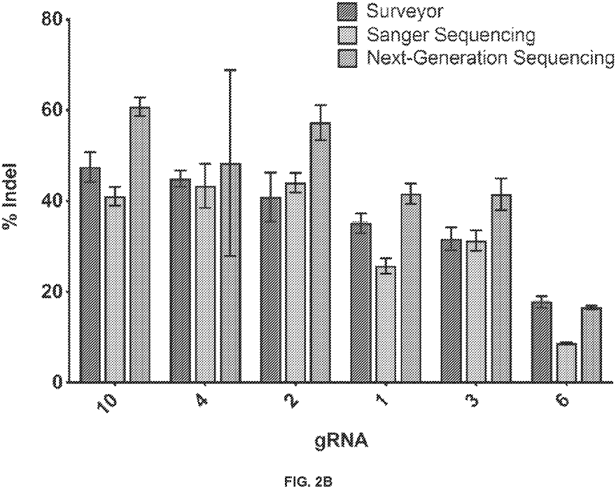

Several gRNAs were designed (TABLE 1 and TABLE 2), based on targeting regions in the intronic region between exon 30 and exon 31 in chromosomal NF1 gene, low predicted off-target activity, and activity in both mice and humans. Editing efficiency was measured using the Surveyor Assay, which uses the T7 endonuclease 1 (T7E1 )(Sentmanat et al. Scientific Reports 2018, 8, 888). T7E1 is a structure-selective enzyme that detects structural deformities in heteroduplexed DNA. To detect CRISPR-Cas9-mediated gene editing, CRISPR-Cas9 reagents were transfected into cells, and the genomic DNA surrounding the target locus was amplified by PCR several days later. The PCR product was denatured and recomplexed by heating and subsequent slow cooling. If an aberrant NHEJ event occurred after CRISPR-Cas9 cleavage, a heteroduplex formed between amplicons of different lengths (for example, mutant and wild-type amplicons), leading to a DNA distortion that was recognized and cleaved by T7E1. The banding patterns of the cut products were compared between control and experimental samples to determine the frequency of mutations. Results are shown in FIG. 2A as a graph of percent indels for each gRNA, showing the gene editing efficiency for six SaCas9 gRNAs (gRNAs 1-4, 6, and 10) and five gRNAs for use with a different PAM and KKH SaCas9 (gRNAs 5, 7, 8, 9, and 11). KKH SaCas9 is a Cas9 protein with a relaxed PAM requirement of NNNRRT (SEQ ID NO: 14) instead of NNGRRT (SEQ ID NO: 9)( Kleinstiver et al. Nature Biotechnology 2015, 33, 1293-1298). The editing efficiency with gRNAs 1-4, 6, and 10 was confirmed using Sanger sequencing analyzed by Tracking of Indels by Decomposition (TIDE) and Next-Generation Sequencing analysis by the CRISPResso software pipeline. Editing efficiency results are shown in FIG. 2B as a graph of percent indels for gRNAs 1-4, 6, and 10.

### Example 2

### In-Out PCR and Deep Sequencing Confirm Donor Sequence Insertion In Vitro

Donor cassettes containing the 5′ half of the NF1 cDNA were constructed for each of the top four performing gRNAs from Example 1 with the highest levels of editing activity. The gRNA and donor cassette pairs were then delivered with SaCas9 in HEK293T cells to evaluate the editing activity. Genomic DNA and RNA were harvested.

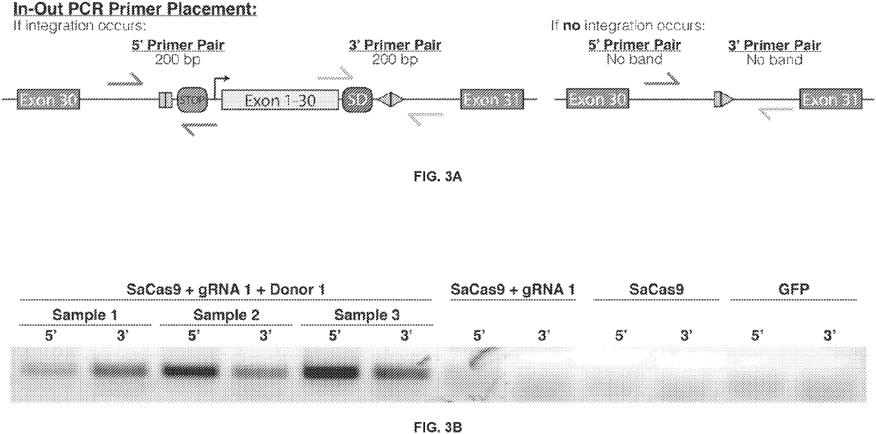

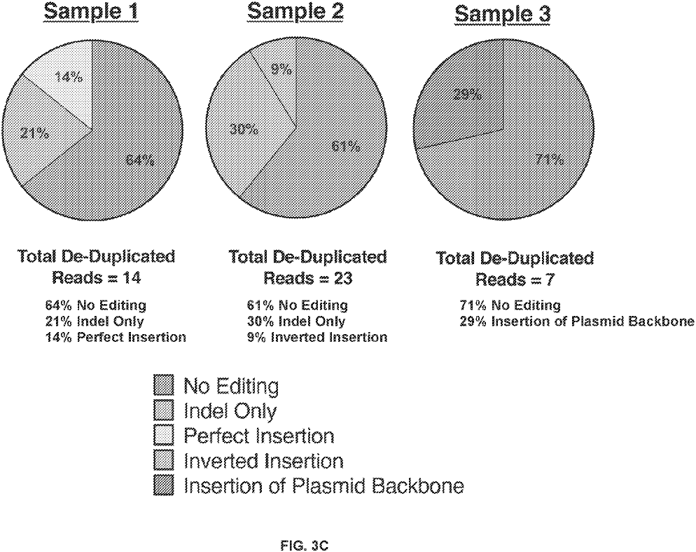

Insertion of the donor sequence was confirmed with In-Out PCR and deep sequencing. Shown in FIG. 3A is schematic diagram of the location of the PCR primers used to confirm the integration of the donor sequence with In-Out PCR. The 5′ primer pair (purple/darker arrows) had a forward primer specific to the intronic sequence upstream of the cut site, and a reverse primer specific to the 5′ half of the donor sequence. The 3′ primer pair (green/lighter arrows) had a forward primer specific to the 3′ half of the donor sequence, and a reverse primer specific to the intronic sequence downstream of the cut site. Accordingly, if an integration occurred in the correct orientation, then the 5′ primer pair and the 3′ primer pair produced a 200 bp amplicon, whereas no amplicon was produced if integration did not occur.

Shown in FIG. 3B are results of In-Out PCR to confirm donor integration. Three biological replicates in HEK293T cells were transfected with SaCas9, gRNA 1, and Donor 1. Additionally, one biological replicate was transfected for each negative control: SaCas9 + gRNA 1, SaCas9 only, and GFP. Genomic DNA was isolated after three days. Results from In-Out PCR showed insertion for both the 5′ and 3′ primer pairs for all three replicates of SaCas9, gRNA 1, and Donor 1. Negative controls did not produce a band as expected.

Shown in FIG. 3C are pie graphs of the classification of next-generation sequencing reads of the three biological replicates of SaCas9, gRNA 1, and Donor 1. Genomic DNA was sequenced with a low-biased Tn5-based next-generation sequencing approach (Nelson et al. Nature Medicine 2019, 25, 427-432). Reads were filtered to include reads at the correct locus and de-duplicated based on the location of the inserted transposon. Sequencing of the genomic DNA with targeted Tn5-based sequencing confirmed that the intended insertion occurred in ~8% of alleles for the top performing gRNA and donor cassette pair.

### Example 3

### Donor Sequence Is Transcribed and Spliced to Chromosomal Exon 31

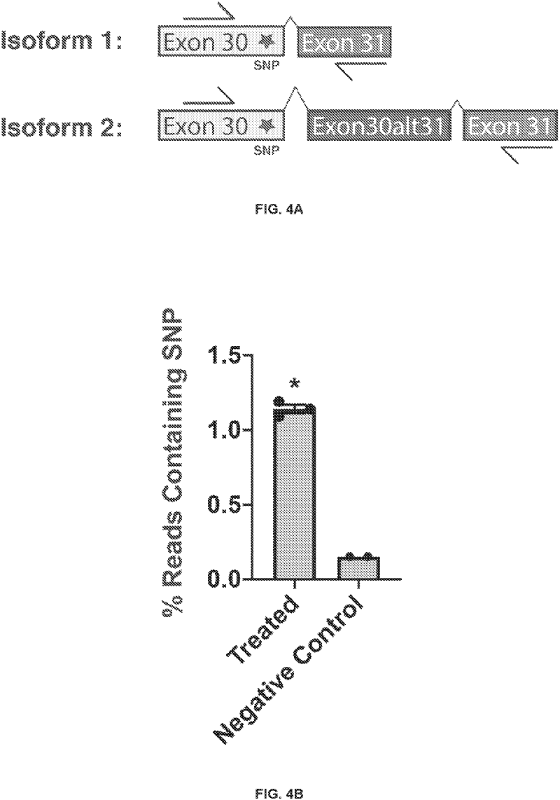

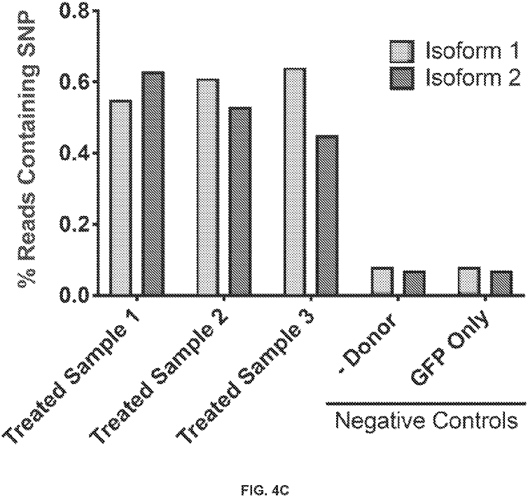

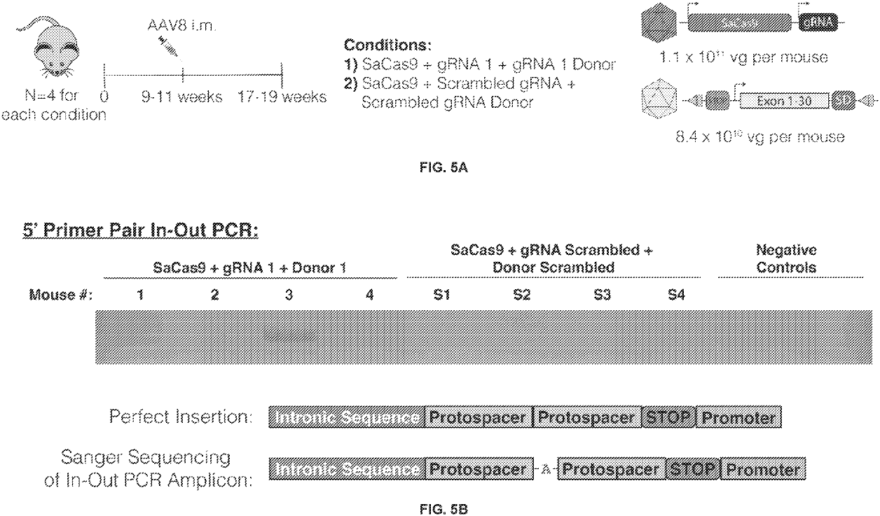

Shown in FIG. 4A is schematic diagram of the location of the PCR primers used to quantify and classify RNA transcripts from the integrated donor sequence. A single nucleotide polymorphism (SNP, shown as the star) was introduced into exon 30 of the donor sequence to differentiate transcripts with the integrated donor and from the unedited gene. HEK293T cells have two main isoforms of NF1, with Isoform 2 containing an alternately expressed exon, Exon30alt31.

Shown in FIG. 4B is a graph with the results from deep sequencing, confirming that the donor neurofibromin-coding sequence was transcribed and correctly spliced into endogenous exon 31 of NF1. Three biological replicates of HEK293T cells were transfected with SaCas9, gRNA 1, and Donor 1. Additionally, one biological replicate was transfected for each negative control: SaCas9 and gRNA 1, and GFP. Next-generation sequencing was used to count the number of reads that contained the SNP. Percent of negative control reads containing the SNP was consistent with the error rate of next-generation sequencing. FIG. 4C is a graph showing the percent of Isoform 1 or Isoform 2 reads containing the donor sequence. The classification of reads by isoform type showed that donor integration had no effect on the ratio of the two isoforms compared to the ration in unedited cells.

### Example 4

### In-Out PCR Confirms Donor Insertion In Vivo

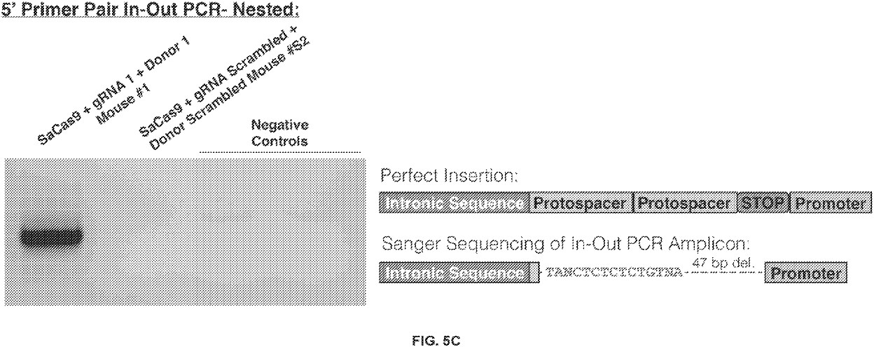

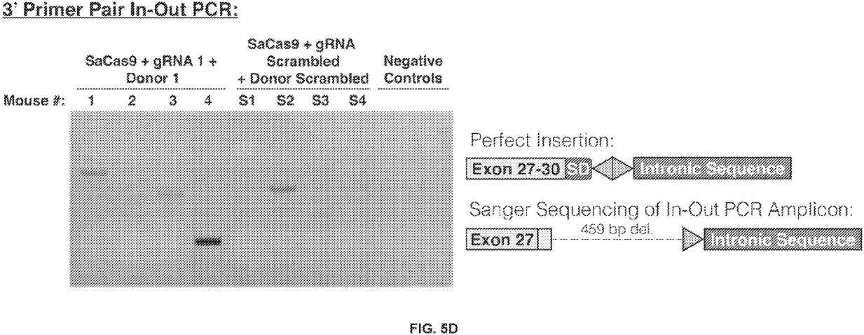

FIG. 5A is a schematic of the experiment to evaluate donor insertion in vivo. 9-11-week-old mice (N=4 per condition) were injected with one AAV8 vector containing SaCas9 and gRNA 1 or a scrambled gRNA, and another AAV8 vector containing the donor sequence for gRNA 1 or the scrambled gRNA. The two vectors were co-delivered by intramuscular injection into the wild-type mice. The mice were sacrificed after 8 weeks, and genomic DNA was isolated from muscle tissue. PCR was used across the insertion to confirm the targeted integration of the 5′ half of the NF1 cDNA in the skeletal muscle.

FIG. 5B is a gel showing the amplified band from In-Out PCR using the 5′ primer pair for mouse #1-4. In-Out PCR using the 5′ primer pair (as detailed above) for the scrambled condition and negative controls (unedited mouse genomic DNA and primer only) did not produce a band as expected for mouse #1, #2, and #4. In-Out PCR using the 5′ primer pair confirmed insertion of the donor sequence in mouse #3 treated with SaCas9, gRNA 1, and donor 1. The amplicon produced by mouse #3 was sequenced using Sanger sequencing and showed a one base pair insertion at the site of integration (bottom of FIG. 5B).

FIG. 5C is a gel showing the amplified band from Nested In-Out PCR from mouse #1. Nested In-Out PCR revealed integration of the donor sequence in mouse #1. The amplicon was sequenced using Sanger sequencing and revealed both a 15 bp insertion and a 47 bp deletion at the site of integration (bottom of FIG. 5C).

FIG. 5D is a gel showing the amplified band from In-Out PCR using the 3′ primer pair for mouse #1-4. In-Out PCR using the 3′ primer pair confirmed insertion of the donor sequence in mouse #4 that treated with SaCas9, gRNA 1, and Donor 1. The amplicon was sequenced using Sanger sequencing and revealed a 459 bp deletion of the donor sequence at the site of integration (bottom of FIG. 5D). All other amplicons were unable to be sequenced.

As detailed herein, donor sequence integration has been confirmed, and transcription and correct splicing of the donor sequence into the remaining downstream portion of the gene has been shown. Donor insertion rate has been measured to be 5% of alleles.

The editing efficiency will be quantified in Schwann cells where the disease originates. The strategy will be further evaluated in human patient cell lines and in an NF1 mouse model to show that restoration of the correct NF1 sequence results in a phenotypic change and reduces Ras signaling as well as the number and size of neurofibromas.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A CRISPR/Cas-based genome editing system comprising: (a) a polynucleotide sequence encoding a guide RNA (gRNA) targeting a fragment of a mutant NF1 gene; (b) a polynucleotide sequence encoding a Cas protein or a fusion protein comprising the Cas protein; and (c) a polynucleotide sequence encoding a donor sequence comprising a fragment of a wild-type NF1 gene.

Clause 2. The system of clause 1, wherein the system comprises one or more vectors.

Clause 3. The system of clause 2, wherein the system comprises a first vector and a second vector, wherein the first vector comprises the polynucleotide sequence encoding the gRNA, and the polynucleotide sequence encoding the Cas protein or the fusion protein, and wherein the second vector comprises the polynucleotide sequence encoding the donor sequence.

Clause 4. The system of clause 3, wherein the polynucleotide sequence encoding the gRNA and the polynucleotide sequence encoding the Cas protein or the fusion protein are operably linked.

Clause 5. The system of any one of clauses 1-4, wherein the mutant NF1 gene comprises a mutation in the 5′ portion of the mutant NF1 gene, and wherein the polynucleotide sequence encoding the donor sequence further comprises a stop codon that is 5′ to the donor sequence.

Clause 6. The system of clause 5, wherein the gRNA targets a sequence upstream of the stop codon that is 5′ to the donor sequence and targets a sequence downstream of the donor sequence.

Clause 7. The system of clause 5 or 6, wherein the polynucleotide sequence encoding the donor sequence further comprises a promoter in between the stop codon and the donor sequence.

Clause 8. The system of clause 7, wherein the stop codon, the promoter, and the donor sequence are flanked on both ends (the 5′ and 3′ ends) with a sequence the gRNA targets.

Clause 9. The system of any one of clauses 5-8, wherein the stop codon, the donor sequence, and the 3′ portion of the mutant NF1 gene are in the same reading frame.

Clause 10. The system of any one of clauses 1-4, wherein the mutant NF1 gene comprises a mutation in the 3′ portion of the mutant NF1 gene, and wherein the polynucleotide sequence encoding the donor sequence further comprises a stop codon that is 3′ to the donor sequence.

Clause 11. The system of clause 10, wherein the gRNA targets a sequence upstream of the donor sequence and targets a sequence downstream of the stop codon that is 3′ to the donor sequence.

Clause 12. The system of clause 10 or 11, wherein the 5′ portion of the mutant NF1 gene, the donor sequence, and the stop codon are in the same reading frame.

Clause 13. The system of any one of clauses 5-12, wherein the gRNA targets a sequence flanking both sides of the polynucleotide sequence encoding the donor sequence and the stop codon.

Clause 14. The system of any one of clauses 1-13, wherein the donor sequence comprises multiple exons of the wild-type NF1 gene or a functional equivalent thereof.

Clause 15. The system of any one of clauses 1-14, wherein the donor sequence comprises one or more exons selected from exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, and exon 57 of the wild-type NF1 gene or a functional equivalent thereof.

Clause 16. The system of clause 15, wherein the donor sequence comprises one or more contiguous exons of the wild-type NF1 gene or a functional equivalent thereof.

Clause 17. The system of any one of clauses 1-16, wherein the donor sequence comprises exons 1-30 of the wild-type NF1 gene, and wherein the gRNA targets a fragment of a mutant NF1 gene between exon 30 and exon 31.

Clause 18. The system of any one of clauses 1-17, wherein the gRNA comprises a polynucleotide sequence selected from SEQ ID NOs: 71-81 or a complement thereof or a truncation thereof.

Clause 19. The system of any one of clauses 1-17, wherein the gRNA is encoded by a polynucleotide sequence selected from SEQ ID NOs: 60-70 or a complement thereof or a truncation thereof, and/or hybridizes to a polynucleotide sequence selected from SEQ ID NOs: 49-59 or a complement thereof or a truncation thereof.

Clause 20. The system of any one of clauses 1-19, wherein the donor sequence comprises a polynucleotide sequence of SEQ ID NO: 82.

Clause 21. The system of any one of clauses 1-20, wherein the Cas protein is a Streptococcus pyogenes Cas9 protein or a Staphylococcus aureus Cas9 protein.

Clause 22. The system of clause 21, wherein the Cas protein comprises an amino acid sequence of SEQ ID NO: 20 or SEQ ID NO: 21.

Clause 23. The system of any one of clauses 2-22, wherein the vector is a viral vector.

Clause 24. The system of clause 23, wherein the vector is an Adeno-associated virus (AAV) vector.

Clause 25. The system of clause 24, wherein the AAV vector is AAV1, AAV2, AAV3, AAV4, AAV5, AAV6, AAV7, AAV8, AAV9, AAV-10, AAV-11, AAV-12, AAV-13, or AAVrh.74 vector.

Clause 26. The system of clause 24, wherein one of the one or more vectors comprises a polynucleotide sequence selected from SEQ ID NOs: 83-102.

Clause 27. The system of any one of clauses 1-26, wherein the molar ratio between the gRNA and the donor sequence is 1:1, or 1:5, or from 5:1 to 1:10, or from 1:1 to 1:5.

Clause 28. A cell comprising the system of any one of clauses 1-27.

Clause 29. A composition for restoring NF1 function in a cell having a mutant NF1 gene, the composition comprising the system of any one of clauses 1-27 or the cell of clause 28.

Clause 30. A kit comprising the system of any one of clauses 1-27, the cell of clause 28, or the composition of clause 29.

Clause 31. A method for restoring NF1 function in a cell or a subject having a mutant NF1 gene, the method comprising contacting the cell or the subject with the system of any one of clauses 1-27, the cell of clause 28, or the composition of clause 29.

Clause 32. The method of clause 31, wherein NF1 function is restored by inserting one or more wild-type exons of NF1 gene corresponding to the mutant NF1 gene.

### SEQUENCES

SEQ ID NO: 1

NRG

(R = A or G; N can be any nucleotide residue, e.g., any of A, G, C, or T)

SEQ ID NO: 2

NGG

(N can be any nucleotide residue, e.g., any of A, G, C, or T)

SEQ ID NO: 3

NAG

(N can be any nucleotide residue, e.g., any of A, G, C, or T)

SEQ ID NO: 4

GGNG

(N can be any nucleotide residue, e.g., any of A, G, C, or T)

SEQ ID NO: 5

NNAGAAW

(W = A or T; N can be any nucleotide residue, e.g., any of A, G, C, or T)

SEQ ID NO: 6

NAAR

(R = A or G; N can be any nucleotide residue, e.g., any of A, G, C, or T)

SEQ ID NO: 7

NNGRR

(R = A or G; N can be any nucleotide residue, e.g., any of A, G, C, or T)

SEQ ID NO: 8

NNGRRN

(R = A or G; N can be any nucleotide residue, e.g., any of A, G, C, or T)

SEQ ID NO: 9

NNGRRT

(R = A or G; N can be any nucleotide residue, e.g., any of A, G, C, or T)

SEQ ID NO: 10

NNGRRV

(R = A or G; N can be any nucleotide residue, e.g., any of A, G, C, or T; V = A or C or G)

SEQ ID NO: 11

NNNNGATT

(N can be any nucleotide residue, e.g., any of A, G, C, or T)

SEQ ID NO: 12

NNNNGNNN

(N can be any nucleotide residue, e.g., any of A, G, C, or T)

SEQ ID NO: 13

NGA

(N can be any nucleotide residue, e.g., any of A, G, C, or T)

SEQ ID NO: 14

NNNRRT

(R = A or G; N can be any nucleotide residue, e.g., any of A, G, C, or T)

SEQ ID NO: 15

ATTCCT

SEQ ID NO: 16

NGAN

(N can be any nucleotide residue, e.g., any of A, G, C, or T)

SEQ ID NO: 17

NGNG

(N can be any nucleotide residue, e.g., any of A, G, C, or T)

SEQ ID NO: 18 DNA sequence of the gRNA constant region

gtttaagagctatgctggaaacagcatagcaagtttaaataaggctagtc
cgttateaacttgaaaaagtggcaccgagtcggtgc

SEQ ID NO: 19 RNA sequence of the gRNA constant region

guuuaagagcuaugcuggaaaeagcauagcaaguuuaaauaaggcuaguc
cguuaucaacuugaaaaaguggcaccgagucggugc

SEQ ID NO: 20 Streptococcus pyogenes Cas9

MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKWDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITUHVAQILDSRMNTKYDENDKLIR
EVKVITLKSKLVSDFRKDFQFYKVREINNYHHAHDAYLNAWGTALIKKYP
KLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQT
GGFSKESILPKRNSDKLIARKKDWDPKKYGGFDSPTVAYSVLWAKVEKGK
SKKLKSVKELLGITIMERSSFEKNPIDFLEAKGYKEVKKDLIIKLPKYSL
FELENGRKRMLASAGELQKGNELALPSKYVNFLYLASHYEKLKGSPEDNE
QKQLFVEQHKHYLDEIIEQISEFSKRVILADANLDKVLSAYNKHRDKPIR
EQAENIIHLFTLTNLGAPAAFKYFDTTIDRKRYTSTKEVLDATLIHQSIT
GLYETRIDLSQLGGD

SEQ ID NO: 21 Staphylococcus aureus Cas9

MKRNYILGLDIGITSVGYGIIDYETRDVID-AGVRLFKEANVENNEGRRS
KRGARRLKRRRRHRIQRVKKLLFDYNLLTDHSELSGINPYEARVKGLSQK
LSEEEFSAALLHLAKRRGVHNVNEVEEDTGNELSTKEQISRNSKALEEKY
VAELQLERLKKDGEVRGSINRFKTSDYVKEAKQLLKVQKAYHQLDQSFID
TYIDLLETRRTYYEGPGEGSPFGWKDIKEWYEMLMGHCTYFPEELRSVKY
AYNADLYNALNDLNNLVITRDENEKLEYYEKFQIIENVFKQKKKPTLKQI
AKEILVNEEDIKGYRVTSTGKPEFTNLKVYHDIKDITARKEIIENAELLD
QIAKILTIYQSSEDIQEELTNLNSELTQEEIEQISNLKGYTGTHNLSLKA
INLILDELWHTNDNQIAIFNRLKLVPKKVDLSQQKEIPTTLVDDFILSPW
KRSFIQSIKVINAIIKKYGLPNDIIIELAREKNSKDAQKMINEMQKRNRQ
TNERIEEIIRTTGKENAKYLIEKIKLHDMQEGKCLYSLEAIPLEDLLNNP
FNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTR
YATRGLMNLLRSYFRVNNLDVKVKSINGGFTSFLRRKWKFKKERNKGYKH
HAEDALIIANADFIFKEWKKLDKAKKVMENQMFEEKQAESMPEIETEQEY
KEIFITPHQIKHIKDFKDYKYSHRVDKKPNRELINDTLYSTRKDDKGNTL
IVNNLNGLYDKDNDKLKKLINKSPEKLLMYHHDPQTYQKLKLIMEQYGDE
KNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEA
KKLKKISNQAEFIASFYNNDLIKINGELYRVIGVNNDLLNRIEVNMIDIT
YREYLENMNDKRPPRIIKTIASKTQSIKKYSTDILGNLYEVKSKKHPQII
KKG

SEQ ID NO: 22 Streptococcus pyogenes Cas9 (with D10A)

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAWGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLWAKVEKG
KSKKLKSVKELLGITIMERSSFEKNPIDFLEAKGYKEVKKDLIIKLPKYS
LFELENGRKRMLASAGELQKGNELALPSKYVNFLYLASHYEKLKGSPEDN
EQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSI
TGLYETRIDLSQLGGD

SEQ ID NO: 23 Streptococcus pyogenes Cas9 (with D10A, H849A)

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEM4DGTEELLVKLNREDLLR
KQRTFDNGSIPHQIHLGELHAILRRQEDFYPFLKDNREKIEKILTFRIPY
YVGPLARGNSRFAWMTRKSEETITPWNFEEWDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKWDELVKVMG
RHKPENIVIEMARENQTTQKGQKNSRERMKRIEEGIKELGSQILKEHPVE
NTQLQNEKLYLYYLQNGRDMYVDQELDINRLSDYDVDAIVPQSFLKDDSI
DNKVLTRSDKNRGKSDNVPSEEVVKKMKNYWRQLLNAKLITQRKFDNLTK
AERGGLSELDKAGFIKRQLVETRQITKHVAQILDSRMNTKYDENDKLIRE
VKVITLKSKLVSDFRKDFQFYKVREINNYHHAHDAYLNAVVGTALIKKYP
KLESEFVYGDYKVYDVRKMIAKSEQEIGKATAKYFFYSNIMNFFKTEITL
ANGEIRKRPLIETNGETGEIVWDKGRDFATVRKVLSMPQVNIVKKTEVQT
GGFSKESILPKRNSDKLIARKKDWDPKKYGGFDSPTVAYSVLWAKVEKGK
SKKLKSVKELLGITIMERSSFEKNPIDFLEAKGYKEVKKDLIIKLPKYSL
FELENGRKRMLASAGELQKGNELALPSKYVNFLYLASHYEKLKGSPEDNE
QKQLFVEQHKHYLDE11EQISEFSKRVILADANLDKVLSAYNKHRDKPIR
EQAENIIHLFTLTNLGAPAAFKYFDTTIDRKRYTSTKEVLDATLIHQSIT
GLYETRIDLSQLGGD

SEQ ID NO: 24 Polynucleotide sequence of D10A mutant of S. aureus Cas9

atgaaaagga actacattct ggggctggcc atcgggatta caagcg
tggg gtatgggatt attgactatg aaacaaggga cgtgatcgac g
caggcgtca gactgttcaa ggaggccaac gtggaaaaca atgaggg
acg gagaagcaag aggggagcca ggcgcctgaa acgacggaga ag
gcacagaa tccagagggt gaagaaactg ctgttcgatt acaacctg
ct gaccgaccat tctgagctga gtggaattaa tccttatgaa gcc
agggtga aaggcctgag tcagaagctg tcagaggaag agttttccg
c agctctgctg cacctggcta agcgccgagg agtgcataac gtca
atgagg tggaagagga caccggcaac gagctgtcta caaaggaaca
gatctcacgc aatagcaaag ctctggaaga gaagtatgtc gcaga
gctgc agctggaacg gctgaagaaa gatggcgagg tgagagggtc
aattaatagg ttcaagacaa gcgactacgt caaagaagcc aagcag
ctgc tgaaagtgca gaaggcttac caccagctgg atcagagctt c
atcgatact tatatcgacc tgctggagac tcggagaacc tactatg
agg gaccaggaga agggagcccc ttcggatgga aagacatcaa gg
aatggtac gagatgctga tgggacattg cacctatttt ccagaaga
gc tgagaagcgt caagtacgct tataacgcag atctgtacaa cgc
cctgaat gacctgaaca acctggtcat caccagggat gaaaacgag
a aactggaata ctatgagaag ttccagatca tcgaaaacgt gttt
aagcag aagaaaaagc: ctacactgaa acagattgc taaggagatc
c tggtcaacga agaggacatc aagggctacc gggtgacaag cact
ggaaa accagagttca ccaatctgaa agtgtatcac gatattaagg
acatcacagc acggaaaga aatcattgaga acgccgaact gctgg
atcag attgctaaga tcctgactat ctaccagag ctccgaggaca
tccaggaaga gctgactaac ctgaacagcg agctgaccca ggaaga
gat cgaacagatta gtaatctgaa ggggtacacc ggaacacaca a
cctgtccct gaaagctat caatctgattc tggatgagct gtggcat
aca aacgacaatc agattgcaat ctttaaccg gctgaagctgg tc
ccaaaaaa ggtggacctg agtcagcaga aagagatccc aaccacac
t ggtggacgatt tcattctgtc acccgtggtc aagcggagct tca
tccagag catcaaagt gatcaacgcca tcatcaagaa gtacggcct
g cccaatgata tcattatcga gctggctag ggagaagaaca gcaa
ggacgc acagaagatg atcaatgaga tgcagaaacg aaaccggca
gaccaatgaac gcattgaaga gattatccga actaccggga aagag
aacgc aaagtacct gattgaaaaaa tcaagctgca cgatatgcag
gagggaaagt gtctgtattc tctggaggc catccccctgg aggacc
tgct gaacaatcca ttcaactacg aggtcgatca tattatccc ca
gaagcgtgt ccttcgacaa ttcctttaac aacaaggtgc tggtcaa
gca ggaagagaa ctctaaaaagg gcaataggac tcctttccag ta
cctgtcta gttcagattc caagatctc ttacgaaacctttaaaaagc
a cattctgaat ctggccaaag gaaagggccg catcagcaag acca
aaaagg agtacctgct ggaagagcgg gacatcaaca gattctccgt
ccagaaggat tttattaacc ggaatctggt ggacacaaga tacgc
tactc gcggcctgat gaatctgctg cgatcctatt tccgggtgaa
caatctggat gtgaaagtca agtccatcaa cggcgggttc acatct
tttc tgaggcgcaa atggaagttt aaaaaggagc gcaacaaagg g
tacaagcac catgccgaag atgctctgat tatcgcaaat gccgact
tca tctttaagga gtggaaaaag ctggacaaag ccaagaaagt ga
tggagaac cagatgttcg aagagaagca ggccgaatct atgcccga
aa tcgagacaga acaggagtac aaggagattt tcatcactcc tca
ccagatc aagcatatca aggatttcaa ggactacaag tactctcac
c gggtggataa aaagcccaac agagagctga tcaatgacac cctg
tatagt acaagaaaag acgataaggg gaataccctg attgtgaaca
atctgaacgg actgtacgac aaagataatg acaagctgaa aaagc
tgatc aacaaaagtc ccgagaagct gctgatgtac caccatgatc
ctcagacata tcagaaactg aagctgatta tggagcagta cggcga
cgag aagaacccac tgtataagta ctatgaagag actgggaact a
cctgaccaa gtatagcaaa aaggataatg gccccgtgat caagaag
atc aagtactatg ggaacaagct gaatgcccat ctggacatca ca
gacgatta ccctaacagt cgcaacaagg tggtcaagct gtcactga
ag ccatacagat tcgatgtcta tctggacaac ggcgtgtata aat
ttgtgac tgtcaagaat ctggatgtca tcaaaaagga gaactacta
t gaagtgaata gcaagtgcta cgaagaggct aaaaagctga aaaa
gattag caaccaggca gagttcatcg cctcctttta caacaacgac
ctgattaaga tcaatggcga actgtatagg gtcatcgggg tgaac
aatga tctgctgaac cgcattgaag tgaatatgat tgacatcact
taccgagagt atctggaaaa catgaatgat aagcgccccc ctcgaa
ttat caaaacaatt gcctctaaga ctcagagtat caaaaagtac t
caaccgaca ttctgggaaa cctgtatgag gtgaagagca aaaagca
ccc tcagattatc aaaaagggc

SEQ ID NO: 25 Polynucleotide sequence of N580A mutant of S. aureus Cas9

atgaaaagga actacattct ggggctggac atcgggatta caagcg
tggg gtatgggatt attgactatg aaacaaggga cgtgatcgac g
caggcgtca gactgttcaa ggaggccaac gtggaaaaca atgaggg
acg gagaagcaag aggggagcca ggcgcctgaa acgacggaga ag
gcacagaa tccagagggt gaagaaactg ctgttcgatt acaacctg
ct gaccgaccat tctgagctga gtggaattaa tccttatgaa gcc
agggtga aaggcctgag tcagaagctg tcagaggaag agttttccg
c agctctgctg cacctggcta agcgccgagg agtgcataac gtca
atgagg tggaagagga caccggcaac gagctgtcta caaaggaaca
gatctcacgc aatagcaaag ctctggaaga gaagtatgtc gcaga
gctgc agctggaacg gctgaagaaa gatggcgagg tgagagggtc
aattaatagg ttcaagacaa gcgactacgt caaagaagcc aagcag
ctgc tgaaagtgca gaaggcttac caccagctgg atcagagctt c
atcgatact tatatcgacc tgctggagac tcggagaacc tactatg
agg gaccaggaga agggagcccc ttcggatgga aagacatcaa gg
aatggtac gagatgctga tgggacattg cacctatttt ccagaaga
gc tgagaagcgt caagtacgct tataacgcag atctgtacaa cgc
cctgaat gacctgaaca acctggtcat caccagggat gaaaacgag
a aactggaata ctatgagaag ttccagatca tcgaaaacgt gttt
aagcag aagaaaaagc ctacactgaa acagattgct aaggagatcc
tggtcaacga agaggacatc aagggctacc gggtgacaag cactg
gaaaa ccagagttca ccaatctgaa agtgtatcac gatattaagg
acatcacagc acggaaagaa atcattgaga acgccgaact gctgga
tcag attgctaaga tcctgactat ctaccagagc tccgaggaca t
ccaggaaga gctgactaac ctgaacagcg agctgaccca ggaagag
atc gaacagatta gtaatctgaa ggggtacacc ggaacacaca ac
ctgtccct gaaagctatc aatctgattc tggatgagct gtggcata
ca aacgacaatc agattgcaat ctttaaccgg ctgaagctgg tcc
caaaaaa ggtggacctg agtcagcaga aagagatccc aaccacact
g gtggacgatt tcattctgtc acccgtggtc aagcggagct tcat
ccagag catcaaagtg atcaacgcca tcatcaagaa gtacggcctg
cccaatgata tcattatcga gctggctagg gagaagaaca gcaag
gacgc acagaagatg atcaatgaga tgcagaaacg aaaccggcag
accaatgaac gcattgaaga gattatccga actaccggga aagaga
acgc aaagtacctg attgaaaaaa tcaagctgca cgatatgcag g
agggaaagt gtctgtattc tctggaggcc atccccctgg aggacct
gct gaacaatcca ttcaactacg aggtcgatca tattatcccc ag
aagcgtgt ccttcgacaa ttcctttaac aacaaggtgc tggtcaag
ca ggaagaggcc tctaaaaagg gcaataggac tcctttccag tac
ctgtcta gttcagattc caagatctct tacgaaacct ttaaaaagc
a cattctgaat ctggccaaag gaaagggccg catcagcaag acca
aaaagg agtacctgct ggaagagcgg gacatcaaca gattctccgt
ccagaaggat tttattaacc ggaatctggt ggacacaaga tacgc
tactc gcggcctgat gaatctgctg cgatcctatt tccgggtgaa
caatctggat gtgaaagtca agtccatcaa cggcgggttc acatct
tttc tgaggcgcaa atggaagttt aaaaaggagc gcaacaaagg g
tacaagcac catgccgaag atgctctgat tatcgcaaat gccgact
tca tctttaagga gtggaaaaag ctggacaaag ccaagaaagt ga
tggagaac cagatgttcg aagagaagca ggccgaatct atgcccga
aa tcgagacaga acaggagtac aaggagattt tcatcactcc tca
ccagatc aagcatatca aggatttcaa ggactacaag tactctcac
c gggtggataa aaagcccaac agagagctga tcaatgacac cctg
tatagt acaagaaaag acgataaggg gaataccctg attgtgaaca
atctgaacgg actgtacgac aaagataatg acaagctgaa aaagc
tgatc aacaaaagtc ccgagaagct gctgatgtac caccatgatc
ctcagacata tcagaaactg aagctgatta tggagcagta cggcga
cgag aagaacccac tgtataagta ctatgaagag actgggaact a
cctgaccaa gtatagcaaa aaggataatg gccccgtgat caagaag
atc aagtactatg ggaacaagct gaatgcccat ctggacatca ca
gacgatta ccctaacagt cgcaacaagg tggtcaagct gtcactga
ag ccatacagat tcgatgtcta tctggacaac ggcgtgtata aat
ttgtgac tgtcaagaat ctggatgtca tcaaaaagga gaactacta
t gaagtgaata gcaagtgcta cgaagaggct aaaaagctga aaaa
gattag caaccaggca gagttcatcg cctcctttta caacaacgac
ctgattaaga tcaatggcga actgtatagg gtcatcgggg tgaac
aatga tctgctgaac cgcattgaag tgaatatgat tgacatcact
taccgagagt atctggaaaa catgaatgat aagcgccccc ctcgaa
ttat caaaacaatt gcctctaaga ctcagagtat caaaaagtac t
caaccgaca ttctgggaaa cctgtatgag gtgaagagca aaaagca
ccc tcagattatc aaaaagggc

SEQ ID NO: 26 codon optimized polynucleotide encoding S. pyogenes Cas9

atggataaaa agtacagcat cgggctggac atcggtacaa actcag
tggg gtgggccgtg attacggacg agtacaaggt accctccaaa a
aatttaaag tgctgggtaa cacggacaga cactctataa agaaaaa
tct tattggagcc ttgctgttcg actcaggcga gacagccgaa gc
cacaaggt tgaagcggac cgccaggagg cggtatacca ggagaaag
aa ccgcatatgc tacctgcaag aaatcttcag taacgagatg gca
aaggttg acgatagctt tttccatcgc ctggaagaat cctttcttg
t tgaggaagac aagaagcacg aacggcaccc catctttggc aata
ttgtcg acgaagtggc atatcacgaa aagtacccga ctatctacca
cctcaggaag aagctggtgg actctaccga taaggcggac ctcag
actta tttatttggc actcgcccac atgattaaat ttagaggaca
tttcttgatc gagggcgacc tgaacccgga caacagtgac gtcgat
aagc tgttcatcca acttgtgcag acctacaatc aactgttcga a
gaaaaccct ataaatgctt caggagtcga cgctaaagca atcctgt
ccg cgcgcctctc aaaatctaga agacttgaga atctgattgc tc
agttgccc ggggaaaaga aaaatggatt gtttggcaac ctgatcgc
cc: tcagtctcgg actgacccca aatttcaaaa gtaacttcga cc
tggccga agacgctaagc tccagctgtc caaggacaca tacgatga
cg acctcgacaa tctgctggc ccagattgggg atcagtacgc cga
tctcttt ttggcagcaa agaacctgtc cgacgccat cctgttgagc
g atatcttgag agtgaacacc gaaattacta aagcacccct tagc
gcatc tatgatcaagc ggtacgacga gcatcatcag gatctgaccc
tgctgaaggc tcttgtgag gcaacagctcc: ccgaaaaata caag
gaaatc ttctttgacc agagcaaaaa cggctacg ctggctatatag
atggtggggc cagtcaggag gaattctata aattcatcaa gccca
ttc tcgagaaaatgg acggcacaga ggagttgctg gtcaaactta
acagggagga cctgctgc ggaagcagcgga cctttgacaa cgggtc
tatc ccccaccaga ttcatctggg cgaactgc acgcaatcctga g
gaggcagga ggatttttat ccttttctta aagataaccg cgagaaa
a tagaaaagattc ttacattcag gatcccgtac tacgtgggac ct
ctcgcccg gggcaatt cacggtttgcct ggatgacaag gaagtcag
ag gagactatta caccttggaa cttcgaag aagtggtggaca agg
gtgcatc tgcccagtct ttcatcgagc ggatgacaaa ttttgaca
agaacctcccta atgagaaggt gctgcccaaa cattctctgc tcta
cgagta ctttaccg tctacaatgaac tgactaaagt caagtacgtc
accgagggaa tgaggaagcc ggcattcc ttagtggagaac agaag
aaggc gattgtagac ctgttgttca agaccaacag gaaggtga ct
gtgaagcaac ttaaagaaga ctactttaag aagatcgaat gttttg
acag tgtggaaa tttcaggggttg aagaccgctt caatgcgtca t
tggggactt accatgatct tctcaaga tcataaaggaca aagactt
cct ggacaacgaa gaaaatgagg atattctcga agacatcg tcct
caccctga ccctgttcga agacagggaa atgatagaag agcgcttg
aa aacctatg cccacctcttcg acgataaagt tatgaagcag ctg
aagcgca ggagatacac aggatggg gaagattgtcaa ggaagctga
t caatggaatt aaggataaac agagtggcaa gaccatac tggatt
tcctca aatctgatgg cttcgccaat aggaacttca tgcaactgat
tcacgatg actctcttacct tcaaggagga cattcaaaag gctca
ggtga gcgggcaggg agactccc ttcatgaacaca tcgcgaattt
ggcaggttcc cccgctatta aaaagggcat ccttcaaa ctgtcaag
gtgg tggatgaatt ggtcaaggta atgggcagac ataagccaga a
aatattg tgatcgagatgg cccgcgaaaa ccagaccaca cagaagg
gcc agaaaaatag tagagagc ggatgaagagga tcgaggaggg ca
tcaaagag ctgggatctc agattctcaa agaacacc ccgtagaaaa
ca cacagctgca gaacgaaaaa ttgtacttgt actatctgca gaa
cggca gagacatgtacg tcgaccaaga acttgatatt aatagactg
t ccgactatga cgtagacc atatcgtgcccc agtccttcct gaag
gacgac tccattgata acaaagtctt gacaagaa gcgacaagaaca
ggggtaaaag tgataatgtg cctagcgagg aggtggtgaa aaaaa
tga agaactactggc gacagctgct taatgcaaag ctcattacac
aacggaagtt cgataatc tgacgaaagcag agagaggtgg cttgtc
tgag ttggacaagg cagggtttat taagcggc agctggtggaaa c
taggcagat cacaaagcac gtggcgcaga ttttggacag ccggatg
a acacaaaatacg acgaaaatga taaactgata cgagaggtca aa
gttatcac gctgaaaa gcaagctggtgt ccgattttcg gaaagact
tc cagttctaca aagttcgcga gattaata actaccatcatg ctc
acgatgc gtacctgaac gctgttgtcg ggaccgcctt gataaaga
agtacccaaagc tggaatccga gttcgtatac ggggattaca aagt
gtacga tgtgagga aaatgatagcca agtccgagca ggagattgga
aaggccacag ctaagtactt cttttatt ctaacatcatga atttt
tttaa gacggaaatt accctggcca acggagagat cagaaagc gg
ccccttatag agacaaatgg tgaaacaggt gaaatcgtct gggata
aggg cagggatt tcgctactgtga ggaaggtgct gagtatgcca c
aggtaaata tcgtgaaaaa aaccgaag tacagaccggag gattttc
caa ggaaagcatt ttgcctaaaa gaaactcaga caagctca tcgc
ccgcaaga aagattggga ccctaagaaa tacgggggat ttgactca
cc caccgtag cctattctgtgc tggtggtagc taaggtggaa aaa
ggaaagt ctaagaagct gaagtccg tgaaggaactct tgggaatca
c tatcatggaa agatcatcct ttgaaaagaa ccctatcg atttcc
tggagg ctaagggtta caaggaggtc aagaaagacc tcatcattaa
actgccaa aatactctctct tcgagctgga aaatggcagg aagag
aatgt tggccagcgc cggagagc tgcaaaagggaa acgagcttgc
tctgccctcc aaatatgtta attttctcta tctcgctt cccactat
gaaa agctgaaagg gtctcccgaa gataacgagc agaagcagct g
ttcgtcg aacagcacaagc actatctgga tgaaataatc gaacaaa
taa gcgagttcag caaaaggg ttatcctggcgg atgctaattt gg
acaaagta ctgtctgctt ataacaagca ccgggata agcctattag
gg aacaagccga gaatataatt cacctcttta cactcacgaa tct
cggag cccccgccgcct tcaaatactt tgatacgact atcgaccgg
a aacggtatac cagtacca aagaggtcctcg atgccaccct catc
caccag tcaattactg gcctgtacga aacacgga tcgacctctctc
aactgggcgg cgactag

SEQ ID NO: 27 codon optimized nucleic acid sequences encoding S. aureus Cas9

atgaaaagga actacattct ggggctggac atcgggatta caagcg
tggg gtatgggatt attgactatg aaacaaggga cgtgatcgac g
caggcgtca gactgttcaa ggaggccaac gtggaaaaca atgaggg
acg gagaagcaag aggggagcca ggcgcctgaa acgacggaga ag
gcacagaa tccagagggt gaagaaactg ctgttcgatt acaacctg
ct gaccgaccat tctgagctga gtggaattaa tccttatgaa gcc
agggtga aaggcctgag tcagaagctg tcagaggaag agttttccg
c agctctgctg cacctggcta agcgccgagg agtgcataac gtca
atgagg tggaagagga caccggcaac gagctgtcta caaaggaaca
gatctcacgc aatagcaaag ctctggaaga gaagtatgtc gcaga
gctgc agctggaacg gctgaagaaa gatggcgagg tgagagggtc
aattaatagg ttcaagacaa gcgactacgt caaagaagcc aagcag
ctgc tgaaagtgca gaaggcttac caccagctgg atcagagctt c
atcgatact tatatcgacc tgctggagac tcggagaacc tactatg
agg gaccaggaga agggagcccc ttcggatgga aagacatcaa gg
aatggtac gagatgctga tgggacattg cacctatttt ccagaaga
gc tgagaagcgt caagtacgct tataacgcag atctgtacaa cgc
cctgaat gacctgaaca acctggtcat caccagggat gaaaacgag
a aactggaata ctatgagaag ttccagatca tcgaaaacgt gttt
aagcag aagaaaaagc ctacactgaa acagattgct aaggagatcc
tggtcaacga agaggacatc aagggctacc gggtgacaag cactg
gaaaa ccagagttca ccaatctgaa agtgtatcac gatattaagg
acatcacagc acggaaagaa atcattgaga acgccgaact gctgga
tcag attgctaaga tcctgactat ctaccagagc tccgaggaca t
ccaggaaga gctgactaac ctgaacagcg agctgaccca ggaagag
atc gaacagatta gtaatctgaa ggggtacacc ggaacacaca ac
ctgtccct gaaagctatc aatctgattc tggatgagct gtggcata
ca aacgacaatc agattgcaat ctttaaccgg ctgaagctgg tcc
caaaaaa ggtggacctg agtcagcaga aagagatccc aaccacact
g gtggacgatt tcattctgtc acccgtggtc aagcggagct tcat
ccagag catcaaagtg atcaacgcca tcatcaagaa gtacggcctg
cccaatgata tcattatcga gctggctagg gagaagaaca gcaag
gacgc acagaagatg atcaatgaga tgcagaaacg aaaccggcag
accaatgaac gcattgaaga gattatccga actaccggga aagaga
acgc aaagtacctg attgaaaaaa tcaagctgca cgatatgcag g
agggaaagt gtctgtattc tctggaggcc tccccctgg aggacctg
ct gaacaatcca ttcaactacg aggtcgatca tattatcccca ga
agcgtgt ccttcgacaa ttcctttaac aacaaggtgc tggtcaagc
a ggaagagaact ctaaaaagg gcaataggac tcctttccag tacc
tgtcta gttcagattc caagatctctt acgaaacct ttaaaaagca
cattctgaat ctggccaaag gaaagggccg catcagcaaga ccaa
aaagg agtacctgct ggaagagcgg gacatcaaca gattctccgt
ccagaaggatt ttattaacc ggaatctggt ggacacaaga tacgct
actc gcggcctgat gaatctgctgc gatcctatt tccgggtgaa c
aatctggat gtgaaagtca agtccatcaa cggcgggttca catctt
ttc tgaggcgcaa atggaagttt aaaaaggagc gcaacaaagg gt
acaagcacc atgccgaag atgctctgat tatcgcaaat gccgactt
ca tctttaagga gtggaaaaagc tggacaaag ccaagaaagt gat
ggagaac cagatgttcg aagagaagca ggccgaatcta tgcccgaa
a tcgagacaga acaggagtac aaggagattt tcatcactcc tcac
cagatca agcatatca aggatttcaa ggactacaag tactctcacc
gggtggataa aaagcccaaca gagagctga tcaatgacac cctgt
atagt acaagaaaag acgataaggg gaataccctga ttgtgaaca
atctgaacgg actgtacgac aaagataatg acaagctgaa aaagct
gatca acaaaagtc ccgagaagct gctgatgtac caccatgatc c
tcagacata tcagaaactga agctgatta tggagcagta cggcgac
gag aagaacccac tgtataagta ctatgaagaga ctgggaact ac
ctgaccaa gtatagcaaa aaggataatg gccccgtgat caagaaga
tca agtactatg ggaacaagct gaatgcccat ctggacatca cag
acgatta ccctaacagtc gcaacaagg tggtcaagct gtcactgaa
g ccatacagat tcgatgtcta tctggacaacg gcgtgtata aatt
tgtgac tgtcaagaat ctggatgtca tcaaaaagga gaactactat
g aagtgaata gcaagtgcta cgaagaggct aaaaagctga aaaag
attag caaccaggcag agttcatcg cctcctttta caacaacgac
ctgattaaga tcaatggcga actgtataggg tcatcgggg tgaaca
atga tctgctgaac cgcattgaag tgaatatgat tgacatcactt
accgagagt atctggaaaa catgaatgat aagcgccccc ctcgaat
tat caaaacaattg cctctaaga ctcagagtat caaaaagtac tc
aaccgaca ttctgggaaa cctgtatgagg tgaagagca aaaagcac
cc tcagattatc aaaaagggc

SEQ ID NO: 28 codon optimized nucleic acid sequences encoding S. aureus Cas9

atgaagcgga actacatcct gggcctggac atcggcatca ccagcg
tggg ctacggcatc atcgactacg agacacggga cgtgatcgat g
ccggcgtgc ggctgttcaa agaggccaac gtggaaaaca acgaggg
cag gcggagcaag agaggcgcca gaaggctgaa gcggcggagg cg
gcatagaa tccagagagt gaagaagctg ctgttcgact acaacctg
ct gaccgaccac agcgagctga gcggcatcaa cccctacgag gcc
agagtga agggcctgag ccagaagctg agcgaggaag agttctctg
c cgccctgctg cacctggcca agagaagagg cgtgcacaac gtga
acgagg tggaagagga caccggcaac gagctgtcca ccaaagagca
gatcagccgg aacagcaagg ccctggaaga gaaatacgtg gccga
actgc agctggaacg gctgaagaaa gacggcgaag tgcggggcag
catcaacaga ttcaagacca gcgactacgt gaaagaagcc aaacag
ctgc tgaaggtgca gaaggcctac caccagctgg accagagctt c
atcgacacc tacatcgacc tgctggaaac ccggcggacc tactatg
agg gacctggcga gggcagcccc ttcggctgga aggacatcaa ag
aatggtac gagatgctga tgggccactg cacctacttc cccgagga
ac tgcggagcgt gaagtacgcc tacaacgccg acctgtacaa cgc
cctgaac gacctgaaca atctcgtgat caccagggac gagaacgag
a agctggaata ttacgagaag ttccagatca tcgagaacgt gttc
aagcag aagaagaagc ccaccctgaa gcagatcgcc aaagaaatcc
tcgtgaacga agaggatatt aagggctaca gagtgaccag caccg
gcaag cccgagttca ccaacctgaa ggtgtaccac gacatcaagg
acattaccgc ccggaaagag attattgaga acgccgagct gctgga
tcag attgccaaga tcctgaccat ctaccagagc agcgaggaca t
ccaggaaga actgaccaat ctgaactccg agctgaccca ggaagag
atc gagcagatct ctaatctgaa gggctatacc ggcacccaca ac
ctgagcct gaaggccatc aacctgatcc tggacgagct gtggcaca
cc aacgacaacc agatcgctat cttcaaccgg ctgaagctgg tgc
ccaagaa ggtggacctg tcccagcaga aagagatccc caccaccct
g gtggacgact tcatcctgag ccccgtcgtg aagagaagct tcat
ccagag catcaaagtg atcaacgcca tcatcaagaa gtacggcctg
cccaacgaca tcattatcga gctggcccgc gagaagaact ccaag
gacgc ccagaaaatg atcaacgaga tgcagaagcg gaaccggcag
accaacgagc ggatcgagga aatcatccgg accaccggca aagaga
acgc caagtacctg atcgagaaga tcaagctgca cgacatgcag g
aaggcaagt gcctgtacag cctggaagcc atccctctgg aagatct
gct gaacaacccc ttcaactatg aggtggacca catcatcccc ag
aagcgtgt ccttcgacaa cagcttcaac aacaaggtgc tcgtgaag
ca ggaagaaaac agcaagaagg gcaaccggac cccattccag tac
ctgagca gcagcgacag caagatcagc tacgaaacct tcaagaagc
a catcctgaat ctggccaagg gcaagggcag aatcagcaag acca
agaaag agtatctgct ggaagaacgg gacatcaaca ggttctccgt
gcagaaagac ttcatcaacc ggaacctggt ggataccaga tacgc
cacca gaggcctgat gaacctgctg cggagctact tcagagtgaa
caacctggac gtgaaagtga agtccatcaa tggcggcttc accagc
tttc tgcggcggaa gtggaagttt aagaaagagc ggaacaaggg g
tacaagcac cacgccgagg acgccctgat cattgccaac gccgatt
tca tcttcaaaga gtggaagaaa ctggacaagg ccaaaaaagt ga
tggaaaac cagatgttcg aggaaaagca ggccgagagc atgcccga
ga tcgaaaccga gcaggagtac aaagagatct tcatcacccc cca
ccagatc aagcacatta aggacttcaa ggactacaag tacagccac
c gggtggacaa gaagcctaat agagagctga ttaacgacac cctg
tactcc acccggaagg acgacaaggg caacaccctg atcgtgaaca
atctgaacgg cctgtacgac aaggacaatg acaagctgaa aaagc
tgatc aacaagagcc ccgaaaagct gctgatgtac caccacgacc
cccagaccta ccagaaactg aagctgatta tggaacagta cggcga
cgag aagaatcccc tgtacaagta ctacgaggaa accgggaact a
cctgaccaa gtactccaaa aaggacaacg gccccgtgat caagaag
att aagtattacg gcaacaaact gaacgcccat ctggacatca cc
gacgacta ccccaacagc agaaacaagg tcgtgaagct gtccctga
ag ccctacagat tcgacgtgta cctggacaat ggcgtgtaca agt
tcgtgac cgtgaagaat ctggatgtga tcaaaaaaga aaactacta
c gaagtgaata gcaagtgcta tgaggaagct aagaagctga agaa
gatcag caaccaggcc gagtttatcg cctccttcta caacaacgat
ctgatcaaga tcaacggcga gctgtataga gtgatcggcg tgaac
aacga cctgctgaac cggatcgaag tgaacatgat cgacatcacc
taccgcgagt acctggaaaa catgaacgac aagaggcccc ccagga
tcat taagacaatc gcctccaaga cccagagcat taagaagtac a
gcacagaca ttctgggcaa cctgtatgaa gtgaaatcta agaagca
ccc tcagatcatc aaaaagggc

SEQ ID NO: 29 codon optimized nucleic acid sequence encoding S. aureus Cas9

atgaagcgca actacatcct cggactggac atcggcatta cctccg
tggg atacggcatc atcgattacg aaactaggga tgtgatcgac g
ctggagtca ggctgttcaa agaggcgaac gtggagaaca acgaggg
gcg gcgctcaaag aggggggccc gccggctgaa gcgccgccgc ag
acatagaa tccagcgcgt gaagaagctg ctgttcgact acaacctt
ct gaccgaccac tccgaacttt ccggcatcaa cccatatgag gct
agagtga agggattgtc ccaaaagctg tccgaggaag agttctccg
c cgcgttgctc cacctcgcca agcgcagggg agtgcacaat gtga
acgaag tggaagaaga taccggaaac gagctgtcca ccaaggagca
gatcagccgg aactccaagg ccctggaaga gaaatacgtg gcgga
actgc aactggagcg gctgaagaaa gacggagaag tgcgcggctc
gatcaaccgc ttcaagacct cggactacgt gaaggaggcc aagcag
ctcc tgaaagtgca aaaggcctat caccaacttg accagtcctt t
atcgatacc tacatcgatc tgctcgagac tcggcggact tactacg
agg gtccagggga gggctcccca tttggttgga aggatattaa gg
agtggtac gaaatgctga tgggacactg cacatacttc cctgagga
gc tgcggagcgt gaaatacgca tacaacgcag acctgtacaa cgc
gctgaac gacctgaaca atctcgtgat cacccgggac gagaacgaa
a agctcgagta ttacgaaaag ttccagatta ttgagaacgt gttc
aaacag aagaagaagc cgacactgaa gcagattgcc aaggaaatcc
tcgtgaacga agaggacatc aagggctatc gagtgacctc aacgg
gaaa gccggagttca ccaatctgaa ggtctaccac gacatcaaag
acattaccgc ccggaagga gatcattgaga acgcggagct gttgga
ccag attgcgaaga ttctgaccat ctaccaatc ctccgaggata t
tcaggaaga actcaccaac ctcaacagcg aactgaccca ggaggag
at agagcaaatct ccaacctgaa gggctacacc ggaactcata ac
ctgagcct gaaggccat caacttgatcc tggacgagct gtggcaca
cc aacgataacc agatcgctat tttcaatcg gctgaagctgg tcc
ccaagaa agtggacctc tcacaacaaa aggagatccc tactaccct
tgtggacgatt tcattctgtc ccccgtggtc aagagaagct tcat
acagtc aatcaaagt gatcaatgcca ttatcaagaa atacggtctg
cccaacgaca ttatcattga gctcgcccg cgagaagaact cgaag
gacgc ccagaagatg attaacgaaa tgcagaagag gaaccgaca g
actaacgaac ggatcgaaga aatcatccgg accaccggga aggaaa
acgc gaagtacct gatcgaaaaga tcaagctcca tgacatgcag g
aaggaaagt gtctgtactc gctggaggc cattccgctgg aggactt
gct gaacaaccct tttaactacg aagtggatca tatcattcc gag
gagcgtgt cattcgacaa ttccttcaac aacaaggtcc tcgtgaag
ca ggaggaaaa ctcgaagaagg gaaaccgcac gccgttccag tac
ctgagca gcagcgactc caagatttc ctacgaaacct tcaagaagc
a catcctcaac ctggcaaagg ggaagggtcg catctccaa gacca
agaagg aatatctgct ggaagaaaga gacatcaaca gattctccgt
gcaaaagga cttcatcaacc gcaacctcgt ggatactaga tacgc
tactc ggggtctgat gaacctcct gagaagctact ttagagtgaa
caatctggac gtgaaggtca agtcgattaa cggaggttt cacctcc
ttcc tgcggcgcaa gtggaagttc aagaaggaac ggaacaaggg c
tacaagca ccacgccgagg acgccctgat cattgccaac gccgact
tca tcttcaaaga atggaagaa acttgacaagg ctaagaaggt ca
tggaaaac cagatgttcg aagaaaagca ggccgagtc tatgcctga
aa tcgagactga acaggagtac aaggaaatct ttattacgcc aca
ccagat caaacacatca aggatttcaa ggattacaag tactcacat
c gcgtggacaa aaagccgaa cagggaactga tcaacgacac cctc
tactcc acccggaagg atgacaaagg gaataccct catcgtcaaca
accttaacgg cctgtacgac aaggacaacg ataagctgaa gaagc
tcat taacaagtcgc ccgaaaagtt gctgatgtac caccacgacc
ctcagactta ccagaagct caagctgatca tggagcagta tgggga
cgag aaaaacccgt tgtacaagta ctacgaaga aactgggaatt a
tctgactaa gtactccaag aaagataacg gccccgtgat taagaag
at taagtactacg gcaacaagct gaacgcccat ctggacatca cc
gatgacta ccctaattc ccgcaacaagg tcgtcaagct gagcctca
ag ccctaccggt ttgatgtgta ccttgacaa tggagtgtaca agt
tcgtgac tgtgaagaac cttgacgtga tcaagaagga gaactacta
cgaagtcaact ccaagtgcta cgaggaagca aagaagttga agaa
gatctc gaaccaggc cgagttcattg cctccttcta taacaacgac
ctgattaaga tcaacggcga actgtaccg cgtcattggcg tgaac
aacga tctcctgaac cgcatcgaag tgaacatgat cgacatcac t
taccgggaat acctggagaa tatgaacgac aagcgcccgc cccgga
tcat taagactat cgcctcaaaga cccagtcgat caagaagtac a
gcaccgaca tcctgggcaa cctgtacga ggtcaaatcga agaagca
ccc ccagatcatc aagaaggga

SEQ ID NO: 30 codon optimized nucleic acid sequence encoding S. aureus Cas9

atggccccaaagaagaagcggaaggtcggtatccacggagtcccagcaac
caagcggaactacatcctgggcctggacatcggcatcaccagcgtgggct
acggcatcatcgactacgagacacgggacgtgatcgatgccggcgtgcgg
ctgttcaaagaggccaacgtggaaaacaacgagggcaggcggagcaagag
aggcgccagaaggctgaagcggcggaggcggcatagaatccagagagtga
agaagctgctgttcgactacaacctgctgaccgaccacagcgagctgagc
ggcatcaacccctacgaggccagagtgaagggcctgagccagaagctgag
cgaggaagagttctctgccgccctgctgcacctggccaagagaagaggcg
tgcacaacgtgaacgaggtggaagaggacaccggcaacgagctgtccacc
agagagcagatcagccggaacagcaaggccctggaagagaaatacgtggc
cgaactgcagctggaacggctgaagaaagacggcgaagtgcggggcagca
tcaacagattcaagaccagcgactacgtgaaagaagccaaacagctgctg
aaggtgcagaaggcctaccaccagctggaccagagcttcatcgacaccta
catcgacctgctggaaacccggcggacctactatgagggacctggcgagg
gcagccccttcggctggaaggacatcaaagaatggtacgagatgctgatg
ggccactgcacctacttccccgaggaactgcggagcgtgaagtacgccta
caacgccgacctgtacaacgccctgaacgacctgaacaatctcgtgatca
ccagggacgagaacgagaagctggaatattacgagaagttccagatcatc
gagaacgtgttcaagcagaagaagaagcccaccctgaagcagatcgccaa
agaaatcctcgtgaacgaagaggatattaagggctacagagtgaccagca
ccggcaagcccgagttcaccaacctgaaggtgtaccacgacatcaaggac
attaccgcccggaaagagattattgagaacgccgagctgctggatcagat
tgccaagatcctgaccatctaccagagcagcgaggacatccaggaagaac
tgaccaatctgaactccgagctgacccaggaagagatcgagcagatctct
aatctgaagggctataccggcacccacaacctgagcctgaaggccatcaa
cctgatcctggacgagctgtggcacaccaacgacaaccagatcgctatct
tcaaccggctgaagctggtgcccaagaaggtggacctgtcccagcagaaa
gagatccccaccaccctggtggacgacttcatcctgagccccgtcgtcga
agagaagcttcatccagagcatcaaagtgatcaacgccatcatcaagaag
tacggcctgcccaacgacatcattatcgagctggcccgcgagaagaactc
caaggacgcccagaaaatgatcaacgagatgcagaagcggaaccggcaga
ccaacgagcggatcgaggaaatcatccggaccaccggcaaagagaacgcc
aagtacctgatcgagaagatcaagctgcacgacatgcaggaaggcaagtg
cctgtacagcctggaagccatccctctggaagatctgctgaacaacccct
tcaactatgaggtggaccacatcatccccagaagcgtgtccttcgacaac
agcttcaacaacaaggtgctcgtgaagcaggaagaaaacagcaagaaggg
caaccggaccccattccagtacctgagcagcagcgacagcaagatcagct
acgaaaccttcaagaagcacatcctgaatctggccaagggcaagggcaga
atcagcaagaccaagaaagagtatctgctggaagaacgggacatcaacag
gttctccgtgcagaaagacttcatcaaccggaacctggtggataccagat
acgccaccagaggcctgatgaacctgctgcggagctacttcagagtgaac
aacctggacgtgaaagtgaagtccatcaatggcggcttcaccagctttct
gcggcggaagtggaagtttaagaaagagcggaacaaggggtacaagcacc
acgccgaggacgccctgatcattgccaacgccgatttcatcttcaaagag
tggaagaaactggacaaggccaaaaaagtgatggaaaaccagatgttcga
ggaaaggcaggccgagagcatgcccgagatcgaaaccgagcaggagtaca
aagagatcttcatcaccccccaccagatcaagcacattaaggacttcaag
gactacaagtacagccaccgggtggacaagaagcctaatagagagctgat
taacgacaccctgtactccacccggaaggacgacaagggcaacaccctga
tcgtgaacaatctgaacggcctgtacgacaaggacaatgacaagctgaaa
aagctgatcaacaagagccccgaaaagctgctgatgtaccaccacgaccc
ccagacctaccagaaactgaagctgattatggaacagtacggcgacgaga
agaatcccctgtacaagtactacgaggaaaccgggaactacctgaccaag
tactccaaaaaggacaacggccccgtgatcaagaagattaagtattacgg
caacaaactgaacgcccatctggacatcaccgacgactaccccaacagca
gaaacaaggtcgtgaagctgtccctgaagccctacagattcgacgtgtac
ctggacaatggcgtgtacaagttcgtgaccgtgaagaatctggatgtgat
caaaaaagaaaactactacgaagtgaatagcaagtgctatgaggaagcta
agaagctgaagaagatcagcaaccaggccgagtttatcgcctccttctac
aacaacgatctgatcaagatcaacggcgagctgtatagagtgatcggcgt
gaacaacgacctgctgaaccggatcgaagtgaacatgatcgacatcacct
accgcgagtacctggaaaacatgaacgacaagaggccccccaggatcatt
aagacaatcgcctccaagacccagagcattaagaagtacagcacagacat
tctgggcaacctgtatgaagtgaaatctaagaagcaccctcagatcatca
aaaagggcaaaaggccggcggccacgaaaaaggccggccaggcaaaaaag
aaaaag

SEQ ID NO: 31 codon optimized nucleic acid sequence encoding S. aureus Cas9

accggtgcca ccatgtaccc atacgatgtt ccagattacg cttcgc
cgaa gaaaaagcgc aaggtcgaag cgtccatgaa aaggaactac a
ttctggggc tggacatcgg gattacaagc gtggggtatg ggattat
tga ctatgaaaca agggacgtga tcgacgcagg cgtcagactg tt
caaggagg ccaacgtgga aaacaatgag ggacggagaa gcaagagg
gg agccaggcgc ctgaaacgac ggagaaggca cagaatccag agg
gtgaaga aactgctgtt cgattacaac ctgctgaccg accattctg
a gctgagtgga attaatcctt atgaagccag ggtgaaaggc ctga
gtcaga agctgtcaga ggaagagttt tccgcagctc tgctgcacct
ggctaagcgc cgaggagtgc ataacgtcaa tgaggtggaa gagga
caccg gcaacgagct gtctacaaag gaacagatct cacgcaatag
caaagctctg gaagagaagt atgtcgcaga gctgcagctg gaacgg
ctga agaaagatgg cgaggtgaga gggtcaatta ataggttcaa g
acaagcgac tacgtcaaag aagccaagca gctgctgaaa gtgcaga
agg cttaccacca gctggatcag agcttcatcg atacttatat cg
acctgctg gagactcgga gaacctacta tgagggacca ggagaagg
ga gccccttcgg atggaaagac atcaaggaat ggtacgagat gct
gatggga cattgcacct attttccaga agagctgaga agcgtcaag
t acgcttataa cgcagatct acaacgccc tgaatgacct gaacaa
cctg gtcatcacca gggatgaaaa cgagaaactg aatactatg ag
aagttcca gatcatcgaa aacgtgttta agcagaagaa aaagccta
ca tgaaacaga ttgctaagga gatcctggtc aacgaagagg acat
caaggg ctaccgggtg caagcactg gaaaaccaga gttcaccaat
ctgaaagtgt atcacgatat taaggacatc cagcacgga aagaaat
cat tgagaacgcc gaactgctgg atcagattgc taagatcctg ct
atctacc agagctccga ggacatccag gaagagctga ctaacctga
a cagcgagctg cccaggaag agatcgaaca gattagtaat ctgaa
ggggt acaccggaac acacaacctg ccctgaaag ctatcaatct g
attctggat gagctgtggc atacaaacga caatcagatt caatctt
ta accggctgaa gctggtccca aaaaaggtgg acctgagtca gca
gaaagag tcccaacca cactggtgga cgatttcatt ctgtcacccg
tggtcaagcg gagcttcatc agagcatca aagtgatcaa cgccat
catc aagaagtacg gcctgcccaa tgatatcatt tcgagctgg ct
agggagaa gaacagcaag gacgcacaga agatgatcaa tgagatgc
ag aacgaaacc ggcagaccaa tgaacgcatt gaagagatta tccg
aactac cgggaaagag acgcaaagt acctgattga aaaaatcaag
ctgcacgata tgcaggaggg aaagtgtctg attctctgg aggccat
ccc cctggaggac ctgctgaaca atccattcaa ctacgaggtc at
catatta tccccagaag cgtgtccttc gacaattcct ttaacaaca
a ggtgctggtc agcaggaag agaactctaa aaagggcaat aggac
tcctt tccagtacct gtctagttca attccaaga tctcttacga a
acctttaaa aagcacattc tgaatctggc caaaggaaag gccgcat
ca gcaagaccaa aaaggagtac ctgctggaag agcgggacat caa
cagattc ccgtccaga aggattttat taaccggaat ctggtggaca
caagatacgc tactcgcggc tgatgaatc tgctgcgatc ctattt
ccgg gtgaacaatc tggatgtgaa agtcaagtcc tcaacggcg gg
ttcacatc ttttctgagg cgcaaatgga agtttaaaaa ggagcgca
ac aagggtaca agcaccatgc cgaagatgct ctgattatcg caaa
tgccga cttcatcttt aggagtgga aaaagctgga caaagccaag
aaagtgatgg agaaccagat gttcgaagag agcaggccg aatctat
gcc cgaaatcgag acagaacagg agtacaagga gattttcatc ct
cctcacc agatcaagca tatcaaggat ttcaaggact acaagtact
c tcaccgggtg ataaaaagc ccaacagaga gctgatcaat gacac
cctgt atagtacaag aaaagacgat aggggaata ccctgattgt g
aacaatctg aacggactgt acgacaaaga taatgacaag tgaaaaa
gc tgatcaacaa aagtcccgag aagctgctga tgtaccacca tga
tcctcag catatcaga aactgaagct gattatggag cagtacggcg
acgagaagaa cccactgtat agtactatg aagagactgg gaacta
cctg accaagtata gcaaaaagga taatggcccc tgatcaaga ag
atcaagta ctatgggaac aagctgaatg cccatctgga catcacag
ac attacccta acagtcgcaa caaggtggtc aagctgtcac tgaa
gccata cagattcgat tctatctgg acaacggcgt gtataaattt
gtgactgtca agaatctgga tgtcatcaaa aggagaact actatga
agt gaatagcaag tgctacgaag aggctaaaaa gctgaaaaag tt
agcaacc aggcagagtt catcgcctcc ttttacaaca acgacctga
t taagatcaat gcgaactgt atagggtcat cggggtgaac aatga
tctgc tgaaccgcat tgaagtgaat tgattgaca tcacttaccg a
gagtatctg gaaaacatga atgutaagcg cccccctcga ttatcaa
aa caattgcctc taagactcag agtatcaaaa agtactcaac cga
cattctg gaaacctgt atgaggtgaa gagcaaaaag caccctcaga
ttatcaaaaa gggctaagaa ttc

SEQ ID NO: 32 codon optimized nucleic acid sequences encoding S. aureus Cas9

atggccccaaagaagaagcggaaggtcggtatccacggagtcccagcagc
caagcggaactacatcctgggcctggacatcggcatcaccagcgtgggct
acggcatcatcgactacgagacacgggacgtgatcgatgccggcgtgcgg
ctgttcaaagaggccaacgtggaaaacaacgagggcaggcggagcaagag
aggcgccagaaggctgaagcggcggaggcggcatagaatccagagagtga
agaagctgctgttcgactacaacctgctgaccgaccacagcgagctgagc
ggcatcaacccctacgaggccagagtgaagggcctgagccagaagctgag
cgaggaagagttctctgccgccctgctgcacctggccaagagaagaggcg
tgcacaacgtgaacgaggtggaagaggacaccggcaacgagctgtccacc
aaagagcagatcagccggaacagcaaggccctggaagagaaatacgtggc
cgaactgcagctggaacggctgaagaaagacggcgaagtgcggggcagca
tcaacagattcaagaccagcgactacgtgaaagaagccaaacagctgctg
aaggtgcagaaggcctaccaccagctggaccagagcttcatcgacaccta
catcgacctgctggaaacccggcggacctactatgagggacctggcgagg
gcagccccttcggctggaaggacatcaaagaatggtacgagatgctgatg
ggccactgcacctacttccccgaggaactgcggagcgtgaagtacgccta
caacgccgacctgtacaacgccctgaacgacctgaacaatctcgtgatca
ccagggacgagaacgagaagctggaatattacgagaagttccagatcatc
gagaacgtgttcaagcagaagaagaagcccaccctgaagcagatcgccaa
agaaatcctcgtgaacgaagaggatattaagggctacagagtgaccagca
ccggcaagcccgagttcaccaacctgaaggtgtaccacgacatcaaggac
attaccgcccggaaagagattattgagaacgccgagctgctggatcagat
tgccaagatcctgaccatctaccagagcagcgaggacatccaggaagaac
tgaccaatctgaactccgagctgacccaggaagagatcgagcagatctct
aatctgaagggctataccggcacccacaacctgagcctgaaggccatcaa
cctgatcctggacgagctgtggcacaccaacgacaaccagatcgctatct
tcaaccggctgaagctggtgcccaagaaggtggacctgtcccagcagaaa
gagatccccaccaccctggtggacgacttcatcctgagccccgtcgtgaa
gagaagcttcatccagagcatcaaagtgatcaacgccatcatcaagaagt
acggcctgcccaacgacatcattatcgagctggcccgcgagaagaactcc
aaggacgcccagaaaatgatcaacgagatgcagaagcggaaccggcagac
caacgagcggatcgaggaaatcatccggaccaccggcaaagagaacgcca
agtacctgatcgagaagatcaagctgcacgacatgcaggaaggcaagtgc
ctgtacagcctggaagccatccctctggaagatctgctgaacaacccctt
caactatgaggtggaccacatcatccccagaagcgtgtccttcgacaaca
gcttcaacaacaaggtgctcgtgaagcaggaagaaaacagcaagaagggc
aaccggaccccattccagtacctgagcagcagcgacagcaagatcagcta
cgaaaccttcaagaagcacatcctgaatctggccaagggcaagggcagaa
tcagcaagaccaagaaagagtatctgctggaagaacgggacatcaacagg
ttctccgtgcagaaagacttcatcaaccggaacctggtggataccagata
cgccaccagaggcctgatgaacctgctgcggagctacttcagagtgaaca
acctggacgtgaaagtgaagtccatcaatggcggcttcaccagctttctg
cggcggaagtggaagtttaagaaagagcggaacaaggggtacaagcacca
cgccgaggacgccctgatcattgccaacgccgatttcatcttcaaagagt
ggaagaaactggacaaggccaaaaaagtgatggaaaaccagatgttcgag
gaaaagcaggccgagagcatgcccgagatcgaaaccgagcaggagtacaa
agagatcttcatcaccccccaccagatcaagcacattaaggacttcaagg
actacaagtacagccaccgggtggacaagaagcctaatagagagctgatt
aacgacaccctgtactccacccggaaggacgacaagggcaacaccctgat
cgtgaacaatctgaacggcctgtacgacaaggacaatgacaagctgaaaa
agctgatcaacaagagccccgaaaagctgctgatgtaccaccacgacccc
cagacctaccagaaactgaagctgattatggaacagtacggcgacgagaa
gaatcccctgtacaagtactacgaggaaaccgggaactacctgaccaagt
actccaaaaaggacaacggccccgtgatcaagaagattaagtattacggc
aacaaactgaacgcccatctggacatcaccgacgactaccccaacagcag
aaacaaggtcgtgaagctgtccctgaagccctacagattcgacgtgtacc
tggacaatggcgtgtacaagttcgtgaccgtgaagaatctggatgtgatc
aaaaaagaaaactactacgaagtgaatagcaagtgctatgaggaagctaa
gaagctgaagaagatcagcaaccaggccgagtttatcgcctccttctaca
acaacgatctgatcaagatcaacggcgagctgtatagagtgatcggcgtg
aacaacgacctgctgaaccggatcgaagtgaacatgatcgacatcaccta
ccgcgagtacctggaaaacatgaacgacaagaggccccccaggatcatta
agacaatcgcctccaagacccagagcattaagaagtacagcacagacatt
ctgggcaacctgtatgaagtgaaatctaagaagcaccctcagatcatcaa
aaagggcaaaaggccggcggccacgaaaaaggccggccaggcaaaaaaga
aaaag

SEQ ID NO: 33 codon optimized nucleic acid sequences encoding S. aureus Cas9

aagcggaactacatcctgggcctggacatcggcatcaccagcgtgggcta
cggcatcatcgactacgagacacgggacgtgatcgatgccggcgtgcggc
tgttcaaagaggccaacgtggaaaacaacgagggcaggcggagcaagaga
ggcgccagaaggctgaagcggcggaggcggcatagaatccagagagtgaa
gaagctgctgttcgactacaacctgctgaccgaccacagcgagctgagcg
gcatcaacccctacgaggccagagtgaagggcctgagccagaagctgagc
gaggaagagttctctgccgccctgctgcacctggccaagagaagaggcgt
gcacaacgtgaacgaggtggaagaggacaccggcaacgagctgtccacca
aagagcagatcagccggaacagcaaggccctggaagagaaatacgtggcc
gaactgcagctggaacggctgaagaaagacggcgaagtgcggggcagcat
caacagattcaagaccagcgactacgtgaaagaagccaaacagctgctga
aggtgcagaaggcctaccaccagctggaccagagcttcatcgacacctac
atcgacctgctggaaacccggcggacctactatgagggacctggcgaggg
cagccccttcggctggaaggacatcaaagaatggtacgagatgctgatgg
gccactgcacctacttccccgaggaactgcggagcgtgaagtacgcctac
aacgccgacctgtacaacgccctgaacgacctgaacaatctcgtgatcac
cagggacgagaacgagaagctggaatattacgagaagttccagatcatcg
agaacgtgttcaagcagaagaagaagcccaccctgaagcagatcgccaaa
gaaatcctcgtgaacgaagaggatattaagggctacagagtgaccagcac
cggcaagcccgagttcaccaacctgaaggtgtaccacgacatcaaggaca
ttaccgcccggaaagagattattgagaacgccgagctgctggatcagatt
gccaagatcctgaccatctaccagagcagcgaggacatccaggaagaact
gaccaatctgaactccgagctgacccaggaagagatcgagcagatctcta
atctgaagggctataccggcacccacaacctgagcctgaaggccatcaac
ctgatcctggacgagctgtggcacaccaacgacaaccagatcgctatctt
caaccggctgaagctggtgcccaagaaggtggacctgtcccagcagaaag
agatccccaccaccctggtggacgacttcatcctgagccccgtcgtgaag
agaagcttcatccagagcatcaaagtgatcaacgccatcatcaagaagta
cggcctgcccaacgacatcattatcgagctggcccgcgagaagaactcca
aggacgcccagaaaatgatcaacgagatgcagaagcggaaccggcagacc
aacgagcggatcgaggaaatcatccggaccaccggcaaaaagaacgccaa
gtacctgatcgagaagatcaagctgcacgacatgcaggaaggcaagtgcc
tgtacagcctggaagccatccctctggaagatctgctgaacaaccccttc
aactatgaggtggaccacatcatccccagaagcgtgtccttcgacaacag
cttcaacaacaaggtgctcgtgaagcaggaagaaaacagcaagaagggca
accggaccccattccagtacctgagcagcagcgacagcaagatcagctac
gaaaccttcaagaagcacatcctgaatctggccaagggcaagggcagaat
cagcaagaccaagaaagagtatctgctggaagaacgggacatcaacaggt
tctccgtgcagaaagacttcatcaaccggaacctggtggataccagatac
gccaccagaggcctgatgaacctgctgcggagctacttcagagtgaacaa
cctggacgtgaaagtgaagtccatcaatggcggcttcaccagctttctgc
ggcggaagtggaagtttaagaaagagcggaacaaggggtacaagcaccac
gccgaggacgccctgatcattgccaacgccgatttcatcttcaaagagtg
gaagaaactggacaaggccaaaaaagtgatggaaaaccagatgttcgagg
aaaagcaggccgagagcatgcccgagatcgaaaccgagcaggagtacaaa
gagatcttcatcaccccccaccagatcaagcacattaaggacttcaagga
ctacaagtacagccaccgggtggacaagaagcctaatagagagctgatta
acgacaccctgtactccacccggaaggacgacaagggcaacaccctgatc
gtgaacaatctgaacggcctgtacgacaaggacaatgacaagctgaaaaa
gctgatcaacaagagccccgaaaagctgctgatgtaccaccacgaccccc
agacctaccagaaactgaagctgattatggaacagtacggcgacgagaag
aatcccctgtacaagtactacgaggaaaccgggaactacctgaccaagta
ctccaaaaaggacaacggccccgtgatcaagaagattaagtattacggca
acaaactgaacgcccatctggacatcaccgacgactaccccaacagcaga
aacaaggtcgtgaagctgtccctgaagccctacagattcgacgtgtacct
ggacaatggcgtgtacaagttcgtgaccgtgaagaatctggatgtgatca
aaaaagaaaactactacgaagtgaatagcaagtgctatgaggaagctaag
aagctgaagaagatcagcaaccaggccgagtttatcgcctccttctacaa
caacgatctgatcaagatcaacggcgagctgtatagagtgatcggcgtga
acaacgacctgctgaaccggatcgaagtgaacatgatcgacatcacctac
cgcgagtacctggaaaacatgaacgacaagaggccccccaggatcattaa
gacaatcgcctccaagacccagagcattaagaagtacagcacagacattc
tgggcaacctgtatgaagtgaaatctaagaagcaccctcagatcatcaaa
aagggc

SEQ ID NO: 34 Vector (pDO242) encoding codon optimized nucleic acid sequence encoding S. aureus Cas9

ctaaattgtaagcgttaatattttgttaaaattcgcgttaaatttttgtt
aaatcagctcattttttaaccaataggccgaaatcggcaaaatcccttat
aaatcaaaagaatagaccgagatagggttgagtgttgttccagtttggaa
caagagtccactattaaagaacgtggactccaacgtcaaagggcgaaaaa
ccgtctatcagggcgatggcccactacgtgaaccatcaccctaatcaagt
tttttggggtcgaggtgccgtaaagcactaaatcggaaccctaaagggag
cccccgatttagagcttgacggggaaagccggcgaacgtggcgagaaagg
aagggaagaaagcgaaaggagcgggcgctagggcgctggcaagtgtagcg
gtcacgctgcgcgtaaccaccacacccgccgcgcttaatgcgccgctaca
gggcgcgtcccattcgccattcaggctgcgcaactgttgggaagggcgat
cggtgcgggcctcttcgctattacgccagctggcgaaagggggatgtgct
gcaaggcgattaagttgggtaacgccagggttttcccagtcacgacgttg
taaaacgacggccagtgagcgcgcgtaatacgactcactatagggcgaat
tgggtacCtttaattctagtactatgcaTgcgttgacattgattattgac
tagttattaatagtaatcaattacggggtcattagttcatagcccatata
tggagttccgcgttacataacttacggtaaatggcccgcctggctgaccg
cccaacgacccccgcccattgacgtcaataatgacgtatgttcccatagt
aacgccaatagggactttccattgacgtcaatgggtggagtatttacggt
aaactgcccacttggcagtacatcaagtgtatcatatgccaagtacgccc
cctattgacgtcaatgacggtaaatggcccgcctggcattatgcccagta
catgaccttatgggactttcctacttggcagtacatctacgtattagtca
tcgctattaccatggtgatgcggttttggcagtacatcaatgggcgtgga
tagcggtttgactcacggggatttccaagtctccaccccattgacgtcaa
tgggagtttgttttggcaccaaaatcaacgggactttccaaaatgtcgta
acaactccgccccattgacgcaaatgggcggtaggcgtgtacggtgggag
gtctatataagcagagctctctggctaactaccggtgccaccATGAAAAG
GAACTACATTCTGGGGCTGGACATCGGGATTACAAGCGTGGGGTATGGGA
TTATTGACTATGAAACAAGGGACGTGATCGACGCAGGCGTCAGACTGTTC
AAGGAGGCCAACGTGGAAAACAATGAGGGACGGAGAAGCAAGAGGGGAGC
CAGGCGCCTGAAACGACGGAGAAGGCACAGAATCCAGAGGGTGAAGAAAC
TGCTGTTCGATTACAACCTGCTGACCGACCATTCTGAGCTGAGTGGAATT
AATCCTTATGAAGCCAGGGTGAAAGGCCTGAGTCAGAAGCTGTCAGAGGA
AGAGTTTTCCGCAGCTCTGCTGCACCTGGCTAAGCGCCGAGGAGTGCATA
ACGTCAATGAGGTGGAAGAGGACACCGGCAACGAGCTGTCTACAAAGGAA
CAGATCTCACGCAATAGCAAAGCTCTGGAAGAGAAGTATGTCGCAGAGCT
GCAGCTGGAACGGCTGAAGAAAGATGGCGAGGTGAGAGGGTCAATTAATA
GGTTCAAGACAAGCGACTACGTCAAAGAAGCCAAGCAGCTGCTGAAAGTG
CAGAAGGCTTACCACCAGCTGGATCAGAGCTTCATCGATACTTATATCGA
CCTGCTGGAGACTCGGAGAACCTACTATGAGGGACCAGGAGAAGGGAGCC
CCTTCGGATGGAAAGACATCAAGGAATGGTACGAGATGCTGATGGGACAT
TGCACCTATTTTCCAGAAGAGCTGAGAAGCGTCAAGTACGCTTATAACGC
AGATCTGTACAACGCCCTGAATGACCTGAACAACCTGGTCATCACCAGGG
ATGAAAACGAGAAACTGGAATACTATGAGAAGTTCCAGATCATCGAAAAC
GTGTTTAAGCAGAAGAAAAAGCCTACACTGAAACAGATTGCTAAGGAGAT
CCTGGTCAACGAAGAGGACATCAAGGGCTACCGGGTGACAAGCACTGGAA
AACCAGAGTTCACCAATCTGAAAGTGTATCACGATATTAAGGACATCACA
GCACGGAAAGAAATCATTGAGAACGCCGAACTGCTGGATCAGATTGCTAA
GATCCTGACTATCTACCAGAGCTCCGAGGACATCCAGGAAGAGCTGACTA
ACCTGAACAGCGAGCTGACCCAGGAAGAGATCGAACAGATTAGTAATCTG
AAGGGGTACACCGGAACACACAACCTGTCCCTGAAAGCTATCAATCTGAT
TCTGGATGAGCTGTGGCATACAAACGACAATCAGATTGCAATCTTTAACC
GGCTGAAGCTGGTCCCAAAAAAGGTGGACCTGAGTCAGCAGAAAGAGATC
CCAACCACACTGGTGGACGATTTCATTCTGTCACCCGTGGTCAAGCGGAG
CTTCATCCAGAGCATCAAAGTGATCAACGCCATCATCAAGAAGTACGGCC
TGCCCAATGATATCATTATCGAGCTGGCTAGGGAGAAGAACAGCAAGGAC
GCACAGAAGATGATCAATGAGATGCAGAAACGAAACCGGCAGACCAATGA
ACGCATTGAAGAGATTATCCGAACTACCGGGAAAGAGAACGCAAAGTACC
TGATTGAAAAAATCAAGCTGCAGATATGCAGGAGGGAAAGTGTCTGTATT
CTTGGAGGCCATCCCCCTGGAGGACCTGCTGAACAATCCATTCAACTACG
AGGTCGATCATATTATCCCCAGAAGCGTGTCCTTCGACAATTCCTTTAAC
AACAAGGTGCTGGTCAAGCAGGAAGAGAACTCTAAAAAGGGCAATAGGAC
TCCTTTCCAGTACCTGTCTAGTTCAGATTCCAAGATCTCTTACGAAACCT
TTAAAAAGCACATTCTGAATCTGGCCAAAGGAAAGGGCCGCATCAGCAAG
ACCAAAAAGGAGTACCTGCTGGAAGAGCGGGACATCAACAGATTCTCCGT
CCAGAAGGATTTTATTAACCGGAATCTGGTGGACACAAGATACGCTACTC
GCGGCCTGATGAATCTGCTGCGATCCTATTTCCGGGTGAACAATCTGGAT
GTGAAAGTCAAGTCCATCAACGGCGGGTTCACATCTTTTCTGAGGCGCAA
ATGGAAGTTTAAAAAGGAGCGCAACAAAGGGTACAAGCACCATGCCGAAG
ATGCTCTGATTATCGCAAATGCCGACTTCATCTTTAAGGAGTGGAAAAAG
CTGGACAAAGCCAAGAAAGTGATGGAGAACCAGATGTTCGAAGAGAAGCA
GGCCGAATCTATGCCCGAAATCGAGACAGAACAGGAGTACAAGGAGATTT
TCATCACTCCTCACCAGATCAAGCATATCAAGGATTTCAAGGACTACAAG
TACTCTCACCGGGTGGATAAAAAGCCCAACAGAGAGCTGATCAATGACAC
CCTGTATAGTACAAGAAAAGACGATAAGGGGAATACCCTGATTGTGAACA
ATCTGAACGGACTGTACGACAAAGATAATGACAAGCTGAAAAAGCTGATC
AACAAAAGTCCCGAGAAGCTGCTGATGTACCACCATGATCCTCAGACATA
TCAGAAACTGAAGCTGATTATGGAGCAGTACGGCGACGAGAAGAACCCAC
TGTATAAGTACTATGAAGAGACTGGGAACTACCTGACCAAGTATAGCAAA
AAGGATAATGGCCCCGTGATCAAGAAGATCAAGTACTATGGGAACAAGCT
GAATGCCCATCTGGACATCACAGACGATTACCCTAACAGTCGCAACAAGG
TGGTCAAGCTGTCACTGAAGCCATACAGATTCGATGTCTATCTGGACAAC
GGCGTGTATAAATTTGTGACTGTCAAGAATCTGGATGTCATCAAAAAGGA
GAACTACTATGAAGTGAATAGCAAGTGCTACGAAGAGGCTAAAAAGCTGA
AAAAGATTAGCAACCAGGCAGAGTTCATCGCCTCCTTTTACAACAACGAC
CTGATTAAGATCAATGGCGAACTGTATAGGGTCATCGGGGTGAACAATGA
TCTGCTGAACCGCATTGAAGTGAATATGATTGACATCACTTACCGAGAGT
ATCTGGAAAACATGAATGATAAGCGCCCCCCTCGAATTATCAAAACAATT
GCCTCTAAGACTCAGAGTATCAAAAAGTACTCAACCGACATTCTGGGAAA
CCTGTATGAGGTGAAGAGCAAAAAGCACCCTCAGATTATCAAAAAGGGCa
gcggaggcaagcgtcctgctgctactaagaaagctggtcaagctaagaaa
aagaaaggatcctacccatacgatgttccagattacgcttaagaattcct
agagctcgctgatcagcctcgactgtgccttctagttgccagccatctgt
tgtttgcccctcccccgtgccttccttgaccctggaaggtgccactccca
ctgtcctttcctaataaaatgaggaaattgcatcgcattgtctgagtagg
tgtcattctattctggggggtggggtggggcaggacagcaagggggagga
ttgggaagagaatagcaggcatgctggggaggtagcggccgcCCgcggtg
gagctccagcttttgttccctttagtgagggttaattgcgcgcttggcgt
aatcatggtcatagctgtttcctgtgtgaaattgttatccgctcacaatt
ccacacaacatacgagccggaagcataaagtgtaaagcctggggtgccta
atgagtgagctaactcacattaattgcgttgcgctcactgcccgctttcc
agtcgggaaacctgtcgtgccagctgcattaatgaatcggccaacgcgcg
gggagaggcggtttgcgtattgggcgctcttccgcttcctcgctcactga
ctcgctgcgctcggtcgttcggctgcggcgagcggtatcagctcactcaa
aggcggtaatacggttatccacagaatcaggggataacgcaggaaagaac
atgtgagcaaaaggccagcaaaaggccaggaaccgtaaaaaggccgcgtt
gctggcgtttttccataggctccgcccccctgacgagcatcacaaaaatc
gacgctcaagtcagaggtggcgaaacccgacaggactataaagataccag
gcgtttccccctggaagctccctcgtgcgctctcctgttccgaccctgcc
gcttaccggatacctgtccgcctttctcccttcgggaagcgtggcgcttt
ctcatagctcacgctgtaggtatctcagttcggtgtaggtcgttcgctcc
aagctgggctgtgtgcacgaaccccccgttcagcccgaccgctgcgcctt
atccggtaactatcgtcttgagtccaacccggtaagacacgacttatcgc
cactggcagcagccactggtaacaggattagcagagcgaggtatgtaggc
ggtgctacagagttcttgaagtggtggcctaactacggctacactagaag
gacagtatttggtatctgcgctctgctgaagccagttaccttcggaaaaa
gagttggtagctcttgatccggcaaacaaaccaccgctggtagcggtggt
ttttttgtttgcaagcagcagattacgcgcagaaaaaaaggatctcaaga
agatcctttgatcttttctacggggtctgacgctcagtggaacgaaaact
cacgttaagggattttggtcatgagattatcaaaaaggatcttcacctag
atccttttaaattaaaaatgaagttttaaatcaatctaaagtatatatga
gtaaacttggtctgacagttaccaatgcttaatcagtgaggcacctatct
cagcgatctgtctatttcgttcatccatagttgcctgactccccgtcgtg
tagataactacgatacgggagggcttaccatctggccccagtgctgcaat
gataccgcgagacccacgctcaccggctccagatttatcagcaataaacc
agccagccggaagggccgagcgcagaagtggtcctgcaactttatccgcc
tccatccagtctattaattgttgccgggaagctagagtaagtagttcgcc
agttaatagtttgcgcaacgttgttgccattgctacaggcatcgtggtgt
cacgctcgtcgtttggtatggcttcattcagctccggttcccaacgatca
aggcgagttacatgatcccccatgttgtgcaaaaaagcggttagctcctt
cggtcctccgtcgttgtcagaagtaagttggccgcagtgttatcactcat
ggttatggcagcactgcataattctcttactgtcatgccatccgtaagat
gcttttctgtgactggtgagtactcaaccaagtcattctgagaatagtgt
atgcggcgaccgagttgctcttgcccggcgtcaatacgggataataccgc
gccacatagcagaactttaaaagtgctcatcattggaaaacgttcttcgg
ggcgaaaactctcaaggatcttaccgctgttgagatccagttcgatgtaa
cccactcgtgcacccaactgatcttcagcatcttttactttcaccagcgt
ttctgggtgagcaaaaacaggaaggcaaaatgccgcaaaaaagggaataa
gggcgacacggaaatgttgaatactcatactcttcctttttcaatattat
tgaagcatttatcagggttattgtctcatgagcggatacatatttgaatg
tatttagaaaaataaacaaataggggttccgcgcacatttccccgaaaag
tgccac

SEQ ID NO: 35 SV40 NLS (Pro-Lys-Lys-Lys-Arg-Lys-Val)

| | | | |
|---|---|---|---|
| | PAM (Human) | PAM (Mouse) | Sequence the gRNA binds to (5′ to 3′) |
| g1 | TTGGGT (SEQ ID NO: 36) | TTGGGT (SEQ ID NO: 36) | TGTCTACTATTTCTGGC (SEQ ID NO: 49) |
| g2 | CTGAAT (SEQ ID NO: 37) | CTGAAT (SEQ ID NO: 37) | TAGGGAGTGGCAAGTTGCCT (SEQ ID NO: 50) |
| g3 | CAGGGT (SEQ ID NO: 38) | CAGGGT (SEQ ID NO: 38) | GAATTAAGTTACATTGAAAC (SEQ ID NO: 51) |
| g4 | GAGAAT (SEQ ID NO: 39) | GAGAAT (SEQ ID NO: 39) | TGTGTTCCTTTGGTTTGATT (SEQ ID NO: 52) |
| g5 | TTTGAT (SEQ ID NO: 40) | TTTGAT (SEQ ID NO: 40) | CCAAAGGAACACAGAGAATGT (SEQ ID NO: 53) |
| g6 | TAGAAT (SEQ ID NO: 41) | TAGAAT (SEQ ID NO: 41) | CTAATTCTGGCACAAAATAGC (SEQ ID NO: 54) |
| g7 | TTCAAT (SEQ ID NO: 42) | TTCAAT (SEQ ID NO: 42) | ACATTCTCTGTGTTCCTTTGG (SEQ ID NO: 55) |
| g8 | AATGAT (SEQ ID NO: 43) | AATGAT (SEQ ID NO: 43) | CTACTAATTCTGGCACAAAAT (SEQ ID NO: 56) |
| g9 | AGAGAT (SEQ ID NO: 44) | AGAGAT (SEQ ID NO: 44) | GTTCTGTAGGCAACTTGCCAC (SEQ ID NO: 57) |
| g10 | ATGAAT (SEQ ID NO: 45) | ATAAGT (SEQ ID NO: 47) | AGCCAGAAATAGTAGACATGA (SEQ ID NO: 58) |
| g11 | TGAAAT (SEQ ID NO: 46) | CGAAAT (SEQ ID NO: 48) | ATTAACCCTGGAATTAAGTTA (SEQ ID NO: 59) |

| | | |
|---|---|---|
| | DNA encoding gRNA (5′ to 3′) | gRNA (5′ to 3′) |
| g1 | GCCAGAAATAGTAGACA (SEQ ID NO: 60) | GCCAGAAAUAGUAGACA (SEQ ID NO: 71) |
| g2 | AGGCAACTTGCCACTCCCTA (SEQ ID NO: 61) | AGGCAACUUGCCACUCCCUA (SEQ ID NO: 72) |
| g3 | GTTTCAATGTAACTTAATTC (SEQ ID NO: 62) | GUUUCAAUGUAACUUAAUUC (SEQ ID NO: 73) |
| g4 | AATCAAACCAAAGGAACACA (SEQ ID NO: 63) | AAUCAAACCAAAGGAACACA (SEQ ID NO: 74) |
| g5 | ACATTCTCTGTGTTCCTTTGG (SEQ ID NO: 64) | ACAUUCUCUGUGUUCCUUUGG (SEQ ID NO: 75) |
| g6 | GCTATTTTGTGCCAGAATTAG (SEQ ID NO: 65) | GCUAUUUUGUGCCAGAAUUAG (SEQ ID NO: 76) |
| g7 | CCAAAGGAACACAGAGAATGT (SEQ ID NO: 66) | CCAAAGGAACACAGAGAAUGU (SEQ ID NO: 77) |
| g8 | ATTTTGTGCCAGAATTAGTAG (SEQ ID NO: 67) | AUUUUGUGCCAGAAUUAGUAG (SEQ ID NO: 78) |
| g9 | GTGGCAAGTTGCCTACAGAAC (SEQ ID NO: 68) | GUGGCAAGUUGCCUACAGAAC (SEQ ID NO: 79) |
| g10 | TCATGTCTACTATTTCTGGCT (SEQ ID NO: 69) | UCAUGUCUACUAUUUCUGGCU (SEQ ID NO: 80) |
| g11 | TAACTTAATTCCAGGGTTAAT (SEQ ID NO: 70) | UAACUUAAUUCCAGGGUUAAU (SEQ ID NO: 81) |

SEQ ID NO: 82 Polynucleotide donor sequence

tgactgacctgagcgtaatacgactcactatagggcgaattgggtaccgc
taggtcttgaaaggagtgggaattggctccggtgcccgtcagtgggcaga
gcgcacatcgcccacagtccccgagaagttggggggaggggtcggcaatt
gatccggtgcctagagaaggtggcgcggggtaaactgggaaagtgatgtc
gtgtactggctccgcctttttcccgagggtgggggagaaccgtatataag
tgcagtagtcgccgtgaacgttctttttcgcaacgggtttgccgccagaa
cacaggaccggtgccaccatggccgcccacaggccggtggaatgggtcca
ggccgtggtcagccgcttcgacgagcagcttccaataaaaacaggacagc
agaacacacataccaaagtcagtactgagcacaacaaggaatgtctaatc
aatatttccaaatacaagttttctttggttataagcggcctcactactat
tttaaagaatgttaacaatatgagaatatttggagaagctgctgaaaaaa
atttatatctctctcagttgattatattggatacactggaaaaatgtctt
gctgggcaaccaaaggacacaatgagattagatgaaacgatgctggtcaa
acagttgctgccagaaatctgccattttcttcacacctgtcgtgaaggaa
accagcatgcagctgaacttcggaattctgcctctggggttttattttct
ctcagctgcaacaacttcaatgcagtctttagtcgcatttctaccaggtt
acaggaattaactgtttgttcagaagacaatgttgatgttcatgatatag
aattgttacagtatatcaatgtggattgtgcaaaattaaaacgactcctg
aaggaaacagcatttaaatttaaagccctaaagaaggttgcgcagttagc
agttataaatagcctggaaaaggcattttggaactgggtagaaaattatc
cagatgaatttacaaaactgtaccagatcccacagactgatatggctgaa
tgtgcagaaaagctatttgacttggtggatggttttgctgaaagcaccaa
acgtaaagcagcagtttggccactacaaatcattctccttatcttgtgtc
cagaaataatccaggatatatccaaagacgtggttgatgaaaacaacatg
aataagaagttatttctggacagtctacgaaaagctcttgctggccatgg
aggaagtaggcagctgacagaaagtgctgcaattgcctgtgtcaaactgt
gtaaagcaagtacttacatcaattgggaagataactctgtcattttccta
cttgttcagtccatggtggttgatcttaagaacctgctttttaatccaag
taagccattctcaagaggcagtcagcctgcagatgtggatctaatgattg
actgccttgtttcttgctttcgtataagccctcacaacaaccaacacttt
aagatctgcctggctcagaattcaccttctacatttcactatgtgctggt
aaattcactccatcgaatcatcaccaattccgcattggattggtggccta
agattgatgctgtgtattgtcactcggttgaacttcgaaatatgtttggt
gaaacacttcataaagcagtgcaaggttgtggagcacacccagcaatacg
aatggcaccgagtcttacatttaaagaaaaagtaacaagccttaaattta
aagaaaaacctacagacctggagacaagaagctataagtatcttctcttg
tccatggtgaaactaattcatgcagatccaaagctcttgctttgtaatcc
aagaaaacaggggcccgaaacccaaggcagtacagcagaattaattacag
ggctcgtccaactggtccctcagtcacacatgccagagattgctcaggaa
gcaatggaggctctgctggttcttcatcagttagatagcattgatttgtg
gaatcctgatgctcctgtagaaacattttgggagattagctcacaaatgc
ttttttacatctgcaagaaattaactagtcatcaaatgcttagtagcaca
gaaattctcaagtggttgcgggaaatattgatctgcaggaataaatttct
tcttaaaaataagcaggcagatagaagttcctgtcactttctcctttttt
acggggtaggatgtgatattccttctagtggaaataccagtcaaatgtcc
atggatcatgaagaattactacgtactcctggagcctctctccggaaggg
aaaagggaactcctctatggatagtgcagcaggatgcagcggaacccccc
cgatttgccgacaagcccagaccaaactagaagtggccctgtacatgttt
ctgtggaaccctgacactgaagctgttctggttgccatgtcctgtttccg
ccacctctgtgaggaagcagatatccggtgtggggtggatgaagtgtcag
tgcataacctcttgcccaactataacacattcatggagtttgcctctgtc
agcaatatgatgtcaacaggaagagcagcacttcagaaaagagtgatggc
actgctgaggcgcattgagcatcccactgcaggaaacactgaggcttggg
aagatacacatgcaaaatgggaacaagcaacaaagctaatccttaactat
ccaaaagccaaaatggaagatggccaggctgctgaaagccttcacaagac
cattgttaagaggcgaatgtcccatgtgagtggaggaggatccatagatt
tgtctgacacagactccctacaggaatggatcaacatgactggcttcctt
tgtgcccttgggggagtgtgcctccagcagagaagcaattctggcctggc
aacctatagcccacccatgggtccagtcagtgaacgtaagggttctatga
tttcagtgatgtcttcagagggaaacgcagatacacctgtcagcaaattt
atggatcggctgttgtccttaatggtgtgtaaccatgagaaagtgggact
tcaaatacggaccaatgttaaggatctggtgggtctagaattgagtcctg
ctctgtatccaatgctatttaacaaattgaagaataccatcagcaagttt
tttgactcccaaggacaggttttattgactgataccaatactcaatttgt
agaacaaaccatagctataatgaagaacttgctagataatcatactgaag
gcagctctgaacatctagggcaagctagcattgaaacaatgatgttaaat
ctggtcaggtatgttcgtgtgcttgggaatatggtccatgcaattcaaat
aaaaacgaaactgtgtcaattagttgaagtaatgatggcaaggagagatg
acctctcattttgccaagagatgaaatttaggaataagatggtagaatac
ctgacagactgggttatgggaacatcaaaccaagcagcagatgatgatgt
aaaatgtcttacaagagatttggaccaggcaagcatggaagcagtagttt
cacttctagctggtctccctctgcagcctgaagaaggagatggtgtggaa
ttgatggaagccaaatcacagttatttcttaaatacttcacattatttat
gaaccttttgaatgactgcagtgaagttgaagatgaaagtgcgcaaacag
gtggcaggaaacgtggcatgtctcggaggctggcatcactgaggcactgt
acggtccttgcaatgtcaaacttactcaatgccaacgtagacagtggtct
catgcactccataggcttaggttaccacaaggatctccagacaagagcta
catttatggaagttctgacaaaaatccttcaacaaggcacagaatttgac
acacttgcagaaacagtattggctgatcggtttgagagattggtggaact
ggtcacaatgatgggtgatcaaggagaactccctatagcgatggctctgg
ccaatgtggttccttgttctcagtgggatgaactagctcgagttctggtt
actctgtttgattctcggcatttactctaccaactgctctggaacatgtt
ttctaaagaagtagaattggcagactccatgcagactctcttccgaggca
acagcttggccagtaaaataatgacattctgtttcaaggtatatggtgct
acctatctacaaaaactcctggatcctttattacgaattgtgatcacatc
ctctgattggcaacatgttagctttgaagtggatcctaccaggttagaac
catcagagagccttgaggaaaaccagcggaacctccttcagatgactgaa
aagttcttccatgccatcatcagttcctcctcagaattcccccctcaact
tcgaagtgtgtgccactgcttataccaggtaagtatggaggaaggaattg
cgtgttatgttgacccgtgcctaggtattacgca

SEQ ID NO: 83 (Plasmid 16) Polynucleotide sequence of the donor sequence flanked by gRNA 1 target sites (7472 bp)

cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcctgcggcctctagacggatgtg
ctgACCCAATCATGTCTACTATTTCTGGCTGACTGACCTGAgcgtaatac
gactcactatagggcgaattgggtacCGCTAGGTCTTGAAAGGAGTGGGA
ATTGGCTCCGGTGCCCGTCAGTGGGCAGAGCGCACATCGCCCACAGTCCC
CGAGAAGTTGGGGGGAGGGGTCGGCAATTGATCCGGTGCCTAGAGAAGGT
GGCGCGGGGTAAACTGGGAAAGTGATGTCGTGTACTGGCTCCGCCTTTTT
CCCGAGGGTGGGGGAGAACCGTATATAAGTGCAGTAGTCGCCGTGAACGT
TCTTTTTCGCAACGGGTTTGCCGCCAGAACACAGGACCGGTGCCACCATG
GCCGCCCACAGGCCGGTGGAATGGGTCCAGGCCGTGGTCAGCCGCTTCGA
CGAGCAGCTTCCAATAAAAACAGGACAGCAGAACACACATACCAAAGTCA
GTACTGAGCACAACAAGGAATGTCTAATCAATATTTCCAAATACAAGTTT
TCTTTGGTTATAAGCGGCCTCACTACTATTTTAAAGAATGTTAACAATAT
GAGAATATTTGGAGAAGCTGCTGAAAAAAATTTATATCTCTCTCAGTTGA
TTATATTGGATACACTGGAAAAATGTCTTGCTGGGCAACCAAAGGACACA
ATGAGATTAGATGAAACGATGCTGGTCAAACAGTTGCTGCCAGAAATCTG
CCATTTTCTTCACACCTGTCGTGAAGGAAACCAGCATGCAGCTGAACTTC
GGAATTCTGCCTCTGGGGTTTTATTTTCTCTCAGCTGCAACAACTTCAAT
GCAGTCTTTAGTCGCATTTCTACCAGGTTACAGGAATTAACTGTTTGTTC
AGAAGACAATGTTGATGTTCATGATATAGAATTGTTACAGTATATCAATG
TGGATTGTGCAAAATTAAAACGACTCCTGAAGGAAACAGCATTTAAATTT
AAAGCCCTAAAGAAGGTTGCGCAGTTAGCAGTTATAAATAGCCTGGAAAA
GGCATTTTGGAACTGGGTAGAAAATTATCCAGATGAATTTACAAAACTGT
ACCAGATCCCACAGACTGATATGGCTGAATGTGCAGAAAAGCTATTTGAC
TTGGTGGATGGTTTTGCTGAAAGCACCAAACGTAAAGCAGCAGTTTGGCC
ACTACAAATCATTCTCCTTATCTTGTGTCCAGAAATAATCCAGGATATAT
CCAAAGACGTGGTTGATGAAAACAACATGAATAAGAAGTTATTTCTGGAC
AGTCTACGAAAAGCTCTTGCTGGCCATGGAGGAAGTAGGCAGCTGACAGA
AAGTGCTGCAATTGCCTGTGTCAAACTGTGTAAAGCAAGTACTTACATCA
ATTGGGAAGATAACTCTGTCATTTTCCTACTTGTTCAGTCCATGGTGGTT
GATCTTAAGAACCTGCTTTTTAATCCAAGTAAGCCATTCTCAAGAGGCAG
TCAGCCTGCAGATGTGGATCTAATGATTGACTGCCTTGTTTCTTGCTTTC
GTATAAGCCCTCACAACAACCAACACTTTAAGATCTGCCTGGCTCAGAAT
TCACCTTCTACATTTCACTATGTGCTGGTAAATTCACTCCATCGAATCAT
CACCAATTCCGCATTGGATTGGTGGCCTAAGATTGATGCTGTGTATTGTC
ACTCGGTTGAACTTCGAAATATGTTTGGTGAAACACTTCATAAAGCAGTG
CAAGGTTGTGGAGCACACCCAGCAATACGAATGGCACCGAGTCTTACATT
TAAAGAAAAAGTAACAAGCCTTAAATTTAAAGAAAAACCTACAGACCTGG
AGACAAGAAGCTATAAGTATCTTCTCTTGTCCATGGTGAAACTAATTCAT
GCAGATCCAAAGCTCTTGCTTTGTAATCCAAGAAAACAGGGGCCCGAAAC
CCAAGGCAGTACAGCAGAATTAATTACAGGGCTCGTCCAACTGGTCCCTC
AGTCACACATGCCAGAGATTGCTCAGGAAGCAATGGAGGCTCTGCTGGTT
CTTCATCAGTTAGATAGCATTGATTTGTGGAATCCTGATGCTCCTGTAGA
AACATTTTGGGAGATTAGCTCACAAATGCTTTTTTACATCTGCAAGAAAT
TAACTAGTCATCAAATGCTTAGTAGCACAGAAATTCTCAAGTGGTTGCGG
GAAATATTGATCTGCAGGAATAAATTTCTTCTTAAAAATAAGCAGGCAGA
TAGAAGTTCCTGTCACTTTCTCCTTTTTTACGGGGTAGGATGTGATATTC
CTTCTAGTGGAAATACCAGTCAAATGTCCATGGATCATGAAGAATTACTA
CGTACTCCTGGAGCCTCTCTCCGGAAGGGAAAAGGGAACTCCTCTATGGA
TAGTGCAGCAGGATGCAGCGGAACCCCCCCGATTTGCCGACAAGCCCAGA
CCAAACTAGAAGTGGCCCTGTACATGTTTCTGTGGAACCCTGACACTGAA
GCTGTTCTGGTTGCCATGTCCTGTTTCCGCCACCTCTGTGAGGAAGCAGA
TATCCGGTGTGGGGTGGATGAAGTGTCAGTGCATAACCTCTTGCCCAACT
ATAACACATTCATGGAGTTTGCCTCTGTCAGCAATATGATGTCAACAGGA
AGAGCAGCACTTCAGAAAAGAGTGATGGCACTGCTGAGGCGCATTGAGCA
TCCCACTGCAGGAAACACTGAGGCTTGGGAAGATACACATGCAAAATGGG
AACAAGCAACAAAGCTAATCCTTAACTATCCAAAAGCCAAAATGGAAGAT
GGCCAGGCTGCTGAAAGCCTTCACAAGACCATTGTTAAGAGGCGAATGTC
CCATGTGAGTGGAGGAGGATCCATAGATTTGTCTGACACAGACTCCCTAC
AGGAATGGATCAACATGACTGGCTTCCTTTGTGCCCTTGGGGGAGTGTGC
CTCCAGCAGAGAAGCAATTCTGGCCTGGCAACCTATAGCCCACCCATGGG
TCCAGTCAGTGAACGTAAGGGTTCTATGATTTCAGTGATGTCTTCAGAGG
GAAACGCAGATACACCTGTCAGCAAATTTATGGATCGGCTGTTGTCCTTA
ATGGTGTGTAACCATGAGAAAGTGGGACTTCAAATACGGACCAATGTTAA
GGATCTGGTGGGTCTAGAATTGAGTCCTGCTCTGTATCCAATGCTATTTA
ACAAATTGAAGAATACCATCAGCAAGTTTTTTGACTCCCAAGGACAGGTT
TTATTGACTGATACCAATACTCAATTTGTAGAACAAACCATAGCTATAAT
GAAGAACTTGCTAGATAATCATACTGAAGGCAGCTCTGAACATCTAGGGC
AAGCTAGCATTGAAACAATGATGTTAAATCTGGTCAGGTATGTTCGTGTG
CTTGGGAATATGGTCCATGCAATTCAAATAAAAACGAAACTGTGTCAATT
AGTTGAAGTAATGATGGCAAGGAGAGATGACCTCTCATTTTGCCAAGAGA
TGAAATTTAGGAATAAGATGGTAGAATACCTGACAGACTGGGTTATGGGA
ACATCAAACCAAGCAGCAGATGATGATGTAAAATGTCTTACAAGAGATTT
GGACCAGGCAAGCATGGAAGCAGTAGTTTCACTTCTAGCTGGTCTCCCTC
TGCAGCCTGAAGAAGGAGATGGTGTGGAATTGATGGAAGCCAAATCACAG
TTATTTCTTAAATACTTCACATTATTTATGAACCTTTTGAATGACTGCAG
TGAAGTTGAAGATGAAAGTGCGCAAACAGGTGGCAGGAAACGTGGCATGT
CTCGGAGGCTGGCATCACTGAGGCACTGTACGGTCCTTGCAATGTCAAAC
TTACTCAATGCCAACGTAGACAGTGGTCTCATGCACTCCATAGGCTTAGG
TTACCACAAGGATCTCCAGACAAGAGCTACATTTATGGAAGTTCTGACAA
AAATCCTTCAACAAGGCACAGAATTTGACACACTTGCAGAAACAGTATTG
GCTGATCGGTTTGAGAGATTGGTGGAACTGGTCACAATGATGGGTGATCA
AGGAGAACTCCCTATAGCGATGGCTCTGGCCAATGTGGTTCCTTGTTCTC
AGTGGGATGAACTAGCTCGAGTTCTGGTTACTCTGTTTGATTCTCGGCAT
TTACTCTACCAACTGCTCTGGAACATGTTTTCTAAAGAAGTAGAATTGGC
AGACTCCATGCAGACTCTCTTCCGAGGCAACAGCTTGGCCAGTAAAATAA
TGACATTCTGTTTCAAGGTATATGGTGCTACCTATCTACAAAAACTCCTG
GATCCTTTATTACGAATTGTGATCACATCCTCTGATTGGCAACATGTTAG
CTTTGAAGTGGATCCTACCAGGTTAGAACCATCAGAGAGCCTTGAGGAAA
ACCAGCGGAACCTCCTTCAGATGACTGAAAAGTTCTTCCATGCCATCATC
AGTTCCTCCTCAGAATTCCCCCCTCAACTTCGAAGTGTGTGCCACTGCTT
ATACCAGGTAAGTATGGAGGAAGGAATTGCGTGTTATGTTGACCCGTGCC
TAGGTATTACGCAACCCAATCATGTCTACTATTTCTGGCGCggccgcCCg
cggtggagctccagcttttgttccctttggccgcaggaacccctagtgat
ggagttggccactccctctctgcgcgctcgctcgctcactgaggccgggc
gaccaaaggtcgcccgacgcccgggctttgcccgggcggcctcagtgagc
gagcgagcgcgcagctgcctgcaggggcgcctgatgcggtattttctcct
tacgcatctgtgcggtatttcacaccgcatacgtcaaagcaaccatagta
cgcgccctgtagcggcgcattaagcgcggcgggtgtggtggttacgcgca
gcgtgaccgctacacttgccagcgccctagcgcccgctcctttcgctttc
ttcccttcctttctcgccacgttcgccggctttccccgtcaagctctaaa
tcgggggctccctttagggttccgatttagtgctttacggcacctcgacc
ccaaaaaacttgatttgggtgatggttcacgtagtgggccatcgccctga
tagacggtttttcgccctttgacgttggagtccacgttctttaatagtgg
actcttgttccaaactggaacaacactcaaccctatctcgggctattctt
ttgatttataagggattttgccgatttcggcctattggttaaaaaatgag
ctgatttaacaaaaatttaacgcgaattttaacaaaatattaacgtttac
aattttatggtgcactctcagtacaatctgctctgatgccgcatagttaa
gccagccccgacacccgccaacacccgctgacgcgccctgacgggcttgt
ctgctcccggcatccgcttacagacaagctgtgaccgtctccgggagctg
catgtgtcagaggttttcaccgtcatcaccgaaacgcgcgagacgaaagg
gcctcgtgatacgcctatttttataggttaatgtcatgataataatggtt
tcttagacgtcaggtggcacttttcggggaaatgtgcgcggaacccctat
ttgtttatttttctaaatacattcaaatatgtatccgctcatgagacaat
aaccctgataaatgcttcaataatattgaaaaaggaagagtatgagtatt
caacatttccgtgtcgcccttattcccttttttgcggcattttgccttcc
tgtttttgctcacccagaaacgctggtgaaagtaaaagatgctgaagatc
agttgggtgcacgagtgggttacatcgaactggatctcaacagcggtaag
atccttgagagttttcgccccgaagaacgttttccaatgatgagcacttt
taaagttctgctatgtggcgcggtattatcccgtattgacgccgggcaag
agcaactcggtcgccgcatacactattctcagaatgacttggttgagtac
tcaccagtcacagaaaagcatcttacggatggcatgacagtaagagaatt
atgcagtgctgccataaccatgagtgataacactgcggccaacttacttc
tgacaacgatcggaggaccgaaggagctaaccgcttttttgcacaacatg
ggggatcatgtaactcgccttgatcgttgggaaccggagctgaatgaagc
cataccaaacgacgagcgtgacaccacgatgcctgtagcaatggcaacaa
cgttgcgcaaactattaactggcgaactacttactctagcttcccggcaa
caattaatagactggatggaggcggataaagttgcaggaccacttctgcg
ctcggcccttccggctggctggtttattgctgataaatctggagccggtg
agcgtggaagccgcggtatcattgcagcactggggccagatggtaagccc
tcccgtatcgtagttatctacacgacggggagtcaggcaactatggatga
acgaaatagacagatcgctgagataggtgcctcactgattaagcattggt
aactgtcagaccaagtttactcatatatactttagattgatttaaaactt
catttttaatttaaaaggatctaggtgaagatcctttttgataatctcat
gaccaaaatcccttaacgtgagttttcgttccactgagcgtcagaccccg
tagaaaagatcaaaggatcttcttgagatcctttttttctgcgcgtaatc
tgctgcttgcaaacaaaaaaaccaccgctaccagcggtggtttgtttgcc
ggatcaagagctaccaactctttttccgaaggtaactggcttcagcagag
cgcagataccaaatactgtccttctagtgtagccgtagttaggccaccac
ttcaagaactctgtagcaccgcctacatacctcgctctgctaatcctgtt
accagtggctgctgccagtggcgataagtcgtgtcttaccgggttggact
caagacgatagttaccggataaggcgcagcggtcgggctgaacggggggt
tcgtgcacacagcccagcttggagcgaacgacctacaccgaactgagata
cctacagcgtgagctatgagaaagcgccacgcttcccgaagggagaaagg
cggacaggtatccggtaagcggcagggtcggaacaggagagcgcacgagg
gagcttccagggggaaacgcctggtatctttatagtcctgtcgggtttcg
ccacctctgacttgagcgtcgatttttgtgatgctcgtcaggggggcgga
gcctatggaaaaacgccagcaacgcggcctttttacggttcctggccttt
tgctggccttttgctcacatgt

SEQ ID NO: 84 (Plasmid 47) Polynucleotide sequence of the donor sequence flanked by gRNA 3 target sites (7465 bp)

cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcctgcggcctctagacggatgtg
ctgGTTTCAATGTAACTTAATTCCAGGGTTGACTGACCTGAgcgtaatac
gactcactatagggcgaattgggtacCGCTAGGTCTTGAAAGGAGTGGGA
ATTGGCTCCGGTGCCCGTCAGTGGGCAGAGCGCACATCGCCCACAGTCCC
CGAGAAGTTGGGGGGAGGGGTCGGCAATTGATCCGGTGCCTAGAGAAGGT
GGCGCGGGGTAAACTGGGAAAGTGATGTCGTGTACTGGCTCCGCCTTTTT
CCCGAGGGTGGGGGAGAACCGTATATAAGTGCAGTAGTCGCCGTGAACGT
TCTTTTTCGCAACGGGTTTGCCGCCAGAACACAGGACCGGTGCCACCATG
GCCGCCCACAGGCCGGTGGAATGGGTCCAGGCCGTGGTCAGCCGCTTCGA
CGAGCAGCTTCCAATAAAAACAGGACAGCAGAACACACATACCAAAGTCA
GTACTGAGCACAACAAGGAATGTCTAATCAATATTTCCAAATACAAGTTT
TCTTTGGTTATAAGCGGCCTCACTACTATTTTAAAGAATGTTAACAATAT
GAGAATATTTGGAGAAGCTGCTGAAAAAAATTTATATCTCTCTCAGTTGA
TTATATTGGATACACTGGAAAAATGTCTTGCTGGGCAACCAAAGGACACA
ATGAGATTAGATGAAACGATGCTGGTCAAACAGTTGCTGCCAGAAATCTG
CCATTTTCTTCACACCTGTCGTGAAGGAAACCAGCATGCAGCTGAACTTC
GGAATTCTGCCTCTGGGGTTTTATTTTCTCTCAGCTGCAACAACTTCAAT
GCAGTCTTTAGTCGCATTTCTACCAGGTTACAGGAATTAACTGTTTGTTC
AGAAGACAATGTTGATGTTCATGATATAGAATTGTTACAGTATATCAATG
TGGATTGTGCAAAATTAAAACGACTCCTGAAGGAAACAGCATTTAAATTT
AAAGCCCTAAAGAAGGTTGCGCAGTTAGCAGTTATAAATAGCCTGGAAAA
GGCATTTTGGAACTGGGTAGAAAATTATCCAGATGAATTTACAAAACTGT
ACCAGATCCCACAGACTGATATGGCTGAATGTGCAGAAAAGCTATTTGAC
TTGGTGGATGGTTTTGCTGAAAGCACCAAACGTAAAGCAGCAGTTTGGCC
ACTACAAATCATTCTCCTTATCTTGTGTCCAGAAATAATCCAGGATATAT
CCAAAGACGTGGTTGATGAAAACAACATGAATAAGAAGTTATTTCTGGAC
AGTCTACGAAAAGCTCTTGCTGGCCATGGAGGAAGTAGGCAGCTGACAGA
AAGTGCTGCAATTGCCTGTGTCAAACTGTGTAAAGCAAGTACTTACATCA
ATTGGGAAGATAACTCTGTCATTTTCCTACTTGTTCAGTCCATGGTGGTT
GATCTTAAGAACCTGCTTTTTAATCCAAGTAAGCCATTCTCAAGAGGCAG
TCAGCCTGCAGATGTGGATCTAATGATTGACTGCCTTGTTTCTTGCTTTC
GTATAAGCCCTCACAACAACCAACACTTTAAGATCTGCCTGGCTCAGAAT
TCACCTTCTACATTTCACTATGTGCTGGTAAATTCACTCCATCGAATCAT
CACCAATTCCGCATTGGATTGGTGGCCTAAGATTGATGCTGTGTATTGTC
ACTCGGTTGAACTTCGAAATATGTTTGGTGAAACACTTCATAAAGCAGTG
CAAGGTTGTGGAGCACACCCAGCAATACGAATGGCACCGAGTCTTACATT
TAAAGAAAAAGTAACAAGCCTTAAATTTAAAGAAAAACCTACAGACCTGG
AGACAAGAAGCTATAAGTATCTTCTCTTGTCCATGGTGAAACTAATTCAT
GCAGATCCAAAGCTCTTGCTTTGTAATCCAAGAAAACAGGGGCCCGAAAC
CCAAGGCAGTACAGCAGAATTAATTACAGGGCTCGTCCAACTGGTCCCTC
AGTCACACATGCCAGAGATTGCTCAGGAAGCAATGGAGGCTCTGCTGGTT
CTTCATCAGTTAGATAGCATTGATTTGTGGAATCCTGATGCTCCTGTAGA
AACATTTTGGGAGATTAGCTCACAAATGCTTTTTTACATCTGCAAGAAAT
TAACTAGTCATCAAATGCTTAGTAGCACAGAAATTCTCAAGTGGTTGCGG
GAAATATTGATCTGCAGGAATAAATTTCTTCTTAAAAATAAGCAGGCAGA
TAGAAGTTCCTGTCACTTTCTCCTTTTTTACGGGGTAGGATGTGATATTC
CTTCTAGTGGAAATACCAGTCAAATGTCCATGGATCATGAAGAATTACTA
CGTACTCCTGGAGCCTCTCTCCGGAAGGGAAAAGGGAACTCCTCTATGGA
TAGTGCAGCAGGATGCAGCGGAACCCCCCCGATTTGCCGACAAGCCCAGA
CCAAACTAGAAGTGGCCCTGTACATGTTTCTGTGGAACCCTGACACTGAA
GCTGTTCTGGTTGCCATGTCCTGTTTCCGCCACCTCTGTGAGGAAGCAGA
TATCCGGTGTGGGGTGGATGAAGTGTCAGTGCATAACCTCTTGCCCAACT
ATAACACATTCATGGAGTTTGCCTCTGTCAGCAATATGATGTCAACAGGA
AGAGCAGCACTTCAGAAAAGAGTGATGGCACTGCTGAGGCGCATTGAGCA
TCCCACTGCAGGAAACACTGAGGCTTGGGAAGATACACATGCAAAATGGG
AACAAGCAACAAAGCTAATCCTTAACTATCCAAAAGCCAAAATGGAAGAT
GGCCAGGCTGCTGAAAGCCTTCACAAGACCATTGTTAAGAGGCGAATGTC
CCATGTGAGTGGAGGAGGATCCATAGATTTGTCTGACACAGACTCCCTAC
AGGAATGGATCAACATGACTGGCTTCCTTTGTGCCCTTGGGGGAGTGTGC
CTCCAGCAGAGAAGCAATTCTGGCCTGGCAACCTATAGCCCACCCATGGG
TCCAGTCAGTGAACGTAAGGGTTCTATGATTTCAGTGATGTCTTCAGAGG
GAAACGCAGATACACCTGTCAGCAAATTTATGGATCGGCTGTTGTCCTTA
ATGGTGTGTAACCATGAGAAAGTGGGACTTCAAATACGGACCAATGTTAA
GGATCTGGTGGGTCTAGAATTGAGTCCTGCTCTGTATCCAATGCTATTTA
ACAAATTGAAGAATACCATCAGCAAGTTTTTTGACTCCCAAGGACAGGTT
TTATTGACTGATACCAATACTCAATTTGTAGAACAAACCATAGCTATAAT
GAAGAACTTGCTAGATAATCATACTGAAGGCAGCTCTGAACATCTAGGGC
AAGCTAGCATTGAAACAATGATGTTAAATCTGGTCAGGTATGTTCGTGTG
CTTGGGAATATGGTCCATGCAATTCAAATAAAAACGAAACTGTGTCAATT
AGTTGAAGTAATGATGGCAAGGAGAGATGACCTCTCATTTTGCCAAGAGA
TGAAATTTAGGAATAAGATGGTAGAATACCTGACAGACTGGGTTATGGGA
ACATCAAACCAAGCAGCAGATGATGATGTAAAATGTCTTACAAGAGATTT
GGACCAGGCAAGCATGGAAGCAGTAGTTTCACTTCTAGCTGGTCTCCCTC
TGCAGCCTGAAGAAGGAGATGGTGTGGAATTGATGGAAGCCAAATCACAG
TTATTTCTTAAATACTTCACATTATTTATGAACCTTTTGAATGACTGCAG
TGAAGTTGAAGATGAAAGTGCGCAAACAGGTGGCAGGAAACGTGGCATGT
CTCGGAGGCTGGCATCACTGAGGCACTGTACGGTCCTTGCAATGTCAAAC
TTACTCAATGCCAACGTAGACAGTGGTCTCATGCACTCCATAGGCTTAGG
TTACCACAAGGATCTCCAGACAAGAGCTACATTTATGGAAGTTCTGACAA
AAATCCTTCAACAAGGCACAGAATTTGACACACTTGCAGAAACAGTATTG
GCTGATCGGTTTGAGAGATTGGTGGAACTGGTCACAATGATGGGTGATCA
AGGAGAACTCCCTATAGCGATGGCTCTGGCCAATGTGGTTCCTTGTTCTC
AGTGGGATGAACTAGCTCGAGTTCTGGTTACTCTGTTTGATTCTCGGCAT
TTACTCTACCAACTGCTCTGGAACATGTTTTCTAAAGAAGTAGAATTGGC
AGACTCCATGCAGACTCTCTTCCGAGGCAACAGCTTGGCCAGTAAAATAA
TGACATTCTGTTTCAAGGTATATGGTGCTACCTATCTACAAAAACTCCTG
GATCCTTTATTACGAATTGTGATCACATCCTCTGATTGGCAACATGTTAG
CTTTGAAGTGGATCCTACCAGGTTAGAACCATCAGAGAGCCTTGAGGAAA
ACCAGCGGAACCTCCTTCAGATGACTGAAAAGTTCTTCCATGCCATCATC
AGTTCCTCCTCAGAATTCCCCCCTCAACTTCGAAGTGTGTGCCACTGCTT
ATACCAGGTAAGTTATGGAGGAAGGAATTGCGTGTTATGTTGACCCGTGC
CTAGGTATTACGCGTTTCAATGTAACTTAATTCCAGGGTCAAGCTTGTCA
gctccagcttttgttccctttggccgcaggaacccctagtgatggagttg
gccactccctctctgcgcgctcgctcgctcactgaggccaggcgaccaaa
ggtcgcccgacgcccggactttacccgggcggcctcagtgagcgagcgag
cgcgcagctgcctgcaggggcgcctgatgcggtattttctccttacgcat
ctgtgcggtatttcacaccgcatacgtcaaagcaaccatagtacgcgccc
tgtagcggcgcattaagcgcggcgggtgtggtggttacgcgcagcgtgac
cgctacacttgccagcgccctagcgcccgctcctttcgctttcttccctt
cctttctcgccacgttcgccggctttccccgtcaagctctaaatcggggg
ctccctttagggttccgatttagtgctttacggcacctcgaccccaaaaa
acttgatttgggtgatggttcacgtagtgggccatcgccctgatagacgg
tttttcgccctttgacgttggagtccacgttctttaatagtggactcttg
ttccaaactggaacaacactcaaccctatctcgggctattcttttgattt
ataagggattttgccgatttcggcctattggttaaaaaatgagctgattt
aacaaaaatttaacgcgaattttaacaaaatattaacgtttacaatttta
tggtgcactctcagtacaatctgctctgatgccgcatagttaagccagcc
ccgacacccgccaacacccgctgacgcgccctgacgggcttgtctgctcc
cggcatccgcttacagacaagctgtgaccgtctccgggagctgcatgtgt
cagaggttttcaccgtcatcaccgaaacgcgcgagacgaaagggcctcgt
gatacgcctatttttataggttaatgtcatgataataatggtttcttaga
cgtcaggtggcacttttcggggaaatgtgcgcggaacccctatttgttta
tttttctaaatacattcaaatatgtatccgctcatgagacaataaccctg
ataaatgcttcaataatattgaaaaaggaagagtatgagtattcaacatt
tccgtgtcgcccttattcccttttttgcggcattttgccttcctgttttt
gctcacccagaaacgctggtgaaagtaaaagatgctgaagatcagttggg
tgcacgagtgggttacatcgaactggatctcaacagcggtaagatccttg
agagttttcgccccgaagaacgttttccaatgatgagcacttttaaagtt
ctgctatgtggcgcggtattatcccgtattgacgccgggcaagagcaact
cggtcgccgcatacactattctcagaatgacttggttgagtactcaccag
tcacagaaaagcatcttacggatggcatgacagtaagagaattatgcagt
gctgccataaccatgagtgataacactgcggccaacttacttctgacaac
gatcggaggaccgaaggagctaaccgcttttttgcacaacatgggggatc
atgtaactcgccttgatcgttgggaaccggagctgaatgaagccatacca
aacgacgagcgtgacaccacgatgcctgtagcaatggcaacaacgttgcg
caaactattaactggcgaactacttactctagcttcccggcaacaattaa
tagactggatggaggcggataaagttgcaggaccacttctgcgctcggcc
cttccggctggctggtttattgctgataaatctggagccggtgagcgtgg
aagccgcggtatcattgcagcactggggccagatggtaagccctcccgta
tcgtagttatctacacgacggggagtcaggcaactatggatgaacgaaat
agacagatcgctgagataggtgcctcactgattaagcattggtaactgtc
agaccaagtttactcatatatactttagattgatttaaaacttcattttt
aatttaaaaggatctaggtgaagatcctttttgataatctcatgaccaaa
atcccttaacgtgagttttcgttccactgagcgtcagaccccgtagaaaa
gatcaaaggatcttcttgagatcctttttttctgcgcgtaatctgctgct
tgcaaacaaaaaaaccaccgctaccagcggtggtttgtttgccggatcaa
gagctaccaactctttttccgaaggtaactggcttcagcagagcgcagat
accaaatactgtccttctagtgtagccgtagttaggccaccacttcaaga
actctgtagcaccgcctacatacctcgctctgctaatcctgttaccagtg
gctgctgccagtggcgataagtcgtgtcttaccgggttggactcaagacg
atagttaccggataaggcgcagcggtcgggctgaacggggggttcgtgca
cacagcccagcttggagcgaacgacctacaccgaactgagatacctacag
cgtgagctatgagaaagcgccacgcttcccgaagggagaaaggcggacag
gtatccggtaagcggcagggtcggaacaggagagcgcacgagggagcttc
cagggggaaacgcctggtatctttatagtcctgtcgggtttcgccacctc
tgacttgagcgtcgatttttgtgatgctcgtcaggggggcggagcctatg
gaaaaacgccagcaacgcggcctttttacggttcctggccttttgctggc
cttttgctcacatgt

SEQ ID NO: 85 (Plasmid 48) Polynucleotide sequence of the donor sequence flanked by gRNA 4 target sites (7472 bp)

cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcctgcggcctctagacggatgtg
ctgAATCAAACCAAAGGAACACAGAGAATTGACTGACCTGAgcgtaatac
gactcactatagggcgaattgggtacCGCTAGGTCTTGAAAGGAGTGGGA
ATTGGCTCCGGTGCCCGTCAGTGGGCAGAGCGCACATCGCCCACAGTCCC
CGAGAAGTTGGGGGGAGGGGTCGGCAATTGATCCGGTGCCTAGAGAAGGT
GGCGCGGGGTAAACTGGGAAAGTGATGTCGTGTACTGGCTCCGCCTTTTT
CCCGAGGGTGGGGGAGAACCGTATATAAGTGCAGTAGTCGCCGTGAACGT
TCTTTTTCGCAACGGGTTTGCCGCCAGAACACAGGACCGGTGCCACCATG
GCCGCCCACAGGCCGGTGGAATGGGTCCAGGCCGTGGTCAGCCGCTTCGA
CGAGCAGCTTCCAATAAAAACAGGACAGCAGAACACACATACCAAAGTCA
GTACTGAGCACAACAAGGAATGTCTAATCAATATTTCCAAATACAAGTTT
TCTTTGGTTATAAGCGGCCTCACTACTATTTTAAAGAATGTTAACAATAT
GAGAATATTTGGAGAAGCTGCTGAAAAAAATTTATATCTCTCTCAGTTGA
TTATATTGGATACACTGGAAAAATGTCTTGCTGGGCAACCAAAGGACACA
ATGAGATTAGATGAAACGATGCTGGTCAAACAGTTGCTGCCAGAAATCTG
CCATTTTCTTCACACCTGTCGTGAAGGAAACCAGCATGCAGCTGAACTTC
GGAATTCTGCCTCTGGGGTTTTATTTTCTCTCAGCTGCAACAACTTCAAT
GCAGTCTTTAGTCGCATTTCTACCAGGTTACAGGAATTAACTGTTTGTTC
AGAAGACAATGTTGATGTTCATGATATAGAATTGTTACAGTATATCAATG
TGGATTGTGCAAAATTAAAACGACTCCTGAAGGAAACAGCATTTAAATTT
AAAGCCCTAAAGAAGGTTGCGCAGTTAGCAGTTATAAATAGCCTGGAAAA
GGCATTTTGGAACTGGGTAGAAAATTATCCAGATGAATTTACAAAACTGT
ACCAGATCCCACAGACTGATATGGCTGAATGTGCAGAAAAGCTATTTGAC
TTGGTGGATGGTTTTGCTGAAAGCACCAAACGTAAAGCAGCAGTTTGGCC
ACTACAAATCATTCTCCTTATCTTGTGTCCAGAAATAATCCAGGATATAT
CCAAAGACGTGGTTGATGAAAACAACATGAATAAGAAGTTATTTCTGGAC
AGTCTACGAAAAGCTCTTGCTGGCCATGGAGGAAGTAGGCAGCTGACAGA
AAGTGCTGCAATTGCCTGTGTCAAACTGTGTAAAGCAAGTACTTACATCA
ATTGGGAAGATAACTCTGTCATTTTCCTACTTGTTCAGTCCATGGTGGTT
GATCTTAAGAACCTGCTTTTTAATCCAAGTAAGCCATTCTCAAGAGGCAG
TCAGCCTGCAGATGTGGATCTAATGATTGACTGCCTTGTTTCTTGCTTTC
GTATAAGCCCTCACAACAACCAACACTTTAAGATCTGCCTGGCTCAGAAT
TCACCTTCTACATTTCACTATGTGCTGGTAAATTCACTCCATCGAATCAT
CACCAATTCCGCATTGGATTGGTGGCCTAAGATTGATGCTGTGTATTGTC
ACTCGGTTGAACTTCGAAATATGTTTGGTGAAACACTTCATAAAGCAGTG
CAAGGTTGTGGAGCACACCCAGCAATACGAATGGCACCGAGTCTTACATT
TAAAGAAAAAGTAACAAGCCTTAAATTTAAAGAAAAACCTACAGACCTGG
AGACAAGAAGCTATAAGTATCTTCTCTTGTCCATGGTGAAACTAATTCAT
GCAGATCCAAAGCTCTTGCTTTGTAATCCAAGAAAACAGGGGCCCGAAAC
CCAAGGCAGTACAGCAGAATTAATTACAGGGCTCGTCCAACTGGTCCCTC
AGTCACACATGCCAGAGATTGCTCAGGAAGCAATGGAGGCTCTGCTGGTT
CTTCATCAGTTAGATAGCATTGATTTGTGGAATCCTGATGCTCCTGTAGA
AACATTTTGGGAGATTAGCTCACAAATGCTTTTTTACATCTGCAAGAAAT
TAACTAGTCATCAAATGCTTAGTAGCACAGAAATTCTCAAGTGGTTGCGG
GAAATATTGATCTGCAGGAATAAATTTCTTCTTAAAAATAAGCAGGCAGA
TAGAAGTTCCTGTCACTTTCTCCTTTTTTACGGGGTAGGATGTGATATTC
CTTCTAGTGGAAATACCAGTCAAATGTCCATGGATCATGAAGAATTACTA
CGTACTCCTGGAGCCTCTCTCCGGAAGGGAAAAGGGAACTCCTCTATGGA
TAGTGCAGCAGGATGCAGCGGAACCCCCCCGATTTGCCGACAAGCCCAGA
CCAAACTAGAAGTGGCCCTGTACATGTTTCTGTGGAACCCTGACACTGAA
GCTGTTCTGGTTGCCATGTCCTGTTTCCGCCACCTCTGTGAGGAAGCAGA
TATCCGGTGTGGGGTGGATGAAGTGTCAGTGCATAACCTCTTGCCCAACT
ATAACACATTCATGGAGTTTGCCTCTGTCAGCAATATGATGTCAACAGGA
AGAGCAGCACTTCAGAAAAGAGTGATGGCACTGCTGAGGCGCATTGAGCA
TCCCACTGCAGGAAACACTGAGGCTTGGGAAGATACACATGCAAAATGGG
AACAAGCAACAAAGCTAATCCTTAACTATCCAAAAGCCAAAATGGAAGAT
GGCCAGGCTGCTGAAAGCCTTCACAAGACCATTGTTAAGAGGCGAATGTC
CCATGTGAGTGGAGGAGGATCCATAGATTTGTCTGACACAGACTCCCTAC
AGGAATGGATCAACATGACTGGCTTCCTTTGTGCCCTTGGGGGAGTGTGC
CTCCAGCAGAGAAGCAATTCTGGCCTGGCAACCTATAGCCCACCCATGGG
TCCAGTCAGTGAACGTAAGGGTTCTATGATTTCAGTGATGTCTTCAGAGG
GAAACGCAGATACACCTGTCAGCAAATTTATGGATCGGCTGTTGTCCTTA
ATGGTGTGTAACCATGAGAAAGTGGGACTTCAAATACGGACCAATGTTAA
GGATCTGGTGGGTCTAGAATTGAGTCCTGCTCTGTATCCAATGCTATTTA
ACAAATTGAAGAATACCATCAGCAAGTTTTTTGACTCCCAAGGACAGGTT
TTATTGACTGATACCAATACTCAATTTGTAGAACAAACCATAGCTATAAT
GAAGAACTTGCTAGATAATCATACTGAAGGCAGCTCTGAACATCTAGGGC
AAGCTAGCATTGAAACAATGATGTTAAATCTGGTCAGGTATGTTCGTGTG
CTTGGGAATATGGTCCATGCAATTCAAATAAAAACGAAACTGTGTCAATT
AGTTGAAGTAATGATGGCAAGGAGAGATGACCTCTCATTTTGCCAAGAGA
TGAAATTTAGGAATAAGATGGTAGAATACCTGACAGACTGGGTTATGGGA
ACATCAAACCAAGCAGCAGATGATGATGTAAAATGTCTTACAAGAGATTT
GGACCAGGCAAGCATGGAAGCAGTAGTTTCACTTCTAGCTGGTCTCCCTC
TGCAGCCTGAAGAAGGAGATGGTGTGGAATTGATGGAAGCCAAATCACAG
TTATTTCTTAAATACTTCACATTATTTATGAACCTTTTGAATGACTGCAG
TGAAGTTGAAGATGAAAGTGCGCAAACAGGTGGCAGGAAACGTGGCATGT
CTCGGAGGCTGGCATCACTGAGGCACTGTACGGTCCTTGCAATGTCAAAC
TTACTCAATGCCAACGTAGACAGTGGTCTCATGCACTCCATAGGCTTAGG
TTACCACAAGGATCTCCAGACAAGAGCTACATTTATGGAAGTTCTGACAA
AAATCCTTCAACAAGGCACAGAATTTGACACACTTGCAGAAACAGTATTG
GCTGATCGGTTTGAGAGATTGGTGGAACTGGTCACAATGATGGGTGATCA
AGGAGAACTCCCTATAGCGATGGCTCTGGCCAATGTGGTTCCTTGTTCTC
AGTGGGATGAACTAGCTCGAGTTCTGGTTACTCTGTTTGATTCTCGGCAT
TTACTCTACCAACTGCTCTGGAACATGTTTTCTAAAGAAGTAGAATTGGC
AGACTCCATGCAGACTCTCTTCCGAGGCAACAGCTTGGCCAGTAAAATAA
TGACATTCTGTTTCAAGGTATATGGTGCTACCTATCTACAAAAACTCCTG
GATCCTTTATTACGAATTGTGATCACATCCTCTGATTGGCAACATGTTAG
CTTTGAAGTGGATCCTACCAGGTTAGAACCATCAGAGAGCCTTGAGGAAA
ACCAGCGGAACCTCCTTCAGATGACTGAAAAGTTCTTCCATGCCATCATC
AGTTCCTCCTCAGAATTCCCCCCTCAACTTCGAAGTGTGTGCCACTGCTT
ATACCAGGTAAGTATGGAGGAAGGAATTGCGTGTTATGTTGACCCGTGCC
TAGGTATTACGCAATCAAACCAAAGGAACACAGAGAATCGCggccgcCCg
cggtggagctccagcttttgttccctttggccgcaggaacccctagtgat
ggagttggccactccctctctgcgcgctcgctcgctcactgaggccgggc
gaccaaaggtcgcccgacgcccgggctttgcccgggcggcctcagtgagc
gagcgagcgcgcagctgcctgcaggggcgcctgatgcggtattttctcct
tacgcatctgtgcggtatttcacaccgcatacgtcaaagcaaccatagta
cgcgccctgtagcggcgcattaagcgcggcgggtgtggtggttacgcgca
gcgtgaccgctacacttgccagcgccctagcgcccgctcctttcgctttc
ttcccttcctttctcgccacgttcgccggctttccccgtcaagctctaaa
tcgggggctccctttagggttccgatttagtgctttacggcacctcgacc
ccaaaaaacttgatttgggtgatggttcacgtagtgggccatcgccctga
tagacggtttttcgccctttgacgttggagtccacgttctttaatagtgg
actcttgttccaaactggaacaacactcaaccctatctcgggctattctt
ttgatttataagggattttgccgatttcggcctattggttaaaaaatgag
ctgatttaacaaaaatttaacgcgaattttaacaaaatattaacgtttac
aattttatggtgcactctcagtacaatctgctctgatgccgcatagttaa
gccagccccgacacccgccaacacccgctgacgcgccctgacgggcttgt
ctgctcccggcatccgcttacagacaagctgtgaccgtctccgggagctg
catgtgtcagaggttttcaccgtcatcaccgaaacgcgcgagacgaaagg
gcctcgtgatacgcctatttttataggttaatgtcatgataataatggtt
tcttagacgtcaggtggcacttttcggggaaatgtgcgcggaacccctat
ttgtttatttttctaaatacattcaaatatgtatccgctcatgagacaat
aaccctgataaatgcttcaataatattgaaaaaggaagagtatgagtatt
caacatttccgtgtcgcccttattcccttttttgcggcattttgccttcc
tgtttttgctcacccagaaacgctggtgaaagtaaaagatgctgaagatc
agttgggtgcacgagtgggttacatcgaactggatctcaacagcggtaag
atccttgagagttttcgccccgaagaacgttttccaatgatgagcacttt
taaagttctgctatgtggcgcggtattatcccgtattgacgccgggcaag
agcaactcggtcgccgcatacactattctcagaatgacttggttgagtac
tcaccagtcacagaaaagcatcttacggatggcatgacagtaagagaatt
atgcagtgctgccataaccatgagtgataacactgcggccaacttacttc
tgacaacgatcggaggaccgaaggagctaaccgcttttttgcacaacatg
ggggatcatgtaactcgccttgatcgttgggaaccggagctgaatgaagc
cataccaaacgacgagcgtgacaccacgatgcctgtagcaatggcaacaa
cgttgcgcaaactattaactggcgaactacttactctagcttcccggcaa
caattaatagactggatggaggcggataaagttgcaggaccacttctgcg
ctcggcccttccggctggctggtttattgctgataaatctggagccggtg
agcgtggaagccgcggtatcattgcagcactggggccagatggtaagccc
tcccgtatcgtagttatctacacgacggggagtcaggcaactatggatga
acgaaatagacagatcgctgagataggtgcctcactgattaagcattggt
aactgtcagaccaagtttactcatatatactttagattgatttaaaactt
catttttaatttaaaaggatctaggtgaagatcctttttgataatctcat
gaccaaaatcccttaacgtgagttttcgttccactgagcgtcagaccccg
tagaaaagatcaaaggatcttcttgagatcctttttttctgcgcgtaatc
tgctgcttgcaaacaaaaaaaccaccgctaccagcggtggtttgtttgcc
ggatcaagagctaccaactctttttccgaaggtaactggcttcagcagag
cgcagataccaaatactgtccttctagtgtagccgtagttaggccaccac
ttcaagaactctgtagcaccgcctacatacctcgctctgctaatcctgtt
accagtggctgctgccagtggcgataagtcgtgtcttaccgggttggact
caagacgatagttaccggataaggcgcagcggtcgggctgaacggggggt
tcgtgcacacagcccagcttggagcgaacgacctacaccgaactgagata
cctacagcgtgagctatgagaaagcgccacgcttcccgaagggagaaagg
cggacaggtatccggtaagcggcagggtcggaacaggagagcgcacgagg
gagcttccagggggaaacgcctggtatctttatagtcctgtcgggtttcg
ccacctctgacttgagcgtcgatttttgtgatgctcgtcaggggggcgga
gcctatggaaaaacgccagcaacgcggcctttttacggttcctggccttt
tgctggccttttgctcacatgt

SEQ ID NO: 86 (Plasmid 49) Polynucleotide sequence of the donor sequence flanked by gRNA 6 target sites (7474 bp)

cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcctgcggcctctagacgggggat
gtgGCTATTTTGTGCCAGAATTAGTAGAATTGACTGACCTGAgcgtaata
cgactcactatagggcgaattgggtacCGCTAGGTCTTGAAAGGAGTGGG
AATTGGCTCCGGTGCCCGTCAGTGGGCAGAGCGCACATCGCCCACAGTCC
CCGAGAAGTTGGGGGGAGGGGTCGGCAATTGATCCGGTGCCTAGAGAAGG
TGGCGCGGGGTAAACTGGGAAAGTGATGTCGTGTACTGGCTCCGCCTTTT
TCCCGAGGGTGGGGGAGAACCGTATATAAGTGCAGTAGTCGCCGTGAACG
TTCTTTTTCGCAACGGGTTTGCCGCCAGAACACAGGACCGGTGCCACCAT
GGCCGCCCACAGGCCGGTGGAATGGGTCCAGGCCGTGGTCAGCCGCTTCG
ACGAGCAGCTTCCAATAAAAACAGGACAGCAGAACACACATACCAAAGTC
AGTACTGAGCACAACAAGGAATGTCTAATCAATATTTCCAAATACAAGTT
TTCTTTGGTTATAAGCGGCCTCACTACTATTTTAAAGAATGTTAACAATA
TGAGAATATTTGGAGAAGCTGCTGAAAAAAATTTATATCTCTCTCAGTTG
ATTATATTGGATACACTGGAAAAATGTCTTGCTGGGCAACCAAAGGACAC
AATGAGATTAGATGAAACGATGCTGGTCAAACAGTTGCTGCCAGAAATCT
GCCATTTTCTTCACACCTGTCGTGAAGGAAACCAGCATGCAGCTGAACTT
CGGAATTCTGCCTCTGGGGTTTTATTTTCTCTCAGCTGCAACAACTTCAA
TGCAGTCTTTAGTCGCATTTCTACCAGGTTACAGGAATTAACTGTTTGTT
CAGAAGACAATGTTGATGTTCATGATATAGAATTGTTACAGTATATCAAT
GTGGATTGTGCAAAATTAAAACGACTCCTGAAGGAAACAGCATTTAAATT
TAAAGCCCTAAAGAAGGTTGCGCAGTTAGCAGTTATAAATAGCCTGGAAA
AGGCATTTTGGAACTGGGTAGAAAATTATCCAGATGAATTTACAAAACTG
TACCAGATCCCACAGACTGATATGGCTGAATGTGCAGAAAAGCTATTTGA
CTTGGTGGATGGTTTTGCTGAAAGCACCAAACGTAAAGCAGCAGTTTGGC
CACTACAAATCATTCTCCTTATCTTGTGTCCAGAAATAATCCAGGATATA
TCCAAAGACGTGGTTGATGAAAACAACATGAATAAGAAGTTATTTCTGGA
CAGTCTACGAAAAGCTCTTGCTGGCCATGGAGGAAGTAGGCAGCTGACAG
AAAGTGCTGCAATTGCCTGTGTCAAACTGTGTAAAGCAAGTACTTACATC
AATTGGGAAGATAACTCTGTCATTTTCCTACTTGTTCAGTCCATGGTGGT
TGATCTTAAGAACCTGCTTTTTAATCCAAGTAAGCCATTCTCAAGAGGCA
GTCAGCCTGCAGATGTGGATCTAATGATTGACTGCCTTGTTTCTTGCTTT
CGTATAAGCCCTCACAACAACCAACACTTTAAGATCTGCCTGGCTCAGAA
TTCACCTTCTACATTTCACTATGTGCTGGTAAATTCACTCCATCGAATCA
TCACCAATTCCGCATTGGATTGGTGGCCTAAGATTGATGCTGTGTATTGT
CACTCGGTTGAACTTCGAAATATGTTTGGTGAAACACTTCATAAAGCAGT
GCAAGGTTGTGGAGCACACCCAGCAATACGAATGGCACCGAGTCTTACAT
TTAAAGAAAAAGTAACAAGCCTTAAATTTAAAGAAAAACCTACAGACCTG
GAGACAAGAAGCTATAAGTATCTTCTCTTGTCCATGGTGAAACTAATTCA
TGCAGATCCAAAGCTCTTGCTTTGTAATCCAAGAAAACAGGGGCCCGAAA
CCCAAGGCAGTACAGCAGAATTAATTACAGGGCTCGTCCAACTGGTCCCT
CAGTCACACATGCCAGAGATTGCTCAGGAAGCAATGGAGGCTCTGCTGGT
TCTTCATCAGTTAGATAGCATTGATTTGTGGAATCCTGATGCTCCTGTAG
AAACATTTTGGGAGATTAGCTCACAAATGCTTTTTTACATCTGCAAGAAA
TTAACTAGTCATCAAATGCTTAGTAGCACAGAAATTCTCAAGTGGTTGCG
GGAAATATTGATCTGCAGGAATAAATTTCTTCTTAAAAATAAGCAGGCAG
ATAGAAGTTCCTGTCACTTTCTCCTTTTTTACGGGGTAGGATGTGATATT
CCTTCTAGTGGAAATACCAGTCAAATGTCCATGGATCATGAAGAATTACT
ACGTACTCCTGGAGCCTCTCTCCGGAAGGGAAAAGGGAACTCCTCTATGG
ATAGTGCAGCAGGATGCAGCGGAACCCCCCCGATTTGCCGACAAGCCCAG
ACCAAACTAGAAGTGGCCCTGTACATGTTTCTGTGGAACCCTGACACTGA
AGCTGTTCTGGTTGCCATGTCCTGTTTCCGCCACCTCTGTGAGGAAGCAG
ATATCCGGTGTGGGGTGGATGAAGTGTCAGTGCATAACCTCTTGCCCAAC
TATAACACATTCATGGAGTTTGCCTCTGTCAGCAATATGATGTCAACAGG
AAGAGCAGCACTTCAGAAAAGAGTGATGGCACTGCTGAGGCGCATTGAGC
ATCCCACTGCAGGAAACACTGAGGCTTGGGAAGATACACATGCAAAATGG
GAACAAGCAACAAAGCTAATCCTTAACTATCCAAAAGCCAAAATGGAAGA
TGGCCAGGCTGCTGAAAGCCTTCACAAGACCATTGTTAAGAGGCGAATGT
CCCATGTGAGTGGAGGAGGATCCATAGATTTGTCTGACACAGACTCCCTA
CAGGAATGGATCAACATGACTGGCTTCCTTTGTGCCCTTGGGGGAGTGTG
CCTCCAGCAGAGAAGCAATTCTGGCCTGGCAACCTATAGCCCACCCATGG
GTCCAGTCAGTGAACGTAAGGGTTCTATGATTTCAGTGATGTCTTCAGAG
GGAAACGCAGATACACCTGTCAGCAAATTTATGGATCGGCTGTTGTCCTT
AATGGTGTGTAACCATGAGAAAGTGGGACTTCAAATACGGACCAATGTTA
AGGATCTGGTGGGTCTAGAATTGAGTCCTGCTCTGTATCCAATGCTATTT
AACAAATTGAAGAATACCATCAGCAAGTTTTTTGACTCCCAAGGACAGGT
TTTATTGACTGATACCAATACTCAATTTGTAGAACAAACCATAGCTATAA
TGAAGAACTTGCTAGATAATCATACTGAAGGCAGCTCTGAACATCTAGGG
CAAGCTAGCATTGAAACAATGATGTTAAATCTGGTCAGGTATGTTCGTGT
GCTTGGGAATATGGTCCATGCAATTCAAATAAAAACGAAACTGTGTCAAT
TAGTTGAAGTAATGATGGCAAGGAGAGATGACCTCTCATTTTGCCAAGAG
ATGAAATTTAGGAATAAGATGGTAGAATACCTGACAGACTGGGTTATGGG
AACATCAAACCAAGCAGCAGATGATGATGTAAAATGTCTTACAAGAGATT
TGGACCAGGCAAGCATGGAAGCAGTAGTTTCACTTCTAGCTGGTCTCCCT
CTGCAGCCTGAAGAAGGAGATGGTGTGGAATTGATGGAAGCCAAATCACA
GTTATTTCTTAAATACTTCACATTATTTATGAACCTTTTGAATGACTGCA
GTGAAGTTGAAGATGAAAGTGCGCAAACAGGTGGCAGGAAACGTGGCATG
TCTCGGAGGCTGGCATCACTGAGGCACTGTACGGTCCTTGCAATGTCAAA
CTTACTCAATGCCAACGTAGACAGTGGTCTCATGCACTCCATAGGCTTAG
GTTACCACAAGGATCTCCAGACAAGAGCTACATTTATGGAAGTTCTGACA
AAAATCCTTCAACAAGGCACAGAATTTGACACACTTGCAGAAACAGTATT
GGCTGATCGGTTTGAGAGATTGGTGGAACTGGTCACAATGATGGGTGATC
AAGGAGAACTCCCTATAGCGATGGCTCTGGCCAATGTGGTTCCTTGTTCT
CAGTGGGATGAACTAGCTCGAGTTCTGGTTACTCTGTTTGATTCTCGGCA
TTTACTCTACCAACTGCTCTGGAACATGTTTTCTAAAGAAGTAGAATTGG
CAGACTCCATGCAGACTCTCTTCCGAGGCAACAGCTTGGCCAGTAAAATA
ATGACATTCTGTTTCAAGGTATATGGTGCTACCTATCTACAAAAACTCCT
GGATCCTTTATTACGAATTGTGATCACATCCTCTGATTGGCAACATGTTA
GCTTTGAAGTGGATCCTACCAGGTTAGAACCATCAGAGAGCCTTGAGGAA
AACCAGCGGAACCTCCTTCAGATGACTGAAAAGTTCTTCCATGCCATCAT
CAGTTCCTCCTCAGAATTCCCCCCTCAACTTCGAAGTGTGTGCCACTGCT
TATACCAGGTAAGTATGGAGGAAGGAATTGCGTGTTATGTTGACCCGTGC
CTAGGTATTACGCGCTATTTTGTGCCAGAATTAGTAGAATCGCggccgcC
Cgcggtggagctccagcttttgttccctttggccgcaggaacccctagtg
atggagttggccactccctctctgcgcgctcgctcgctcactgaggccgg
gcgaccaaaggtcgcccgacgcccgggctttgcccgggcggcctcagtga
gcgagcgagcgcgcagctgcctgcaggggcgcctgatgcggtattttctc
cttacgcatctgtgcggtatttcacaccgcatacgtcaaagcaaccatag
tacgcgccctgtagcggcgcattaagcgcggcgggtgtggtggttacgcg
cagcgtgaccgctacacttgccagcgccctagcgcccgctcctttcgctt
tcttcccttcctttctcgccacgttcgccggctttccccgtcaagctcta
aatcgggggctccctttagggttccgatttagtgctttacggcacctcga
ccccaaaaaacttgatttgggtgatggttcacgtagtgggccatcgccct
gatagacggtttttcgccctttgacgttggagtccacgttctttaatagt
ggactcttgttccaaactggaacaacactcaaccctatctcgggctattc
ttttgatttataagggattttgccgatttcggcctattggttaaaaaatg
agctgatttaacaaaaatttaacgcgaattttaacaaaatattaacgttt
acaattttatggtgcactctcagtacaatctgctctgatgccgcatagtt
aagccagccccgacacccgccaacacccgctgacgcgccctgacgggctt
gtctgctcccggcatccgcttacagacaagctgtgaccgtctccgggagc
tgcatgtgtcagaggttttcaccgtcatcaccgaaacgcgcgagacgaaa
gggcctcgtgatacgcctatttttataggttaatgtcatgataataatgg
tttcttagacgtcaggtggcacttttcggggaaatgtgcgcggaacccct
atttgtttatttttctaaatacattcaaatatgtatccgctcatgagaca
ataaccctgataaatgcttcaataatattgaaaaaggaagagtatgagta
ttcaacatttccgtgtcgcccttattcccttttttgcggcattttgcctt
cctgtttttgctcacccagaaacgctggtgaaagtaaaagatgctgaaga
tcagttgggtgcacgagtgggttacatcgaactggatctcaacagcggta
agatccttgagagttttcgccccgaagaacgttttccaatgatgagcact
tttaaagttctgctatgtggcgcggtattatcccgtattgacgccgggca
agagcaactcggtcgccgcatacactattctcagaatgacttggttgagt
actcaccagtcacagaaaagcatcttacggatggcatgacagtaagagaa
ttatgcagtgctgccataaccatgagtgataacactgcggccaacttact
tctgacaacgatcggaggaccgaaggagctaaccgcttttttgcacaaca
tgggggatcatgtaactcgccttgatcgttgggaaccggagctgaatgaa
gccataccaaacgacgagcgtgacaccacgatgcctgtagcaatggcaac
aacgttgcgcaaactattaactggcgaactacttactctagcttcccggc
aacaattaatagactggatggaggcggataaagttgcaggaccacttctg
cgctcggcccttccggctggctggtttattgctgataaatctggagccgg
tgagcgtggaagccgcggtatcattgcagcactggggccagatggtaagc
cctcccgtatcgtagttatctacacgacggggagtcaggcaactatggat
gaacgaaatagacagatcgctgagataggtgcctcactgattaagcattg
gtaactgtcagaccaagtttactcatatatactttagattgatttaaaac
ttcatttttaatttaaaaggatctaggtgaagatcctttttgataatctc
atgaccaaaatcccttaacgtgagttttcgttccactgagcgtcagaccc
cgtagaaaagatcaaaggatcttcttgagatcctttttttctgcgcgtaa
tctgctgcttgcaaacaaaaaaaccaccgctaccagcggtggtttgtttg
ccggatcaagagctaccaactctttttccgaaggtaactggcttcagcag
agcgcagataccaaatactgtccttctagtgtagccgtagttaggccacc
acttcaagaactctgtagcaccgcctacatacctcgctctgctaatcctg
ttaccagtggctgctgccagtggcgataagtcgtgtcttaccgggttgga
ctcaagacgatagttaccggataaggcgcagcggtcgggctgaacggggg
gttcgtgcacacagcccagcttggagcgaacgacctacaccgaactgaga
tacctacagcgtgagctatgagaaagcgccacgcttcccgaagggagaaa
ggcggacaggtatccggtaagcggcagggtcggaacaggagagcgcacga
gggagcttccagggggaaacgcctggtatctttatagtcctgtcgggttt
cgccacctctgacttgagcgtcgatttttgtgatgctcgtcaggggggcg
gagcctatggaaaaacgccagcaacgcggcctttttacggttcctggcct
tttgctggccttttgctcacatgt

SEQ ID NO: 87 Polynucleotide sequence of gRNA 1 in AAV vector (7450 bp)

cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcctgcggcctctagactcgaggc
gttgacattgattattgactagttattaatagtaatcaattacggggtca
ttagttcatagcccatatatggagttccgcgttacataacttacggtaaa
tggcccgcctggctgaccgcccaacgacccccgcccattgacgtcaataa
tgacgtatgttcccatagtaacgccaatagggactttccattgacgtcaa
tgggtggagtatttacggtaaactgcccacttggcagtacatcaagtgta
tcatatgccaagtacgccccctattgacgtcaatgacggtaaatggcccg
cctggcattatgcccagtacatgaccttatgggactttcctacttggcag
tacatctacgtattagtcatcgctattaccatggtgatgcggttttggca
gtacatcaatgggcgtggatagcggtttgactcacggggatttccaagtc
tccaccccattgacgtcaatgggagtttgttttggcaccaaaatcaacgg
gactttccaaaatgtcgtaacaactccgccccattgacgcaaatgggcgg
taggcgtgtacggtgggaggtctatataagcagagctctctggctaacta
ccggtgccaccATGGCCCCAAAGAAGAAGCGGAAGGTCGGTATCCACGGA
GTCCCAGCAGCCAAGCGGAACTACATCCTGGGCCTGGACATCGGCATCAC
CAGCGTGGGCTACGGCATCATCGACTACGAGACACGGGACGTGATCGATG
CCGGCGTGCGGCTGTTCAAAGAGGCCAACGTGGAAAACAACGAGGGCAGG
CGGAGCAAGAGAGGCGCCAGAAGGCTGAAGCGGCGGAGGCGGCATAGAAT
CCAGAGAGTGAAGAAGCTGCTGTTCGACTACAACCTGCTGACCGACCACA
GCGAGCTGAGCGGCATCAACCCCTACGAGGCCAGAGTGAAGGGCCTGAGC
CAGAAGCTGAGCGAGGAAGAGTTCTCTGCCGCCCTGCTGCACCTGGCCAA
GAGAAGAGGCGTGCACAACGTGAACGAGGTGGAAGAGGACACCGGCAACG
AGCTGTCCACCAAAGAGCAGATCAGCCGGAACAGCAAGGCCCTGGAAGAG
AAATACGTGGCCGAACTGCAGCTGGAACGGCTGAAGAAAGACGGCGAAGT
GCGGGGCAGCATCAACAGATTCAAGACCAGCGACTACGTGAAAGAAGCCA
AACAGCTGCTGAAGGTGCAGAAGGCCTACCACCAGCTGGACCAGAGCTTC
ATCGACACCTACATCGACCTGCTGGAAACCCGGCGGACCTACTATGAGGG
ACCTGGCGAGGGCAGCCCCTTCGGCTGGAAGGACATCAAAGAATGGTACG
AGATGCTGATGGGCCACTGCACCTACTTCCCCGAGGAACTGCGGAGCGTG
AAGTACGCCTACAACGCCGACCTGTACAACGCCCTGAACGACCTGAACAA
TCTCGTGATCACCAGGGACGAGAACGAGAAGCTGGAATATTACGAGAAGT
TCCAGATCATCGAGAACGTGTTCAAGCAGAAGAAGAAGCCCACCCTGAAG
CAGATCGCCAAAGAAATCCTCGTGAACGAAGAGGATATTAAGGGCTACAG
AGTGACCAGCACCGGCAAGCCCGAGTTCACCAACCTGAAGGTGTACCACG
ACATCAAGGACATTACCGCCCGGAAAGAGATTATTGAGAACGCCGAGCTG
CTGGATCAGATTGCCAAGATCCTGACCATCTACCAGAGCAGCGAGGACAT
CCAGGAAGAACTGACCAATCTGAACTCCGAGCTGACCCAGGAAGAGATCG
AGCAGATCTCTAATCTGAAGGGCTATACCGGCACCCACAACCTGAGCCTG
AAGGCCATCAACCTGATCCTGGACGAGCTGTGGCACACCAACGACAACCA
GATCGCTATCTTCAACCGGCTGAAGCTGGTGCCCAAGAAGGTGGACCTGT
CCCAGCAGAAAGAGATCCCCACCACCCTGGTGGACGACTTCATCCTGAGC
CCCGTCGTGAAGAGAAGCTTCATCCAGAGCATCAAAGTGATCAACGCCAT
CATCAAGAAGTACGGCCTGCCCAACGACATCATTATCGAGCTGGCCCGCG
AGAAGAACTCCAAGGACGCCCAGAAAATGATCAACGAGATGCAGAAGCGG
AACCGGCAGACCAACGAGCGGATCGAGGAAATCATCCGGACCACCGGCAA
AGAGAACGCCAAGTACCTGATCGAGAAGATCAAGCTGCACGACATGCAGG
AAGGCAAGTGCCTGTACAGCCTGGAAGCCATCCCTCTGGAAGATCTGCTG
AACAACCCCTTCAACTATGAGGTGGACCACATCATCCCCAGAAGCGTGTC
CTTCGACAACAGCTTCAACAACAAGGTGCTCGTGAAGCAGGAAGAAAACA
GCAAGAAGGGCAACCGGACCCCATTCCAGTACCTGAGCAGCAGCGACAGC
AAGATCAGCTACGAAACCTTCAAGAAGCACATCCTGAATCTGGCCAAGGG
CAAGGGCAGAATCAGCAAGACCAAGAAAGAGTATCTGCTGGAAGAACGGG
ACATCAACAGGTTCTCCGTGCAGAAAGACTTCATCAACCGGAACCTGGTG
GATACCAGATACGCCACCAGAGGCCTGATGAACCTGCTGCGGAGCTACTT
CAGAGTGAACAACCTGGACGTGAAAGTGAAGTCCATCAATGGCGGCTTCA
CCAGCTTTCTGCGGCGGAAGTGGAAGTTTAAGAAAGAGCGGAACAAGGGG
TACAAGCACCACGCCGAGGACGCCCTGATCATTGCCAACGCCGATTTCAT
CTTCAAAGAGTGGAAGAAACTGGACAAGGCCAAAAAAGTGATGGAAAACC
AGATGTTCGAGGAAAAGCAGGCCGAGAGCATGCCCGAGATCGAAACCGAG
CAGGAGTACAAAGAGATCTTCATCACCCCCCACCAGATCAAGCACATTAA
GGACTTCAAGGACTACAAGTACAGCCACCGGGTGGACAAGAAGCCTAATA
GAGAGCTGATTAACGACACCCTGTACTCCACCCGGAAGGACGACAAGGGC
AACACCCTGATCGTGAACAATCTGAACGGCCTGTACGACAAGGACAATGA
CAAGCTGAAAAAGCTGATCAACAAGAGCCCCGAAAAGCTGCTGATGTACC
ACCACGACCCCCAGACCTACCAGAAACTGAAGCTGATTATGGAACAGTAC
GGCGACGAGAAGAATCCCCTGTACAAGTACTACGAGGAAACCGGGAACTA
CCTGACCAAGTACTCCAAAAAGGACAACGGCCCCGTGATCAAGAAGATTA
AGTATTACGGCAACAAACTGAACGCCCATCTGGACATCACCGACGACTAC
CCCAACAGCAGAAACAAGGTCGTGAAGCTGTCCCTGAAGCCCTACAGATT
CGACGTGTACCTGGACAATGGCGTGTACAAGTTCGTGACCGTGAAGAATC
TGGATGTGATCAAAAAAGAAAACTACTACGAAGTGAATAGCAAGTGCTAT
GAGGAAGCTAAGAAGCTGAAGAAGATCAGCAACCAGGCCGAGTTTATCGC
CTCCTTCTACAACAACGATCTGATCAAGATCAACGGCGAGCTGTATAGAG
TGATCGGCGTGAACAACGACCTGCTGAACCGGATCGAAGTGAACATGATC
GACATCACCTACCGCGAGTACCTGGAAAACATGAACGACAAGAGGCCCCC
CAGGATCATTAAGACAATCGCCTCCAAGACCCAGAGCATTAAGAAGTACA
GCACAGACATTCTGGGCAACCTGTATGAAGTGAAATCTAAGAAGCACCCT
CAGATCATCAAAAAGGGCAAAAGGCCGGCGGCCACGAAAAAGGCCGGCCA
GGCAAAAAAGAAAAAGggatcctacccatacgatgttccagattacgctt
acccatacgatgttccagattacgcttacccatacgatgttccagattac
gcttaagaattcctagagctcgctgatcagcctcgactgtgccttctagt
tgccagccatctgttgtttgcccctcccccgtgccttccttgaccctgga
aggtgccactcccactgtcctttcctaataaaatgaggaaattgcatcgc
attgtctgagtaggtgtcattctattctggggggtggggtggggcaggac
agcaagggggaggattgggaagagaatagcaggcatgctggggaggtacC
AAGCTTgcctatttcccatgattccttcatatttgcatatacgatacaag
gctgttagagagataattggaattaatttgactgtaaacacaaagatatt
agtacaaaatacgtgacgtagaaagtaataatttcttgggtagtttgcag
ttttaaaattatgttttaaaatggactatcatatgcttaccgtaacttga
aagtatttcgatttcttggctttatatatcttgTGGAAAGGACGAAACAC
CGCCAGAAATAGTAGACATGAgttttagtactctggaaacagaatctact
aaaacaaggcaaaatgccgtgtttatctcgtcaacttgttggcgagattt
ttttGCggccgcaggaacccctagtgatggagttggccactccctctctg
cgcgctcgctcgctcactgaggccgggcgaccaaaggtcgcccgacgccc
gggctttgcccgggcggcctcagtgagcgagcgagcgcgcagctgcctgc
aggggcgcctgatgcggtattttctccttacgcatctgtgcggtatttca
caccgcatacgtcaaagcaaccatagtacgcgccctgtagcggcgcatta
agcgcggcgggtgtggtggttacgcgcagcgtgaccgctacacttgccag
cgccctagcgcccgctcctttcgctttcttcccttcctttctcgccacgt
tcgccggctttccccgtcaagctctaaatcgggggctccctttagggttc
cgatttagtgctttacggcacctcgaccccaaaaaacttgatttgggtga
tggttcacgtagtgggccatcgccctgatagacggtttttcgccctttga
cgttggagtccacgttctttaatagtggactcttgttccaaactggaaca
acactcaaccctatctcgggctattcttttgatttataagggattttgcc
gatttcggcctattggttaaaaaatgagctgatttaacaaaaatttaacg
cgaattttaacaaaatattaacgtttacaattttatggtgcactctcagt
acaatctgctctgatgccgcatagttaagccagccccgacacccgccaac
acccgctgacgcgccctgacgggcttgtctgctcccggcatccgcttaca
gacaagctgtgaccgtctccgggagctgcatgtgtcagaggttttcaccg
tcatcaccgaaacgcgcgagacgaaagggcctcgtgatacgcctattttt
ataggttaatgtcatgataataatggtttcttagacgtcaggtggcactt
ttcggggaaatgtgcgcggaacccctatttgtttatttttctaaatacat
tcaaatatgtatccgctcatgagacaataaccctgataaatgcttcaata
atattgaaaaaggaagagtatgagtattcaacatttccgtgtcgccctta
ttcccttttttgcggcattttgccttcctgtttttgctcacccagaaacg
ctggtgaaagtaaaagatgctgaagatcagttgggtgcacgagtgggtta
catcgaactggatctcaacagcggtaagatccttgagagttttcgccccg
aagaacgttttccaatgatgagcacttttaaagttctgctatgtggcgcg
gtattatcccgtattgacgccgggcaagagcaactcggtcgccgcataca
ctattctcagaatgacttggttgagtactcaccagtcacagaaaagcatc
ttacggatggcatgacagtaagagaattatgcagtgctgccataaccatg
agtgataacactgcggccaacttacttctgacaacgatcggaggaccgaa
ggagctaaccgcttttttgcacaacatgggggatcatgtaactcgccttg
atcgttgggaaccggagctgaatgaagccataccaaacgacgagcgtgac
accacgatgcctgtagcaatggcaacaacgttgcgcaaactattaactgg
cgaactacttactctagcttcccggcaacaattaatagactggatggagg
cggataaagttgcaggaccacttctgcgctcggcccttccggctggctgg
tttattgctgataaatctggagccggtgagcgtggaagccgcggtatcat
tgcagcactggggccagatggtaagccctcccgtatcgtagttatctaca
cgacggggagtcaggcaactatggatgaacgaaatagacagatcgctgag
ataggtgcctcactgattaagcattggtaactgtcagaccaagtttactc
atatatactttagattgatttaaaacttcatttttaatttaaaaggatct
aggtgaagatcctttttgataatctcatgaccaaaatcccttaacgtgag
ttttcgttccactgagcgtcagaccccgtagaaaagatcaaaggatcttc
ttgagatcctttttttctgcgcgtaatctgctgcttgcaaacaaaaaaac
caccgctaccagcggtggtttgtttgccggatcaagagctaccaactctt
tttccgaaggtaactggcttcagcagagcgcagataccaaatactgtcct
tctagtgtagccgtagttaggccaccacttcaagaactctgtagcaccgc
ctacatacctcgctctgctaatcctgttaccagtggctgctgccagtggc
gataagtcgtgtcttaccgggttggactcaagacgatagttaccggataa
ggcgcagcggtcgggctgaacggggggttcgtgcacacagcccagcttgg
agcgaacgacctacaccgaactgagatacctacagcgtgagctatgagaa
agcgccacgcttcccgaagggagaaaggcggacaggtatccggtaagcgg
cagggtcggaacaggagagcgcacgagggagcttccagggggaaacgcct
ggtatctttatagtcctgtcgggtttcgccacctctgacttgagcgtcga
tttttgtgatgctcgtcaggggggcggagcctatggaaaaacgccagcaa
cgcggcctttttacggttcctggccttttgctggccttttgctcacatgt

SEQ ID NO: 88 Polynucleotide sequence of gRNA 3 in AAV vector (7450 bp)

cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcctgcggcctctagactcgaggc
gttgacattgattattgactagttattaatagtaatcaattacggggtca
ttagttcatagcccatatatggagttccgcgttacataacttacggtaaa
tggcccgcctggctgaccgcccaacgacccccgcccattgacgtcaataa
tgacgtatgttcccatagtaacgccaatagggactttccattgacgtcaa
tgggtggagtatttacggtaaactgcccacttggcagtacatcaagtgta
tcatatgccaagtacgccccctattgacgtcaatgacggtaaatggcccg
cctggcattatgcccagtacatgaccttatgggactttcctacttggcag
tacatctacgtattagtcatcgctattaccatggtgatgcggttttggca
gtacatcaatgggcgtggatagcggtttgactcacggggatttccaagtc
tccaccccattgacgtcaatgggagtttgttttggcaccaaaatcaacgg
gactttccaaaatgtcgtaacaactccgccccattgacgcaaatgggcgg
taggcgtgtacggtgggaggtctatataagcagagctctctggctaacta
ccggtgccaccATGGCCCCAAAGAAGAAGCGGAAGGTCGGTATCCACGGA
GTCCCAGCAGCCAAGCGGAACTACATCCTGGGCCTGGACATCGGCATCAC
CAGCGTGGGCTACGGCATCATCGACTACGAGACACGGGACGTGATCGATG
CCGGCGTGCGGCTGTTCAAAGAGGCCAACGTGGAAAACAACGAGGGCAGG
CGGAGCAAGAGAGGCGCCAGAAGGCTGAAGCGGCGGAGGCGGCATAGAAT
CCAGAGAGTGAAGAAGCTGCTGTTCGACTACAACCTGCTGACCGACCACA
GCGAGCTGAGCGGCATCAACCCCTACGAGGCCAGAGTGAAGGGCCTGAGC
CAGAAGCTGAGCGAGGAAGAGTTCTCTGCCGCCCTGCTGCACCTGGCCAA
GAGAAGAGGCGTGCACAACGTGAACGAGGTGGAAGAGGACACCGGCAACG
AGCTGTCCACCAAAGAGCAGATCAGCCGGAACAGCAAGGCCCTGGAAGAG
AAATACGTGGCCGAACTGCAGCTGGAACGGCTGAAGAAAGACGGCGAAGT
GCGGGGCAGCATCAACAGATTCAAGACCAGCGACTACGTGAAAGAAGCCA
AACAGCTGCTGAAGGTGCAGAAGGCCTACCACCAGCTGGACCAGAGCTTC
ATCGACACCTACATCGACCTGCTGGAAACCCGGCGGACCTACTATGAGGG
ACCTGGCGAGGGCAGCCCCTTCGGCTGGAAGGACATCAAAGAATGGTACG
AGATGCTGATGGGCCACTGCACCTACTTCCCCGAGGAACTGCGGAGCGTG
AAGTACGCCTACAACGCCGACCTGTACAACGCCCTGAACGACCTGAACAA
TCTCGTGATCACCAGGGACGAGAACGAGAAGCTGGAATATTACGAGAAGT
TCCAGATCATCGAGAACGTGTTCAAGCAGAAGAAGAAGCCCACCCTGAAG
CAGATCGCCAAAGAAATCCTCGTGAACGAAGAGGATATTAAGGGCTACAG
AGTGACCAGCACCGGCAAGCCCGAGTTCACCAACCTGAAGGTGTACCACG
ACATCAAGGACATTACCGCCCGGAAAGAGATTATTGAGAACGCCGAGCTG
CTGGATCAGATTGCCAAGATCCTGACCATCTACCAGAGCAGCGAGGACAT
CCAGGAAGAACTGACCAATCTGAACTCCGAGCTGACCCAGGAAGAGATCG
AGCAGATCTCTAATCTGAAGGGCTATACCGGCACCCACAACCTGAGCCTG
AAGGCCATCAACCTGATCCTGGACGAGCTGTGGCACACCAACGACAACCA
GATCGCTATCTTCAACCGGCTGAAGCTGGTGCCCAAGAAGGTGGACCTGT
CCCAGCAGAAAGAGATCCCCACCACCCTGGTGGACGACTTCATCCTGAGC
CCCGTCGTGAAGAGAAGCTTCATCCAGAGCATCAAAGTGATCAACGCCAT
CATCAAGAAGTACGGCCTGCCCAACGACATCATTATCGAGCTGGCCCGCG
AGAAGAACTCCAAGGACGCCCAGAAAATGATCAACGAGATGCAGAAGCGG
AACCGGCAGACCAACGAGCGGATCGAGGAAATCATCCGGACCACCGGCAA
AGAGAACGCCAAGTACCTGATCGAGAAGATCAAGCTGCACGACATGCAGG
AAGGCAAGTGCCTGTACAGCCTGGAAGCCATCCCTCTGGAAGATCTGCTG
AACAACCCCTTCAACTATGAGGTGGACCACATCATCCCCAGAAGCGTGTC
CTTCGACAACAGCTTCAACAACAAGGTGCTCGTGAAGCAGGAAGAAAACA
GCAAGAAGGGCAACCGGACCCCATTCCAGTACCTGAGCAGCAGCGACAGC
AAGATCAGCTACGAAACCTTCAAGAAGCACATCCTGAATCTGGCCAAGGG
CAAGGGCAGAATCAGCAAGACCAAGAAAGAGTATCTGCTGGAAGAACGGG
ACATCAACAGGTTCTCCGTGCAGAAAGACTTCATCAACCGGAACCTGGTG
GATACCAGATACGCCACCAGAGGCCTGATGAACCTGCTGCGGAGCTACTT
CAGAGTGAACAACCTGGACGTGAAAGTGAAGTCCATCAATGGCGGCTTCA
CCAGCTTTCTGCGGCGGAAGTGGAAGTTTAAGAAAGAGCGGAACAAGGGG
TACAAGCACCACGCCGAGGACGCCCTGATCATTGCCAACGCCGATTTCAT
CTTCAAAGAGTGGAAGAAACTGGACAAGGCCAAAAAAGTGATGGAAAACC
AGATGTTCGAGGAAAAGCAGGCCGAGAGCATGCCCGAGATCGAAACCGAG
CAGGAGTACAAAGAGATCTTCATCACCCCCCACCAGATCAAGCACATTAA
GGACTTCAAGGACTACAAGTACAGCCACCGGGTGGACAAGAAGCCTAATA
GAGAGCTGATTAACGACACCCTGTACTCCACCCGGAAGGACGACAAGGGC
AACACCCTGATCGTGAACAATCTGAACGGCCTGTACGACAAGGACAATGA
CAAGCTGAAAAAGCTGATCAACAAGAGCCCCGAAAAGCTGCTGATGTACC
ACCACGACCCCCAGACCTACCAGAAACTGAAGCTGATTATGGAACAGTAC
GGCGACGAGAAGAATCCCCTGTACAAGTACTACGAGGAAACCGGGAACTA
CCTGACCAAGTACTCCAAAAAGGACAACGGCCCCGTGATCAAGAAGATTA
AGTATTACGGCAACAAACTGAACGCCCATCTGGACATCACCGACGACTAC
CCCAACAGCAGAAACAAGGTCGTGAAGCTGTCCCTGAAGCCCTACAGATT
CGACGTGTACCTGGACAATGGCGTGTACAAGTTCGTGACCGTGAAGAATC
TGGATGTGATCAAAAAAGAAAACTACTACGAAGTGAATAGCAAGTGCTAT
GAGGAAGCTAAGAAGCTGAAGAAGATCAGCAACCAGGCCGAGTTTATCGC
CTCCTTCTACAACAACGATCTGATCAAGATCAACGGCGAGCTGTATAGAG
TGATCGGCGTGAACAACGACCTGCTGAACCGGATCGAAGTGAACATGATC
GACATCACCTACCGCGAGTACCTGGAAAACATGAACGACAAGAGGCCCCC
CAGGATCATTAAGACAATCGCCTCCAAGACCCAGAGCATTAAGAAGTACA
GCACAGACATTCTGGGCAACCTGTATGAAGTGAAATCTAAGAAGCACCCT
CAGATCATCAAAAAGGGCAAAAGGCCGGCGGCCACGAAAAAGGCCGGCCA
GGCAAAAAAGAAAAAGggatcctacccatacgatgttccagattacgctt
acccatacgatgttccagattacgcttacccatacgatgttccagattac
gcttaagaattcctagagctcgctgatcagcctcgactgtgccttctagt
tgccagccatctgttgtttgcccctcccccgtgccttccttgaccctgga
aggtgccactcccactgtcctttcctaataaaatgaggaaattgcatcgc
attgtctgagtaggtgtcattctattctggggggtggggtggggcaggac
agcaagggggaggattgggaagagaatagcaggcatgctggggaggtacC
AAGCTTgcctatttcccatgattccttcatatttgcatatacgatacaag
gctgttagagagataattggaattaatttgactgtaaacacaaagatatt
agtacaaaatacgtgacgtagaaagtaataatttcttgggtagtttgcag
ttttaaaattatgttttaaaatggactatcatatgcttaccgtaacttga
aagtatttcgatttcttggctttatatatcttgTGGAAAGGACGAAACAC
CGTTTCAATGTAACTTAATTCgttttagtactctggaaacagaatctact
aaaacaaggcaaaatgccgtgtttatctcgtcaacttgttggcgagattt
ttttGCggccgcaggaacccctagtgatggagttggccactccctctctg
cgcgctcgctcgctcactgaggccgggcgaccaaaggtcgcccgacgccc
gggctttgcccgggcggcctcagtgagcgagcgagcgcgcagctgcctgc
aggggcgcctgatgcggtattttctccttacgcatctgtgcggtatttca
caccgcatacgtcaaagcaaccatagtacgcgccctgtagcggcgcatta
agcgcggcgggtgtggtggttacgcgcagcgtgaccgctacacttgccag
cgccctagcgcccgctcctttcgctttcttcccttcctttctcgccacgt
tcgccggctttccccgtcaagctctaaatcgggggctccctttagggttc
cgatttagtgctttacggcacctcgaccccaaaaaacttgatttgggtga
tggttcacgtagtgggccatcgccctgatagacggtttttcgccctttga
cgttggagtccacgttctttaatagtggactcttgttccaaactggaaca
acactcaaccctatctcgggctattcttttgatttataagggattttgcc
gatttcggcctattggttaaaaaatgagctgatttaacaaaaatttaacg
cgaattttaacaaaatattaacgtttacaattttatggtgcactctcagt
acaatctgctctgatgccgcatagttaagccagccccgacacccgccaac
acccgctgacgcgccctgacgggcttgtctgctcccggcatccgcttaca
gacaagctgtgaccgtctccgggagctgcatgtgtcagaggttttcaccg
tcatcaccgaaacgcgcgagacgaaagggcctcgtgatacgcctattttt
ataggttaatgtcatgataataatggtttcttagacgtcaggtggcactt
ttcggggaaatgtgcgcggaacccctatttgtttatttttctaaatacat
tcaaatatgtatccgctcatgagacaataaccctgataaatgcttcaata
atattgaaaaaggaagagtatgagtattcaacatttccgtgtcgccctta
ttcccttttttgcggcattttgccttcctgtttttgctcacccagaaacg
ctggtgaaagtaaaagatgctgaagatcagttgggtgcacgagtgggtta
catcgaactggatctcaacagcggtaagatccttgagagttttcgccccg
aagaacgttttccaatgatgagcacttttaaagttctgctatgtggcgcg
gtattatcccgtattgacgccgggcaagagcaactcggtcgccgcataca
ctattctcagaatgacttggttgagtactcaccagtcacagaaaagcatc
ttacggatggcatgacagtaagagaattatgcagtgctgccataaccatg
agtgataacactgcggccaacttacttctgacaacgatcggaggaccgaa
ggagctaaccgcttttttgcacaacatgggggatcatgtaactcgccttg
atcgttgggaaccggagctgaatgaagccataccaaacgacgagcgtgac
accacgatgcctgtagcaatggcaacaacgttgcgcaaactattaactgg
cgaactacttactctagcttcccggcaacaattaatagactggatggagg
cggataaagttgcaggaccacttctgcgctcggcccttccggctggctgg
tttattgctgataaatctggagccggtgagcgtggaagccgcggtatcat
tgcagcactggggccagatggtaagccctcccgtatcgtagttatctaca
cgacggggagtcaggcaactatggatgaacgaaatagacagatcgctgag
ataggtgcctcactgattaagcattggtaactgtcagaccaagtttactc
atatatactttagattgatttaaaacttcatttttaatttaaaaggatct
aggtgaagatcctttttgataatctcatgaccaaaatcccttaacgtgag
ttttcgttccactgagcgtcagaccccgtagaaaagatcaaaggatcttc
ttgagatcctttttttctgcgcgtaatctgctgcttgcaaacaaaaaaac
caccgctaccagcggtggtttgtttgccggatcaagagctaccaactctt
tttccgaaggtaactggcttcagcagagcgcagataccaaatactgtcct
tctagtgtagccgtagttaggccaccacttcaagaactctgtagcaccgc
ctacatacctcgctctgctaatcctgttaccagtggctgctgccagtggc
gataagtcgtgtcttaccgggttggactcaagacgatagttaccggataa
ggcgcagcggtcgggctgaacggggggttcgtgcacacagcccagcttgg
agcgaacgacctacaccgaactgagatacctacagcgtgagctatgagaa
agcgccacgcttcccgaagggagaaaggcggacaggtatccggtaagcgg
cagggtcggaacaggagagcgcacgagggagcttccagggggaaacgcct
ggtatctttatagtcctgtcgggtttcgccacctctgacttgagcgtcga
tttttgtgatgctcgtcaggggggcggagcctatggaaaaacgccagcaa
cgcggcctttttacggttcctggccttttgctggccttttgctcacatgt

SEQ ID NO: 89 Polynucleotide sequence of gRNA 4 in AAV vector (7451 bp)

cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcctgcggcctctagactcgaggc
gttgacattgattattgactagttattaatagtaatcaattacggggtca
ttagttcatagcccatatatggagttccgcgttacataacttacggtaaa
tggcccgcctggctgaccgcccaacgacccccgcccattgacgtcaataa
tgacgtatgttcccatagtaacgccaatagggactttccattgacgtcaa
tgggtggagtatttacggtaaactgcccacttggcagtacatcaagtgta
tcatatgccaagtacgccccctattgacgtcaatgacggtaaatggcccg
cctggcattatgcccagtacatgaccttatgggactttcctacttggcag
tacatctacgtattagtcatcgctattaccatggtgatgcggttttggca
gtacatcaatgggcgtggatagcggtttgactcacggggatttccaagtc
tccaccccattgacgtcaatgggagtttgttttggcaccaaaatcaacgg
gactttccaaaatgtcgtaacaactccgccccattgacgcaaatgggcgg
taggcgtgtacggtgggaggtctatataagcagagctctctggctaacta
ccggtgccaccATGGCCCCAAAGAAGAAGCGGAAGGTCGGTATCCACGGA
GTCCCGCAGCCAAGCGGAACTACATCCTGGGCCTGGACATCGGCATCACC
AGCGTGGGCTACGGCATCATCGACTACGAGACACGGGACGTGATCGATGC
CGGCGTGCGGCTGTTCAAAGAGGCCAACGTGGAAAACAACGAGGGCAGGC
GGAGCAAGAGAGGCGCCAGAAGGCTGAAGCGGCGGAGGCGGCATAGAATC
CAGAGAGTGAAGAAGCTGCTGTTCGACTACAACCTGCTGACCGACCACAG
CGAGGGCCAGAGTGTGTTCGACTGAGCGGCATCAAGGGCCTGAGCCAGAA
GCTGAGCGAGGAAGAGTTCTCTGCCGCCCTGCTGCACCTGGCCAAGAGAA
GAGGCGTGCACAACGTGAACGAGGTGGAAGAGGACACCGGCAACGAGCTG
TCCACCAAAGAGCAGATCAGCCGGAACAGCAAGGCCCTGGAAGAGAAATA
CGTGGCCGAACTGCAGCTGGAACGGCTGAAGAAAGACGGCGAAGTGCGGG
GCAGCATCAACAGATTCAAGACCAGCGACTACGTGAAAGAAGCCAAACAG
CTGCTGAAGGTGCAGAAGGCCTACCACCAGCTGGACCAGAGCTTCATCGA
CACCTACATCGACCTGCTGGAAACCCGGCGGACCTACTATGAGGGACCTG
GCGAGGGCAGCCCCTTCGGCTGGAAGGACATCAAAGAATGGTACGAGATG
CTGATGGGCCACTGCACCTACTTCCCCGAGGAACTGCGGAGCGTGAAGTA
CGCCTACAACGCCGACCTGTACAACGCCCTGAACGACCTGAACAATCTCG
TGATCACCAGGACGAGAACGAGAAGCTGGAATATTACGAGAAGTTCCAGA
TCATCGAGAACGTGTTCAAGCAGAAGAAGAAGCCCACCCTGAAGCAGATC
GCCAAAGAAATCCTCGTGAACGAAGAGGATATTAAGGGCTACAGAGTGAC
CAGCACCGGCAAGCCCGAGTTCACCAACCTGAAGGTGTACCACGACATCA
AGGACATTACCGCCCGGAAAGAGATTATTGAGAACGCCGAGCTGCTGGAT
CAGATTGCCAAGATCCTGACCATCTACCAGAGCAGCGAGGACATCCAGGA
AGAACTGACCAATCTGAACTCCGAGCTGACCCAGGAAGAGATCGAGCAGA
TCTCTAATCTGAAGGGCTATACCGGCACCCACAACCTGAGCCTGAAGGCC
ATCAACCTGATCCTGGACGAGCTGTGGCACACCAACGACAACCAGATCGC
TATCTTCAACCGGCTGAAGCTGGTGCCCAAGAAGGTGGACCTGTCCCAGC
AGAAAGAGATCCCCACCACCCTGGTGGACGACTTCATCCTGAGCCCCGTC
GTGAAGAGAAGCTTCATCCAGAGCATCAAAGTGATCAACGCCATCATCAA
GAAGTACGGCCTGCCCAACGACATCATTATCGAGCTGGCCCGCGAGAAGA
ACTCCAAGGACGCCCAGAAAATGATCAACGAGATGCAGAAGCGGAACCGG
CAGACCAACGAGCGGATCGAGGAAATCATCCGGACCACCGGCAAAGAGAA
CGCCAAGTACCTGATCGAGAAGATCAAGCTGCACGACATGCAGGAAGGCA
AGTGCCTGTACAGCCTGGAAGCCATCCCTCTGGAAGATCTGCTGAACAAC
CCCTTCAACTATGAGGTGGACCACATCATCCCCAGAAGCGTGTCCTTCGA
CAACAGCTTCAACAACAAGGTGCTCGTGAAGCAGGAAGAAAACAGCAAGA
AGGGCAACCGGACCCCATTCCAGTACCTGAGCAGCAGCGACAGCAAGATC
AGCTACGAAACCTTCAAGAAGCACATCCTGAATCTGGCCAAGGGCAAGGG
CAGAATCAGCAAGACCAAGAAAGAGTATCTGCTGGAAGAACGGGACATCA
ACAGGTTCTCCGTGCAGAAAGACTTCATCAACCGGAACCTGGTGGATACC
AGATACGCCACCAGAGGCCTGATGAACCTGCTGCGGAGCTACTTCAGAGT
GAACAACCTGGACGTGAAAGTGAAGTCCATCAATGGCGGCTTCACCAGCT
TTCTGCGGCGGAAGTGGAAGTTTAAGAAAGAGCGGAACAAGGGGTACAAG
CACCACGCCGAGGACGCCCTGATCATTGCCAACGCCGATTTCATCTTCAA
AGAGTGGAAGAAACTGGACAAGGCCAAAAAAGTGATGGAAAACCAGATGT
TCGAGGAAAAGCAGGCCGAGAGCATGCCCGAGATCGAAACCGAGCAGGAG
TACAAAGAGATCTTCATCACCCCCCACCAGATCAAGCACATTAAGGACTT
CAAGGACTACAAGTACAGCCACCGGGTGGACAAGAAGCCTAATAGAGAGC
TGATTAACGACACCCTGTACTCCACCCGGAAGGACGACAAGGGCAACACC
CTGATCGTGAACAATCTGAACGGCCTGTACGACAAGGACAATGACAAGCT
GAAAAAGCTGATCAACAAGAGCCCCGAAAAGCTGCTGATGTACCACCACG
ACCCCCAGACCTACCAGAAACTGAAGCTGATTATGGAACAGTACGGCGAC
GAGAAGAATCCCCTGTACAAGTACTACGAGGAAACCGGGAACTACCTGAC
CAAGTACTCCAAAAAGGACAACGGCCCCGTGATCAAGAAGATTAAGTATT
ACGGCAACAAACTGAACGCCCATCTGGACATCACCGACGACTACCCCAAC
AGCAGAAACAAGGTCGTGAAGCTGTCCCTGAAGCCCTACAGATTCGACGT
GTACCTGGACAATGGCGTGTACAAGTTCGTGACCGTGAAGAATCTGGATG
TGATCAAAAAAGAAAACTACTACGAAGTGAATAGCAAGTGCTATGAGGAA
GCTAAGAAGCTGAAGAAGATCAGCAACCAGGCCGAGTTTATCGCCTCCTT
CTACAACAACGATCTGATCAAGATCAACGGCGAGCTGTATAGAGTGATCG
GCGTGAACAACGACCTGCTGAACCGGATCGAAGTGAACATGATCGACATC
ACCTACCGCGAGTACCTGGAAAACATGAACGACAAGAGGCCCCCCAGGAT
CATTAAGACAATCGCCTCCAAGACCCAGAGCATTAAGAAGTACAGCACAG
ACATTCTGGGCAACCTGTATGAAGTGAAATCTAAGAAGCACCCTCAGATC
ATCAAAAAGGGCAAAAGGCCGGCGGCCACGAAAAAGGCCGGCCAGGCAAA
AAAGAAAAAGggatcctacccatacgatgttccagattacgcttacccat
acgatgttccagattacgcttacccatacgatgttccagattacgcttaa
gaattcctagagctcgctgatcagcctcgactgtgccttctagttgccag
ccatctgttgtttgcccctcccccgtgccttccttgaccctggaaggtgc
cactcccactgtcctttcctaataaaatgaggaaattgcatcgcattgtc
tgagtaggtgtcattctattctggggggtggggtggggcaggacagcaag
gcctatttcccatgattccttcatatttgcatatacgatacaaggctgtt
agagagataattggaattaatttgactgtaaacacaaagatattagtaca
aaatacgtgacgtagaaagtaataatttcttgggtagtttgcagttttaa
aattatgttttaaaatggactatcatatgcttaccgtaacttgaaagtat
ttcgatttcttggctttatatatcttgTGGAAAGGACGAAACACCGAATC
AAACCAAAGGAACACAgttttagtactctggaaacagaatctactaaaac
aaggcaaaatgccgtgtttatctcgtcaacttgttggcgagatttttttG
Cggccgcaggaacccctagtgatggagttggccactccctctctgcgcgc
tcgctcgctcactgaggccgggcgaccaaaggtcgcccgacgcccgggct
ttgcccgggcggcctcagtgagcgagcgagcgcgcagctgcctgcagggg
cgcctgatgcggtattttctccttacgcatctgtgcggtatttcacaccg
catacgtcaaagcaaccatagtacgcgccctgtagcggcgcattaagcgc
ggcgggtgtggtggttacgcgcagcgtgaccgctacacttgccagcgccc
tagcgcccgctcctttcgctttcttcccttcctttctcgccacgttcgcc
ggctttccccgtcaagctctaaatcgggggctccctttagggttccgatt
tagtgctttacggcacctcgaccccaaaaaacttgatttgggtgatggtt
cacgtagtgggccatcgccctgatagacggtttttcgccctttgacgttg
gagtccacgttctttaatagtggactcttgttccaaactggaacaacact
caaccctatctcgggctattcttttgatttataagggattttgccgattt
cggcctattggttaaaaaatgagctgatttaacaaaaatttaacgcgaat
tttaacaaaatattaacgtttacaattttatggtgcactctcagtacaat
ctgctctgatgccgcatagttaagccagccccgacacccgccaacacccg
ctgacgcgccctgacgggcttgtctgctcccggcatccgcttacagacaa
gctgtgaccgtctccgggagctgcatgtgtcagaggttttcaccgtcatc
accgaaacgcgcgagacgaaagggcctcgtgatacgcctatttttatagg
ttaatgtcatgataataatggtttcttagacgtcaggtggcacttttcgg
ggaaatgtgcgcggaacccctatttgtttatttttctaaatacattcaaa
tatgtatccgctcatgagacaataaccctgataaatgcttcaataatatt
gaaaaaggaagagtatgagtattcaacatttccgtgtcgcccttattccc
ttttttgcggcattttgccttcctgtttttgctcacccagaaacgctggt
gaaagtaaaagatgctgaagatcagttgggtgcacgagtgggttacatcg
aactggatctcaacagcggtaagatccttgagagttttcgccccgaagaa
cgttttccaatgatgagcacttttaaagttctgctatgtggcgcggtatt
atcccgtattgacgccgggcaagagcaactcggtcgccgcatacactatt
ctcagaatgacttggttgagtactcaccagtcacagaaaagcatcttacg
gatggcatgacagtaagagaattatgcagtgctgccataaccatgagtga
taacactgcggccaacttacttctgacaacgatcggaggaccgaaggagc
taaccgcttttttgcacaacatgggggatcatgtaactcgccttgatcgt
tgggaaccggagctgaatgaagccataccaaacgacgagcgtgacaccac
gatgcctgtagcaatggcaacaacgttgcgcaaactattaactggcgaac
tacttactctagcttcccggcaacaattaatagactggatggaggcggat
aaagttgcaggaccacttctgcgctcggcccttccggctggctggtttat
tgctgataaatctggagccggtgagcgtggaagccgcggtatcattgcag
cactggggccagatggtaagccctcccgtatcgtagttatctacacgacg
gggagtcaggcaactatggatgaacgaaatagacagatcgctgagatagg
tgcctcactgattaagcattggtaactgtcagaccaagtttactcatata
tactttagattgatttaaaacttcatttttaatttaaaaggatctaggtg
aagatcctttttgataatctcatgaccaaaatcccttaacgtgagttttc
gttccactgagcgtcagaccccgtagaaaagatcaaaggatcttcttgag
atcctttttttctgcgcgtaatctgctgcttgcaaacaaaaaaaccaccg
ctaccagcggtggtttgtttgccggatcaagagctaccaactctttttcc
gaaggtaactggcttcagcagagcgcagataccaaatactgtccttctag
tgtagccgtagttaggccaccacttcaagaactctgtagcaccgcctaca
tacctcgctctgctaatcctgttaccagtggctgctgccagtggcgataa
gtcgtgtcttaccgggttggactcaagacgatagttaccggataaggcgc
agcggtcgggctgaacggggggttcgtgcacacagcccagcttggagcga
acgacctacaccgaactgagatacctacagcgtgagctatgagaaagcgc
cacgcttcccgaagggagaaaggcggacaggtatccggtaagcggcaggg
tcggaacaggagagcgcacgagggagcttccagggggaaacgcctggtat
ctttatagtcctgtcgggtttcgccacctctgacttgagcgtcgattttt
gtgatgctcgtcaggggggcggagcctatggaaaaacgccagcaacgcgg
cctttttacggttcctggccttttgctggccttttgctcacatgt

SEQ ID NO: 90 Polynucleotide sequence of gRNA 6 in AAV vector (7451 bp)

cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcctgcggcctctagactcgaggc
gttgacattgattattgactagttattaatagtaatcaattacggggtca
ttagttcatagcccatatatggagttccgcgttacataacttacggtaaa
tggcccgcctggctgaccgcccaacgacccccgcccattgacgtcaataa
tgacgtatgttcccatagtaacgccaatagggactttccattgacgtcaa
tgggtggagtatttacggtaaactgcccacttggcagtacatcaagtgta
tcatatgccaagtacgccccctattgacgtcaatgacggtaaatggcccg
cctggcattagcccagtacatgaccttatgggactttcctacttggcagt
acatctacgtattagtcatcgctattaccatggtgatgcggttttggcag
tacatcaatgggcgtggatagcggtttgactcacggggatttccaagtct
ccaccccattgacgtcaatgggagtttgttttggcaccaaaatcaacggg
actttccaaaatgtcgtaacaactccgccccattgacgcaaatgggcggt
aggcgtgtacggtgggaggtctatataagcagagctctctggctaactac
cggtgccaccATGGCCCCAAAGAAGAAGCGGAAGGTCGGTATCCACGGAG
TCCCACAGCCAAGCGGAACTACATCCTGGGCCTGGACATCGGCATCACCA
GCGTGGGCTACGGCATCATCGACTACGAGACACGGGACGTGATCGATGCC
GGCGTGCGGCTGTTCAAAGAGGCCAACGTGGAAAACAACGAGGGCAGGCG
GAGCAAGAGAGGCGCCAGAAGGCTGAAGCGGCGGAGGCGGCATAGAATCC
AGAGAGTGAAGAAGCTGCTGTTCGACTACAACCTGCTGACCGACCACAGC
GAGCTGAGCGGCATCAACCCCTACGAGGCCAGAGTGAAGGGCCTGAGCCA
GAAGCTGAGCGAGGAAGAGTTCTCTGCCGCCCTGCTGCACCTGGCCAAGA
GAAGAGGCGTGCACAACGTGAACGAGGTGGAAGAGGACACCGGCAACGAG
CTGTCCACCAAAGAGCAGATCAGCCGGAACAGCAAGGCCCTGGAAGAGAA
ATACGTGGCCGAACTGCAGCTGGAACGGCTGAAGAAAGACGGCGAAGTGC
GGGGCAGCATCAACAGATTCAAGACCAGCGACTACGTGAAAGAAGCCAAA
CAGCTGCTGAAGGTGCAGAAGGCCTACCACCAGCTGGACCAGAGCTTCAT
CGACACCTACATCGACCTGCTGGAAACCCGGCGGACCTACTATGAGGGAC
CTGGCGAGGGCAGCCCCTTCGGCTGGAAGGACATCAAAGAATGGTACGAG
ATGCTGATGGGCCACTGCACCTACTTCCCCGAGGAACTGCGGAGCGTGAA
GTACGCCTACAACGCCGACCTGTACAACGCCCTGAACGACCTGAACAATC
TCGTGATCACCAGGGACGAGAACGAGAAGCTGGAATATTACGAGAAGTTC
CAGATCATCGAGAACGTGTTCAAGCAGAAGAAGAAGCCCACCCTGAAGCA
GATCGCCAAAGAAATCCTCGTGAACGAAGAGGATATTAAGGGCTACAGAG
TGACCAGCACCGGCAAGCCCGAGTTCACCAACCTGAAGGTGTACCACGAC
ATCAAGGACATTACCGCCCGGAAAGAGATTATTGAGAACGCCGAGCTGCT
GGATCAGATTGCCAAGATCCTGACCATCTACCAGAGCAGCGAGGACATCC
AGGAAGAACTGACCAATCTGAACTCCGAGCTGACCCAGGAAGAGATCGAG
CAGATCTCTAATCTGAAGGGCTATACCGGCACCCACAACCTGAGCCTGAA
GGCCATCAACCTGATCCTGGACGAGCTGTGGCACACCAACGACAACCAGA
TCGCTATCTTCAACCGGCTGAAGCTGGTGCCCAAGAAGGTGGACCTGTCC
CAGCAGAAAGAGATCCCCACCACCCTGGTGGACGACTTCATCCTGAGCCC
CGTCGTGAAGAGAAGCTTCATCCAGAGCATCAAAGTGATCAACGCCATCA
TCAAGAAGTACGGCCTGCCCAACGACATCATTATCGAGCTGGCCCGCGAG
AAGAACTCCAAGGACGCCCAGAAAATGATCAACGAGATGCAGAAGCGGAA
CCGGCAGACCAACGAGCGGATCGAGGAAATCATCCGGACCACCGGCAAAG
AGAACGCCAAGTACCTGATCGAGAAGATCAAGCTGCACGACATGCAGGAA
GGCAAGTGCCTGTACAGCCTGGAAGCCATCCCTCTGGAAGATCTGCTGAA
CAACCCCTTCAACTATGAGGTGGACCACATCATCCCCAGAAGCGTGTCCT
TCGACAACAGCTTCAACAACAAGGTGCTCGTGAAGCAGGAAGAAAACAGC
AAGAAGGGCAACCGGACCCCATTCCAGTACCTGAGCAGCAGCGACAGCAA
GATCAGCTACGAAACCTTCAAGAAGCACATCCTGAATCTGGCCAAGGGCA
AGGGCAGAATCAGCAAGACCAAGAAAGAGTATCTGCTGGAAGAACGGGAC
ATCAACAGGTTCTCCGTGCAGAAAGACTTCATCAACCGGAACCTGGTGGA
TACCAGATACGCCACCAGAGGCCTGATGAACCTGCTGCGGAGCTACTTCA
GAGTGAACAACCTGGACGTGAAAGTGAAGTCCATCAATGGCGGCTTCACC
AGCTTTCTGCGGCGGAAGTGGAAGTTTAAGAAAGAGCGGAACAAGGGGTA
CAAGCACCACGCCGAGGACGCCCTGATCATTGCCAACGCCGATTTCATCT
TCAAAGAGTGGAAGAAACTGGACAAGGCCAAAAAAGTGATGGAAAACCAG
ATGTTCGAGGAAAAGCAGGCCGAGAGCATGCCCGAGATCGAAACCGAGCA
GGAGTACAAAGAGATCTTCATCACCCCCCACCAGATCAAGCACATTAAGG
ACTTCAAGGACTACAAGTACAGCCACCGGGTGGACAAGAAGCCTAATAGA
GAGCTGATTAACGACACCCTGTACTCCACCCGGAAGGACGACAAGGGCAA
CACCCTGATCGTGAACAATCTGAACGGCCTGTACGACAAGGACAATGACA
AGCTGAAAAAGCTGATCAACAAGAGCCCCGAAAAGCTGCTGATGTACCAC
CACGACCCCCAGACCTACCAGAAACTGAAGCTGATTATGGAACAGTACGG
CGACGAGAAGAATCCCCTGTACAAGTACTACGAGGAAACCGGGAACTACC
TGACCAAGTACTCCAAAAAGGACAACGGCCCCGTGATCAAGAAGATTAAG
TATTACGGCAACAAACTGAACGCCCATCTGGACATCACCGACGACTACCC
CAACAGCAGAAACAAGGTCGTGAAGCTGTCCCTGAAGCCCTACAGATTCG
ACGTGTACCTGGACAATGGCGTGTACAAGTTCGTGACCGTGAAGAATCTG
GATGTGATCAAAAAAGAAAACTACTACGAAGTGAATAGCAAGTGCTATGA
GGAAGCTAAGAAGCTGAAGAAGATCAGCAACCAGGCCGAGTTTATCGCCT
CCTTCTACAACAACGATCTGATCAAGATCAACGGCGAGCTGTATAGAGTG
ATCGGCGTGAACAACGACCTGCTGAACCGGATCGAAGTGAACATGATCGA
CATCACCTACCGCGAGTACCTGGAAAACATGAACGACAAGAGGCCCCCCA
GGATCATTAAGACAATCGCCTCCAAGACCCAGAGCATTAAGAAGTACAGC
ACAGACATTCTGGGCAACCTGTATGAAGTGAAATCTAAGAAGCACCCTCA
GATCATCAAAAAGGGCAAAAGGCCGGCGGCCACGAAAAAGGCCGGCCAGG
CAAAAAAGAAAAAGggatcctacccatacgatgttccagattacgcttac
ccatacgatgttccagattacgcttacccatacgatgttccagattacgc
ttaagaattcctagagctcgctgatcagcctcgactgtgcttctagttgc
cagccatctgttgtttgcccctcccccgtgccttccttgaccctggaagg
tgccactcccactgtcctttcctaataaaatgaggaaattgcatcgcatt
gtctgagtaggtgtcattctattctggggggtggggtggggcaggacagc
aagggggaggattgggaagagaatagcaggcatgctggggaggtacCAAG
CTTgcctatttcccatgattccttcatatttgcatatacgatacaaggct
gttagagagataattggaattaatttgactgtaaacacaaagatattagt
acaaaatacgtgacgtagaaagtaataatttcttgggtagtttgcagttt
taaaattatgttttaaaatggactatcatatgcttaccgtaacttgaaag
tatttcgatttcttggctttatatatcttgTGGAAAGGACGAAACACCGC
TATTTTGTGCCAGAATTAGgttttagtactctggaaacagaatctactaa
aacaaggcaaaatgccgtgtttatctcgtcaacttgttggcgagattttt
ttGCggccgcaggaacccctagtgatggagttggccactccctctctgcg
cgctcgctcgctcactgaggccgggcgaccaaaggtcgcccgacgcccgg
gctttgcccgggcggcctcagtgagcgagcgagcgcgcagctgcctgcag
gggcgcctgatgcggtattttctccttacgcatctgtgcggtatttcaca
ccgcatacgtcaaagcaaccatagtacgcgccctgtagcggcgcattaag
cgcggcgggtgtggtggttacgcgcagcgtgaccgctacacttgccagcg
ccctagcgcccgctcctttcgctttcttcccttcctttctcgccacgttc
gccggctttccccgtcaagctctaaatcgggggctccctttagggttccg
atttagtgctttacggcacctcgaccccaaaaaacttgatttgggtgatg
gttcacgtagtgggccatcgccctgatagacggtttttcgccctttgacg
ttggagtccacgttctttaatagtggactcttgttccaaactggaacaac
actcaaccctatctcgggctattcttttgatttataagggattttgccga
tttcggcctattggttaaaaaatgagctgatttaacaaaaatttaacgcg
aattttaacaaaatattaacgtttacaattttatggtgcactctcagtac
aatctgctctgatgccgcatagttaagccagccccgacacccgccaacac
ccgctgacgcgccctgacgggcttgtctgctcccggcatccgcttacaga
caagctgtgaccgtctccgggagctgcatgtgtcagaggttttcaccgtc
atcaccgaaacgcgcgagacgaaagggcctcgtgatacgcctatttttat
aggttaatgtcatgataataatggtttcttagacgtcaggtggcactttt
cggggaaatgtgcgcggaacccctatttgtttatttttctaaatacattc
aaatatgtatccgctcatgagacaataaccctgataaatgcttcaataat
attgaaaaaggaagagtatgagtattcaacatttccgtgtcgcccttatt
cccttttttgcggcattttgccttcctgtttttgctcacccagaaacgct
ggtgaaagtaaaagatgctgaagatcagttgggtgcacgagtgggttaca
tcgaactggatctcaacagcggtaagatccttgagagttttcgccccgaa
gaacgttttccaatgatgagcacttttaaagttctgctatgtggcgcggt
attatcccgtattgacgccgggcaagagcaactcggtcgccgcatacact
attctcagaatgacttggttgagtactcaccagtcacagaaaagcatctt
acggatggcatgacagtaagagaattatgcagtgctgccataaccatgag
tgataacactgcggccaacttacttctgacaacgatcggaggaccgaagg
agctaaccgcttttttgcacaacatgggggatcatgtaactcgccttgat
cgttgggaaccggagctgaatgaagccataccaaacgacgagcgtgacac
cacgatgcctgtagcaatggcaacaacgttgcgcaaactattaactggcg
aactacttactctagcttcccggcaacaattaatagactggatggaggcg
gataaagttgcaggaccacttctgcgctcggcccttccggctggctggtt
tattgctgataaatctggagccggtgagcgtggaagccgcggtatcattg
cagcactggggccagatggtaagccctcccgtatcgtagttatctacacg
acggggagtcaggcaactatggatgaacgaaatagacagatcgctgagat
aggtgcctcactgattaagcattggtaactgtcagaccaagtttactcat
atatactttagattgatttaaaacttcatttttaatttaaaaggatctag
gtgaagatcctttttgataatctcatgaccaaaatcccttaacgtgagtt
ttcgttccactgagcgtcagaccccgtagaaaagatcaaaggatcttctt
gagatcctttttttctgcgcgtaatctgctgcttgcaaacaaaaaaacca
ccgctaccagcggtggtttgtttgccggatcaagagctaccaactctttt
tccgaaggtaactggcttcagcagagcgcagataccaaatactgtccttc
tagtgtagccgtagttaggccaccacttcaagaactctgtagcaccgcct
acatacctcgctctgctaatcctgttaccagtggctgctgccagtggcga
taagtcgtgtcttaccgggttggactcaagacgatagttaccggataagg
cgcagcggtcgggctgaacggggggttcgtgcacacagcccagcttggag
cgaacgacctacaccgaactgagatacctacagcgtgagctatgagaaag
cgccacgcttcccgaagggagaaaggcggacaggtatccggtaagcggca
gggtcggaacaggagagcgcacgagggagcttccagggggaaacgcctgg
tatctttatagtcctgtcgggtttcgccacctctgacttgagcgtcgatt
tttgtgatgctcgtcaggggggcggagcctatggaaaaacgccagcaacg
cggcctttttacggttcctggccttttgctggccttttgctcacatgt

SEQ ID NO: 91 Polynucleotide sequence of gRNA 1 in a plasmid cloning vector pDO240, used for evaluating editing efficiency with Surveyor Assay, TIDE, and next-generation sequencing assays (3235 bp)

ctaaattgtaagcgttaatattttgttaaaattcgcgttaaatttttgtt
aaatcagctcattttttaaccaataggccgaaatcggcaaaatcccttat
aaatcaaaagaatagaccgagatagggttgagtgttgttccagtttggaa
caagagtccactattaaagaacgtggactccaacgtcaaagggcgaaaaa
ccgtctatcagggcgatggcccactacgtgaaccatcaccctaatcaagt
tttttggggtcgaggtgccgtaaagcactaaatcggaaccctaaagggag
cccccgatttagagcttgacggggaaagccggcgaacgtggcgagaaagg
aagggaagaaagcgaaaggagcgggcgctagggcgctggcaagtgtagcg
gtcacgctgcgcgtaaccaccacacccgccgcgcttaatgcgccgctaca
gggcgcgtcccattcgccattcaggctgcgcaactgttgggaagggcgat
cggtgcgggcctcttcgctattacgccagctggcgaaagggggatgtgct
gcaaggcgattaagttgggtaacgccagggttttcccagtcacgacgttg
taaaacgacggccagtgagcgcgcgtaatacgactcactatagggcgaat
tgggtacCAAGCTTgcctatttcccatgattccttcatatttgcatatac
gatacaaggctgttagagagataattggaattaatttgactgtaaacaca
aagatattagtacaaaatacgtgacgtagaaagtaataatttcttgggta
gtttgcagttttaaaattatgttttaaaatggactatcatatgcttaccg
taacttgaaagtatttcgatttcttggctttatatatcttgTGGAAAGGA
CGAAACACCGCCAGAAATAGTAGACATGAgttttagtactctggaaacag
aatctactaaaacaaggcaaaatgccgtgtttatctcgtcaacttgttgg
cgagatttttttGCGGCCGCCCgcggtggagctccagcttttgttccctt
tagtgagggttaattgcgcgcttggcgtaatcatggtcatagctgtttcc
tgtgtgaaattgttatccgctcacaattccacacaacatacgagccggaa
gcataaagtgtaaagcctggggtgcctaatgagtgagctaactcacatta
attgcgttgcgctcactgcccgctttccagtcgggaaacctgtcgtgcca
gctgcattaatgaatcggccaacgcgcggggagaggcggtttgcgtattg
ggcgctcttccgcttcctcgctcactgactcgctgcgctcggtcgttcgg
ctgcggcgagcggtatcagctcactcaaaggcggtaatacggttatccac
agaatcaggggataacgcaggaaagaacatgtgagcaaaaggccagcaaa
aggccaggaaccgtaaaaaggccgcgttgctggcgtttttccataggctc
cgcccccctgacgagcatcacaaaaatcgacgctcaagtcagaggtggcg
aaacccgacaggactataaagataccaggcgtttccccctggaagctccc
tcgtgcgctctcctgttccgaccctgccgcttaccggatacctgtccgcc
tttctcccttcgggaagcgtggcgctttctcatagctcacgctgtaggta
tctcagttcggtgtaggtcgttcgctccaagctgggctgtgtgcacgaac
cccccgttcagcccgaccgctgcgccttatccggtaactatcgtcttgag
tccaacccggtaagacacgacttatcgccactggcagcagccactggtaa
caggattagcagagcgaggtatgtaggcggtgctacagagttcttgaagt
ggtggcctaactacggctacactagaaggacagtatttggtatctgcgct
ctgctgaagccagttaccttcggaaaaagagttggtagctcttgatccgg
caaacaaaccaccgctggtagcggtggtttttttgtttgcaagcagcaga
ttacgcgcagaaaaaaaggatctcaagaagatcctttgatcttttctacg
gggtctgacgctcagtggaacgaaaactcacgttaagggattttggtcat
gagattatcaaaaaggatttcacctagatccttttaaattaaaaatgaag
ttttaaatcaatctaaagtatatargagtaaacttggtctgacagttacc
aatgcttaatcagtgaggcacctatctcagcgatctgtctatttcgttca
tccatagttgcctgactccccgtcgtgtagataactacgatacgggaggg
cttaccatctggccccagtgctgcaatgataccgcgagacccacgctcac
cggctccagatttatcagcaataaaccagccagccggaagggccgagcgc
agaagtggtcctgcaactttatccgcctccatccagtctattaattgttg
ccgggaagctagagtaagtagttcgccagttaatagtttgcgcaacgttg
ttgccattgctacaggcatcgtggtgtcacgctcgtcgtttggtatggct
tcattcagctccggttcccaacgatcaaggcgagttacatgatcccccat
gttgtgcaaaaaagcggttagctccttcggtcctccgatcgttgtcagaa
gtaagttggccgcagtgttatcactcatggttatggcagcactgcataat
tctcttactgtcatgccatccgtaagatgcttttctgtgactggtgagta
ctcaaccaagtcattctgagaatagtgtatgcggcgaccgagttgctctt
gcccggcgtcaatacgggataataccgcgccacatagcagaactttaaaa
gtgctcatcattggaaaacgttcttcggggcgaaaactctcaaggatctt
accgctgttgagatccagttcgatgtaacccactcgtgcacccaactgat
cttcagcatcttttactttcaccagcgtttctgggtgagcaaaaacagga
aggcaaaatgccgcaaaaaagggaataagggcgacacggaaatgttgaat
actcatactcttcctttttcaatattattgaagcatttatcagggttatt
gtctcatgagcggatacatatttgaatgtatttagaaaaataaacaaata
ggggttccgcgcacatttccccgaaaagtgccac

SEQ ID NO: 92 Polynucleotide sequence of gRNA 2 in a plasmid cloning vector (3236 bp)

ctaaattgtaagcgttaatattttgttaaaattcgcgttaaatttttgtt
aaatcagctcattttttaaccaataggccgaaatcggcaaaatcccttat
aaatcaaaagaatagaccgagatagggttgagtgttgttccagtttggaa
caagagtccactattaaagaacgtggactccaacgtcaaagggcgaaaaa
ccgtctatcagggcgatggcccactacgtgaaccatcaccctaatcaagt
tttttggggtcgaggtgccgtaaagcactaaatcggaaccctaaagggag
cccccgatttagagcttgacggggaaagccggcgaacgtggcgagaaagg
aagggaagaaagcgaaaggagcgggcgctagggcgctggcaagtgtagcg
gtcacgctgcgcgtaaccaccacacccgccgcgcttaatgcgccgctaca
gggcgcgtcccattcgccattcaggctgcgcaactgttgggaagggcgat
cggtgcgggcctcttcgctattacgccagctggcgaaagggggatgtgct
gcaaggcgattaagttgggtaacgccagggttttcccagtcacgacgttg
taaaacgacggccagtgagcgcgcgtaatacgactcactatagggcgaat
tgggtacCAAGCTTgcctatttcccatgattccttcatatttgcatatac
gatacaaggctgttagagagataattggaattaatttgactgtaaacaca
aagatattagtacaaaatacgtgacgtagaaagtaataatttcttgggta
gtttgcagttttaaaattatgttttaaaatggactatcatatgcttaccg
taacttgaaagtatttcgatttcttggctttatatatcttgTGGAAAGGA
CGAAACACCGAGGCAACTTGCCACTCCCTAgttttagtactctggaaaca
gaatctactaaaacaaggcaaaatgccgtgtttatctcgtcaacttgttg
cgagarttttttGCGGCCGCCCgcggtggagctccagcttttgttccctt
tagtgagggttaattgcgcgcttggcgtaatcatggtcatagctgtttcc
tgtgtgaaattgttatccgctcacaattccacacaacatacgagccggaa
gcataaagtgtaaagcctggggtgcctaatgagtgagctaactcacatta
attgcgttgcgctcactgcccgctttccagtcgggaaacctgtcgtgcca
gctgcattaatgaatcggccaacgcgcggggagaggcggtttgcgtattg
ggcgctcttccgcttcctcgctcactgactcgctgcgctgcgctcggttc
ggctgcggcgagcggtatcagctcactcaaaggcggtaatacggttatcc
acagaatcaggggataacgcaggaaagaacatgtgagcaaaaggccagca
aaaggccaggaaccgtaaaaaggccgcgttgctggcgtttttccataggc
tccgcccccctgacgagcatcacaaaaatcgacgctcaagtcagaggtgg
cgaaacccgacaggactataaagataccaggcgtttccccctggaagctc
cctcgtgcgctctcctgttccgaccctgccgcttaccggatacctgtccg
cctttctcccttcgggaagcgtggcgctttctcatagctcacgctgtagg
tatctcagttcggtgtaggtcgttcgctccaagctgggctgtgtgcacga
accccccgttcagcccgaccgctgcgccttatccggtaactatcgtcttg
agtccaacccggtaagacacgacttatcgccactggcagcagccactggt
aacaggattagcagagcgaggtatgtaggcggtgctacagagttcttgaa
gtggtggcctaactacggctacactagaaggacagtatttggtatctgcg
ctctgctgaagccagttaccttcggaaaaagagttggtagctcttgatcc
ggcaaacaaaccaccgctggtagcggtggtttttttgtttgcaagcagca
gattacgcgcagaaaaaaaggatctcaagaagatcctttgatcttttcta
cggggtctgacgctcagtggaacgaaaactcacgttaagggattttggtc
atgagattatcaaaaaggatcttcacctagatccttttaaattaaaaatg
aagttttaaatcaatctaaagtatatatgagtaaacttggtctgacagtt
accaatgcttaatcagtgaggcacctatctcagcgatctgtctatttcgt
tcatccatagttgcctgactccccgtcgtgtagataactacgatacggga
gggcttaccatctggccccagtgctgcaatgataccgcgagacccacgct
caccggctccagatttatcagcaataaaccagccagccggaagggccgag
cgcagaagtggtcctgcaactttatccgcctccatccagtctattaattg
ttgccgggaagctagagtaagtagttcgecagttaatagtttgegcaaeg
ttgttgecattgctacaggcatcgtggtgtcacgctcgtcgtttggtatg
gcttcattcagctccggttcccaacgatcaaggcgagttacatgatcccc
catgttgtgcaaaaaagcggttagctccttcggtcctccgatcgttgtca
gaagtaagttggccgcagtgttatcactcatggttatggcagcactgcat
aattctcttactgtcatgccatccgtaagatgcttttctgtgactggtga
gtactcaaccaagtcattctgagaatagtgtatgcggcgaccgagttgct
cttgcccggcgtcaatacgggataataccgcgccacatagcagaacttta
aaagtgctcatcattggaaaacgttcttcggggcgaaaactctcaaggat
cttaccgctgttgagatccagttcgatgtaacccactcgtgcacccaact
gatcttcagcatcttttactttcaccagcgtttctgggtgagcaaaaaca
ggaaggcaaaatgccgcaaaaaagggaataagggcgacacggaaatgttg
aatactcatactcttcctttttcaatattattgaagcatttatcagggtt
attgtctcatgagcggatacatatttgaatgtatttagaaaaataaacaa
ataggggttccgcgcacatttccccgaaaagtgccac

SEQ ID NO: 93 Polynucleotide sequence of gRNA 3 in a plasmid cloning vector (3235 bp)

ctaaattgtaagcgttaatattttgttaaaattcgcgttaaatttttgtt
aaatcagctcattttttaaccaataggccgaaatcggcaaaatcccttat
aaatcaaaagaatagaccgagatagggttgagtgttgttccagtttggaa
caagagtccactattaaagaacgtggactccaacgtcaaagggcgaaaaa
ccgtctatcagggcgatggcccactacgtgaaccatcaccctaatcaagt
tttttggggtcgaggtgccgtaaagcactaaatcggaaccctaaagggag
cccccgatttagagcttgacggggaaagccggcgaacgtggcgagaaagg
aagggaagaaagcgaaaggagcgggcgctagggcgctggcaagtgtagcg
gtcacgctgcgcgtaaccaccacacccgccgcgcttaatgcgccgctaca
gggcgcgtcccattcgccattcaggctgcgcaactgttgggaagggcgat
cggtgcgggcctcttcgctattacgccagctggcgaaagggggatgtgct
gcaaggcgattaagttgggtaacgccagggttttcccagtcacgacgttg
taaaacgacggccagtgagcgcgcgtaatacgactcactatagggcgaat
tgggtacCAAGCTTgcctatttcccatgattccttcatatttgcatatac
gatacaaggctgttagagagataattggaattaatttgactgtaaacaca
aagatattagtacaaaatacgtgacgtagaaagtaataatttcttgggta
gtttgcagttttaaaattatgttttaaaatggactatcatatgcttaccg
taacttgaaagtatttcgatttcttggctttatatatcttgTGGAAAGGA
CGAAACACCGTTTCAATGTAACTTAATTCgttttagtactctggaaacag
aatctactaaaacaaggcaaaatgccgtgtttatctcgtcaacttgttgg
cgagatttttttGCGGCCGCCCgcggtggagctccagcttttgttccctt
tagtgagggttaattgcgcgcttggcgtaatcatggtcatagctgtttcc
tgtgtgaaattgttatccgctcacaattccacacaacatacgagccggaa
gcataaagtgtaaagcctggggtgcctaatgagtgagetaactcaeatta
attgcgttgcgctcaetgccegctttecagtegggaaacetgtcgtgcca
gctgeattaatgaatcggceaacgegcggggagaggcggtttgcgtattg
ggcgctcttccgcttcctcgctcactgactcgctgcgctcggtcgttcgg
ctgcggcgagcggtatcagctcactcaaaggcggtaatacggttatccac
agaatcaggggataacgcaggaaagaacatgtgagcaaaaggccagcaaa
aggccaggaaccgtaaaaaggccgcgttgctggcgtttttccataggctc
cgcccccctgacgagcatcacaaaaatcgacgctcaagtcagaggtggcg
aaacccgacaggactataaagataccaggegtttececctggaagctcce
tcgtgcgctetcctgttecgacectgcegcttaccggataectgtccgec
tttetcccttcgggaagcgtggcgctttctcatagctcacgetgtaggta
tctcagttcggtgtaggtcgttcgctccaagctgggctgtgtgcacgaac
cccccgttcagcccgaccgctgcgccttatccggtaactatcgtcttgag
tccaacccggtaagacacgacttatcgccactggcagcagccactggtaa
caggattagcagagcgaggtatgtaggcggtgctacagagttcttgaagt
ggtggcctaactacggctacactagaaggacagtatttggtatctgcgct
ctgctaagccagttaccttcggaaaaagagttggtagctcttgatccggc
aaacaaaccaccgctggtagcggtggtttrtttgrtttgcaagcagcaga
ttacgcgcagaaaaaaaggatctcaagaagatcctttgatcttttctacg
gggtctgacgctcagtggaacgaaaactcacgttaagggattttggtcat
gagattatcaaaaaggatcttcacctagatccttttaaattaaaaatgaa
gttttaaatcaatctaaagtatatatgagtaaacttggtctgacagttac
caatgcttaatcagtgaggcacctatctcagcgatctgtctatttcgttc
atccatagttgcctgactccccgtcgtgtagataactacgatacgggagg
gcttaccatctggccccagtgctgcaatgataccgcgagacccacgctca
ccggctccagatttatcagcaataaaccagccagccggaagggccgagcg
cagaagtggtcctgcaactttatccgcctccatccagtctattaattgtt
gccgggaagctagagtaagtagttcgccagttaatagtttgcgcaacgtt
gttgccattgctacaggcatcgtggtgtcacgctcgtcgtttggtatggc
ttcattcagctccggttcccaacgatcaaggcgagttacatgatccccca
tgttgtgcaaaaaagcggttagctccttcggtcctccgatcgttgtcaga
agtaagttggccgcagtgttatcactcatggttatggcagcactgcataa
ttctcttactgtcatgccatccgtaagatgcttttctgtgactggtgagt
actcaaccaagtcattctgagaatagtgtatgcggcgaccgagttgctct
tgcccggcgtcaatacgggataataccgcgccacatagcagaactttaaa
agtgctcatcattggaaaacgttcttcggggcgaaaactctcaaggatct
taccgctgttgagatccagttcgatgtaacccactcgtgcacccaactga
tcttcagcatcttttactttcaccagcgtttctgggtgagcaaaaacagg
aaggcaaaatgccgcaaaaaagggaataagggcgacacggaaatgttgaa
tactcatactcttcctttttcaatattattgaagcatttatcagggttat
tgtctcatgagcggatacatatttgaatgtatttagaaaaataaacaaat
aggggttccgcgcacatttccccgaaaagtgccac

SEQ ID NO: 94 Polynucleotide sequence of gRNA 4 in a plasmid cloning vector (3236 bp)

ctaaattgtaagcgttaatattttgttaaaattcgcgttaaatttttgtt
aaatcagctcattttttaaccaataggccgaaatcggcaaaatcccttat
aaatcaaaagaatagaccgagatagggttgagtgttgttccagtttggaa
caagagtccactattaaagaacgtggactccaacgtcaaagggcgaaaaa
ccgtctatcagggcgatggcccactacgtgaaccatcaccctaatcaagt
tttttggggtcgaggtgccgtaaageactaaateggaaecctaaagggag
cccecgatttagagcttgacggggaaagccggcgaaegtggcgagaaagg
aagggaagaaagcgaaaggagcgggcgctagggcgctggcaagtgtagcg
gtcacgctgcgcgtaaccaccacacccgccgcgcttaatgcgccgctaca
gggcgcgtcccattcgccattcaggctgcgcaactgttgggaagggcgat
cggtgcgggcctcttcgctattacgccagctggcgaaagggggatgtgct
gcaaggcgattaagttgggtaacgccagggttttcccagtcacgacgttg
taaaacgacggccagtgagcgcgegtaatacgactcactatagggcgaat
tgggtaeCAAGCTTgcctatttcccatgattccttcatatttgcatatac
gatacaaggctgttagagagataattggaattaatttgactgtaaacaca
aagatattagtacaaaatacgtgacgtagaaagtaataatttcttgggta
gtttgcagttttaaaattatgttttaaaatggactatcatatgcttaccg
taacttgaaagtatttcgatttcttggctttatatatcttgTGGAAAGGA
CGAAACACCGAATCAAACCAAAGGAACACAgttttagtactctggaaaca
gaatctactaaaacaaggcaaaatgccgtgtttatctcgtcaacttgttg
gcgagatttttttGCGGCCGCCCgcggtggagetceagcttttgttccct
ttagtgagggttaattgcgcgcttggcgtaatcatggtcatagctgtttc
ctgtgtgaaattgttatccgctcacaattccacacaacatacgagccgga
agcataaagtgtaaagcctggggtgcctaatgagtgagctaactcacatt
aattgcgttgcgctcactgcccgctttccagtcgggaaacctgtcgtgcc
agctgcattaatgaatcggccaacgcgcggggagaggcggtttgcgtatt
gggcgctcttccgcttcctcgctcactgactcgctgcgctcggtcgttcg
gctgcggcgagcggtatcagctcactcaaaggcggtaatacggttatcca
cagaatcaggggataacgcaggaaagaacatgtgagcaaaaggccagcaa
aaggccaggaaccgtaaaaaggccgcgttgctggcgtttttccataggct
ccgcccccctgacgagcatcacaaaaatcgacgctcaagtcagaggtggc
gaaacccgacaggactataaagataccaggcgtttccccctggaagctcc
ctcgtgcgctctcctgttccgaccctgccgcttaccggatacctgtccgc
ctttctcccttcgggaagcgtggcgctttctcatagctcacgctgtaggt
atctcagttcggtgtaggtcgttcgctccaagctgggctgtgtgcacgaa
ccccccgttcagcccgaccgctgcgccttatccggtaactatcgtcttga
gtccaacccggtaagacacgacttatcgccactggcagcagccactggta
acaggattagcagagcgaggtatgtaggcggtgctacagagttcttgaag
tggtggcctaactacggctacactagaaggacagtatttggtatctgcgc
tctgctgaagccagttaccttcggaaaaagagttggtagctcttgatccg
gcaaacaaaccaccgctggtagcggtggtttttttgtttgcaagcagcag
attacgcgcagaaaaaaaggatctcaagaagatcctttgatcttttctac
ggggtctgacgctcagtggaacgaaaactcacgttaagggattttggtca
tgagattatcaaaaaggacttcacctagatccttttaaattaaaaatgaa
gttttaaatcaatctaaagtatatatgagtaaacttggtctgacagttac
caatgcttaatcagtgaggcacctatctcagcgatctgtctatttcgttc
atccatagttgcctgactccccgtcgtgtagataactacgatacgggagg
gcttaccatctggccccagtgctgcaatgataccgcgagacccacgctca
ccggctccagatttatcagcaataaaccagccagccggaagggccgagcg
cagaagtggtcctgcaactttatccgcctccatccagtctattaattgtt
gccgggaagctagagtaagtagttcgccagttaatagtttgcgcaacgtt
gttgccattgctacaggcatcgtggtgtcacgctcgtcgtttggtatggc
ttcattcagctccggttcccaacgatcaaggcgagttacatgatccccca
tgttgtgcaaaaaagcggttagctccttcggtcctccgatcgttgtcaga
agtaagttggccgcagtgttatcactcatggttatggcagcactgcataa
ttctcttactgtcatgccatccgtaagatgcttttctgtgactggtgagt
actcaaccaagtcattctgagaatagtgtatgcggcgaccgagttgctct
tgcccggcgtcaatacgggataataccgcgccacatagcagaactttaaa
agtgctcatcattggaaaacgttcttcggggcgaaaactctcaaggatct
taccgctgttgagatccagttcgatgtaacccactcgtgcacccaactga
tcttcagcatcttttactttcaccagcgtttctgggtgagcaaaaacagg
aaggcaaaatgccgcaaaaaagggaataagggcgacacggaaatgttgaa
tactcatactcttcctttttcaatattattgaagcatttatcagggttat
tgtctcatgagcggatacatatttgaatgtatttagaaaaataaacaaat
aggggttccgcgcacatttccccgaaaagtgccac

SEQ ID NO: 95 Polynucleotide sequence of gRNA 5 in a plasmid cloning vector (3237 bp)

ctaaattgtaagcgttaatattttgttaaaattcgcgttaaatttttgtt
aaatcagctcattttttaaccaataggccgaaatcggcaaaatcccttat
aaatcaaaagaatagaccgagatagggttgagtgttgttccagtttggaa
caagagtccactattaaagaacgtggactccaacgtcaaagggcgaaaaa
ccgtctatcagggcgatggcccactacgtgaaccatcaccctaatcaagt
tttttggggtcgaggtgccgtaaagcactaaatcggaaccctaaagggag
cccccgatttagagcttgacggggaaagccggcgaacgtggcgagaaagg
aagggaagaaagcgaaaggagcgggcgct.agggcgctggcaagtgtagc
ggtcacgctgcgcgtaaccaccacacccgccgcgcttaatgcgccgctac
agggcgcgtcccattcgccattcaggctgcgcaactgttgggaagggcga
tcggtgcgggcctcttcgctattacgccagctggcgaaagggggatgtgc
tgcaaggcgattaagttgggtaacgccagggttttcccagtcacgacgtt
gtaaaacgacggccagtgagcgcgcgtaatacgactcactatagggcgaa
ttgggtacCAAGCTTgcctatttcccatgattccttcatatttgcatata
cgatacaaggctgttagagagataattggaattaatttgactgtaaacac
aaagatattagtacaaaatacgtgacgtagaaagtaataatttcttgggt
agtttgcagttttaaaattatgttttaaaatggactatcatatgcttacc
gtaacttgaaagtatttcgatttcttggctttatatatcttgTGGAAAGG
ACGAAACACCGACATTCTCTGTGTTCCTTTGGgttttagtactctggaaa
cagaatctactaaaacaaggcaaaatgccgtgtttatctcgtcaacttgt
tggcgagatttttttGCGGCCGCCCgcggtggagctccagcttttgttcc
ctttagtgagggttaattgcgcgcttggcgtaatcatggtcatagctgtt
tcctgtgtgaaattgttatccgctcacaattccacacaacatacgagccg
gaagcataaagtgtaaagcctggggtgcctaatgagtgagctaactcaca
ttaattgcgttgcgctcactgcccgctttccagtcgggaaacctgtcgtg
ccagctgcattaatgaatcggccaacgcgcggggagaggcggtttgcgta
ttgggcgctcttccgcttcctcgctcactgactcgctgcgctcggtcgtt
cggctgcggcgagcggtatcagctcactcaaaggcggtaatacggttatc
cacagaatcaggggataacgcaggaaagaacatgtgagcaaaaggccagc
aaaaggccaggaaccgtaaaaaggccgcgttgctggcgtttttccatagg
ctccgcccccctgacgagcatcacaaaaatcgacgctcaagtcagaggtg
gcgaaacccgacaggactataaagataccaggcgtttccccctggaagct
ccctcgtgcgctctcctgttccgaccctgccgcttaccggatacctgtcc
gcctttctcccttcgggaagcgtggcgctttctcatagctcacgctgtag
gtatctcagttcggtgtaggtcgttcgctccaagctgggctgtgtgcacg
aaccccccgttcagcccgaccgctgcgccttatccggtaactatcgtctt
gagtccaacccggtaagacacgacttatcgccactggcagcagccactgg
taacaggattagcagagcgaggtatgtaggcggtgctacagagttcttga
agtggtggcctaactacggctacactagaaggacagtatttggtatctgc
gctctgctgaagccagttaccttcggaaaaagagttgg tagctcttgat
ccggcaaacaaaccaccgctggtagcggtggtttttttgtttgcaagcag
cagattacgcgcagaaaaaaaggatctcaagaagatcctttgatcttttc
tacggggtctgaegctcagtggaacgaaaaeteacgttaagggattttgg
tcatgagattatcaaaaaggatcttcacctagatccttttaaattaaaaa
tgaagttttaaatcaatctaaagtatatatgagtaaacttggtctgacag
ttaccaatgcttaatcagtgaggcacctatctcagcgatctgtctatttc
gttcatccatagttgcctgactccccgtcgtgtagataactacgatacgg
gagggcttaccatctggccccagtgctgcaatgataccgcgagacccacg
ctcaccggctccagatttatcagcaataaaccagccagccggaagggccg
agcgcagaagtggtcctgcaactttatccgcctccatccagtctattaat
tgttgccgggaagctagagtaagtagttcgccagttaat.agtttgcgca
acgttgttgccattgctacaggcatcgtggtgtcacgctcgtcgtttggt
atggcttcattcagctccggttcccaacgatcaaggcgagttacagatcc
cccatgttgtgcaaaaaagcggttagctccttcggtcctccgatcgttgt
cagaagtaagttggccgcagtgttatcactcatggttatggcagcactgc
ataattctcttactgtcatgccatccgtaagatgcttttctgtgactggt
gagtactcaaccaagtcattctgagaatagtgtatgcggcgaccgagttg
ctcttgcccggcgtcaatacgggataataccgcgccacatagcagaactt
taaaagtgctcatcattggaaaacgttcttcggggcgaaaactctcaagg
atcttaccgctgttgagatccagttcgatgtaacccactcgtgcacccaa
ctgatcttcagcatcttttactttcaccagcgtttctgggtgagcaaaaa
caggaaggcaaaatgccgcaaaaaagggaataagggcgacacggaaatgt
tgaatactcatactcttcctttttcaatattattgaagcatttatcaggg
ttattgtctcatgagcggatacatatttgaatgtatttagaaaaataaac
aaataggggttccgcgcacatttccccgaaaagtgccac

SEQ ID NO: 96 Polynucleotide sequence of gRNA 6 in a plasmid cloning vector (3236 bp)

ctaaattgtaagcgttaatattttgttaaaattcgcgttaaatttttgtt
aaatcagctcattttttaaccaataggccgaaatcggcaaaatcccttat
aaatcaaaagaatagaccgagatagggttgagtgttgttccagtttggaa
caagagtccactattaaagaacgtggactccaacgtcaaagggcgaaaaa
ccgtctatcagggcgatggcccactacgtgaaccatcaccctaatcaagt
tttttggggtcgaggtgccgtaaagcactaaatcggaaccctaaagggag
cccccgatttagagcttgacggggaaagccggcgaacgtggcgagaaagg
aagggaagaaagcgaaaggagcgggcgctagggcgctggcaagtgtagcg
gtcacgctgcgcgtaaccaccacacccgccgcgct.taatgcgccgctac
agggcgcgtcccattcgccattcaggctgcgcaactgttgggaagggcga
tcggtgcgggcctcttcgctattacgccagctggcgaaagggggatgtgc
tgcaaggcgattaagttgggtaacgccagggttttcccagtcacgacgtt
gtaaaacgacggccagtgagcgcgcgtaatacgactcactatagggcgaa
ttgggtacCAAGCTTgcctatttccatgattccttcatatttgcatatac
gatacaaggctgttagagagataattggaattaatttgactgtaaacaca
aagatattagtacaaaatacgtgacgtagaaagtaataatttcttgggta
gtttgcagttttaaaattatgttttaaaatggactat.catat.gcttac
cgtaacttgaaagtatttcgatttcttggctttatatatcttgTGGAAAG
GACGAAACACCGCTATTTTGTGCCAGAATTAGgttttagtactctggaaa
cagaatctactaaaacaaggcaaaatgccgtgtttatctcgtcaacttgt
tggcgagatttttttGCGCCGCCCgcggtggagctccagcttttgttccc
tttagtgagggttaattgcgcgcttggcgtaatcatggtcatagctgttt
cctgtgtgaaattgttatccgctcacaattccacacaacatacgagccgg
aagcataaagtgtaaagcctggggtgcctaatgagtgagctaactcacat
taattgcgttgcgctcactgcccgctttccagtcgggaaacctgtcgtgc
cagctgcattaatgaatcggccaacgcgcggggagaggcggtttgcgtat
tgggcgctcttccgcttcctcgctcactgactcgctgcgctcggtcgttc
ggctgcggcgagcggtatcagctcactcaaaggcggtaatacggttatcc
acagaatcaggggataacgcaggaaagaacatgtgagcaaaaggccagca
aaaggccaggaaccgtaaaaaggccgcgttgctggcgtttttccataggc
tccgcccccctgacgagcatcacaaaaatcgacgctcaagtcagaggtgg
cgaaacccgacaggactataaagataccaggcgtttccccctggaagctc
cctcgtgcgctctcctgttccgaccctgccgcttaccggatacctgtccg
cctttctcccttcgggaagcgtggcgctttctcatagctcacgctgtagg
tatctcagttcggtgtaggtcgttcgctccaagctgggctgtgtgcacga
accccccgttcagcccgaccgctgcgccttatccggtaactatcgtcttg
agtccaacccggtaagacacgacttatcgccactggcagcagccactggt
aacaggattagcagagcgaggtatgtaggcggtgctacagagttcttgaa
gtggtggcctaactacggctacactagaaggacagtatttggtatctgcg
ctctgctgaagccagttaccttcggaaaaagagttggtagctct.tgatc
cggcaaacaaaccaccgctggtagcggtggtttttttgtttgcaagcage
agattacgcgcagaaaaaaaggatctcaagaagatcctttgatcttttct
acggggtctgacgctcagtggaacgaaaactcacgttaagggattttggt
catgagattatcaaaaaggatcttcacctagatccttttaaattaaaaat
gaagttttaaatcaatctaaagtatatatgagtaaacttggtctgacagt
taccaatgcttaatcagtgaggcacctatctcagcgatctgtctatttcg
ttcatccatagttgcctgactccccgtcgtgtagataactacgatacggg
agggcttaccatctggccccagtgctgcaatgataccgcgagacccacgc
tcaccggctccagatttatcagcaataaaccagccagccggaagggccga
gcgeagaagtggtcctgcaactttatecgcctccatecagtetattaatt
gttgecgggaagctagagtaagtagttcgccagttaatagtttgcgcaac
gttgttgccattgctacaggcatcgtggtgtcacgctcgtcgtttggtat
ggcttcattcagctccggttcccaacgatcaaggcgagttacatgatccc
ccatgttgtgcaaaaaagcggttagctccttcggtcctccgatcgttgtc
agaagtaagttggccgcagtgttatcactcatggttatggcagcactgca
taattctcttactgtcatgccatccgtaagatgcttttctgtgactggtg
agtacteaaccaagtcattctgagaatagtgtatgcggcgaccgagttge
tcttgcccggcgtcaatacgggataataccgcgccacatagcagaacttt
aaaagtgctcatcattggaaaacgttcttcggggcgaaaactctcaagga
tcttaccgctgttgagatccagttcgatgtaacccactcgtgcacccaac
tgatcttcagcatcttttactttcaccagcgtttctgggtgagcaaaaac
aggaaggcaaaatgccgcaaaaaagggaataagggcgacacggaaatgtt
gaatactcatactcttcctttttcaatattattgaagcatttatcagggt
tattgtctcatgagcggatacatatttgaatgtatttagaaaaataaaca
aataggggttccgcgcacatttccccgaaaagtgccac

SEQ ID NO: 97 Polynucleotide sequence of gRNA 7 in a plasmid cloning vector (3237 bp)

ctaaattgtaagcgttaatattttgttaaaattcgcgttaaatttttgtt
aaatcagctcattttttaaccaataggccgaaatcggcaaaatcccttat
aaatcaaaagaatagaccgagatagggttgagtgttgttccagtttggaa
caagagtccactattaaagaacgtggactccaacgtcaaagggcgaaaaa
ccgtctatcagggcgatggcccactacgtgaaccatcaccctaatcaagt
tttttggggtcgaggtgccgtaaagcactaaatcggaaccctaaagggag
cccccgatttagagcttgacggggaaagccggcgaacgtggcgagaaagg
aagggaagaaagcgaaaggagcgggcgctagggcgctggcaagtgtagcg
gtcacgctgcgcgtaaccaccacacccgccgcgcttaatgcgccgctaca
gggcgcgtcccattcgccattcaggctgcgcaactgttgggaagggcgat
cggtgcgggcctcttcgctattacgccagctggcgaaagggggatgtgct
gcaaggcgattaagttgggtaacgccagggttttcccagtcacgacgttg
taaaacgacggccagtgagcgcgcgtaatacgactcactatagggcgaat
tgggtacCAAGCTTgcctatttcccatgattcettcatatttgcatatac
gatacaaggctgttagagagataattggaattaatttgactgtaaacaca
aagatattagtacaaaatacgtgacgtagaaagtaataatttcttgggta
gtttgcagttttaaaattatgttttaaaatggactatcatatgcttaccg
taacttgaaagtatttcgatttcttggctttatatatcttgTGGAAAGGA
CGAAACACCGCCAAAGGAACACAGAGAATGTgttttagtactctggaaac
agaatctactaaaacaaggcaaaatgccgtgtttatctcgtcaacttgtt
ggcgagatttttttGCGGCCGCCCgcggtggagctccagcttttgttccc
tttagtgagggttaattgcgcgcttggcgtaatcatggtcatagctgttt
cctgtgtgaaattgttatccgctcacaattccacacaacatacgagccgg
aagcataaagtgtaaagcctggggtgcctaatgagtgagctaactcacat
taattgcgttgcgctcactgcccgctttccagtcgggaaacctgtcgtgc
cagctgcattaatgaatcggccaacgcgcggggagaggcggtttgcgtat
tgggcgctcttccgcttcctcgctcactgactcgctgcgctcggtcgttc
ggctgcggcgagcggtatcagctcactcaaaggcggtaatacggttatcc
acagaatcaggggataacgcaggaaagaacatgtgagcaaaaggecagca
aaaggccaggaaccgtaaaaaggccgcgttgctggcgtttttecataggc
tccgccccectgaegagcatcacaaaaatcgacgcteaagteagaggtgg
cgaaacecgacaggactataaagataccaggcgtttccccctggaagctc
cctcgtgcgctctcctgttccgaccctgccgcttaccggatacctgtccg
cctttctcccttcgggaagcgtggcgctttctcatagctcacgctgtagg
tatctcagttcggtgtaggtcgttcgctccaagctgggctgtgtgcacga
accccccgttcagcccgaccgctgcgccttatccggtaactatcgtcttg
agtccaacccggtaagacacgacttatcgccactggcagcagceactggt
aacaggattageagagegaggtatgtaggcggtgctacagagttettgaa
gtggtggcctaactacggctacactagaaggacagtatttggtatctgcg
ctctgctgaagccagttaccttXcggaaaaagagttggtagctcttgatc
cggcaaacaaaccaccgctggtagcggtggtttttttgtttgcaagcagc
agattacgcgcagaaaaaaaggatctcaagaagatcctttgatcttttct
acggggtctgacgctcagtggaacgaaaactcacgttaagggattttggt
catgagattatcaaaaaggatcttcacctagatccttttaaattaaaaat
gaagttttaaatcaatctaaagtatatatgagtaaacttggtctgacagt
taccaatgcttaatcagtgaggcacctatctcagcgatctgtctatttcg
ttcatccatagttgcctgactccccgtcgtgtagataactacgatacggg
agggcttaccatctggccccagtgctgcaatgataccgcgagacccacgc
tcaccggctccagatttatcagcaataaaccagccagccggaagggccga
gcgcagaagtggtcctgcaactttatccgcctccatccagtctattaatt
gttgccgggaagctagagtaagtagttcgccagttaatagtttgcgcaac
gttgttgccattgctacaggcatcgtggtgtcacgctcgtcgtttggtat
ggcttcattcagctccggttcccaacgatcaaggcgagttacatatcccc
catgttgtgcaaaaaagcggttagctccttcggtcctccgatcgttgtca
gaagtaagttggccgcagtgttatcactcatggttatggcagcactgcat
aattctcttactgtcatgccatccgtaagatgcttttctgtgactggtga
gtactcaaccaagtcattctgagaatagtgtatgcggcgaccgagttgct
cttgcccggcgtcaatacgggataataccgcgccacatagcagaacttta
aaagtgcteateattggaaaaegttcttcggggcgaaaactctcaaggat
ettacegctgttgagatccagttcgatgtaacceaetcgtgcacccaact
gatcttcagcatcttttactttcaccagcgtttctgggtgagcaaaaaca
ggaaggcaaaatgccgcaaaaaagggaataagggcgacacggaaatgttg
aatactcatactcttcctttttcaatattattgaagcatttatcagggtt
attgtctcatgagcggatacatatttgaatgtatttagaaaaataaacaa
ataggggttccgcgcacatttccccgaaaagtgccac

SEQ ID NO: 98 Polynucleotide sequence of gRNA 8 in a plasmid cloning vector (3237 bp)

ctaaattgtaagcgttaatattttgttaaaattcgcgttaaatttttgtt
aaatcagctcattttttaaccaataggccgaaatcggcaaaatcccttat
aaatcaaaagaatagaccgagatagggttgagtgttgttccagtttggaa
caagagtccactattaaagaacgtggactccaacgtcaaagggcgaaaaa
ccgtctatcagggcgatggcccactacgtgaaccatcaccctaatcaagt
tttttggggtcgaggtgccgtaaagcactaaatcggaaccctaaagggag
cccccgatttagagcttgacggggaaagccggcgaacgtggcgagaaagg
aagggaagaaagcgaaaggagcgggcgctagggcgctggcaagtgtagcg
gtcacgctgcgcgtaaccaccacacccgccgcgcttaatgcgccgctaca
gggcgcgtcccattcgccttcaggctgcgcaactgttgggaagggcgatc
ggtgcgggcctcttcgctattacgccagctggcgaaagggggatgtgctg
caaggcgattaagttgggtaacgccagggtttteccagtcacgacgttgt
aaaacgacggccagtgagcgcgcgtaatacgactcactatagggcgaatt
gggtacCAAGCTTgcctatttcccatgattccttcatatttgcatatacg
atacaaggctgttagagagataattggaattaatttgactgtaaacacaa
agatattagtacaaaatacgtgacgtagaaagtaataatttcttgggtag
tttgcagttttaaaattatgttttaaaatggactatcatatgcttaccgt
aacttgaaagtatttcgatttcttggctttatatatcttgTGGAAAGGAC
GAAACACCGATTTTGTGCCAGAATTAGTAGgttttagtactctggaaaca
gaatctactaaaacaaggcaaaatgccgtgtttatctcgtcaacttgttg
gcgagatttttttGCGGCCGCCCgcggtggagctccagcttttgttccct
ttagtgagggttaattgcgcgcttggcgtaatcatggtcatagctgtttc
ctgtgtgaaattgttatccgctcacaattccacacaacatacgagccgga
agcataaagtgtaaagcctggggtgcctaatgagtgagctaactcacatt
aattgcgttgcgctcactgcccgctttccagtcgggaaacctgtcgtgcc
agctgcattaatgaatcggccaacgcgcggggagaggggtttgcgtattg
ggegctcttccgettcctcgcteactgactcgetgegctcggtcgttcgg
etgcggcgagcggtatcagctcactcaaaggcggtaatacggttatccac
agaatcaggggataacgcaggaaagaacatgtgagcaaaaggccagcaaa
aggccaggaaccgtaaaaaggccgcgttgctggcgtttttccataggctc
cgcccccctgacgagcatcacaaaaatcgacgctcaagtcagaggtggcg
aaacccgacaggactataaagataccaggcgtttccccctggaagctccc
tcgtgcgctctcctgttccgaccetgccgcttaceggatacctgtccgcc
tttctccettcgggaagegtggegctttcteatagetcacgctgtaggta
tctcagttcggtgtaggtcgttcgctccaagctgggctgtgtgcacgaac
cccccgttcagcccgaccgctgcgccttatccggtaactatcgtcttgag
tccaacccggtaagacacgacttatcgccactggcagcagccactggtaa
caggattagcagagcgaggtatgtaggcggtgctacagagttcttgaagt
ggtggcctaactacggctacactagaaggaeagtatttggtatotgeget
otgetgaagccagttaccttcggaaaaagagttggtagctcttgatccgg
caaacaaaccaccgctggtagcggtggtttttttgtttgcaagcagcaga
ttacgcgcagaaaaaaaggatctcaagaagatcctttgatcttttctacg
gggtctgacgctcagtggaacgaaaactcacgttaagggattttggtcat
gagattatcaaaaaggatcttcacctagatccttttaaattaaaaatgaa
gttttaaatcaatctaaagtatatatgagtaaacttggtctgacagttac
caatgcttaatcagtgaggcacctatctcagcgatctgtctatttcgttc
atccatagttgcctgactccccgtcgtgtagataactacgatacgggagg
gcttaccatctggccccagtgctgcaatgataccgcgagacccacgctca
ccggctccagatttatcagcaataaaccagccagccggaagggccgagcg
cagaagtggtcctgcaactttatccgcctccatccagtctattaattgtt
gccgggaagctagagtaagtagttcgccagttaatagtttgcgcaacgtt
gttgccattgctacaggcatcgtgtgtcacgctegtcgtttggtatgget
teattcagctceggtteecaaegatcaaggcgagttaeatgatceeccat
gttgtgcaaaaaagcggttagctccttcggtcctccgatcgttgtcagaa
gtaagttggccgcagtgttatcactcatggttatggcagcactgcataat
tctcttactgtcatgccatccgtaagatgcttttctgtgactggtgagta
ctcaaccaagtcattctgagaatagtgtatgcggcgaccgagttgctctt
gcccggcgtcaatacgggataataccgcgccacatagcagaactttaaaa
gtgctcatcattggaaaacgttcttcggggcgaaaactctcaaggatctt
accgctgttgagatccagttcgatgtaacccactcgtgcacccaactgat
cttcagcatcttttactttcaccagcgtttctgggtgagcaaaaacagga
aggcaaaatgccgcaaaaaagggaataagggcgacacggaaatgttgaat
actcatactcttcctttttcaatattattgaagcatttatcagggttatt
gtctcatgagcggatacatatttgaatgtatttagaaaaataaacaaata
ggggttccgcgcacatttccccgaaaagtgccac

SEQ ID NO: 99 Polynucleotide sequence of gRNA 9 in a plasmid cloning vector (3236 bp)

taaattgtaagcgttaatattttgttaaaattcgcgttaaatttttgtta
aatcagctcattttttaaccaataggccgaaatcggcaaaatcccttata
aatcaaaagaafagaccgagaragggttgagtgttgttccagtttggaac
aagagtccactattaaagaacgtggactccaacgtcaaagggcgaaaaac
cgtctatcagggcgatggcccactacgtgaaccatcaccctaatcaagtt
ttttggggtcgaggtgccgtaaagcactaaatcggaaccctaaagggagc
ccccgatttagagcttgacggggaaagccggcgaacgtggcgagaaagga
agggaagaaagcgaaaggagcgggcgctagggcgctggcaagtgtagcgg
tcacgctgcgcgtaaccaccacacccgccgcgcttaatgcgccgctacag
ggcgcgtcccattcgccattcaggctgcgcaactgttgggaagggcgatc
ggtgcgggcctcttcgctattacgccagctggcgaaagggggatgtgctg
caaggcgattaagttgggtaacgccagggttttcccagtcacgacgttgt
aaaacgacggccagtgagcgcgcgtaatacgactcactatagggcgaatt
gggtacCAAGCTTgcctatttcccatgattccttcatatttgcatatacg
atacaaggctgttagagagataattggaattaatttgactgtaaacacaa
agatattagtacaaaatacgtgacgtagaaagtaataatttcttgggtag
tttgcagttttaaaattatgttttaaaatggactatcatatgcttaccgt
aacttgaaagtatttcgatttcttggctttatatatcttgTGGAAAGGAC
GAAACACCGTGGCAAGTTGCCTACAGAACgttttagtactctggaaacag
aatctactaaaacaaggcaaaatgccgtgtttatctcgtcaacttgttgg
cgagatttttttGCGGCCGCCCgcggtggagctccagcttttgttccctt
tagtgagggttaattgcgcgcttggcgtaatcatggtcatagctgtttcc
tgtgtgaaattgttatccgctcacaattccacacaacatacgagccggaa
gcataaagtgtaaagcctggggtgcctaatgagtgagctaactcacatta
attgcgttgcgctcactgcccgctttccagtcgggaaacctgtcgtgcca
gctgcattaatgaatcggccaacgcgcggggagaggcggtttgcgtattg
ggcgctcttccgcttcctcgctcactgactcgctgcgctcggtcgttcgg
ctgcggcgagcggtatcagctcactcaaaggcggtaatacggttatccac
agaatcaggggataacgcaggaaagaacatgtgagcaaaaggccagcaaa
aggccaggaaccgtaaaaaggccgcgttgctggcgtttttccataggctc
cgcccccctgacgagcatcacaaaaatcgacgctcaagtcagaggtggcg
aaacccgacaggactataaagataccaggcgtttccccctggaagctccc
tcgtgcgctctcctgttccgaccctgccgcttaceggataectgtccgec
tttetccettcgggaagegtggegctttcteatagetcacgetgtaggta
tctcagttcggtgtaggtcgttcgctccaagctgggctgtgtgcacgaac
cccccgttcagcccgaccgctgcgccttatccggtaactatcgtcttgag
tccaacccggtaagacacgacttatcgccactggcagcagccactggtaa
caggattagcagagcgaggtatgtaggcggtgctacagagttcttgaagt
ggtggcctaactacggctacactagaaggacagtatttggtatctgcgct
ctgctgaagccagttaccttcggaaaaagagttggtagctcttgatccgg
caaacaaaccaccgctggtagcggtggtttttttgtttgcaagcagcaga
ttacgcgcagaaaaaaaggatctcaagaagatcctttgatcttttctacg
gggtctgacgctcagtggaacgaaaactcacgttaagggattttggtcat
gagattatcaaaaaggatcttcacctagatccttttaaattaaaaatgaa
gttttaaatcaatctaaagtatatatgagtaaacttggtctgacagttac
caatgcttaatcagtgaggcacctatctcagcgatctgtctatttcgttc
atccatagttgcctgactccccgtcgtgtagataactacgatacgggagg
gcttaccatctggccccagtgctgcaatgataccgcgagacccacgctca
ccggctccagatttatcagcaataaaccagccagccggaagggccgagcg
cagaagtggtcctgcaactttatccgcctccatccagtctattaattgtt
gccgggaagctagagtaagtagttcgccagttaatagtttgcgcaacgtt
gttgccattgctacaggcatcgtggtgtcacgctcgtcgtttggtatggc
ttcattcagctccggttcccaacgatcaaggcgagttacatgatccccca
tgttgtgcaaaaaagcggttagctccttcggtcctccgatcgttgtcaga
agtaagttggccgcagtgttatcactcatggttatggcagcagtgcataa
ttctcttactgtcatgccatgcgtaagatggttttctgtgactggtgagt
actcaaccaagtcattctgagaatagtgtatgcggcgaccgagttgctct
tgcccggcgtcaatacgggataataccgcgccacatagcagaactttaaa
agtgctcatcattggaaaacgttcttcggggcgaaaactctcaaggatct
taccgctgttgagatccagttcgatgtaacccactcgtgcacccaactga
tcttcagcatcttttactttcaccagcgtttctgggtgagcaaaaacagg
aaggcaaaatgccgcaaaaaagggaataagggcgacacggaaatgttgaa
tactcatactcttcctttttcaatattattgaagcatttatcagggttat
tgtctcatgagcggatacatatttgaatgtatttagaaaaataaacaaat
aggggttccgcgcacatttccccgaaaagtgccac

SEQ ID NO: 100 Polynucleotide sequence of gRNA 10 in a plasmid cloning vector (3237 bp)

ctaaattgtaagcgttaatattttgttaaaattcgcgttaaatttttgtt
aaatcagctcattttttaaccaataggccgaaatcggcaaaatcccttat
aaatcaaaagaatagaccgagatagggttgagtgttgttccagtttggaa
caagagtccactattaaagaacgtggactccaacgtcaaagggcgaaaaa
ccgtctatcagggcgatggcccactacgtgaaccatcaccctaatcaagt
tttttggggtcgaggtgccgtaaagcactaaatcggaaccctaaagggag
cccccgatttagagcttgacggggaaagccggcgaacgtggcgagaaagg
aagggaagaaagcgaaaggagcgggcgctagggcgctggcaagtgtagcg
gtcacgctgcgcgtaaccaccacacccgccgcgcttaatgcgccgctaca
gggcgcgtcccattcgccattcaggctgcgcaactgttgggaagggcgat
cggtgcgggcctcttcgctattacgccagctggcgaaagggggatgtgct
gcaaggcgattaagttgggtaacgccagggttttcccagtcacgacgttg
taaaacgacggccagtgagcgcgcgtaatacgactcactatagggcgaat
tgggtacCAAGCTTgcctatttcccatgattccttcatatttgcatatac
gatacaaggctgttagagagataattggaattaatttgactgtaaacaca
aagatattagtacaaaatacgtgacgtagaaagtaataatttcttgggta
gtttgcagttttaaaattatgttttaaaatggactatcatatgcttaccg
taacttgaaagtatttcgatttcttggctttatatatcttgTGGAAAGGA
CGAAACACCGTCATGTCTACTATTTCTGGCTgttttagtactctggaaac
agaatctactaaaacaaggcaaaatgccgtgtttatctcgtcaacttgtt
ggcgagatttttttGCGGCCGCCCgcggtggagctccagcttttgttccc
tttagtgagggttaattgcgcgcttggcgtaatcatggtcatagctgttt
cctgtgtgaaattgttatccgctcacaattccacacaacatacgagccgg
aagcataaagtgtaaagcctggggtgcctaatgagtgagctaactcacat
taattgcgttgcgctcactgcccgctttccagtcgggaaacctgtcgtgc
cagctgcattaatgaatcggccaacgcgcggggagaggcggtttgcgtat
tgggcgctcttccgcttcctcgctcactgactcgctgcgctcggtcgttc
ggctgcggcgagcggtatcagctcactcaaaggcggtaatacggttatcc
acagaatcaggggataacgcaggaaagaacatgtgagcaaaaggccagca
aaaggccaggaaccgtaaaaaggccgcgttgctggcgtttttccataggc
tccgcccccctgacgagcatcacaaaaatcgacgctcaagtcagaggtgg
cgaaacccgacaggactataaagataccaggcgtttccccctggaagctc
cctcgtgcgctctcctgttccgaccctgccgcttaccggatacctgtccg
cctttctcccttcgggaagcgtggcgctttctcatagctcacgctgtagg
tatctcagttcggtgtaggtcgttcgctccaagctgggctgtgtgcacga
accccccgttcagcccgaccgctgcgccttatccggtaactatcgtcttg
agtccaacccggtaagacacgacttatcgccactggcagcagccactggt
aacaggattagcagagcgaggtatgtaggcggtgctacagagttcttgaa
gtggtggeetaaetaeggctaeactagaaggacagtatttggtatetgeg
ctctgctgaagccagttaeettcggaaaaagagttggtagctcttgatcc
ggcaaacaaaccaccgctggtagcggtggtttttttgtttgcaagcagca
gattacgcgcagaaaaaaaggatctcaagaagatcctttgatcttttcta
cggggttgacgctcagtggaacgaaaactcacgttaagggattttggtca
tgagattatcaaaaaggatcttcacctagatccttttaaattaaaaatga
agttttaaatcaatctaaagtatatatgagtaaacttggtctgacagtta
eeaatgcttaatcagtgaggeacctatcteagegatctgtctatttcgtt
catecatagttgcctgactccccgtcgtgtagataactacgatacgggag
ggcttaccatctggccccagtgctgcaatgataccgcgagacccacgctc
accggctccagatttatcagcaataaaccagccagccggaagggccgagc
gcagaagtggtcctgcaactttatccgcctccatccagtctattaattgt
tgccgggaagctagagtaagtagttcgccagttaatagtttgcgcaacgt
tgttgccattgctacaggcatcgtgtgtcacgctcgtcgtttggtatggc
ttcattcagctccggttcccaacgatcaaggcgagttacatgatccccca
tgttgtgcaaaaaagcggttagctccttcggtcctccgatcgttgtcaga
agtaagttggccgcagtgttatcactcatggttatggcagcactgcataa
ttctcttactgtcatgccatccgtaagatgcttttctgtgactggtgagt
actcaaccaagtcattctgagaatagtgtatgcggcgaccgagttgctct
tgcccggcgtcaatacgggataataccgcgccacatagcagaactttaaa
agtgctcatcattggaaaacgttcttcggggcgaaaactctcaaggatct
taccgctgttgagatccagttcgatgtaacccactcgtgcacccaactga
tcttcagcatcttttactttcaccagcgtttctgggtgagcaaaaacagg
aaggcaaaatgccgcaaaaaagggaataagggcgacacggaaatgttgaa
taatcataatcttcctttttcaatattattgaagcatttatcagggttat
tgtctcatgagcggatacatatttgaatgtatttagaaaaataaacaaat
aggggttccgcgcacatttccccgaaaagtgccac

SEQ lD NO: 101 Polynucleotide sequence of gRNA 11 in a plasmid cloning vector (3237 bp)

ctaaattgtaagcgttaatattttgttaaaattcgcgttaaatttttgtt
aaatcagctcattttttaaccaataggccgaaatcggcaaaatcccttat
aaatcaaaagaatagaccgagatagggttgagtgttgttccagtttggaa
caagagtccactattaaagaacgtggactccaacgtcaaagggcgaaaaa
ccgtctatcagggcgatggcccactacgtgaaccatcaccctaatcaagt
tttttggggtcgaggtgccgtaaagcactaaatcggaaccctaaagggag
cccccgatttagagcttgacggggaaagccggcgaacgtggcgagaaagg
aagggaagaaagcgaaaggagcgggcgctagggcgctggcaagtgtagcg
gtcacgctgcgcgtaaccaccacacccgccgcgcttaatgcgccgctaca
gggcgcgtcccattcgccattcaggctgcgcaactgttgggaagggcgat
cggtgcgggcctcttcgctattacgccagctggcgaaagggggatgtgct
gcaaggcgattaagttgggtaacgccagggttttcccagtcacgacgttg
taaaacgacggccagtgagcgcgcgtaatacgactcactatagggcgaat
tgggtacCAAGCTTgcctatttcccatgattccttcatatttgcatatac
gatacaaggctgttagagagataattggaattaatttgaetgtaaacaca
aagatattagtacaaaatacgtgacgtagaaagtaataatttcttgggta
gtttgcagttttaaaattatgttttaaaatggactatcatatgcttaccg
taacttgaaagtatttcgatttcttggctttatatatcttgTGGAAAGGA
CGAAACACCGTAACTTAATTCCAGGGTTAATgttttagtactctggaaac
agaatctactaaaacaaggcaaaatgccgtgtttatctcgtcaacttgtt
ggcgagatttttttGCGGCCGCCCgcggtggagctceagcttttgttecc
tttagtgagggttaattgcgegcttggcgtaatcatggtcatagctgttt
cctgtgtgaaattgttatccgctcacaattccacacaacatacgagccgg
aagcataaagtgtaaagcctggggtgcctaatgagtgagctaactcacat
taattgcgttgcgctcactgcccgctttccagtcgggaaacctgtcgtgc
cagctgcattaatgaatcggccaacgcgcggggagagcggtttgcgtatt
gggcgctcttccgcttcctcgctcactgactcgctgcgctcggtcgttcg
gctgcggcgagcggtatcagctcactcaaaggcggtaatacggttatcca
cagaatcaggggataacgcaggaaagaacatgtgagcaaaaggccagcaa
aaggccaggaaccgtaaaaaggccgcgttgctggcgtttttccataggct
ccgcccccctgacgagcatcacaaaaatcgacgctcaagtcagaggtggc
gaaacccgacaggactataaagataccaggcgtttccccctggaagctcc
ctcgtgcgctctcctgttccgaccctgccgcttaccggatacctgtccgc
ctttctcccttcgggaagcgtggcgctttctcatagctcacgctgtaggt
atctcagttcggtgtaggtcgttcgctccaagctgggctgtgtgcacgaa
cccccgttcagcccgaccgctgcgccttatccggtaactatcgtcttgag
tccaacccggtaagacacgacttatcgccactggcagcagccactggtaa
caggattagcagagcgaggtatgtaggcggtgctacagagttcttgaagt
ggtggcctaactacggctacactagaaggacagtatttggtatctgcgct
ctgctgaagccagttaccttcggaaaaagagttggtagctcttgatccgg
caaacaaaccaccgctggtagcggtggtttttttgtttgcaagcagcaga
ttacgcgcagaaaaaaaggatctcaagaagatcctttgatcttttctacg
gggtctgacgctcagtggaacgaaaactcacgttaagggattttggtcat
gagattatcaaaaaggatcttcacctagatccttttaaattaaaaatgaa
gttttaaatcaatctaaagtataatgagtaaacttggtctgacagttacc
aatgcttaatcagtgaggcacctatctcagcgatctgtctatttcgttca
tccatagttgcctgactccccgtcgtgtagataactacgatacgggaggg
cttaccatctggccccagtgctgcaatgataccgcgagacccacgctcac
cggctccagatttatcagcaataaaccagccagccggaagggccgagcgc
agaagtggtcctgcaactttatccgcctccatccagtctattaattgttg
ccgggaagctagagtaagtagttcgccagttaaragttrgcgcaacgtfg
ttgccattgctacaggcatcgrggtgrcacgctcgtcgtttggtatggct
tcattcagctccggttcccaacgatcaaggcgagttacatgatcccccat
gttgtgcaaaaaagcggttagctccttcggtcctccgatcgttgtcagaa
gtaagttggccgcagtgttatcactcatggttatggcagcactgcataat
tctcttactgtcatgccatccgtaagatgcttttctgtgactggtgagta
ctcaaccaagtcattctgagaatagtgtatgcggcgaccgagttgctctt
gcccggcgtcaatacgggataataccgcgccacatagcagaactttaaaa
gtgctcatcattggaaaacgttcttcggggcgaaaactctcaaggatctt
accgctgttgagatccagttcgatgtaacccactcgtgcacccaactgat
cttcagcatcttttactttcaccagcgtttctgggtgagcaaaaacagga
aggcaaaatgccgcaaaaaagggaataagggcgacacggaaatgttgaat
actcatactcttcctttttcaatattattgaagcatttatcagggttatt
gtctcatgagcggatacatatttgaatgtatttagaaaaataaacaaata
ggggttccgcgcacatttccccgaaaagtgccac

SEQ lD NO: 102 (Plasmid 39) Polynucleotide sequence of the donor sequence flanked by scrambled gRNA target sites (7472 bp)

cctgcaggcagctgcgcgctcgctcgctcactgaggccgcccgggcgtcg
ggcgacctttggtcgcccggcctcagtgagcgagcgagcgcgcagagagg
gagtggccaactccatcactaggggttcctgcggcctctagacggatgtg
ctgATTCCTTGAGTTAGCTCTGGTAGTGCTGACTGACCTGAgcgtaatac
gactcactatagggcgaattgggtacCGCTAGGTCTTGAAAGGAGTGGGA
ATTGGCTCCGGTGCCCGTCAGTGGGCAGAGCGCACATCGCCCACAGTCCC
CGAGAAGTTGGGGGGAGGGGTCGGCAATTGATCCGGTGCCTAGAGAAGGT
GGCGCGGGGTAAACTGGGAAAGTGATGTCGTGTACTGGCTCCGCCTTTTT
CCCGAGGGTGGGGGAGAACCGTATATAAGTGCAGTAGTCGCCGTGAACGT
TCTTTTTCGCAACGGGTTTGCCGCCAGAACACAGGACCGGTGCCACCATG
GCCGCCCACAGGCCGGTGGAATGGGTCCAGGCCGTGGTCAGCCGCTTCGA
CGAGCAGCTTCCAATAAAAACAGGACAGCAGAACACACATACCAAAGTCA
GTACTGAGCACAACAAGGAATGTCTAATCAATATTTCCAAATACAAGTTT
TCTTTGGTTATAAGCGGCCTCACTACTATTTTAAAGAATGTTAACAATAT
GAGAATATTTGGAGAAGCTGCTGAAAAAAATTTATATCTCTCTCAGTTGA
TTATATTGGATACACTGGAAAAATGTCTTGCTGGGCAACCAAAGGACACA
ATGAGATTAGATGAAACGATGCTGGTCAAACAGTTGCTGCCAGAAATCTG
CCATTTTCTTCACACCTGTCGTGAAGGAAACCAGCATGCAGCTGAACTTC
GGAATTCTGCCTCTGGGGTTTTATTTTCTCTCAGCTGCAACAACTTCAAT
GCAGTCTTTAGTCGCATTTCTACCAGGTTACAGGAATTAACTGTTTGTTC
AGAAGACAATGTTGATGTTCATGATATAGAATTGTTACAGTATATCAATG
TGGATTGTGCAAAATTAAAACGACTCCTGAAGGAAACAGCATTTAAATTT
AAAGCCCTAAAGAAGGTTGCGCAGTTAGCAGTTATAAATAGCCTGGAAAA
GGCATTTTGGAACTGGGTAGAAAATTATCCAGATGAATTTACAAAACTGT
ACCAGATCCCACAGACTGATATGGCTGAATGTGCAGAAAAGCTATTTGAC
TTGGTGGATGGTTTTGCTGAAAGCACCAAACGTAAAGCAGCAGTTTGGCC
ACTACAAATCATTCTCCTTATCTTGTGTCCAGAAATAATCCAGGATATAT
CCAAAGACGTGGTTGATGAAAACAACATGAATAAGAAGTTATTTCTGGAC
AGTCTACGAAAAGCTCTTGCTGGCCATGGAGGAAGTAGGCAGCTGACAGA
AAGTGCTGCAATTGCCTGTGTCAAACTGTGTAAAGCAAGTACTTACATCA
ATTGGGAAGATAACTCTGTCATTTTCCTACTTGTTCAGTCCATGGTGGTT
GATCTTAAGAACCTGCTTTTTAATCCAAGTAAGCCATTCTCAAGAGGCAG
TCAGCCTGCAGATGTGGATCTAATGATTGACTTTCTGCTTTCGTATAAGC
CTCCAACACTGCCTTGTTCTCACAACACCAACACCTTTAGATCTGCCTGG
VTCAGAATTCACCTTCTACATTTCACTATGTGCTGGTAAATTCACTCCAT
CGAATCATCACCAATTCCGCATTGGATTGGTGGCCTAAGATTGATGCTGT
GTATTGTCACTCGGTTGAACTTCGAAATATGTTTGGTGAAACACTTCATA
AAGCAGTGCAAGGTTGTGGAGCACACCCAGCAATACGAATGGCACCGAGT
CTTACATTTAAAGAAAAAGTAACAAGCCTTAAATTTAAAGAAAAACCTAC
AGACCTGGAGACAAGAAGCTATAAGTATCTTCTCTTGTCCATGGTGAAAC
TAATTCATGCAGATCCAAAGCTCTTGCTTTGTAATCCAAGAAAACAGGGG
CCCGAAACCCAAGGCAGTACAGCAGAATTAATTACAGGGCTCGTCCAACT
GGTCCCCAGTCACACATGCCAGAGATTGCTCAGGAAGCAATGGAGGCTCT
GCTGGTTCTTCATCAGTTAGATAGCATTGATTTGTGGAATCCTGATGCTC
CTGTAGAAACATTTTGGGAGATTAGCTCACAAATGCTTTTTTACATCTGC
AAGAAATTAACTAGTCATCAAATGCTTAGTAGCACAGAAATTCTCAAGTG
GTTGCGGGAAATATTGATCTGCAGGAATAAATTTCTTCTTAAAAATAAGC
AGGCAGATAGAAGTTCCTGTCACTTTCTCCTTTTTTACGGGGTAGGATGT
GATATTCCTTCTAGTGGAAATACCAGTCAAATGTCCATGGATCATGAAGA
ATTACTACGTACTCCTGGAGCCTCTCTCCGGAAGGGAAAAGGGAACTCCT
CTATGGATAGTGCAGCAGGATGCAGCGGAACCCCCCCGATTTGCCGACAA
GCCCAGACCAAACTAGAAGTGGCCCTGTACATGTTTCTGTGGAACCCTGA
CACTGAAGCTGTTCTGGTTGCCATGTCCTGTTTCCGCCACCTCTGTGAGG
AAGCAGATATCCGGTGTGGGGTGGATGAAGTGTCAGTGCATAACCTCTTG
CCCAACTATAACACATTCATGGAGTTTGCCTCTGTCAGCAATATGATGTC
AACAGGAAGAGCAGCACTTCAGAAAAGAGTGATGGCACTGCTGAGGCGCA
TTGAGCATCCCACTGCAGGAAACACTGAGGCTTGGGAAGATACACATGCA
AAATGGGAACAAGCAACAAAGCTAATCCTTAACTATCCAAAAGCCAAAAT
GGAAGATGGCCAGGCTGCTGAAAGCCTTCACAAGACCATTGTTAAGAGGC
GAATGTCCCATGTGAGTGGAGGAGGATCCATAGATTTGTCTGACACAGAC
TCCCTACAGGAATGGATCAACATGACTGGCTTCCTTTGTGCCCTTGGGGG
AGTGTGCCTCCAGCAGAGAAGCAATTCTGGCCTGGCAACCTATAGCCCAC
CCATGGGTCCAGTCAGTGAACGTAAGGGTTCTATGATTTCAGTGATGTCT
TCAGAGGGAAACGCAGATACACCTGTCAGCAAATTTATGGATCGGCTGTT
GTCCTTAATGGTGTGTAACCATGAGAAAGTGGGACTTCAAATACGGACCA
ATGTTAAGGATCTGGTGGGTCTAGAATTGAGTCCTGCTCTGTATCCAATG
CTATTTAACAAATTGAAGAATACCATCAGCAAGTTTTTTGACTCCCAAGG
ACAGGTTTTATTGACTGATACCAATACTCAATTTGTAGAACAAACCATAG
CTATAATGAAGAACTTGCTAGATAATCATACTGAAGGCAGCTCTGAACAT
CTAGGGCAAGCTAGCATTGAAACAATGATGTTAAATCTGGTCAGGTATGT
TCGTGTGCTTGGGAATATGGTCCATGCAATTCAAATAAAAACGAAACTGT
GTCAATTAGTTGAAGTAATGATGGCAAGGAGAGATGACCTCTCATTTTGC
CAAGAGATGAAATTTAGGAATAAGATGGTAGAATACCTGACAGACTGGGT
TATGGGAACATCAAACCAAGCAGCAGATGATGATGTAAAATGTCTTACAA
GAGATTTGGACCAGGCAAGCATGGAAGCAGTAGTTTCACTTCTACTGGTC
TCCCTCTGCAGCCTGAAGAAGGAGATGGTGTGGAATTGATGGAAGCCAAA
TCACAGTTATTTCTTAAATACTTCACATTATTTATGAACCTTTTGAATGA
CTGCAGTGAAGTTGAAGATGAAAGTGCGCAAACAGGTGGCAGGAAACGTG
GCATGTCTCGGAGGCTGGCATCACTGAGGCACTGTACGGTCCTTGCAATG
TCAAACTTACTCAATGCCAACGTAGACAGTGGTCTCATGCACTCCATAGG
CTTAGGTTACCACAAGGATCTCCAGACAAGAGCTACATTTATGGAAGTTC
TGACAAAAATCCTTCAACAAGGCACAGAATTTGACACACTTGCAGAAACA
GTATTGGCTGATCGGTTTGAGAGATTGGTGGAACTGGTCACAATGATGGG
TGATCAAGGAGAACTCCCTATAGCGATGGCTCTGGCCAATGTGGTTCCTT
GTTCTCAGTGGGATGAACTAGCTCGAGTTCTGGTTACTCTGTTTGATTCT
CGGCATTTACTCTACCAACTGCTCTGGAACATGTTTTCTAAAGAAGTAGA
ATTGGCAGACTCCATGCAGACTCTCTTCCGAGGCAACAGCTTGGCCAGTA
AAATAATGACATTCTGTTTCAAGGTATATGGTGCTACCTATCTACAAAAA
CTCCTGGATCCTTTATTACGAATTGTGATCACATCCTCTGATTGGCAACA
TGTTAGCTTTGAAGTGGATCCTACCAGGTTAGAACCATCAGAGAGCCTTG
AGGAAAACCAGCGGAACCTCCTTCAGATGACTGAAAAGTTCTTCCATGCC
ATCATCAGTTCCTCCTCAGAATTCCCCCCTCAACTTCGAAGTGTGTGCCA
CTGCTTATACCAGGTAAGTATGGAGGAAGGAATTGCGTGTTATGTTGACC
CGTGCCTAGGTATTACGCAATTCCTTGAGTTAGCTCTGGTAGTGCGCggc
cgcCCgcggtggagctccagcttttgttccctttggccgcaggaacccct
agtgatggagttggccactccctctctgcgcgctcgctcgctcactgagg
ccgggcgaccaaaggtcgcccgacgcccgggctttgcccgggcggcctca
gtgagcgagcgagcgcgcagctgcctgcaggggcgcctgatgcggtattt
tctccttacgcatctgtgcggtatttcacaccgcatacgtcaaagcaacc
atagtacgcgccctgtagcggcgcattaagcgcggcgggtgtggtggtta
cgcgcagcgtgaccgctacacttgccagcgcctagcgcccgctcctttcg
ctttcttcccttcctttctcgccacgttcgccggctttccccgtcaagct
ctaaatcgggggctccctttagggttccgatttagtgctttacggcacct
cgaccccaaaaaacttgatttgggtgatggttcacgtagtgggccatcgc
cctgatagacggtttttcgccctttgacgttggagtccacgttctttaat
agtggactcttgttccaaactggaacaacactcaaccctatctcgggcta
ttcttttgatttataagggattttgccgatttcggcctattggttaaaaa
atgagctgatttaacaaaaatttaacgcgaattttaacaaaatattaacg
tttacaattttatggtgcactctcagtacaatctgctctgatgccgcata
gttaagccagccccgacacccgccaacacccgctgacgcgccctgacggg
cttgtctgctcccggcatccgcttacagacaagctgtgaccgtctccggg
agctgcatgtgtcagaggttttcaccgtcatcaccgaaacgcgcgagacg
aaagggcctcgtgatacgcctatttttataggttaatgtcatgataataa
tggtttcttagacgtcaggtggcacttttcggggaaatgtgcgcggaacc
cctatttgtttatttttctaaatacattcaaatatgtatccgctcatgag
acaataaccctgataaatgcttcaataatattgaaaaaggaagagtatga
gtattcaacatttccgtgtcgcecttattcecttttttgcggcattttgc
ettcctgtttttgctcacccagaaacgctggtgaaagtaaaagatgctga
agatcagttgggtgcacgagtgggttacatcgaactggatctcaacagcg
gtaagatccttgagagttttcgccccgaagaacgttttccaatgatgagc
acttttaaagttctgctatgtggcgcggtattatcccgtattgacgccgg
gcaagagcaactcggtcgccgcatacactattctcagaatgacttggttg
agtactcaccagtcacagaaaagcatcttacggatggcatgacagtaaga
gaattatgcagtgctgccataaccatgagtgataacactgcggccaactt
acttctgacaacgatcggaggaccgaaggagctaaccgcttttttgcaca
acatgggggatcatgtaactcgccttgatcgttgggaaccggagctgaat
gaagccataccaaacgacgagcgtgacaccacgatgcctgtagcaatggc
aacaacgttgcgcaaactattaactggcgaactacttactctagcttccc
ggcaacaattaatagactggatggaggcggataaagttgcaggaccactt
ctgcgctcggcccttccggctggctggtttattgctgataaatctggagc
cggtgagcgtggaagccgcggtatcattgcagcactggggccagatggta
agccctcccgtatcgtagttatctacacgacggggagtcaggcaactatg
gatgaacgaaatagacagatcgctgagataggtgcctcactgattaagca
ttggtaactgtcagaccaagtttactcatatatactttagattgatttaa
aacttcatttttaatttaaaaggatctaggtgaagatcctttttgataat
ctcatgaccaaaatcccttaacgtgagttttcgttccactgagcgtcaga
ccccgtagaaaagatcaaaggatcttcttgagatcctttttttctgcgcg
taatctgctgcttgcaaacaaaaaaaccaccgctaccagcggtggtttgt
ttgccggatcaagagctaccaactctttttccgaaggtaactggcttcag
cagagcgcagataccaaatactgtccttctagtgtagccgtagttaggcc
accacttcaagaactctgtagcaccgcctacatacctcgctctgctaatc
ctgttaccagtggctgctgccagtggcgataagtcgtgtcttaccgggtt
ggactcaagacgatagttaccggataaggcgcagcggtcgggctgaacgg
ggggttcgtgcacacagcccagcttggagcgaacgacctacaccgaactg
agatacctacagcgtgagctatgagaaagcgccacgcttcccgaagggag
aaaggcggacaggtatccggtaagcggcagggtcggaacaggagagcgca
cgagggagcttccagggggaaacgcctggtatctttatagtcctgtcggg
tttggccagctctgacttgagcgtcgatttttgtgatgctcgtcaggggg
gcggagcctatggaaaaacgccagcaacgcggcctttttacggttgctgg
ccttttgctggccttttgctcacatgt.

1. A CRISPR/Cas-based genome editing system comprising:
(a) a polynucleotide sequence encoding a guide RNA (gRNA) targeting a fragment of a mutant NF1 gene;
(b) a polynucleotide sequence encoding a Cas protein or a fusion protein comprising the Cas protein; and
(c) a polynucleotide sequence encoding a donor sequence comprising a fragment of a wild-type NF1 gene.

2. The system of claim 1, wherein the system comprises one or more vectors.

3. The system of claim 2, wherein the system comprises a first vector and a second vector,
wherein the first vector comprises the polynucleotide sequence encoding the gRNA, and the polynucleotide sequence encoding the Cas protein or the fusion protein, and
wherein the second vector comprises the polynucleotide sequence encoding the donor sequence.

4. The system of claim 3, wherein the polynucleotide sequence encoding the gRNA and the polynucleotide sequence encoding the Cas protein or the fusion protein are operably linked.

5. The system of any one of claims 1-4, wherein the mutant NF1 gene comprises a mutation in the 5′ portion of the mutant NF1 gene, and
wherein the polynucleotide sequence encoding the donor sequence further comprises a stop codon that is 5′ to the donor sequence.

6. The system of claim 5, wherein the gRNA targets a sequence upstream of the stop codon that is 5′ to the donor sequence and targets a sequence downstream of the donor sequence.

7. The system of claim 5 or 6, wherein the polynucleotide sequence encoding the donor sequence further comprises a promoter in between the stop codon and the donor sequence.

8. The system of claim 7, wherein the stop codon, the promoter, and the donor sequence are flanked on both ends (the 5′ and 3′ ends) with a sequence the gRNA targets.

9. The system of any one of claims 5-8, wherein the stop codon, the donor sequence, and the 3′ portion of the mutant NF1 gene are in the same reading frame.

10. The system of any one of claims 1-4, wherein the mutant NF1 gene comprises a mutation in the 3′ portion of the mutant NF1 gene, and
wherein the polynucleotide sequence encoding the donor sequence further comprises a stop codon that is 3′ to the donor sequence.

11. The system of claim 10, wherein the gRNA targets a sequence upstream of the donor sequence and targets a sequence downstream of the stop codon that is 3′ to the donor sequence.

12. The system of claim 10 or 11, wherein the 5′ portion of the mutant NF1 gene, the donor sequence, and the stop codon are in the same reading frame.

13. The system of any one of claims 5-12, wherein the gRNA targets a sequence flanking both sides of the polynucleotide sequence encoding the donor sequence and the stop codon.

14. The system of any one of claims 1-13, wherein the donor sequence comprises multiple exons of the wild-type NF1 gene or a functional equivalent thereof.

15. The system of any one of claims 1-14, wherein the donor sequence comprises one or more exons selected from exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, and exon 57 of the wild-type NF1 gene or a functional equivalent thereof.

16. The system of claim 15, wherein the donor sequence comprises one or more contiguous exons of the wild-type NF1 gene or a functional equivalent thereof.

17. The system of any one of claims 1-16, wherein the donor sequence comprises exons 1-30 of the wild-type NF1 gene, and wherein the gRNA targets a fragment of a mutant NF1 gene between exon 30 and exon 31.

18. The system of any one of claims 1-17, wherein the gRNA comprises a polynucleotide sequence selected from SEQ ID NOs: 71-81 or a complement thereof or a truncation thereof.

19. The system of any one of claims 1-17, wherein the gRNA is encoded by a polynucleotide sequence selected from SEQ ID NOs: 60-70 or a complement thereof or a truncation thereof, and/or hybridizes to a polynucleotide sequence selected from SEQ ID NOs: 49-59 or a complement thereof or a truncation thereof.

20. The system of any one of claims 1-19, wherein the donor sequence comprises a polynucleotide sequence of SEQ ID NO: 82.

21. The system of any one of claims 1-20, wherein the Cas protein is a Streptococcus pyogenes Cas9 protein or a Staphylococcus aureus Cas9 protein.

22. The system of claim 21, wherein the Cas protein comprises an amino acid sequence of SEQ ID NO: 20 or SEQ ID NO: 21.

23. The system of any one of claims 2-22, wherein the vector is a viral vector.

24. The system of claim 23, wherein the vector is an Adeno-associated virus (AAV) vector.

25. The system of claim 24, wherein the AAV vector is AAV1, AAV2, AAV3, AAV4, AAV5, AAV6, AAV7, AAV8, AAV9, AAV-10, AAV-11, AAV-12, AAV-13, or AAVrh.74 vector.

26. The system of claim 24, wherein one of the one or more vectors comprises a polynucleotide sequence selected from SEQ ID NOs: 83-102.

27. The system of any one of claims 1-26, wherein the molar ratio between the gRNA and the donor sequence is 1:1, or 1:5, or from 5:1 to 1:10, or from 1:1 to 1:5.

28. A cell comprising the system of any one of claims 1-27.

29. A composition for restoring NF1 function in a cell having a mutant NF1 gene, the composition comprising the system of any one of claims 1-27 or the cell of claim 28.

30. A kit comprising the system of any one of claims 1-27, the cell of claim 28, or the composition of claim 29.

31. A method for restoring NF1 function in a cell or a subject having a mutant NF1 gene, the method comprising contacting the cell or the subject with the system of any one of claims 1-27, the cell of claim 28, or the composition of claim 29.

32. The method of claim 31, wherein NF1 function is restored by inserting one or more wild-type exons of NF1 gene corresponding to the mutant NF1 gene.
